(19) United States
(12) Patent Application Publication
(10) Pub. No.: US 2025/0325401 A1
(43) Pub. Date: Oct. 23, 2025

(54) OCULAR DELIVERY SYSTEMS AND METHODS

(71) Applicant: Sight Sciences, Inc., Menlo Park, CA (US)

(72) Inventors: David Samuel NEEDLEMAN, San Carlos, CA (US); Peter Michael BUGOS, Redwood City, CA (US); Paul BADAWI, Atherton, CA (US); David Y. BADAWI, Glenview, IL (US)

(21) Appl. No.: 19/066,093

(22) Filed: Feb. 27, 2025

Related U.S. Application Data
(60) Provisional application No. 63/558,587, filed on Feb. 27, 2024.

(51) Int. Cl. A61F 9/00

(52) U.S. Cl. CPC A61F 9/0017

(57) ABSTRACT

Disclosed herein are devices for delivering fluid to an eye. The device may include a handle comprising a fluid assembly at least partially contained therein, the fluid assembly comprising a fluid reservoir, a plunger tube, and a displacement rod, where the plunger tube and displacement rod are configured to move relative to the fluid reservoir to deliver fluid during advancement of an elongate member. The device includes a cannula coupled to a distal end of a handle, and the elongate member slidably positioned within the handle. The handle includes a drive assembly where actuation of the drive assembly moves the plunger tube and the displacement rod in opposite directions during advancement of the elongate member. The drive assembly also comprises a first linear gear and a second linear gear, where the first linear gear is configured to move in a direction opposite the second linear gear.

### CROSS REFERENCE TO RELATED APPLICATIONS

This application claims priority to U.S. Provisional Patent Application Ser. No. 63/558,587 filed Feb. 27, 2024, the content of which is incorporated herein by reference in its entirety for all purposes.

### TECHNICAL FIELD

This invention relates generally to fluid delivery systems for treating conditions of the eye, and associated methods for treating such conditions of the eye.

### BACKGROUND

Glaucoma is a potentially blinding disease that affects over 60 million people worldwide, or about 1-2% of the population. Typically, glaucoma is characterized by elevated intraocular pressure. Increased pressure in the eye can cause irreversible damage to the optic nerve which can lead to loss of vision and even progress to blindness if left untreated. Consistent reduction of intraocular pressure can slow down or stop progressive loss of vision associated with glaucoma.

Increased intraocular pressure is generally caused by sub-optimal efflux or drainage of fluid (aqueous humor) from the eye. Aqueous humor or fluid is a clear, colorless fluid that is continuously replenished in the eye. Aqueous humor is produced by the ciliary body, and then ultimately exits the eye primarily through the trabecular meshwork. The trabecular meshwork extends circumferentially around the eye at the anterior chamber angle, or drainage angle, which is formed at the intersection between the peripheral iris or iris root, the anterior sclera or scleral spur and the peripheral cornea. The trabecular meshwork feeds outwardly into Schlemm's canal, a narrow circumferential passageway generally surrounding the exterior border of the trabecular meshwork. Positioned around and radially extending from Schlemm's canal are aqueous veins or collector channels that receive drained fluid. The net drainage or efflux of aqueous humor can be reduced as a result of decreased facility of outflow, decreased outflow through the trabecular meshwork and canal of Schlemm drainage apparatus, increased episcleral venous pressure, or possibly, increased production of aqueous humor. Flow out of the eye can also be restricted by blockages or constriction in the trabecular meshwork and/or Schlemm's canal and its collector channels.

Glaucoma, pre-glaucoma, and ocular hypertension currently can be treated by reducing intraocular pressure using one or more modalities, including medication, incisional surgery, laser surgery, cryosurgery, and other forms of surgery. In general, medications or medical therapy are the first lines of therapy. If medical therapy is not sufficiently effective, more invasive surgical treatments may be used. For example, a standard incisional surgical procedure to reduce intraocular pressure is trabeculectomy, or filtration surgery. This procedure involves creating a new drainage site for aqueous humor. Instead of naturally draining through the trabecular meshwork, a new drainage pathway is created by removing a portion of sclera and trabecular meshwork at the drainage angle. This creates an opening or passage between the anterior chamber and the subconjunctival space that is drained by conjunctival blood vessels and lymphatics. The new opening may be covered with sclera and/or conjunctiva to create a new reservoir called a bleb into which aqueous humor can drain. However, traditional trabeculectomy procedures carry both short- and long-term risks. These risks include blockage of the surgically created opening through scarring or other mechanisms, hypotony or abnormally low intraocular pressure, expulsive hemorrhage, hyphema, intraocular infection or endophthalmitis, shallow anterior chamber angle, macular hypotony, choroidal exudation, suprachoroidal hemorrhage, and others.

One alternative is to implant a device in Schlemm's canal that maintains the patency of the canal or aids flow of aqueous humor from the anterior chamber into the canal. Various stents, shunts, catheters, and procedures have been devised for this purpose and employ an ab-externo (from the outside of the eye) approach to deliver the implant or catheter into Schlemm's canal. This method of placement is invasive and typically prolonged, requiring the creation of tissue flaps and deep dissections to access the canal. Additionally, it is very difficult for many surgeons to find and access Schlemm's canal from this external incisional approach because Schlemm's canal has a small diameter, e.g., approximately 50 to 250 microns in cross-sectional diameter, and it may be even smaller when collapsed. One related non-implant procedure, ab-externo canaloplasty, involves making a deep scleral incision and flap, finding and unroofing Schlemm's canal, circumnavigating all 360 degrees of the canal with a catheter from the outside of the eye, and either employing viscoelastic, a circumferential tensioning suture, or both to help maintain patency of the canal. The procedure is quite challenging and can take anywhere from forty-five minutes to two hours. The long-term safety and efficacy of canaloplasty is very promising, but the procedure remains surgically challenging and invasive.

Another alternative is viscocanalostomy, which involves the injection of a viscoelastic solution into Schlemm's canal to dilate the canal and associated collector channels. Dilation of the canal and collector channels in this manner generally facilitates drainage of aqueous humor from the anterior chamber through the trabecular meshwork and Schlemm's canal, and out through the natural trabeculocanalicular outflow pathway. Viscocanalostomy is similar to canaloplasty (both are invasive and ab-externo), except that viscocanalostomy does not involve a suture and does not restore all 360 degrees of outflow facility. Some advantages of viscocanalostomy are that sudden drops in intraocular pressure, hyphema, hypotony, and flat anterior chambers may be avoided. The risk of cataract formation and infection may also be minimized because of reduced intraocular manipulation and the absence of full eye wall penetration, anterior chamber opening and shallowing, and iridectomy. A further advantage of viscocanalostomy is that the procedure restores the physiologic outflow pathway, thus avoiding the need for external filtration, and its associated short and long term risks, in the majority of eyes. This makes the success of the procedure partly independent of conjunctival or episcleral scarring, which is a leading cause of failure in traditional trabeculectomy procedures. Moreover, the absence of an elevated filtering bleb avoids related ocular discomfort and potentially devastating ocular infections, and the procedure can be carried out in any quadrant of the outflow pathway.

However, ab externo viscocanalostomy and canaloplasty techniques are still very invasive because access to Schlemm's canal must be created by making a deep incision into the sclera, creating a scleral flap, and un-roofing Schlemm's canal. “Ab-externo” generally means “from the outside” and it is inherently more invasive given the location of Schlemm's canal and the amount of tissue disruption required to access it from the outside. On the other hand, “ab-interno” means “from the inside” and is a less invasive approach because of the reduced amount of tissue disruption required to access it from the inside. Consequently, an ab-interno approach to Schlemm's canal offers the surgeon easier access to the canal, but also reduces risk to the patient's eye and reduces patient morbidity. All of these lead to improved patient recovery and rehabilitation. The ab-externo viscocanalostomy and canaloplasty procedures also remain challenging to surgeons, because as previously stated, it is difficult to find and access Schlemm's canal from the outside using a deep incisional approach due to the small diameter of Schlemm's canal. A further drawback still is that at most, viscocanalostomy typically dilates up to 60 degrees of Schlemm's canal, which is a 360-degree ring-shaped outflow vessel-like structure. The more of the canal that can be dilated, the more total aqueous outflow can be restored.

Accordingly, it would be beneficial to have systems that easily and atraumatically provide access to Schlemm's canal using an ab-interno approach for the delivery of tools and fluid compositions. It would also be useful to have systems that deliver tools and compositions into Schlemm's canal expeditiously to decrease procedure time and the risk of infection without compromising safety and precision of the delivery procedure. It would also be useful to have systems that deliver tools and fluid compositions into Schlemm's canal using an ab-interno approach so that cataract surgery and glaucoma surgery can both be accomplished during the same surgical sitting using the very same corneal or scleral incision. Such incisions are smaller and allow for less invasive surgery and more rapid patient recovery. This approach allows for accessing Schlemm's canal through the trabecular meshwork from the inside of the eye, and thus it is called “ab-interno.” Methods of delivering tools and compositions that effectively disrupt the juxtacanalicular meshwork and adjacent wall of Schlemm's canal, also known as the inner wall of Schlemm's canal, maintain the patency of Schlemm's canal, increase outflow, decrease resistance to outflow, or effectively dilate the canal and/or its collector channels using the systems in a minimally invasive, ab-interno manner would also be desirable.

### SUMMARY

Disclosed herein is a device for delivering fluid to the eye. The device may comprise a handle comprising a fluid assembly at least partially contained within the handle. The fluid assembly may comprise a fluid reservoir, a plunger tube, and a displacement rod. The device may also comprise a cannula coupled to a distal end of the handle, and an elongate member slidably positioned within the cannula and configured to be advanced into Schlemm's canal, where the plunger tube and the displacement rod may be configured to move relative to the fluid reservoir to deliver fluid from the fluid reservoir during advancement of the elongate member.

In some variations, the handle further may comprise a drive assembly and actuation of the drive assembly moves the plunger tube and the displacement rod in opposite directions during advancement of the elongate member. In some variations, actuation of the drive assembly may simultaneously move the plunger tube and the displacement rod a same distance in opposite directions. In some variations, the drive assembly may comprise a first linear gear and a second linear gear, and where the first linear gear is configured to move in a direction opposite the second linear gear.

In some variations, the displacement rod may be releasably coupled to the first linear gear and the plunger tube is coupled to the second linear gear. In some variations, the first linear gear may receive at least a portion of the displacement rod and the second linear gear may receive at least a portion of the plunger tube.

In some variations, the first linear gear may receive a first length of the displacement rod during a first advancement and may receive a second length of the displacement rod during a second advancement. In some variations, the second length may be less than the first length. In some variations, a difference between the first length and the second length may correspond to about 3 clock hours to about 5 clock hours of elongate member advancement around the eye. In some variations, the second length of the displacement rod may be about 9 mm to about 15 mm less than the first length of the displacement rod. In some variations, a ratio of the first length to the second length may be about 10:7 to about 2:1.

In some variations, the first linear gear may comprise a channel comprising a stop configured to engage with the portion of the displacement rod during a second advancement of the elongate member. In some variations, the stop may comprise an opening in a sidewall of the channel configured to receive the portion of the displacement rod during a second advancement of the elongate member. In some variations, the stop may comprise a pawl configured to receive the portion of the displacement rod during a second advancement of the elongate member. In some variations, the channel may comprise a distal portion configured to receive the portion of the displacement rod during a first advancement of the elongate member. In some variations, the stop may be positioned a distance proximal from a distal end of the channel, where the distance may correspond to about 3 clock hours to about 5 clock hours of elongate member advancement around the eye. In some variations, the stop may be positioned about 9 mm to about 15 mm proximal of a distal end of the channel. In some variations, the portion of the displacement rod may comprise a bend.

In some variations, movement of the displacement rod towards the fluid reservoir may deliver fluid during advancement of the elongate member. In some variations, movement of the plunger tube towards the fluid reservoir may deliver fluid during retraction of the elongate member. In some variations, the plunger tube may comprise a lumen in fluid communication with a lumen of the elongate member. In some variations, the displacement rod may be solid.

In some variations, the fluid reservoir may comprise a first lumen configured to receive the displacement rod and a second lumen configured to receive the plunger tube, where the first lumen may be in fluid communication with the second lumen. In some variations, the device may be configured to deliver a first volume of fluid during advancement of the elongate member and a second volume of fluid during retraction of the elongate member.

In some variations, the first volume of fluid may be based on at least one geometric difference between the displacement rod and the plunger tube. In some variations, the at least one geometric difference between the displacement rod and the plunger tube may include a difference in diameters of the displacement rod and the plunger tube, a difference in lengths of the displacement rod and the plunger tube, a difference in cross sectional shape of the displacement rod and the plunger tube, or a combination thereof. In some variations, the geometric difference between the displacement rod and the plunger tube may be a difference in diameter.

In some variations, a diameter of the displacement rod may be greater than a diameter of the plunger tube, while in other variations, a diameter of the displacement rod may be smaller than a diameter of the plunger tube. In some variations, a diameter of the displacement rod may be constant along a length of the displacement rod. In some variations, a diameter of the displacement rod may decrease from a distal end of the displacement rod to a proximal end of the displacement rod. In some variations, a diameter of the plunger tube may be constant along a length of the plunger tube.

In some variations, the geometric difference between the displacement rod and the plunger tube may be a difference in length. In some variations, a length of the displacement rod may be greater than a length of the plunger tube. In some variations, a length of the plunger tube may be greater than a length of the displacement rod.

In some variations, a volume of the first volume of fluid delivered during advancement of the elongate member may be based on at least one geometric difference between a first lumen of the fluid reservoir and a second lumen of the fluid reservoir. In some variations, the at least one geometric difference between the first lumen of the fluid reservoir and the second lumen of the fluid reservoir may include a difference in diameters of the first and second lumens, a difference in volumes of the first and second lumens, a difference in the lengths of the first and second lumens, or a combination thereof.

In some variations, the at least one geometric difference between the first lumen of the fluid reservoir and the second lumen of the fluid reservoir may include a difference in diameters of the first and second lumens. In some variations, a diameter of the first lumen may be greater than a diameter of the second lumen. In some variations, a diameter of the second lumen may be greater than a diameter of the first lumen. In some variations, a diameter of the first lumen may be consistent along a length of the first lumen. In some variations, a diameter of the first lumen may be variable along at least a portion of a length of the first lumen. In some variations, the diameter of the first lumen may decrease from a proximal end to a distal end of the first lumen.

In some variations, the diameter of the first lumen may decrease from a distal end to a proximal end of the first lumen. In some variations, a diameter of the second lumen may be consistent along a length of a second lumen. In some variations, the diameter of the second lumen may be variable along at least a portion of the length of the second lumen. In some variations, the diameter of the second lumen may decrease from a proximal end to a distal end of the second lumen.

In some variations, the diameter of the second lumen decreases from a distal end to a proximal end of a second lumen. In some variations, the geometric difference between the first lumen and the second lumen may include a difference in volumes between the first and second lumens. In some variations, a volume of the second lumen may be greater than a volume of the first lumen.

In some variations, the drive assembly may comprise an actuator configured to be contacted by a user, wherein the actuator comprises one or more of a slide, a wheel, or a button. In some variations, the actuator may engage the first linear gear.

Also disclosed herein is a device for delivering fluid to the eye. The device may comprise a handle comprising a drive assembly and a fluid assembly each at least partially contained in the handle. The fluid assembly may comprise a fluid reservoir, a plunger tube, and a displacement rod. The device may comprise a cannula coupled to a distal end of the housing, and an elongate member slidably positioned within the cannula, where actuation of the drive assembly may move the plunger tube and the displacement rod in opposite directions during advancement of the elongate member.

Also disclosed herein is a device for delivering fluid to the eye. The device may comprise a handle comprising a drive assembly and a fluid assembly each at least partially contained therein. The drive assembly may comprise an actuator configured to be contacted by a user, a first linear gear and a second linear gear. The device also may comprise a cannula coupled to a distal end of the housing, and an elongate member slidably positioned within the cannula, where actuation of the actuator may move the first and second linear gears in opposite directions.

In some variations, actuation of the actuator in a first direction may advance the elongate member away from the fluid reservoir, while in other variations, actuation of the actuator in a second direction may retract the elongate member towards the fluid reservoir.

In some variations, the fluid assembly may comprise a plunger tube and a displacement rod, and where actuation of the drive assembly may move the plunger tube and the displacement rod in opposite directions. In some variations, actuation of the drive assembly in a first direction may move the displacement rod towards the fluid assembly and the plunger tube away from the fluid assembly. In some variations, actuation of the drive assembly in a second direction may move the plunger tube towards the fluid assembly. In some variations, the fluid assembly comprises a fluid reservoir, and where actuation of the actuator in a first direction may move the first linear gear in a first direction towards the fluid reservoir and the second linear gear in a second direction away from the fluid reservoir. In some variations, actuation of the actuator in a second, opposite direction may move the first linear gear in a second direction away from the fluid reservoir and the second linear gear in a first direction towards the fluid reservoir.

Also disclosed herein is a device for delivering fluid to the eye. The device may comprise a handle comprising a fluid assembly at least partially contained therein. The fluid assembly may comprise a fluid reservoir, a plunger tube, and a displacement rod. In some variations, the device may comprise a cannula coupled to a distal end of the housing, and an elongate member slidably positioned within the cannula and configured to be advanced into Schlemm's canal, where a volume of fluid delivered during advancement of the elongate member may be based on a geometric difference between the displacement rod and the plunger tube or a geometric difference between first and second lumens of the fluid reservoir. In some variations, the plunger tube may comprise a lumen therein, and the displacement rod may be solid.

Also disclosed herein is a device for delivering fluid to the eye. The device may comprise a handle comprising a fluid reservoir at least partially contained therein. The fluid reservoir may comprise a first lumen, a second lumen, and a passageway fluidically coupling the first and second lumens. The device may comprise a cannula coupled to a distal end of the housing, and an elongate member slidably positioned within the cannula and configured to be advanced into Schlemm's canal, where fluid is displaced from the first lumen through the passageway to the second lumen during advancement of the elongate member.

In some variations, fluid may be delivered from the second lumen to Schlemm's canal during retraction of the elongate member. In some variations, fluid may be delivered during upon advancement of the elongate member and upon retraction of the elongate member. In some variations, fluid may be delivered from the second lumen to Schlemm's canal during advancement of the elongate member.

In some variations, the fluid reservoir may include a fluid reservoir connector in fluid communication with the first lumen. The fluid reservoir connector may be configured to releasably couple with an external fluid device and receive fluid. In some variations, the fluid reservoir may be stationary within the handle.

In some variations, a displacement rod may be slidably positioned at least partially within the first lumen, where movement of the displacement rod within the first lumen may displace fluid from the first lumen to the second lumen. In some variations, a central longitudinal axis of the first lumen and central longitudinal axis of the second lumen may be parallel.

Also disclosed herein is a device for delivering fluid to the eye. The device may comprise a handle comprising a fluid reservoir at least partially contained therein. The fluid reservoir may comprise a first lumen, a second lumen, and a passageway fluidically coupling the first and second lumens. The device also may comprise a drive assembly at least partially contained therein, where the drive assembly may comprise an actuator configured to be contacted by a user, a first linear gear and a second linear gear. The device may comprise a cannula coupled to a distal end of the housing, and an elongate member slidably positioned within the cannula and configured to be advanced into Schlemm's canal, where fluid may be displaced from the first lumen through the passageway to the second lumen during movement of the actuator in a first direction.

In some variations, actuation of the actuator may displace fluid from the second lumen to the elongate member. In some variations, actuation of the actuator in the first direction may move the first linear gear towards the fluid reservoir and the second linear gear away from the fluid reservoir, where movement of the first linear gear towards the fluid reservoir may displace fluid from the first lumen through the passageway to the second lumen.

Also disclosed herein is a device for delivering fluid to the eye. The device may comprise a handle comprising a fluid reservoir at least partially contained therein. The device also may comprise a cannula coupled to a distal end of the handle, and an elongate member slidably positioned within the cannula and configured to be advanced along an arc of Schlemm's canal, where the device may be configured to deliver fluid from the fluid reservoir at a first rate during advancement of the elongate member and a second, different rate during retraction of the elongate member.

In some variations, the fluid reservoir may comprise a first lumen configured to receive a displacement rod and a second lumen configured to receive a plunger tube, where the first lumen and the second lumen may be fluidically coupled. In some variations, the displacement rod and the plunger tube may be configured to move equal rates in opposite directions.

In some variations, the handle may further comprise a drive assembly at least partially contained therein, where movement of an actuator of the drive assembly in a first direction may deliver fluid from the fluid reservoir at the first rate and movement of the actuator in a second opposite direction may deliver fluid at the second different rate. In some variations, the device may be configured to deliver a first total volume during advancement of the elongate member and a second, different total volume during retraction of the elongate member.

In some variations, the first total volume may be less than the second total volume. In some variations, the first total volume may be between about 3 μL and about 9 μL, including the first total volume being about 6 μL. In some variations, the second total volume may be between about 15 μL and 25 μL, including the second total volume being about 17 μL.

In some variations, the device may be configured to deliver a total combined volume during advancement and retraction of the elongate member. In some variations, the total combined volume may be between about 17 μL to about 50 μL including where the total combined volume may be between about 20 μL to about 30 μL or including where the total combined volume may be between about 24 μL to about 31 μL. In some variations, the total combined volume may be at least about 21 μL.

In some variations, after the second total volume of fluid is delivered during retraction, the fluid reservoir may be configured to receive an additional volume of fluid therein from an external fluid device coupled to a fluid reservoir connector in fluid communication with the fluid reservoir, where the device may be configured to deliver the additional volume of fluid during a subsequent retraction of the elongate member. In some variations, after the second total volume of fluid is delivered during retraction, the elongate member may be configured to be retracted without delivery of additional fluid. In some variations, after a first retraction of the elongate member, the elongate member may be configured for subsequent retractions without the delivery of fluid. In some variations, the elongate member may be configured to be advanced along about 270 degrees to about 360 degrees of Schlemm's canal.

Also disclosed herein is a device for delivering fluid to the eye. The device may comprise a handle comprising a drive assembly and a fluid reservoir each at least partially contained in the handle, a cannula coupled to a distal end of the handle, and an elongate member slidably positioned within the cannula and configured to be advanced along an arc of Schlemm's canal, where movement of an actuator of the drive assembly in a first direction may deliver fluid from the fluid reservoir at a first rate and movement of the actuator in a second opposite direction may deliver fluid from the fluid reservoir at a second, different rate.

Also disclosed herein is a method for delivering fluid to an eye. The method may comprise advancing a distal end of a cannula of fluid delivery device through an anterior chamber of the eye and into Schlemm's canal, where the device may comprise a handle comprising a fluid assembly comprising a fluid reservoir at least partially contained therein. In some variations, the fluid reservoir may comprise a first lumen, a second lumen, and a passageway fluidically coupling the first lumen to the second lumen. In some variations, the method may include advancing an elongate member from the cannula and along an arc of Schlemm's canal while simultaneously delivering a first volume of fluid to Schlemm's canal. In some variations, the method may include retracting the elongate member along the arc of Schlemm's canal while simultaneously delivering a second volume of fluid to the eye.

In some variations, delivering the first volume of fluid may comprise fluid flowing from the first lumen, through the passageway to the second lumen, and from the second lumen through the elongate member. In some variations, delivering the second volume of fluid may comprise fluid flowing from the second lumen and through the elongate member.

In some variations, the method may comprise priming the fluid delivery device before the distal end of the cannula is advanced to Schlemm's canal. In some variations, priming the fluid delivery device may comprise receiving fluid in the first lumen from an external fluid delivery device, and transferring fluid from the first lumen to the second lumen via the passageway.

In some variations, the second lumen may at least partially contain a plunger tube. In some variations, the first lumen may at least partially contain a displacement rod. In some variations, the first volume may be about 3 μL to about 9 μL including about 6 μL. In some variations, the second volume may be about 17 μL to about 24 μL including about 21 μL. In some variations, the first volume may be delivered at a first rate and the second volume may be delivered at a second, different rate.

In some variations, actuation of an actuator of a drive assembly of the fluid delivery device may deliver the first and second volumes of fluid. In some variations, the actuator may be actuated by a hand of a user. In some variations, the fluid assembly may include a plunger tube and a displacement rod, where delivery of the first volume of fluid may be based on at least one geometric difference between the plunger tube and the displacement rod.

In some variations, the method may further comprise positioning an implant within Schlemm's canal, wherein the implant comprises one or more of a suture and biological material. In some variations, the cannula may comprise a surface feature, and wherein the implant is positioned using the surface feature of the cannula.

Also disclosed herein is a device for delivering fluid to an eye. The device may comprise a handle comprising an actuator configured to be contacted by a user, a fluid reservoir at least partially contained in the handle, a displacement rod slidably positioned at least partially in the fluid reservoir, a cannula coupled to a distal end of the handle, and an elongate member slidably positioned within the cannula, where movement of the actuator in both a first direction and a second opposite direction may move the displacement rod into the fluid reservoir.

In some variations, movement of the displacement rod into the fluid reservoir may deliver fluid to the elongate member. In some variations, the device may comprise an extendable fluid coupler fluidly coupling the elongate member to the fluid reservoir. In some variations, the extendable fluid coupler may comprise looped tubing. In some variations, advancement of the elongate member may move the fluid coupler from a contracted configuration to an extended configuration.

In some variations, movement of the actuator in both a first direction and a second opposite direction may decrease a volume of a lumen of the fluid reservoir. In some variations, the device may comprise a drive assembly. In some variations, the drive assembly may comprise a linear gear, a first ratchet wheel, and a second ratchet wheel.

In some variations, movement of the actuator in the first direction may engage the first ratchet wheel with the linear gear and may disengage the second ratchet wheel from the linear gear, where movement of the actuator in the second direction may engage the second ratchet wheel with the linear gear and may disengage the first ratchet wheel from the linear gear. In some variations, the first ratchet wheel may be configured to advance the linear gear towards the fluid reservoir at a first rate and the second ratchet may be configured to advance the linear gear towards the fluid reservoir at a second different rate. In some variations, the second rate may be greater than the first rate.

In some variations, the device may comprise a motor coupled to the displacement rod, the motor may be configured to move the displacement rod into the fluid reservoir. In some variations, the motor may be a first motor and the device may further comprise a second motor configured to move the elongate member independently of the displacement rod.

In some variations, the device may further comprise a linear gear positioned within the handle and operably coupled to the actuator, where the displacement rod may be operably coupled to the linear gear. In some variations, the linear gear may be a first linear gear, and the device may further comprise a second linear gear, where the first and second linear gears may be configured to move in opposite directions. In some variations, movement of the actuator in the first direction may advance the elongate member. In some variations, movement of the actuator in the second direction may retract the elongate member.

Also described herein is a device for delivering fluid to an eye. The device may comprise a handle and a fluid assembly at least partially contained in the handle. The fluid assembly may comprise a fluid reservoir and displacement rod. The device may comprise a cannula coupled to a distal end of the handle and an elongate member configured to be advanced from, and retracted into, the cannula. The displacement rod may be configured to move toward the fluid reservoir to deliver fluid to the elongate member during both advancement and retraction of the elongate member.

Also described herein is a device for delivering fluid to the eye. The device may comprise a handle and a first linear gear and a second linear gear, each at least partially contained within the handle. The device may comprise a cannula coupled to a distal end of the handle, an elongate member configured to be advanced from, and retracted into, the cannula, and a clutch operatively coupled to the first and second linear gears. The clutch may be configured to deliver fluid to the elongate member during advancement of the elongate member.

In some variations, the clutch may be configured to selectively engage the first linear gear. In some variations, rotation of the clutch in a first direction may engage the clutch with the first linear gear and may move the first linear gear and the second linear gear and rotation of the clutch in a second opposite direction may disengage the clutch from the first linear gear and may move the second linear gear. In some variations, the clutch may comprise a first pinion gear comprising a first set of teeth and a second pinion gear comprising a second set of teeth, where the first set of teeth may be configured to selectively engage the second set of teeth.

In some variations, rotation of the first pinion in a first direction may engage the first set of teeth with the second set of teeth and rotation of the first pinion gear in a second opposite direction disengages the first set of teeth from the second set of teeth. In some variations, the clutch may further comprise a spring configured to bias the first pinion gear and second pinion gear of the clutch toward engagement.

In some variations, the clutch may comprise a shaft, a first pinion gear coupled to the shaft, the first pinion gear configured to engage the second linear gear, a pawl wheel coupled to the shaft, and a ratchet hub rotatably coupled to the shaft. In some variations, the ratchet hub may comprise a second pinion gear configured to engage the first linear gear, and one or more internal teeth configured to engage the pawl wheel. In some variations, rotation of the shaft in a first direction may engage the pawl wheel with the ratchet teeth of the ratchet hub and rotation of the shaft in a second opposite direction may disengage the pawl wheel from the ratchet teeth.

In some variations, the device may further comprise a fluid reservoir and displacement rod slidably positioned within the fluid reservoir, where the first linear gear may be coupled to the displacement rod. In some variations, rotation of the clutch in the first direction may move the first and second linear gears in opposite directions.

Also described herein is a device for delivering fluid to an eye. The device may comprise a handle, a cannula coupled to a distal end of the handle, and an elongate member slidably positioned within the cannula and configured to be advanced around at least a 240 degree arc of Schlemm's canal. The elongate member may comprise a first stiffening element and a second stiffening element, where a distal end of the first stiffening element may be longitudinally offset from a distal end of the second stiffening element.

In some variations, the distal end of the first stiffening element may be positioned a first distance from a distal tip of the elongate member and the distal end of the second stiffening element may be positioned a second distance from the distal tip of the elongate member. In some variations, the second distance may be about 1.5 times to about 2.5 times the first distance. In some variations, the second distance may be about double the first distance. In some variations, the first distance may correspond to about 2 to about 4 clock hours of travel of the elongate member around the eye. In some variations, the second distance may correspond to about 5 to about 7 clock hours of travel of the elongate member around the eye. In some variations, the second distance may correspond to travel of the elongate member around about one hemisphere of the eye.

In some variations, the first stiffening element may have a first length and the second stiffening element may have a second, different length. In some variations, the distal end of the first stiffening element may be longitudinally offset from the distal end of the second stiffening element by about 6 mm to about 12 mm.

In some variations, the elongate member may comprise a lumen and the first and second stiffening elements may be at least partially positioned within the lumen. In some variations, the first and second stiffening elements may be floating within the lumen. In some variations, the elongate member may comprise a lumen and a wall surrounding the lumen, where the first and second stiffening elements may be at least partially positioned within the wall of the elongate member.

In some variations, proximal ends of the first and second stiffening elements may be longitudinally aligned. In some variations, proximal ends of the first and second stiffening elements may be longitudinally offset. In some variations, proximal ends of the first and second stiffening elements may be positioned proximal to a proximal portion of the elongate member. In some variations, proximal portions of the first and second stiffening elements may be coupled to a hypotube positioned within the handle.

In some variations, the first stiffening element may have an outer diameter of about 0.01 mm to about 0.05 mm.

Also described herein is a device for delivering fluid to an eye. The device may comprise a handle and a drive assembly at least partially positioned within the handle. The drive assembly may comprise a first linear gear and a second linear gear. The device may comprise a cannula rotatably coupled to a distal end of the handle and an elongate member slidably positioned within the cannula, where rotation of the cannula may rotate at least a portion of the drive assembly.

In some variations, the device may further comprise a cannula actuator configured to be contacted by a user to rotate the cannula. In some variations, the device may further comprise a sheath coupled to the cannula, where the second linear may be slidably positioned within the sheath. In some variations, the sheath may comprise one or more internal splines configured to engage and rotate the second linear gear. In some variations, rotation of the cannula may rotate the elongate member. In some variations, the second linear gear may comprise a plurality of circumferential teeth configured to maintain engagement with a pinion gear of the drive assembly. In some variations, the second linear gear may comprise one or more grooves configured to engage one or more internal splines of the sheath.

### BRIEF DESCRIPTION OF THE DRAWINGS

FIG. 1 shows a stylized, cross-sectional view of the eye and some of the structures involved in the flow of aqueous humor out of the eye.

FIGS. 2A and 2B depict a perspective view and a front view, respectively, of an exemplary delivery device.

FIG. 3 depicts a cross-sectional view of an exemplary delivery device.

FIG. 4 depicts a stylized, perspective view of a column support of the elongate member of the delivery device.

FIG. 5A depicts an elongate member extending from a cannula having one or more markings thereon.

FIG. 5B depicts a closeup of the one or more markings of the elongate member in FIG. 5A.

FIGS. 6A and 6B depict a perspective view and cross-sectional view, respectively, of another exemplary delivery device.

FIGS. 7A and 7B depict cross-sectional views of an exemplary delivery device in a first configuration.

FIG. 7C depicts a cross-sectional view of an exemplary fluid reservoir of a delivery device.

FIG. 7D depicts a cross-sectional view of an exemplary fluid assembly and some components of an exemplary drive assembly in a first configuration.

FIG. 8A depicts a perspective view of an exemplary delivery device and an external fluid device for delivering fluid to the delivery device.

FIG. 8B depicts a cross-sectional view of an exemplary fluid reservoir showing fluid flow through the first lumen and second lumen.

FIG. 9A depicts a perspective view of the exemplary delivery device with the housing of the handle removed and FIG. 9B depicts a cross-sectional view of a pre-advance or “primed” configuration of a variation of an exemplary delivery device of FIG. 9A.

FIG. 10A depicts a perspective view of the exemplary delivery device of FIGS. 9A-9B with the housing of the handle removed and FIG. 10B depicts a cross-sectional view of a post-advance/pre-retract configuration of an exemplary delivery device of FIG. 10A. FIG. 10C depicts a cross-sectional view of the fluid assembly and some components of the drive assembly of FIG. 10B.

FIG. 11A depicts a perspective view of an exemplary delivery device of FIGS. 9A-9B with the housing of the handle removed. FIG. 11B depicts a cross-sectional view of a mid-retract configuration of an exemplary delivery device of FIG. 11A. FIG. 11C depicts a cross-sectional view of the fluid assembly and some components of the drive assembly of FIG. 11B.

FIG. 12A depicts a perspective view of an exemplary delivery device of FIGS. 9A-9B with the housing of the handle removed. FIG. 12B depicts a cross-sectional view of a post-retract configuration of an exemplary delivery device. FIG. 12C depicts a cross-sectional view of the fluid assembly and some components of the drive assembly of FIG. 12B.

FIG. 13 depicts a flow chart of an exemplary method of delivering fluid to the eye using the delivery device of the disclosure.

FIG. 14A depicts a perspective view of an exemplary variation of a fluid assembly with a fluid reservoir, a plunger tube and a concentric outer tube with the housing of the handle removed and FIG. 14B depicts a cross-sectional view of the exemplary variation of the fluid assembly in FIG. 14A.

FIG. 15A depicts a perspective view of an exemplary variation of a cannula. FIGS. 15B-15D respectively depict perspective, side, and bottom views of a distal tip of the cannula of FIG. 15A.

FIG. 16 depicts a stylized, cross-sectional view of an exemplary variation of a delivery device comprising an elongate member reinforced with stiffening and/or stabilizing elements.

FIGS. 17A-17C each depict a perspective view of an exemplary variation of an elongate member with markings.

FIG. 18 depicts a perspective view of an exemplary variation of a distal tip of an elongate member.

FIG. 19 depicts a perspective view of another exemplary variation of a distal tip of an elongate member.

FIG. 20A depicts a perspective view of an exemplary variation of a delivery device, with a portion of the housing shown transparently to illustrate internal components. FIG. 20B depicts a perspective view of the internal components and an actuator of the delivery device of FIG. 20A.

FIG. 20C depicts a cross-sectional view of components of an actuator of the delivery device of FIG. 20A.

FIG. 21 depicts a back perspective view of an exemplary variation of a sheath for use with actuator of the deliver device.

FIG. 22 depicts a front perspective view of an exemplary variation of a linear gear of the drive assembly for use with a cannular actuator.

FIG. 23 depicts a perspective view of an exemplary variation of a delivery device with a portion of the housing of the handle removed.

FIG. 24 depicts a top perspective view of an exemplary variation of a linear gear.

FIG. 25 depicts a side perspective view of an exemplary variation of a displacement rod.

FIG. 26A depicts a perspective view of an exemplary variation of a portion of the drive assembly in an advancement configuration. FIG. 26B depicts a perspective view of an exemplary variation of a portion of the drive assembly in a retraction configuration. FIG. 26C depicts a perspective view of an exemplary variation of a portion of the drive assembly in a re-advancement configuration. FIG. 26D depicts a perspective view of an exemplary variation of a portion of the drive assembly in a re-retraction configuration.

FIG. 27A depicts a side view of an exemplary variation of a linear gear including a stop.

FIG. 27B depicts a perspective view of another exemplary variation of a linear gear including a stop.

FIG. 28 depicts a perspective view of an exemplary variation of a delivery device with a portion of the housing of the handle removed to show a ratchet arm and linear gear.

FIGS. 29A and 29B depict a top view and a side view respectively of an exemplary variation of a displacement rod ratchet.

FIG. 30 depicts a side view of an exemplary variation of a linear gear including a notch.

FIG. 31A depicts a side view of the exemplary variation of the linear gear of FIG. 30 and the displacement rod ratchet of FIGS. 29A-29B. FIG. 31B depicts a side view of an exemplary variation of the linear gear of FIG. 30 and the displacement rod ratchet of FIGS. 29A-29B.

FIG. 32 depicts a perspective view of another exemplary variation of a displacement rod ratchet.

FIG. 33 depicts a perspective view of exemplary variation of a linear gear including an engagement features.

FIG. 34A depicts a perspective view of an exemplary variation of a delivery device in an advancement configuration with a portion of the handle housing removed to show a displacement rod ratchet and linear gear. FIG. 34B depicts a perspective view of an exemplary variation of a delivery device in a retraction configuration with a portion of the handle housing removed to show a displacement rod ratchet and linear gear. FIG. 34C depicts a perspective view of an exemplary variation of a delivery device in a re-advancement configuration with a portion of the handle housing removed to show a displacement rod ratchet and linear gear. FIG. 34D depicts a perspective view of an exemplary variation of a delivery device in a re-retraction configuration with a portion of the handle housing removed to show a displacement rod ratchet and linear gear.

FIG. 35 depicts a perspective view of an exemplary variation of a delivery device with a portion of the housing of the handle removed to show an exemplary variation of a clutch.

FIGS. 36A and 36B depict a perspective view and a side view respectively of an exemplary variation of a drive assembly including a clutch.

FIGS. 37A and 37B depict a side view and perspective view respectively of an exemplary variation of a clutch.

FIGS. 38A and 38B depict a side view and a perspective view of an exemplary variation of a pinion gear.

FIGS. 39A and 39B depict a side view and a perspective view respectively of an exemplary variation of another pinion gear.

FIG. 40 depicts a perspective view of an exemplary variation of a delivery device with a portion of the housing of the handle removed to show another exemplary variation of a clutch.

FIG. 41A depicts a side view of an exemplary variation of a drive assembly including a clutch. FIG. 41B depicts a perspective view of an exemplary variation of a drive assembly including a clutch.

FIG. 42A depicts a perspective view of an exemplary variation of a pinion gear. FIG. 42B depicts a perspective view of an exemplary variation of a ratchet hub. FIG. 42C depicts a perspective view of an exemplary variation of a pawl wheel.

FIG. 43A and FIG. 43B depict side views of an exemplary variation of a delivery device including an extendable fluid coupler in a pre-advancement configuration. FIG. 43C is a perspective cross-sectional view of an exemplary variation of a delivery device including an extendable fluid coupler in a pre-advancement configuration.

FIG. 44A depicts a perspective view of an exemplary variation of a delivery device including an extendable fluid coupler in an advancement configuration. FIG. 44B depicts a side view of an exemplary variation of a delivery device including an extendable fluid coupler in an advancement configuration.

FIG. 45A depicts a perspective view of an exemplary variation of a delivery device including an extendable fluid coupler in a retraction configuration. FIG. 45B depicts a side view of an exemplary variation of a delivery device including an extendable fluid coupler in a retraction configuration. FIG. 45C depicts a cross-sectional view of an exemplary variation of a delivery device including an extendable fluid coupler in a retraction configuration.

FIG. 46 depicts a top perspective view of an exemplary variation of a delivery device with a portion of the housing of the handle removed to show an exemplary variation of lock.

FIG. 47 depicts a cross-sectional view of an exemplary variation of a fluid reservoir.

FIG. 48 depicts a stylized top view of an exemplary variation of a device advancing around Schlemm's canal.

FIG. 49 depicts a stylized top view of an exemplary variation of a device exiting the eye.

### DETAILED DESCRIPTION

Described herein are systems and methods for accessing Schlemm's canal and for delivering a fluid composition therein and/or for tearing the trabecular meshwork to reduce intraocular pressure and thereby treat conditions of the eye. The fluids and certain components of the system, e.g., the slidable elongate member (e.g., conduit), may be used to provide a force for disrupting one or more trabeculocanalicular tissues, which include the trabecular meshwork, juxtacanalicular tissue, Schlemm's canal, and the collector channels. As used herein, the term “disrupting” refers to the delivery of a volume of fluid or use of a system component that alters the tissue in a manner that improves flow through the trabeculocanalicular outflow pathway. Examples of tissue disruption include, but are not limited to, dilation of Schlemm's canal, dilation of collector channels, increasing the porosity of the trabecular meshwork, stretching the trabecular meshwork, forming microtears or perforations in juxtacanalicular tissue, removing septae from Schlemm's canal, cutting, tearing, or removal of trabeculocanalicular tissues, and a combination thereof.

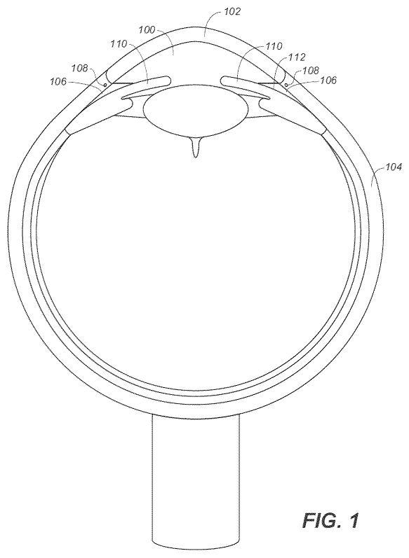

To better understand the systems and methods described here, it may be useful to explain some of the basic eye anatomy. FIG. 1 is a stylized depiction of a normal human eye. The anterior chamber (100) is shown as bounded on its anterior surface by the cornea (102). The cornea (102) is connected on its periphery to the sclera (104), which is a tough fibrous tissue forming the protective white shell of the eye. Trabecular meshwork (106) is located on the outer periphery of the anterior chamber (100). The trabecular meshwork (106) extends 360 degrees circumferentially around the anterior chamber (100). Located on the outer peripheral surface of the trabecular meshwork (106) is Schlemm's canal (108). Schlemm's canal (108) extends 360 degrees circumferentially around the meshwork (106). At the apex formed between the iris (110), meshwork (106), and sclera (104), is the anterior chamber angle (112).

The delivery systems are generally configured for single-handed manipulation and for control by a single operator and include one or more features useful for easily accessing Schlemm's canal with minimal trauma. Once access to the canal has been obtained, the delivery system may deliver a fluid composition and/or tear the trabecular meshwork. For example, the lumen of the elongate member (e.g., conduit) may be configured to deliver a fluid composition to the canal, and the body of the elongate member may be configured to cut or tear through the trabecular meshwork if the system is removed from the eye while the elongate member is extended from the cannula and within Schlemm's canal.

It should be appreciated that in some instances the delivery systems described herein may be used only to deliver a fluid composition to Schlemm's canal (and not to separately tear the trabecular meshwork using, for example, the body of the elongate member) or may be used only to tear the trabecular meshwork (using, for example, the body of the elongate member) without delivering a fluid composition. Moreover, as noted above, in some instances, the delivery systems described herein may be used to both deliver a fluid composition to Schlemm's canal and to disrupt the trabeculocanalicular tissues (e.g., tear the trabecular meshwork).

In some variations, the systems described herein may be used to deliver an ocular implant to Schlemm's canal. In some variations, the methods described herein may comprise implanting a device completely or partially into Schlemm's canal in conjunction with delivering a fluid composition into the canal and/or tearing the trabecular meshwork (the delivery of the ocular device, fluid composition and/or tearing performed in any order). Devices implanted in Schlemm's canal may generally be configured to maintain the patency of the canal without substantially interfering with transmural fluid flow across the canal. This may restore, enable, or enhance normal physiologic efflux of aqueous humor through the trabeculocanalicular tissues. Ocular devices (implants) such as any of those disclosed in U.S. Pat. Nos. 7,909,789, 8,529,622, and 9,095,412 and co-pending U.S. application filed on Feb. 27, 2025 titled “Intraocular Systems, Devices, Kits, and Methods” to Daniel O'Keeffe et al., having an attorney docket number of SGHT-014/01US 328518-2191, each of which is hereby incorporated by reference in its entirety, may be delivered. In some variations, the ocular devices delivered, such as some of the ocular devices described in U.S. Pat. Nos. 7,909,789, 8,529,622, 9,095,412, and co-pending U.S. application filed on Feb. 27, 2025 titled “Intraocular Systems, Devices, Kits, and Methods” to Daniel O'Keeffe et al., having an attorney docket number of SGHT-014/01US 328518-2191, may include a support having a least one fenestration. The support may completely traverse a central core of Schlemm's canal without substantially interfering with transmural fluid flow or longitudinal fluid flow across or along the canal. The ocular device may have minimal surface area contact with the walls of Schlemm's canal (e.g., only 2, only, 3, only 4 points of contact). In some variations, the ocular device may comprise a radius of curvature different (e.g., larger, smaller) than the radius of curvature of the canal, which may decrease surface contact between the ocular device and the canal. The ocular device may also disrupt the juxtacanalicular trabecular meshwork or adjacent inner wall of Schlemm's canal. In some variations, the ocular devices may be coated with a drug useful for treating ocular hypertension, glaucoma, pre-glaucoma, infection, scarring, neovascularization, fibrosis, and/or postoperative inflammation. The ocular device may also be formed to be solid or semi-solid, and/or biodegradable (e.g., bioabsorbable). In some variations, the ocular device may be made of or may comprise a biocompatible material such as collagen or a collagen derivative. The ocular device may be entirely made of and/or comprise modified (e.g., processed/decellularized) scleral tissue. In other variations, the ocular device may be entirely made of and/or comprise a suture. In either of these variations, the ocular device may comprise one or more of: fenestration(s), bead(s), knots and other elements or features described in any of U.S. Pat. Nos. 7,909,789, 8,529,622, and 9,095,412 and co-pending U.S. application filed on Feb. 27, 2025 titled “Intraocular Systems, Devices, Kits, and Methods” to Daniel O'Keeffe et al., having an attorney docket number of SGHT-014/01US 328518-2191, which were previously incorporated by reference herein in their entirety.

### Delivery Devices

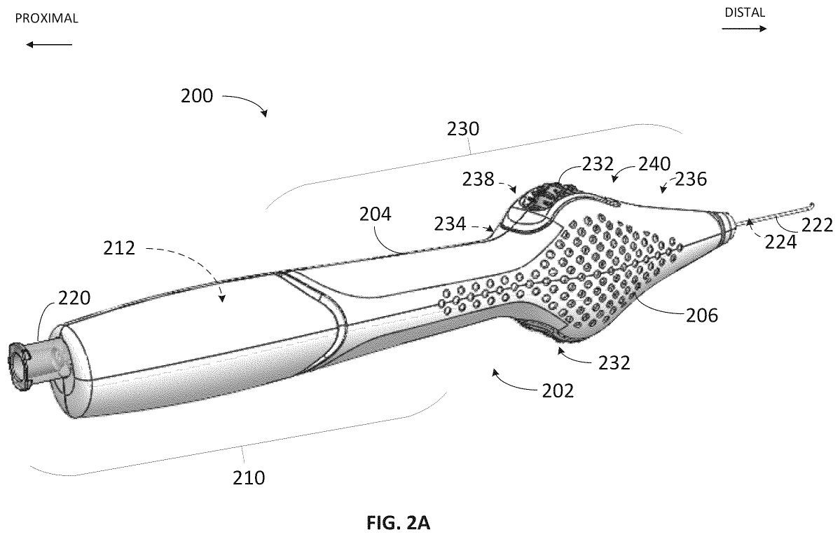

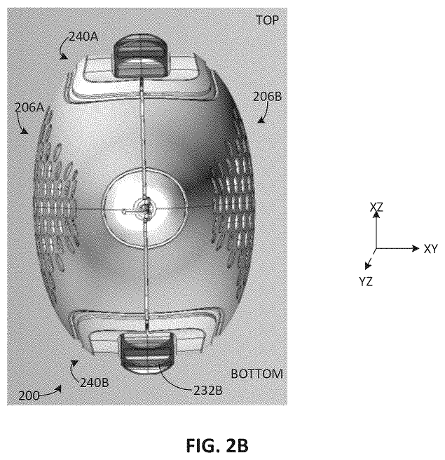

The delivery devices described herein may generally include single-handed, single-operator controlled devices. As depicted in FIG. 2A, the delivery devices 200 described herein may generally comprise a handle 202 having a housing 204 and a fluid assembly 210 at least partially contained in the housing. The handle 202 may include a grip portion 206 configured to receive a user's hand. The fluid assembly 210 may include a fluid reservoir 212. In some variations, the fluid reservoir 212 may comprise a fluid reservoir connector 220 configured to releasably couple with an external fluid device configured to deliver a fluid composition to the fluid assembly 210. In other variations, the fluid reservoir 212 may not include a fluid connector and/or may not be configured to receive a fluid in-situ within the handle 202 (e.g., the fluid reservoir 212 may be configured to be preloaded with a fluid composition or otherwise configured to receive a fluid composition before integration into handle 202). The fluid assembly 210 may include one or more moveable components that together with a drive assembly may deliver a fluid composition from the device to the eye, as will be described in more detail herein. The delivery device may comprise a cannula 222 coupled to and extending from a distal end of the handle and may further comprise an elongate member 224 within a lumen of the cannula 222. In some variations, the elongate member may comprise a lumen therethrough, and the lumen may be in fluid communication with the fluid reservoir. The elongate member may be configured to be advanced into and around at least a portion of Schlemm's canal, as will be described in more detail herein. Fluid compositions such as saline, viscoelastic fluids, including viscoelastic solutions, air, and gas may be delivered using the delivery device. Suitable markings, colorings, or indicators may be included on any portion of the delivery device to assist in identifying the location and/or position of the one or more portions of the delivery device, such as, for example, a distal end of the cannula and/or any portion of the elongate member (e.g., distal end, central portion, proximal end), as will be discussed in more detail herein.

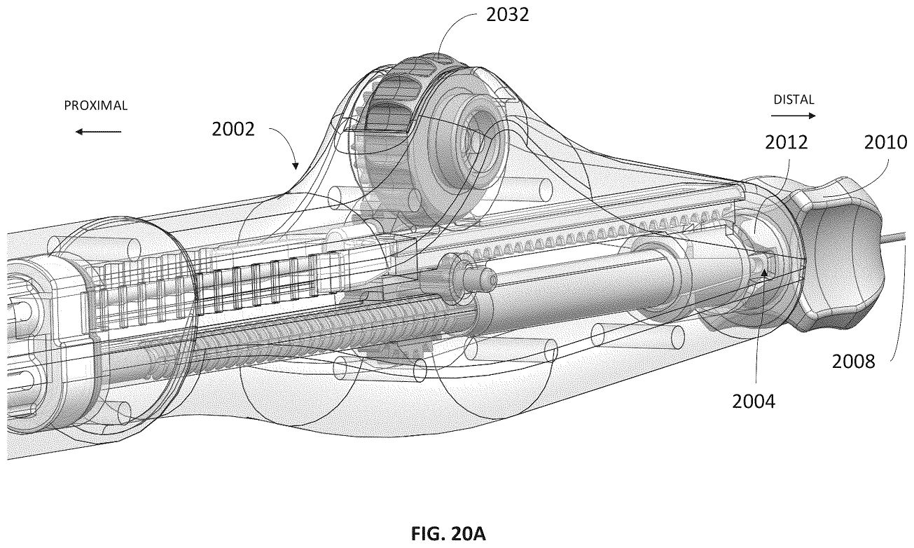

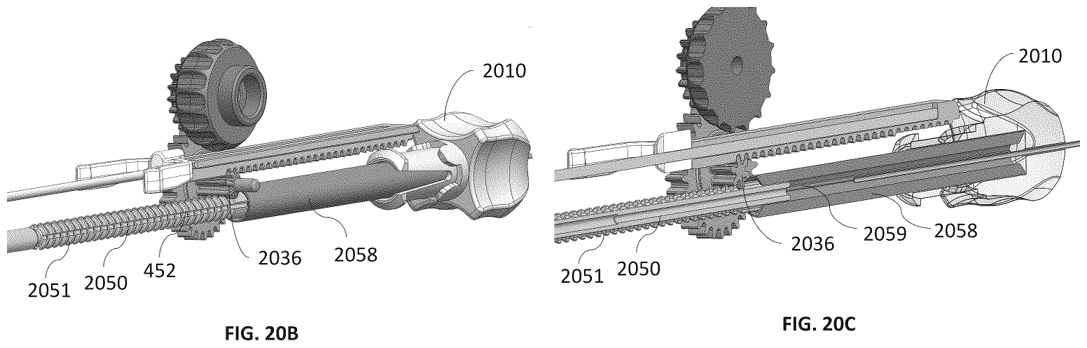

The delivery device 200 may also generally comprise a drive assembly 230. The drive assembly 230 may be at least partially contained within the housing 204 of the handle 202. In some variations, the drive assembly 230 may comprise one or more moveable components 232 including one or more actuators 232 configured to be actuated by the hand of a user to deliver a fluid composition (e.g., while simultaneously advancing or retracting the elongate member 224 within Schlemm's canal or the cannula 222 or separately from advancing and/or retracting the elongate member 224). In some variations, the one or more actuators 232 may include a single actuator. Alternatively, the one or more actuators 232 may comprise a plurality of actuators (e.g., at least one, two, three, four, five, or more than five actuators). In some variations, one or more actuators may be operatively coupled to one or more motors, such that actuation of the one or more actuators of the delivery device may actuate one or more motors to advance and/or retract the elongate member and/or deliver a fluid composition in a motorized fashion. Moreover, the drive assembly 230 may optionally include a cannula actuator (not shown) configured for a function different from the one or more actuators 232. For example, while the actuator(s) 232 may be configured to translate the elongate member 224 longitudinally within the cannula 222 and/or deliver a fluid composition, the drive assembly 230 may include the cannula actuator to rotate the cannula 222 about the longitudinal axis of the delivery device 200. An exemplary such configuration will be described in detail herein with reference to FIGS. 20A-20C.

In some instances, the systems described herein may be used to perform ab-interno trabeculotomy, ab-interno transluminal trabeculotomy, clear corneal trabeculotomy, clear corneal transluminal trabeculotomy, ab-interno canaloplasty, and/or clear corneal canaloplasty, and/or may be used to deliver a fluid composition into, for example, the anterior or posterior segment of the eye.

### Handle

The delivery devices described herein may include delivery devices comprising a handle capable of single-handed use by a single operator, as seen in FIG. 2A. The handle may be configured such that the ability to use the delivery device is independent of which hand a user chooses to use or on which eye a procedure is performed. For example, the handle may be configured for use in the left and right hands and for use on the left and right eyes. The handle may be further configured such that the ability to use the delivery device is independent of which direction around Schlemm's canal the elongate member and/or a fluid composition is delivered, and how much of Schlemm's canal is traversed. For example, the delivery device may be used to deliver a fluid composition in a clockwise direction in an eye, and then with a simple rotation of the handle (or by rotating the cannula itself 180 degrees in another variation) to a second orientation, may be used to deliver a fluid composition in the counterclockwise direction, or vice versa. Additionally, or alternatively, the delivery device may be used to deliver a fluid composition only in the clockwise direction or only in the counterclockwise direction.

Referring to FIG. 2, the handle may generally comprise a housing having a proximal portion comprising a proximal end, and a distal portion comprising a distal end and a grip portion proximal of a distal end. The proximal portion may generally be configured to contain, or at least partially contain, components of the fluid assembly (e.g., the fluid reservoir). The distal portion, and more specifically, the grip portion 206 as seen in FIG. 2, may generally be configured to be held by a user while positioning a cannula 222 (e.g., advancing the cannula across the anterior chamber, puncturing the trabecular meshwork, advancing the cannula into Schlemm's canal), actuating an elongate member, and/or delivering the fluid composition. The proximal and distal portions of the housing 204 may each generally include an interior cavity that may contain (or at least partially contain) internal components of the device, such as components of the drive assembly 230 and fluid assembly 210. The distal portion (e.g., an interior cavity of the distal portion) may contain, or at least partially contain, components of the drive assembly 230 and may house an internal portion of the cannula 222. It should be appreciated that one or more components of the drive assembly 230 and/or the fluid assembly 210 may be configured to translate, and accordingly, may move between the internal cavities of the proximal and distal portions of the housing. In some variations, a cannula actuator (not shown) may be coupled (directly or indirectly) to the internal portion of the cannula 222 at the distal potion of the housing 204 (e.g., as shown in FIGS. 20A-20C). In some variations, the handle may include one or more indicators to communicate the travel distance of the catheter.

In some variations, the proximal portion of the housing (e.g., a proximal end) may include a fluid reservoir connector 220 that may be configured to at least partially (e.g., entirely) fill the fluid reservoir 212 of the fluid assembly 210 with a fluid composition. Additionally, the fluid reservoir connector 220 may be configured to provide a fluid composition for irrigation of the operative field and/or purge air from the system. The distal end of the housing 204 may have the cannula 222 and a cannula actuator coupled thereto. In some variations, the distal end of the housing may also include a lock configured to prevent movement the drive assembly and fluid assembly. The grip portion 206 may be raised, depressed, and/or grooved in certain areas, or otherwise textured to improve grasp of the handle by the user, increase the ergonomic fit of the handle into the hand of a user and control orientation of the handle without requiring wrist rotation, and/or to improve user comfort. The grip portion 206 may be configured to allow the user to grip the handle close or adjacent to the cannula 222 (e.g., within about 3 inches or less), while still allowing the user to actuate the elongate member 224 and/or deliver the fluid composition via the one or more actuators 232. In some variations, the grip portion 206 may be configured to allow the user to grip the handle within about 0.1-3 inches from a proximal end of the cannula 222. For example, the grip portion 206 may be configured to allow the user to grip the handle 202 within about 3 inches within about 2.5 inches, within about 2 inches, within about 1.5 inches, within about 1 inch, within about 0.75 inches, within about 0.5 inches, or within about 0.25 inches from a proximal end of the cannula 222. In some variations, the grip portion 206 may be configured to allow the user to grip the handle 202 within about 0.25-2 inches from the proximal end of the cannula 222. In some variations, the grip portion 206 may be configured to allow the user to grip the handle 202 within about 0.25-1.5 inches from the proximal end of the cannula 222.

The handle 202 or portions thereof (e.g., proximal portion, distal portion including grip portion 206) may be made from or may comprise any suitable material, including without limitation, fluoropolymers; thermoplastics such as polyetheretherketone, polyethylene, polyethylene terephthalate, polyurethane (or as thermoset), nylon, and the like; or silicone. In some variations, the housing 204 or portions thereof may be made from or may comprise transparent materials. Materials with suitable transparency are typically polymers such as acrylic copolymers, acrylonitrile butadiene styrene (ABS), polycarbonate (PC), polystyrene, polyvinyl chloride (PVC), polyethylene terephthalate glycol (PETG), and styrene acrylonitrile (SAN). Acrylic copolymers that may be particular useful include, but are not limited to, polymethyl methacrylate (PMMA) copolymer and styrene methyl methacrylate (SMMA) copolymer (e.g., Zylar 631® acrylic copolymer). In variations in which the handle is reusable, the handle may be made from a material that can be sterilized (e.g., via autoclaving), such as a heat-resistant metal (e.g., stainless steel, aluminum, titanium) or a high-performance engineering polymer such as PEI, PEEK or PEKK.

The length of the handle may generally be between about 1 inch (2.5 cm) to about 20 inches (50.8 cm). In some variations, the length of the universal handle may be between about 4 inches (10.2 cm) and 10 inches (25.4 cm) from a distal end of the handle to a proximal end of the handle and may vary by +/−1.0 inch. In some variations, the length of the handle may be about 4 inches, about 4.1 inches, about 4.2 inches, about 4.3 inches, about 4.4 inches, about 4.5 inches, about 4.6 inches, about 4.7 inches, 4.8 inches, about 4.9 inches, about 5 inches, about 5.1 inches, about 5.2 inches, about 5.3 inches, about 5.4 inches, about 5.5 inches, about 5.6 inches, about 5.7 inches, about 5.8 inches, about 5.9 inches, about 6 inches, about 6.1 inches, about 6.2 inches, about 6.3 inches, about 6.4 inches, about 6.5 inches, about 6.6 inches, about 6.7 inches, about 6.8 inches, about 6.9 inches, or about 7 inches (17.8 cm) from the distal end of the handle to the proximal end of the handle. In some variations, the length of the device may include the length of the handle, the length of a cannula coupled to the handle and the length of a fluid connector. In some variations, the length of the handle and/or the length of the device, may be from about 4 inches to about 12 inches, including about 4 inches, about 4.1 inches, about 4.2 inches, about 4.3 inches, about 4.4 inches, about 4.5 inches, about 4.6 inches, about 4.7 inches, 4.8 inches, about 4.9 inches, about 5 inches, about 5.1 inches, about 5.2 inches, about 5.3 inches, about 5.4 inches, about 5.5 inches, about 5.6 inches, about 5.7 inches, about 5.8 inches, about 5.9 inches, about 6 inches, about 6.1 inches, about 6.2 inches, about 6.3 inches, about 6.4 inches, about 6.5 inches, about 6.6 inches, about 6.7 inches, about 6.8 inches, about 6.9 inches, about 7 inches, about 7.1 inches, about 7.2 inches, about 7.3 inches, about 7.4 inches, about 7.5 inches, about 7.6 inches, about 7.7 inches, about 7.8 inches, about 7.9 inches, about 8.0 inches, about 8.1 inches, about 8.2 inches, about 8.3 inches, about 8.4 inches, about 8.5 inches, about 8.6 inches, about 8.7 inches, about 8.8 inches, about 8.9 inches, about 9.0 inches, about 9.1 inches, about 9.2 inches, about 9.3 inches, about 9.4 inches, about 9.5 inches, about 9.6 inches, about 9.7 inches, about 9.8 inches, about 9.9 inches, about 10.0 inches, about 10.1 inches, about 10.2 inches, about 10.3 inches, about 10.4 inches, about 10.5 inches, about 10.6 inches, about 10.7 inches, about 10.8 inches, about 10.9 inches, about 11.0 inches, about 11.1 inches, about 11.2 inches, about 11.3 inches, about 11.4 inches, about 11.5 inches, about 11.6 inches, about 11.7 inches, about 11.8 inches, about 11.9 inches, or about 12 inches.

The handle 202 may be configured for ambidextrous use with an ergonomic fit in the hand. To that end, one or more of the proximal and distal portions of the handle 202 may be configured to be symmetric across one or more planes (e.g., two planes), such as a YZ-plane and a XZ-plane. Such a configuration may make it easier for a user to rotate the handle (e.g., about 10 degrees to about 180 degrees or more) around a longitudinal axis of the handle during use. In some variations, such a configuration may make it easier for the user to rotate the handle about 15 degrees to about 30 degrees including about 20 degrees around the longitudinal axis of the handle during use of the device (e.g., during delivery of the fluid composition to each hemisphere of Schlemm's canal). In some variations, all portions of the handle may be symmetric across one or more planes (e.g., YZ-plane, XZ-plane, and a XY plane). In some variations, one or more of the distal portion and the proximal portion may be configured to be symmetric across one or more planes and may have a non-circular cross-sectional shape, while in other variations, one or more of the distal portion and the proximal portion may be configured to be symmetric across one or more planes and may have a circular cross-sectional shape. For example, in some variations, one or more portions may have a circular cross-sectional shape (e.g., the proximal portion, the distal end of the distal portion), and one or more portions may have a non-circular cross-sectional shape (e.g., the grip portion of the distal portion). It should be appreciated that the symmetric nature of the portions of the housing 204 may refer only to the shape of the profile of that portion (e.g., outer surface) and may or may not include internal surfaces of the housing (i.e., the profile of the portion of the housing may be symmetric across a plane while the internal surface may include different pins, extensions, or other structures configured to interact with or engage internal components of the device).

In some variations, the handle may be as described in U.S. patent application Ser. No. 18/454,585 filed Aug. 23, 2024, the contents of which are hereby incorporated by reference herein in their entirety.

### Distal Portion of the Handle

Referring to FIG. 2A, the grip portion 206 may comprise a first curved side and a second curved side opposite the first curved side. In some variations, the grip portion 204 may comprise one or more actuators 210, wherein the one or more actuators 210 that may be contacted by the user to actuate one or more linear gears of the drive assembly 230 configured to move the elongate member 224 and/or deliver fluid. The grip portion 206 may be configured to be received in a user's hand, or to otherwise be grasped or held during use of the device, and more specifically, while advancing the device to Schlemm's canal, accessing Schlemm's canal with the cannula, actuating the elongate member, and/or delivering a fluid composition. Although the user may grasp any location on the handle 2022, including during transfer of the fluid composition into the fluid reservoir, the grip portion 202 may be particularly configured to receive a user's hand (e.g., fingers) while accessing Schlemm's canal and/or during delivery of the fluid composition to the eye, and may provide convenient access to the one or more actuators 210 during a procedure. The grip portion 206 may be commensurate in length with the distal portion of the handle 202 or may form a portion or segment of the distal portion. In some variations, the grip portion 206 may be configured enable forward hand positioning of the user relative to the patient, while a proximal portion may rest in the groove between a user's thumb and pointer finger. In general, the handle is designed to enable a user to securely grasp the grip portion close to cannula, wherein close to the cannula includes distal the one or more actuators. In some variations, the grip portion 206 may taper towards the cannula, thereby forming an elongated nose, which may further allow for precise control of the distal portion by providing a surface that easily and comfortably allows for placement of a user's finger(s). Moreover, the grip portion 206 is configured to facilitate the user grasping and maneuvering the device close to the cannula, which may optimize a user's control of the cannula during use (e.g., while accessing Schlemm's canal). The position and shape of the grip portion 206 may also bring the hand of the user closer to the patient, which may improve stability and control of the device. Moreover, the handle, and in particular, the grip portion 206 of the handle, may be configured to provide points of contact for steering and control, as described above, while also allowing a user to access, without repositioning of the hand, the one or more actuators of the device.

Generally, a grip portion may be symmetric across an XY plane and may comprise a top and a bottom, where each of the top and bottom include an actuator configured to be accessed by a user. Each of the top and bottom may comprise a first curved surface and a straight surface, with a second curved surface therebetween. As seen in FIG. 2A, a top of the grip portion 206 may comprise a first curved surface 234 and a straight surface 236, with a second curved surface 238 therebetween. The first curved surface 234 may be proximal of the second curved surface 238 and first curved surface 234 may directly abut the second curved surface 238 (e.g., there may not be any other surface therebetween). Put differently, the distal edge of the first curved surface 234 may be the proximal edge of the second curved surface 238. The first curved surface 234 may be concave while the second curved surface 238 may be convex. An actuator 232 configured to be contacted by a user may be positioned in, through, or otherwise along the second curved surface 238. The straight surface 236 may be distal to both first curved surface 234 and the second curved surface 238. The straight surface 236 may directly abut the second curved surface 238 (e.g., there may not be any other surface therebetween). Put differently, the distal edge of the second curved surface 238 may be the proximal edge of the straight surface 236. The straight surface 236 may be tapered along a longitudinal axis of the handle from a proximal end of the straight surface 236 to a distal end of the straight surface 236. In some variations, the straight surface 236 may terminate at or adjacent to (e.g., just proximal of) the distal end of the handle.

In some variations, the grip portion 206 may comprise different cross-sectional shapes along the longitudinal axis of the handle. For example, proximally, the grip portion 206 may comprise a circular cross-sectional shape with a first diameter, while a central portion of the grip portion 206 comprising the top surface 240 and the bottom surface (partially seen) and the actuators 232 may comprise an oblong or oval cross-sectional shape with a major axis and a minor axis, and a distal portion (including the distal end) of the grip portion 206 may comprise a circular cross-sectional shape with a second diameter. The major and minor axes may increase distally from the proximal portion of the grip portion 206 comprising the circular cross-sectional shape to a maximum major axis and a maximum minor axis, which may be aligned with the location of the actuators 232, and then may decrease distally until the distal portion of the grip portion 206 comprising a circular cross-sectional shape with the second diameter. In some variations, the first diameter may be larger than the second diameter and the maximum major axis may be larger than both the first diameter and the second diameter. In some variations, the major axis may be at least about 1.5 times to at least about 3 times the minor axis. In some variations, the major axis may be at least about 1.5 times to at least about 2.5 times the diameter of the proximal portion of the handle. The first diameter of the grip portion may be substantially equal to the diameter of the proximal portion of the handle. In some variations, the diameter of the proximal portion of the handle may be greater than the first diameter of the grip portion. In some variations, the difference in the diameters of the different cross-sectional shapes along the longitudinal axis of the handle may provide added benefits. For example, in some variations, the smaller the diameter of the handle, the more degrees of rotation may occur per movement, especially at the distal end.

As noted above, in some variations, the grip portion may comprise different cross-sectional shapes along the longitudinal axis of the handle. In some variations, the cross-sectional shapes may include only rounded shapes (e.g., circular, ovular). In some of these variations, proximally, the grip portion 206 may comprise a circular cross-sectional shape (e.g., across the XY plane, and/or across the XZ plane, and/or YZ plane), centrally, the grip portion 206 may comprise an ovular cross-sectional shape (e.g., across the XY plane, and/or across the XZ plane, and/or YZ plane),), and distally, the grip portion may comprise a circular cross-sectional shape. In other variations, one or more of the cross-sectional shapes of the grip portion 206 may include a polygonal shape. For example, proximally, the grip portion 206 may comprise a circular cross-sectional shape with a first diameter while the central portion of the grip portion 206 may comprise a polygonal shape having faceted faces (e.g., two or more faces), and the distal end of the grip portion 206 may comprise a circular cross-sectional shape. The polygonal shape may include a triangular prism, a rectangular prism, a pentagonal prism, a hexagonal prism, a heptagonal prism, an octagonal prism, or the like. In some variations, the faceted faces may collectively comprise a radius of curvature. In some variations, the cross-sectional shape of the housing of the handle at the one or more flat regions may be different from the cross-sectional shape at any of: the distal end of the distal portion, the neck, or the proximal portion.

The shape of the handle, the differences in the diameters of the portions of the handle along the longitudinal axis, and the taper of the distal portion of grip portion may assist in functionally balancing the intentional movement needed to rotate the distal end of the device with the precise control over subtle movement needed during use.

Moreover, the handles described herein may be configured to promote or otherwise facilitate a forward grip (i.e., more distal on the handle), which may provide the user with added control over the cannula. For example, the grip portion of the handle may include a taper (e.g., an elongated nose), as depicted in FIG. 2A. As shown there, the grip portion 206 may comprise a taper from the maximum height to the minimum height of the grip portion 206. The taper in the distal portion may promote a forward grip of the handle by the user (i.e., a grip of the distal portion on the handle). Advantageously, a grip of the user located more distally on the handle places the hand of the user in closer proximity to the eye of the patient, allowing the user more control over the cannula. Furthermore, generally the taper from each of the top surface 240 and the bottom surface (not seen) to a distal end provides a location (e.g., a portion of the grip portion 206) for a user to place an index finger distal the actuators 232. The user may seamlessly move their index finger or thumb between the grip portion 206 and the actuators 232 to access Schlemm's canal (e.g., advance the device to Schlemm's canal, puncture the trabecular meshwork, advance the distal tip of the cannula into Schlemm's canal), actuate the elongate member and deliver the fluid composition. Additionally, the taper from a maximum height to the minimum height of the grip portion 206 may generally provide multiple resting points for fingers of the user, which may additionally assist in reducing user fatigue.

The grip portion 206 may be shaped in a way to ergonomically fit into a hand of the user. To this end, in some variations, the grip portion 206 may be symmetric across a YZ plane, as depicted in FIG. 2B. The YZ plane may divide the grip portion 206 into a first grip portion or half 206A and a second grip portion or half 206B that are symmetric across the YZ plane. The first and second grip portions 206A/206B may each comprise a rounded profile from a first, top surface 240A to a second, bottom surface 240B opposite the top surface 240A. In this manner, the grip portion 206 may comprise a first curved side and a second curved side opposite the first curved side. More specifically, the first grip portion 206A may comprise the first curved side and the second grip portion 206B may comprise the second curved side. In some variations, the first grip portion 206A and the second grip portion 206B may each comprise a continuous curved side (e.g., lacking a planar surface) from the top surface 240A to the bottom surface 240B. For example, the first curved side and the second curved side may each be continuously curved and/or may have a constant radius of curvature from the top surface 240A to the bottom surface 204B, between the actuators, and/or between one or more flat regions each of the top surface 240A and the bottom surface 240B.

In some variations, the first and second grip portions (e.g., the first and second curved sides) may be symmetric across the cannula. In some variations, the first grip portion 206A and the second grip portion 206B may be symmetric across the XZ plane. In some variations, a portion of each of the first curved side and the second curved side may align with the first and second the actuators 232A/232B, respectively. For example, in some variations, the first and/or second actuator may be longitudinally centered along the first and/or second curved side, respectively.

In some variations, the first grip portion 206A (e.g., first curved side) and the second grip portion 206B (e.g., second curved side) may each be convex (e.g., entirely convex) or comprise a convex curve (e.g., from a top surface to a bottom surface, or between a first actuator and a second actuator). In some variations, the convex curve of the first grip portion 206A and the second grip portion 206B may have a radius of curvature of about 0.3 inches to about 1.5 inches. In some variations, the radius of curvature of the convex curve of the first grip portion 206A and the second grip portion 206B may include ranges of about ⅜ inch to about 1.5 inches, about ½ inch to about 1.5 inches, about ¾ inch to about 1.5 inches, about 1 inch to about 1.5 inches, and about 1.25 inches to about 1.5 inches. In some variations, the radius of curvature of the convex curve of the first grip portion 206A and the second grip portion 206B may include ranges of about 0.3 inch to about 1 inch, about ⅜ inch to about ¾ inch include about ½ inch. The radius of curvature of the convex curve of the first grip portion 206A and the second grip portion 206B may include ranges of about 0.3 inch to about 1.5 inches, about 0.3 to about 1.25 inches, about 0.3 inch to about 1.0 inch, about 0.3 inch to about ¾ inch, about 0.3 inch to about ½ inch, and about 0.3 inch to about ⅜ inch. In some variations, the first grip portion 206A may have a first arc (e.g., a convex arc) with a first center, the second grip portion 206B may have a second arc (e.g., a convex arc) with a second center, and the first and second centers may be on opposing sides of a central longitudinal axis of the cannula and/or a central longitudinal axis of the handle, as seen in FIG. 2B. In some variations, utilizing convex first and second grip portions 206A, 206B may allow the user to re-orient or slightly rotate the grip portion between a thumb and one or more opposing fingers, rather than using the wrist to re-orient or rotate the entire handle. This provides an ergonomic benefit to the user and may help reduce arm fatigue. Furthermore, slight rotational movement of the first grip portion 206A and the second grip portion 206B may translate into movement of the distal tip. For example, in some variations, the first grip portion 206A and the second grip portion 206B may allow a limit of orientation by a user's grip alone to be about +/−30 degrees about a central longitudinal axis, without the user having to rotate at the wrist.

The first and second grip portions 206A, 206B (e.g., first and second curved sides) may each have a one or more radii of curvature between the top and bottom surfaces 240A, 240B. For example, the first and second grip portions 206A, 206B may each comprise a first, smaller radius of curvature near or adjacent the top surfaces 240A/240B and a second, larger radius of curvature between (e.g., midway) the top and bottom surfaces 240A/240B, allowing the grip portion 206 to ergonomically fit into the hand of the user, contacting the hand of the user at or along multiple points to increase control of the device. In some variations, one or more of the first and second grip portions 206A/206B may each comprise faceted faces (e.g., polygonal comprising two or more faces) from the first, top surface 240A to the second, bottom surface 240B opposite the top surface 240A. In some variations, the faceted faces may collectively comprise a curve having a radius of curvature within the ranges of radius of curvature described herein. The faceted faces of one or more of the first and second grip portion 206A/206B may be configured to control an angle of rotation of the grip portion within the hand of the user. It should be appreciated that while described in the preceding two paragraphs with respect to the first and second grip portions 206A, 206B, such features are also applicable to the first and second curved sides that may form, in some variations, the first and second grip portions respectively.

As illustrated in FIG. 2B, each of the actuators 232A/232B may be positioned on or through, or may otherwise extend from, the top and/or bottom surfaces 240A, 240B of the grip portion. The actuators 232A/232B may extend a defined distance from the top and bottom surfaces 240A/240B and/or may have a distinct shape so that the actuators 232A/232B are easily distinguishable from each of the surfaces 240A/240B themselves when the user is handling the grip portion 206. The top and bottom surfaces 240A/240B may be substantially flat or may otherwise have a large radius of curvature relative to other portions of the handle (e.g., grip portion, neck, proximal portion, thus allowing a user to easily rest a finger on the top and bottom surfaces 240A/240B, placing the finger close to but not on the actuators 232A/232B.

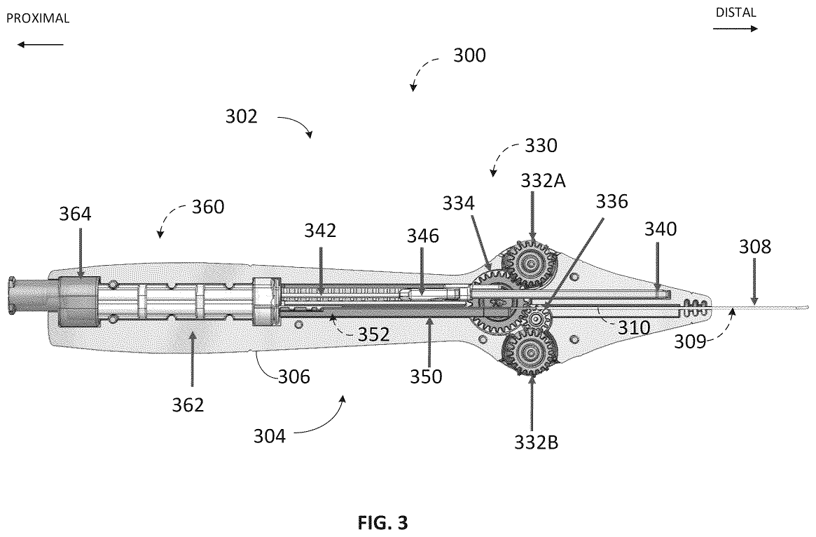

As mentioned above, generally, the handle may be configured such that a user may grasp the handle at the grip portion and still easily access the one or more actuators that may be used to actuate one or more linear gears 210 to move the elongate member 224 and/or deliver the fluid composition to Schlemm's canal. As depicted in FIG. 3, the one or more actuators may include at least two actuators (332A/332B), a first actuator 332A on a top side and a second actuator 332B on a bottom side.

In some variations, the device may be configured such that a user may grip the handle at the grip portion 206 to deliver a fluid composition in a clockwise direction in the eye and then with a simple rotation of the handle 202 (or by rotating the cannula itself 180 degrees in another variation), the device may deliver the fluid composition in the counterclockwise direction in the eye. Moreover, the device may be configured such that a user may grip the handle at the grip portion 206 to deliver a fluid composition in a clockwise direction or a counterclockwise direction without rotating the handle 202 or the cannula.

### Cannula

The cannula may be configured to provide easy and minimally traumatic access to Schlemm's canal, such as during a minimally invasive ab-interno procedure. As illustrated in FIG. 2, the cannula 208 may generally be coupled to and extend from the distal end of the housing 206 of the handle 202. In some variations, the cannula 208 may be fixedly attached to the distal end of the housing 206. In other variations, the cannula 208 may be rotatably attached to the distal end of the housing 206 (e.g., via a cannula actuator such as a rotatable hub or the like) to change the orientation of the tip of the cannula 208. In variations of the delivery systems in which the handle 202 is reusable and the cannula 208 is disposable, the cannula 208 may be removably attached to the distal end of the housing 206.

The cannula (e.g., cannula 208) may include multiple portions having different geometric configurations. For example, in some variations, the cannula (e.g., cannula 208) may comprise a proximal end, a straight portion and a curved portion distal to the straight portion, where the curved portion has a proximal end and a distal end, and a radius of curvature (ROC). In other variations, generally, the cannula may comprise a curved portion, where the curve portion may comprise a first curved portion and a second curved portion. The second curved portion may comprise a radius of curvature greater than the first curved portion. However, it should be appreciated that in other variations, the cannula may be entirely straight (e.g., may comprise only a straight portion and may not comprise a curved portion). The cannula may further include a distal tip through which a lumen extends from the proximal to the distal end—this lumen may be in fluid communication with any fluid assembly described elsewhere herein.

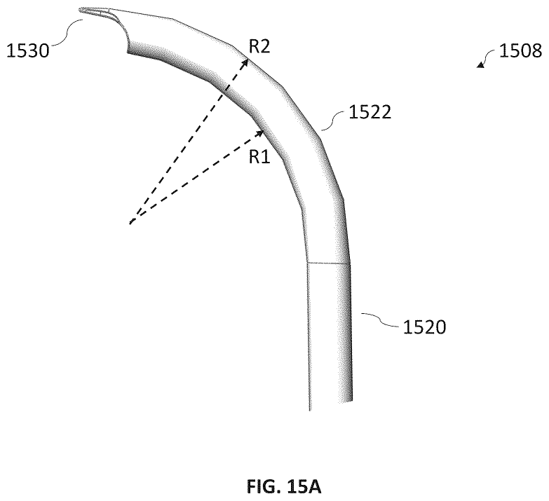

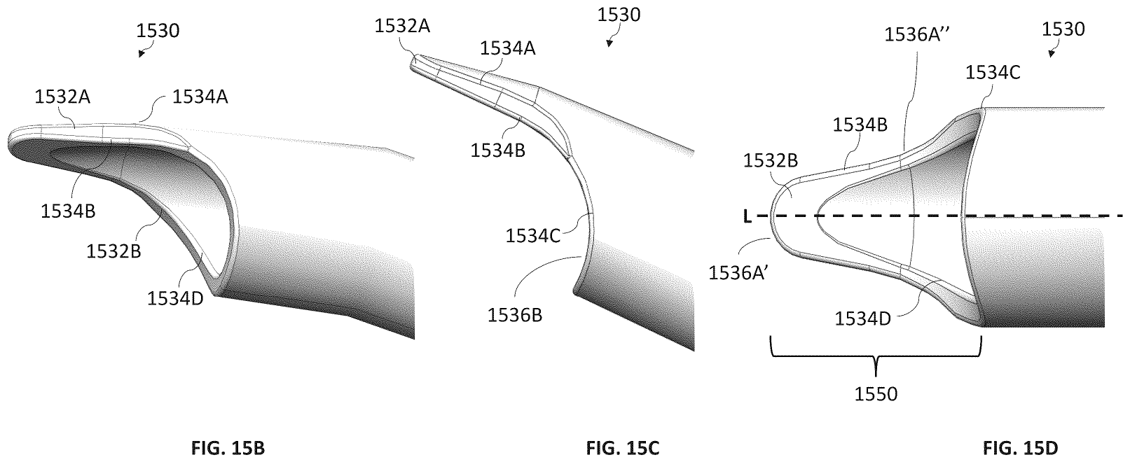

An illustrative variation of a cannula 1508 is provided in FIG. 15A. As shown, the cannula 1508 may comprise a straight portion 1520 and a curved portion 1522 that is distal to the straight portion 1520. The straight portion may extend from a proximal end (not shown) of the cannula 1508, which may be coupled to one or more components of a delivery device (e.g., a cannula actuator, a handle housing). The curved portion 1522 may comprise a first radius of curvature (ROC) defined by the inner radius R1 of its curve. Additionally, the curved portion 1522 may comprise a second ROC defined by the outer radius R2 of the curve. In some variations, the inner radius R1 may be less than the outer radius R2. A difference in length between R1 and R2 may be about 0.1 mm to about 1 mm, or R1 and R2 may be about the same length. In some variations, one or both of R1 and R2 may be about 1 mm to about 5 mm, such as about 1.5 mm to about 4 mm, about 2 mm to about 3.5 mm, or about 2.5 mm to about 3 mm (including all ranges and subranges therebetween). Additionally, the cannula 1508 may comprise a distal tip 1530 and a lumen. The lumen may extend from the cannula's proximal end through the distal tip 1530, passing through both the straight portion 1520 and the curved portion 1522.

In some variations in which the cannula may comprise a first curved portion and a second curved portion (not shown), the second curved portion may define a second inner radius R3 and a second outer radius R4. The second curved portion may be proximal of the first curved portion. A difference in length between the second inner radius R3 and the second outer radius R4 may be about 0.1 mm to about 1 mm or may be about the same length. In some variations, one or both of second inner radius R3 and second outer radius R4 may be about 10 mm to about 50 mm, about 20 mm to about 40 mm, about 25 mm to about 35 mm, or about 28 mm to about 32 mm (including all ranges and subranges therebetween).

The distal tip may be configured to traverse the trabecular meshwork as the cannula is advanced toward Schlemm's canal. In some variations, the distal tip may comprise one or more surfaces (e.g., angled surfaces) to facilitate tissue dissection. Further, the distal tip may comprise a rounded shape and/or blunt edges between the surfaces to enable minimally traumatic access to Schlemm's canal. Alternatively, the distal tip may further include a sharpened shape and/or sharpened edges to form a piercing tip. In some variations, the cannula (e.g., the distal tip of the cannula) may comprise a surface feature configured to receive at least a portion of an ocular device (e.g., implant) for delivery to and/or placement in the eye (e.g., Schlemm's canal). The surface feature may comprise, for example, one or more notches, hooks, slots, recesses, and the like that may receive at least a portion of the ocular device so that the ocular device may be advanced to, for example, Schlemm's canal, and positioned circumferentially therein to maintain the patency of at least a portion of the canal and/or otherwise facilitate fluid flow within/through the canal (e.g., transmural fluid flow across the canal). In some variations, the cannula may comprise a circumferential groove configured to engage with the ocular device.

Various close-up views of the distal tip 1530 are depicted in FIGS. 15B-15D. As shown, the distal tip 1530 may be elongated. In some variations, a width of the distal tip 1530 may increase proximally (or decrease distally) along a longitudinal axis (L, shown in FIG. 15D) of the distal tip 1530. The width variation of the distal tip may be linear or nonlinear. In some variations, at least a portion of the distal tip 1530 may comprise an arched or rounded shape. The distal tip 1530 may comprise a length 1550 of about 0.15 mm to about 1 mm, about 0.3 mm to about 0.6 mm, or about 0.4 mm to about 0.5 mm (including all ranges and subranges therebetween). Additionally, or alternatively, at least a portion of the distal tip 1530 may comprise pointed or beveled shape. In some embodiments, a first (e.g., distalmost) portion of the distal tip 1530 may be tapered at a first angle, and a second (e.g., most-proximal) portion of the distal tip 1530 may be tapered at a second, different (larger or smaller) angle. The distal tip 1530 may comprise one or more surfaces, such as first and second surfaces 1532A and 1532B, one or both of which may be oriented at an angle relative to the longitudinal axis and/or may extend substantially orthogonal to it. For instance, in some variations, the first surface 1532A may be inclined at a non-zero angle (e.g., less than or greater than 90 degrees), whereas the second surface 1532B may be substantially perpendicular (i.e., about 90 degrees) to the longitudinal axis. For example, the non-zero angle of the first surface 1532A may be about 1 degree to about 45 degrees, about 5 degrees to about 30 degrees, about 5 degrees to about 20 degrees, or about 8 degrees to about 12 degrees (including all ranges and subranges therebetween). The angle of the second surface 1532B may be about 60 degrees to about 120 degrees, about 60 degrees to about 100 degrees, about 65 degrees to about 90 degrees, about 70 degrees to about 80 degrees, or about 90 degrees (including all ranges and subranges therebetween). In some variations, one or both surfaces may include one or more curves or contours 1536A′, 1536A″, and 1536B. Curve 1536A′ may at least partially include surface 1532A and may include a radius of curvature of about 0.01 mm to about 0.2 mm, about 0.05 mm to about 0.15 mm, or about 0.07 mm to about 0.11 mm (including all ranges and subranges therebetween). Curve 1536A″ may at least partially include surface 1532A and may include a radius of curvature of about 0.1 mm to about 1 mm, about 0.3 mm to about 0.7 mm, or about 0.4 mm to about 0.5 mm (including all ranges and subranges therebetween). Curve 1536B may at least partially include surface 1532B and may include a radius of curvature of about 0.05 mm to about 0.5 mm, about 0.15 mm to about 0.35 mm, about 0.2 mm to about 0.3 mm, or about 0.23 mm to about 0.27 mm (including all ranges and subranges therebetween).

In some variations, the distal tip may additionally comprise edges 1534A, 1534B, 1534C, and 1534D. The edges 1534A/1534B/1534C/1534D may be formed between the first and second surfaces 1532A, 1532B, and/or between one of the first or second surfaces 1532A, 1532B and an internal or external surface of the cannula just proximal to the distal tip 1530. In some variations, one or more of the edges 1534A/1534B/1534C/1534D (e.g. all edges 1534A/1534B/1534C/1534D) may be blunt to enable gentle advancement through tissue. Alternatively, one or more of the edges 1534A/1534B/1534C/1534D (e.g. all edges 1534A/1534B/1534C/1534D) may be sharpened to facilitate tissue piercing.

Furthermore, the cannula may be made from any suitable material with sufficient stiffness and biocompatibility to allow it to be advanced through the anterior chamber and into Schlemm's canal. For example, the cannula may be formed of a metal such as stainless steel, titanium, aluminum, or alloys thereof (e.g., Nitinol metal alloy), a polymer, ceramic, or a composite. Exemplary polymers include without limitation, polycarbonate, polyetheretherketone (PEEK), PEKK, PEI, polyimide, polyamide, polysulfone, or fluoropolymers. In some instances, it may be advantageous to coat the cannula with, or otherwise incorporate into the cannula, a lubricious polymer to reduce friction between the ocular tissue and the cannula during the procedure. Lubricious polymers include, without limitation, hydrophilic coatings (such as polysaccharides), hydrophobic coatings, polyvinyl alcohol, polyethylene glycol, polyvinyl pyrrolidone, fluorinated polymers (including polytetrafluoroethylene (PTFE or Teflon®)), and polyethylene oxide. In some variations, lubricious polymers may be incorporated into the material of the cannula. For example, the cannula may comprise a lubricious additive formulation, where a chemical additive is compounded or otherwise added into a substrate or base material of the cannula, so that the chemical additive is present at a surface and throughout the material of the cannula. In some variations, the substrate or base material may include a polyamide (e.g., nylon). In variations in which the cannula is reusable, the cannula may be made from a material that can be sterilized (e.g., via autoclaving), such as a heat-resistant metal (e.g., stainless steel, aluminum, titanium).

The cannula may generally have an outer diameter sized to gain access to the lumen of Schlemm's canal while minimally obstructing the surgeon's view. Accordingly, the outer diameter may range from about 50 microns to about 1000 microns. In some variations, the outer diameter may range from about 150 microns to about 800 microns, from about 200 microns to about 700 microns, from about 300 microns to about 600 microns, or from about 400 microns to 500 microns. The cannula also has an inner diameter, which may range from about 50 microns to about 400 microns, from about 100 microns to about 350 microns, or from about 150 microns to about 300 microns. The cannula may also be formed to have any suitable cross-sectional shape, e.g., circular, elliptical, triangular, square, rectangular, or the like. In some variations, the cannula may comprise a tapered profile along its length.

### Elongate Member

Some variations of the delivery devices described herein may comprise an elongate member 310, as seen in FIG. 3 slidably positioned within the cannula 308, and more specifically, within a lumen 309 of the cannula 308. In some variations, the elongate member 310 may comprise a lumen and may be configured to deliver one or more fluid compositions. Additionally, or alternatively, the elongate member 310 may be configured to disrupt the trabecular meshwork and/or other similar tissues. In some variations, the elongate member 310 may be solid and may not comprise a lumen. In these variations, the elongate member 310 may still be configured to disrupt the trabecular meshwork and/or other similar tissues. In some variations the elongate member may be reinforced with one or more stiffening and/or stabilizing elements.

The elongate member 310 may be slidable within the cannula lumen 309 of the delivery systems described here. When the elongate member 310 is in a retracted position relative to the cannula 308, a distal end of the elongate member 310 may be located within (i.e., proximal to) a distal tip of the cannula 308. When the elongate member 310 is in an extended position relative to the cannula 308, the distal end of the elongate member 310 may be located outside of (i.e., distal to) the distal tip of the cannula 308. The length of extension of the elongate member 310 beyond the distal tip of the cannula 308 may correspond to the distance around Schlemm's canal that may be traversed by the elongate member 310 (e.g., in order to disrupt Schlemm's canal and/or surrounding trabeculocanalicular tissues, and/or to deliver a fluid composition). For example, the elongate member 310 may dilate, open, or otherwise modify Schlemm's canal and/or surrounding trabeculocanicular tissue, via the body of the elongate member and/or by delivery of a fluid composition therethrough. When a variation of the delivery systems described herein is used to deliver a fluid composition, the length traversed by the elongate member 310 may correspond to the length around Schlemm's canal to which the fluid composition is delivered. When a variation of the delivery systems described herein is used to mechanically tear or cut the trabecular meshwork independent of fluid delivery, the length traversed by the elongate member 310 may correspond to the length of trabecular meshwork that is cut or torn. In some variations, this length may be between about 1 mm and about 50 mm. In some of these variations, the length may be between about 10 mm and about 40 mm, between about 15 mm and about 25 mm, between about 16 mm and about 20 mm, between about 18 mm and about 20 mm, between about 19 mm and about 20 mm, between about 18 mm and about 22 mm, about 20 mm, between about 30 mm and about 50 mm, between about 35 mm and about 45 mm, between about 38 mm and about 40 mm, between about 39 mm and about 40 mm, or about 40 mm. In some variations, the length may be about 25 mm to about 50 mm including about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm, about 40 mm, about 41 mm, about 42 mm, about 43 mm, about 44 mm, about 45 mm, about 46 mm, about 47 mm, about 48 mm, about 49 mm, or about 59 mm. The elongate member 310 may be moved between extended and retracted positions using a drive assembly of the delivery device, described in more detail herein.

In some variations, the devices described herein may be configured to advance and/or retract the elongate member a distance from the cannula corresponding to between about a 1 degree arc and about a 540 degrees arc of Schlemm's canal, such as between about a 10 degrees arc and about a 420 degrees arc, between about a 30 degrees arc and about a 360 degrees arc, or between about a 60 degrees arc and about a 300 degrees arc (including all values and subranges therebetween).

The elongate member 310 may be configured to deliver a fluid composition. The fluid composition may travel through a lumen of the elongate member 310 and may be delivered through one or more openings of the lumen at the distal end of the elongate member. The fluid composition may be used to disrupt the trabecular meshwork and other surrounding tissues. In some variations, the fluid composition may be delivered while the elongate member is advanced away from the cannula. In some variations, the fluid composition may be delivered while the elongate member is retracted towards the cannula.

The elongate member 310 may be sized so that it can be advanced through the cannula 308 and into a portion of Schlemm's canal (e.g., 0 to 360 degrees of the canal). In some instances, the outer diameter of the elongate member may be configured to disrupt trabeculocanalicular tissues, stent, and/or apply tension to the canal, and/or deliver a fluid composition. The elongate member 310 may be made from any suitable material that imparts the desired flexibility and pushability for introduction of the elongate member through the eye wall, accessing Schlemm's canal, and/or navigation through other ocular tissue structures. For example, the elongate member 310 may comprise a polymer, composites of polymers and metal, or metals such as stainless steel (e.g., spring temper stainless steel), nickel, titanium, aluminum, shape-memory alloys (e.g., Nitinol), or alloys of any of the foregoing. The polymer may be reinforced with a stiffening and/or stabilizing element. Exemplary polymers for the elongate member include without limitation, polycarbonate, polyetheretherketone (PEEK), polyethylene, polypropylene, polyimide, polyamide, polysulfone, polyether block amide (PEBAX), fluoropolymers, and nylon.

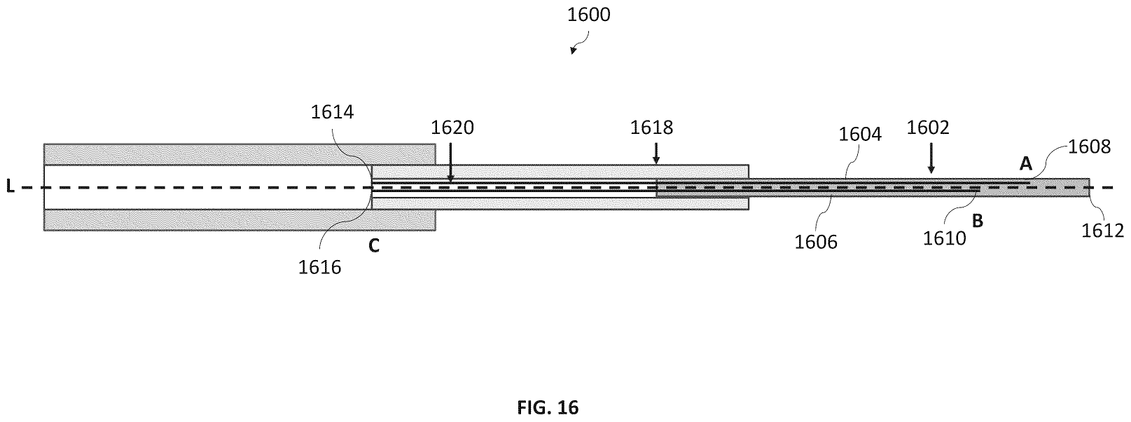

As mentioned above, in some variations, the elongate members described herein may comprise one or more stiffening and/or stabilizing elements configured to increase the stiffness of the elongate member to facilitate advancement of the elongate member around the eye while still maintaining the flexibility to avoid inadvertently damaging the delicate structures of the eye. Exemplary stiffening and/or stabilizing elements include, but are not limited to: a wire, a braid, a coil and a twisted ribbon, and may be made of or otherwise comprise any material suitable to impart stiffness or stabilize the elongate member during advancement and/or retraction, such as, for example, metals (e.g., stainless steel such as, for example, spring temper stainless steel), nickel, titanium, aluminum, shape-memory alloys (e.g., Nitinol), or alloys of any of the foregoing) and polymers (e.g., polycarbonate, polyetheretherketone (PEEK), polyethylene, polypropylene, polyimide, polyamide, polysulfone, polyether block amide (PEBAX), polymethylmethacrylate (PMMA) fluoropolymers, and nylon). In some variations, the stiffening and/or stabilizing element may be at least partially positioned within the lumen of the elongate member, and the stiffening and/or stabilizing element may or may not be constrained (e.g., axially constrained, rotationally constrained, transversely constrained, radially constrained). In some of these variations, the stiffening and/or stabilizing element may be floating in the lumen such that the stiffening element is not fixedly attached to a sidewall of the lumen. In some variations, the stiffening and/or stabilizing member may be flat or substantially planar. A width or diameter of the stiffening and/or stabilizing member may be between about 0.01 mm and about 0.05 mm. In some variations, the stiffening and/or stabilizing member may comprise a tapered width or diameter. Additionally, or alternatively, the stiffening and/or stabilizing member may comprise a curved profile. The stiffening and/or stabilizing member is described in further detail below (e.g., with respect to FIG. 16).

To easily access Schlemm's canal, in some variations it may be advantageous to coat all or a portion of the elongate member 310 with a lubricious coating (e.g., lubricious polymer coating) to reduce friction between the body of the elongate member 310 and the cannula and/or the ocular tissue, as the elongate member 310 moves within the cannula and/or the ocular tissue. In some variations, the lubricious coating may be hydrophilic. In other variations, the lubricious coating may be hydrophobic. In some variations, additionally or alternatively, the elongate member 310 may be composed of one or more materials that have a lower coefficient of friction than the ocular tissues that the elongate member 310 contacts (e.g., the tissue of Schlemm's canal) when used as intended, and/or a lower coefficient of friction than the material of the cannula.

In variations in which the elongate member 310 is reusable, the elongate member 310 may be made from a material that can be sterilized (e.g., via autoclaving), such as a heat-resistant metal (e.g., stainless steel, aluminum, titanium). The elongate member 310 may be straight with enough flexibility and pushability to navigate the ring-shaped Schlemm's canal or may be pre-shaped to about a 2-10 mm radius of curvature or about a 6 mm radius of curvature (i.e., the approximate radius of curvature of Schlemm's canal in an adult human) to more easily circumnavigate Schlemm's canal, partially or in its entirety. In some variations, the elongate member 308 may be configured to be advanced over or along a guidewire.

As noted above, in some variations, the elongate member may comprise one or more stiffening and/or stabilizing elements therein. For example, in some variations, the stiffening and/or stabilizing member may include a wire (e.g., a thin wire having a width or diameter less than a width diameter of the elongate member), which may comprise a shape-memory alloy such as nitinol. The stiffening and/or stabilizing element may help support kink resistance and prevent buckling as the elongate member is advanced or retracted through Schlemm's canal. In particular, the stiffening and/or stabilizing element may facilitate advancement of the elongate member when experiencing increased resistance, such as, for example, in some variations in which the elongate member is advanced past about 6 clock hours of travel around the eye. In some variations, the stiffening and/or stabilizing element may be at least partially positioned within a lumen of the elongate member. In these variations, as also noted above, the stiffening and/or stabilizing element may or may not be constrained (e.g., axially constrained, rotationally constrained, transversely constrained) within the lumen. For example, the stiffening element may be floating in the lumen such that stiffening and/or stabilizing element is not coupled to a sidewall of the lumen. The stiffening and/or stabilizing element may be positioned within the opening of the lumen. A stiffening element floating within the lumen may be coupled to the elongate member or support element at a proximal end of the stiffening element. In other variations, the stiffening element may be at least partially positioned within a wall of the elongate member (e.g., within a wall surrounding a lumen of the elongate member) (e.g., partially or entirely embedded therein). Additionally, or alternatively, the stiffening element may float within, or may be embedded in a wall of one or more other components of a delivery device, such as a support element (e.g., hypotube) configured to engage a proximal portion of the elongate member (as will be described in detail herein). That is, in some variations, a proximal end of the stiffening and/or stabilizing element may be coupled to a proximal end of the elongate member, while in other variations, the proximal end of the stiffening element may be coupled to at least a portion of a support element. In some variations, the stiffening element may be fixedly attached to the elongate member and/or support element (e.g., at opposite ends). In some variations, the stiffening element (e.g., proximal and distal ends thereof) may be attached using one or more of adhesives (e.g., epoxies or cements), friction fits, snap-fit or press-fit engagements, mechanical interlocks or threads, soldering, welding, and over molding/insert molding. For example, the proximal end of the stiffening element may be secured, e.g., using any of the above noted techniques, to a support element, such as, at a proximal end of the support element. The proximal end of the stiffening and/or stabilizing member may be secured within a lumen of the support element or between and outer surface of the support element and a component of the fluid assembly. The proximal end of the stiffening and/or stabilizing element may be secured proximal to a proximal end of the elongate member.

In some variations, the stiffening element(s) may only extend or otherwise be positioned within a portion of the elongate member. Put differently, in these variations, the stiffening and/or stabilizing element(s) may not extend through an entire length of the elongate member. For example, the stiffening element(s) may not reside within a distal portion of the elongate member. The distal portion of the elongate member may include one or more openings for delivering fluid to the canal. Thus because the stiffening and/or stabilizing element(s) do not reside in the distal portion, the distal portion (or any portion of the elongate member not containing the stiffening and/or stabilizing element) of the elongate member may be more flexible than the portion of the elongate member containing the stiffening and/or stabilizing element therein. In variations in which a distal portion of the elongate member may not include a stiffening and/or stabilizing element, a length of this distal, stiffening and/or stabilizing member-free portion may correspond to a particular travel distance of the elongate member around Schlemm's canal. For example, in some variations, the length of this distal portion may correspond to between about 1 to about 6 clock hours of travel around the eye, about 2 to about 4 clock hours of travel of the elongate member around the eye, or about 1 quadrant, about 2 quadrants (1 hemisphere), or about 3 quadrants of travel of the elongate member about the eye (e.g., Schlemm's canal). The distal portion may comprise about 1% to about 45% of a length of the elongate member, such as about 5% to about 40%, about 8% to about 35%, about 10% to about 30%, about 15% to about 25%, or about 18% to about 20% of the length of the elongate member (including all ranges and subranges therebetween).

Furthermore, the elongate member may comprise a plurality of stiffening elements, such as at least two, at least three, at least four, at least five, at least six, at least seven, at least eight, at least nine, or at least ten stiffening elements (e.g., one, two, three, four, five, six, seven, eight, nine, ten, or more than ten stiffening elements). In some variations, a plurality of stiffening elements may be positioned in a staggered configuration within the elongate member. For example, each of a plurality of stiffening elements may terminate at a different distal longitudinal position within the elongate member. Put another way, the distal ends of the stiffening elements may be longitudinally offset from each other. The proximal ends of the stiffening and/or stabilizing elements may be aligned or otherwise originate at about the same proximal longitudinal position proximal to or within the elongate member, or the proximal ends of the stiffening and/or stabilizing elements may also be offset from one another. In some variations, one or more of the stiffening and/or stabilizing elements may have a different length or may otherwise extend a different length within the elongate member, while in other variations, each of the stiffening elements may have the same length or may otherwise extend the same length within the elongate member.

The distal ends of each of the plurality of stiffening elements may be positioned within the elongate member at different positions along its longitudinal axis. In some variations, the distal end of a first stiffening element of the plurality of stiffening elements may be offset a first distance from the distal tip of the elongate member and a distal end of a second stiffening element of the plurality of stiffening elements may be offset a second distance. The second distance may be greater than the first distance. For example, the second distance may be about 1.5 times to about 2.5 times the first distance, or may be about double or triple the first distance. Put another way, the distal end of the first stiffening element may be about one half or one third the distance from the distal tip of the elongate member as a distal end of the second stiffening element. In some variations, each stiffening element may originate at a same longitudinal position within the elongate member or the support element (e.g., hypotube) configured to engage the elongate member. That is, the proximal ends of each stiffening element may be coupled to the elongate member or to the support element at about the same position along the longitudinal axis of the elongate member or support element. Alternatively, in some variations, the proximal ends of one or more of the stiffening elements may be longitudinally offset from one another.

FIG. 16 depicts a stylized, cross-sectional view of a portion of a delivery device 1600 comprising an elongate member 1602 that is reinforced with first and second stiffening and/or stabilizing elements 1604 and 1606. As shown, the first stiffening element 1604 may terminate at a first position (A) along a longitudinal axis (L) of the elongate member 1602, while the second stiffening element 1606 may terminate at a second position (B) along the longitudinal axis. In some variations, the second position (B) may be different than the first position (A). For example, the second position (B) may be proximal to the first position (A). The distance from the distal tip 1612 to the second position (B) may be about double the distance from the distal tip 1612 to the first position (A). The first position may correspond to a distal end 1608 of the first stiffening element 1604, and the second position may correspond to a distal end 1610 of the second stiffening element 1606. Thus, the distal ends 1608, 1610 of the first and second stiffening elements 1604 and 1606 may be staggered relative to each other. In some variations, the first and second positions (A, B) may be defined by degrees of an arc around the eye traveled by the distal tip 1612 of the elongate member 1602, or equivalently by corresponding clock hours of travel of the elongate member 1602 around the eye. For example, the length between the first position and distal tip may correspond to about 30 degrees (deg) to about 240 deg of travel of the elongate member around the eye, such as about 30 deg to about 210 deg, about 30 deg to about 180 deg, about 60 deg to about 180 deg, or about 60 deg to about 120 deg of travel of the elongate member around the eye (including all ranges and subranges therebetween). Put another way, the length between the first position and the distal tip may correspond to about 1 clock hour to about 8 clock hours of travel of the elongate member around the eye, such as about 1 clock hours to about 8 clock hours, about 1 clock hours to about 6 clock hours, about 2 clock hours to about 6 clock hours, or about 2 clock hours about 4 clock hours of travel around the eye. A length between the second position and the distal tip 1612 may correspond to about 60 deg to about 360 deg of travel of the elongate member around the eye, such as about 90 deg to about 330 deg, about 120 deg to about 300 deg, about 120 deg to about 240 deg, or about 150 deg to about 210 deg of travel around the eye (including all ranges and subranges therebetween). Put another way, the length between the second position and the distal tip 1612 may correspond to about 2 clock hours to about 12 clock hours, about 3 clock hours to about 11 clock hours, about 4 clock hours to about 10 clock hours, about 4 clock hours to about 8 clock hours, or about 5 clock hours to about 7 clock hours of travel of the elongate member around the eye. In some variations, the length between first position (A) and the distal tip 1612 may correspond to about 30 deg (i.e., about 1 clock hours) to about 120 deg (i.e., about 4 clock hours) of travel of the elongate member around the eye, such as about 30 deg, about 45 deg, about 60 deg, about 75 deg, about 90 deg, or about 120 deg of travel around the eye (including all ranges and subranges therebetween). In some variations, the length between the second position (B) and the distal tip may correspond to about 90 deg (i.e., about 3 clock hours) to about 240 deg (i.e., about 8 clock hours) of travel of the elongate member around the eye, such as about 90 deg, about 120 deg, about 150 deg, about 165 deg, about 180 deg, about 195 deg, about 210 deg, or about 240 deg of travel around the eye (including all ranges and subranges therebetween). In one example, the distance from the distal tip 1612 to the first position may correspond to about 75 deg/about 2.5 clock hours of travel of the elongate member around the eye, and the distance from the distal tip 1612 to the second position may correspond to about 150 deg/about 5 clock hours of travel of the elongate member around the eye. In another example, the distance between the distal tip and the first position may correspond to about 90 deg or about 3 clock hours of travel of the elongate member around the eye, and the distance between the distal tip and the second position may correspond to about 180 deg or about 6 clock hours of travel of the elongate member around the eye.

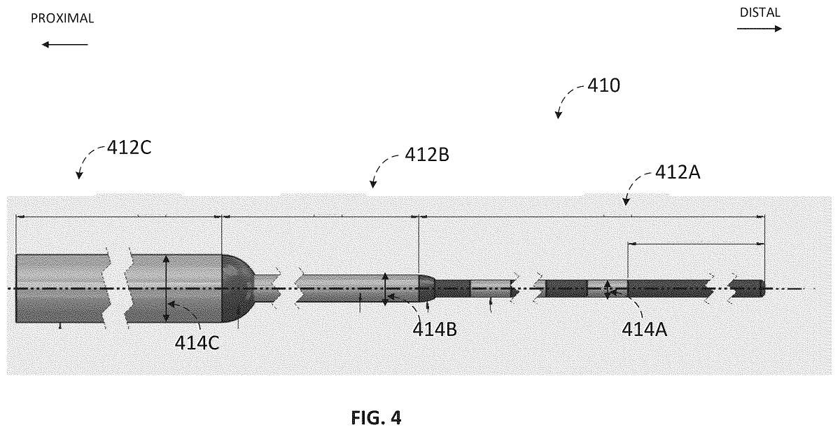

In some embodiments, the elongate member may comprise a support element (e.g., proximal tubular section) that allows the elongate member to be pushed from within the handle and carry a compressive load without buckling. In some variations, the proximal tubular section may comprise a diameter that is greater than a diameter of a distal section of the elongate member. The proximal tubular section may fit within a straight portion of the cannula and may not exit a distal end of the cannula. In some variations, the proximal tubular section may provide a larger internal lumen than a distal portion of the elongate member, where the larger internal lumen may provide a reduced resistance to flow of a fluid composition along a length of the proximal tubular section. For example, as depicted in FIG. 4, the elongate member 410 may comprise a proximal tubular section comprising a first length 412C and a second length 412B, and a distal section 412A. The first length 412C may have a first diameter 414C, the second length 412B may have a second diameter 414B, and the distal section 412A may have a third diameter 414A. The first diameter 414C and the second diameter 414B may be greater than the third diameter 414A. The elongate member 410 may comprise a lumen therethrough with the first diameter 414C and the second diameter 414B allowing for a larger internal lumen, and the distal section 412A with the third diameter 414A having a smaller lumen therethrough. As described above, the larger internal lumen may provide reduced resistance to fluid flow along the first length 412C and/or the second length 412B.

Turing back to FIG. 16, a proximal end 1614 of the first stiffening and/or stabilizing element 1604 and a proximal end 1616 of the second stiffening and/or stabilizing element 1606 may each be coupled within a lumen 1620 of a support element 1618. As shown in FIG. 16, the proximal ends 1614, 1616 may be coupled to the support element at a same third longitudinal position (C) within the delivery device. The support element 1618 may be configured to receive the elongate member 1602 within its lumen 1620 to stabilize and guide the elongate member 1602 as it is advanced by the delivery device 1600.

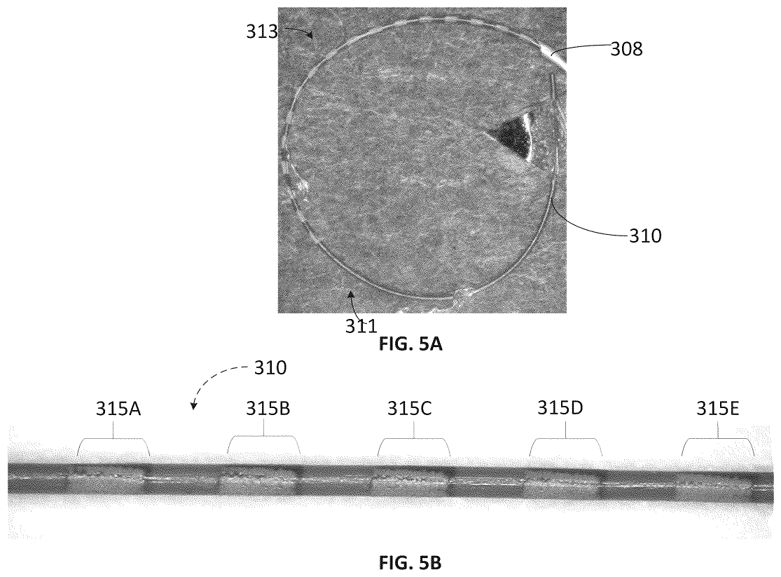

It may, in some variations, it may be desirable for the elongate member to have one or more features to improve visualization of the distal end of the elongate member as it is extended from the cannula. For example, as seen in FIG. 5A, the elongate member 310 may have one or more features to improve visualization of the elongate member 310 when the elongate member 310 is extended from the cannula 308. For example, the elongate member 310 may be colored (e.g., red, orange, yellow, green, blue, purple, etc.) and/or may have colored segments and/or distinguishable designs thereon spaced along the length of the elongate member 310. Additionally, or alternatively, visualization may be improved using an illuminated beacon, a fiber optic, side illuminating fiber optic, luminescence, fluorescence, or the like. For example, a fiber optic may travel along the body of the elongate member 310 to deliver light to the distal tip of the elongate member 310, which may improve visualization of the distal tip of the elongate member 310 as it is advanced or retracted about Schlemm's canal. Put differently, in some variations, a portion (e.g., distal end, central portion) of the elongate member 310 may be illuminated or may otherwise comprise an illumination device to assist in visualizing movement of the elongate member 310 within Schlemm's canal.

Additionally, or alternatively, in some variations, as seen in FIGS. 5A and 5B, the elongate member 310 may comprise one or more materials configured to react when etched with a laser to generate one or more markings. For example, the one or more materials may be added to the material used to form the elongate member 310 during manufacturing. The elongate member, with these one or more additional materials, may then be exposed to a laser at particular points or portions along the length of the elongate member to generate laser markings on the elongate member. Exemplary materials that can be used to facilitate generation of laser markings include but are not limited to UV-reactive additives compounded into a substrate material of the elongate member, a thin outer layer of material that is burned off through a reaction with one or more lasers, and/or the like. Additionally, or alternatively, photochromic ink, which changes color with UV exposure, may be used. The one or more markings may help create contrast between different portions of the elongate member without significantly modifying the material properties of the elongate member. In some variations, the one or more markings may include repeatable patterns (e.g., one or more of repeating lines, repeating bars, repeating dots, repeating shapes, repeating letters, repeating numbers or the like), such as repeating opaque bars (e.g., 315A-315E) as depicted in FIG. 5B. In some variations, the same repeatable pattern or different repeatable patterns may be depicted on the elongate member. It can be appreciated that any number of the element of the pattern (e.g., 3, 4, 5, 6 or more repeating lines) or any number of repeatable patterns or markings (e.g., 2, 3, 4, 5, or 6 distinct repeatable patterns) may be provided on (e.g., etched on) the elongate member.

In some variations, the one or more markings including repeatable patterns may be provided (e.g., etched) on only a portion of the elongate member or on the entirety of the elongate member. For example, the one or more markings may be provided on a proximal portion (e.g., proximal half, proximal third, proximal quarter, or the like, a distal portion (e.g., distal half, distal third, distal quarter, or the like), a portion therebetween, or the entire length. As seen in FIG. 5A, one or more markings are etched on a distal portion 313 of the elongate member 310 that is extended from the cannula 308. In some variations, the one or more markings provided the elongate member may indicate to the user to length of travel of the elongate member when the elongate member is deployed to Schlemm's canal. For example, the one or more markings on the elongate member seen in FIG. 5A are etched on a distal portion and the proximal portion 311 of the elongate member 310 is void of markings (e.g., is unetched). The unetched portion may correspond to a first length of travel around Schlemm's canal, while the etched portion with the one or more markings may correspond to a second length of travel or additional length of travel that may be the same or different from the first length of travel indicated by the unetched portion. For example, the unetched portion may indicate a first length of travel of approximately 180 degrees around Schlemm's canal so that when a user visualizes the one or more markings, the user knows the elongate member has traveled at least 180 degrees around Schlemm's canal. The markings may be positioned to indicate any desired first length of travel, including but not limited to about 10 degrees, about 15, degrees, about 20 degrees, about 30 degrees, about 40 degrees, about 50 degrees, about 60 degrees, about 70 degrees, about 80 degrees, about 90 degrees, about 100 degrees, about 110 degrees, about 120 degrees, about 130 degrees, about 140 degrees, about 150 degrees, about 160 degrees, about 170 degrees, about 180 degrees, about 190 degrees, about 200 degrees, about 210 degrees, about 220 degrees, about 230 degrees, about 240 degrees, about 250 degrees, about 260 degrees, about 270 degrees, about 280 degrees, about 290 degrees, about 300 degrees, about 310 degrees, about 320 degrees, about 330 degrees, about 340 degrees, about 350 degrees, or about 360 degrees. In some variations, the marking may be positioned to indicate any range of desired first length of travel including but not limited to from about 10 degrees to about 90 degrees, from about 10 degrees to about 180 degrees, from about 10 degrees to about 270 degrees, or about 10 degrees to about 360 degrees.

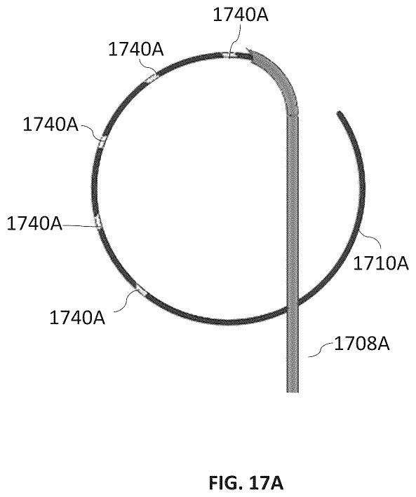

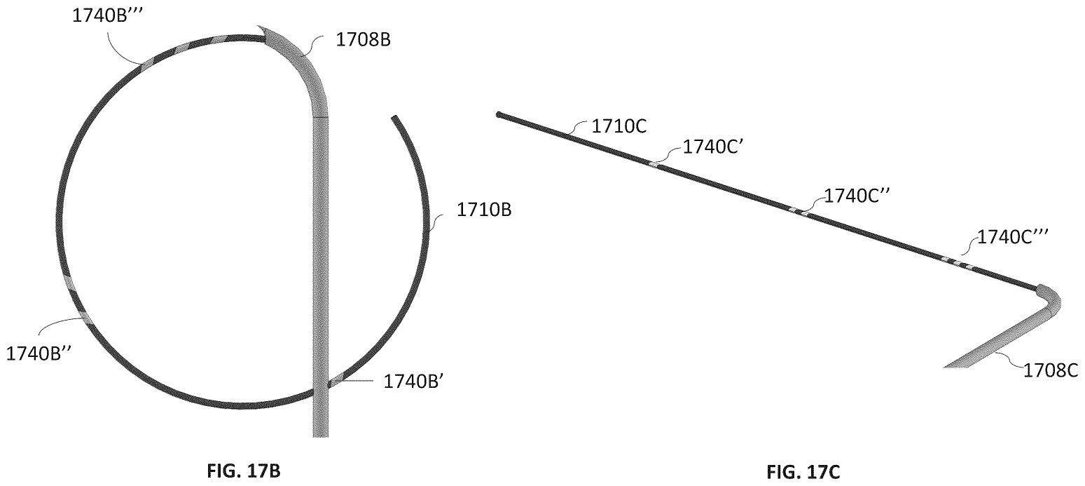

As an example, the markings may be positioned on the elongate member to indicate each quadrant of travel around Schlemm's canal. FIGS. 17A-17C depict perspective views of elongate members 1710A, 1710B, and 1710C in an advanced configuration relative to cannulas 1708A, 1708B and 1708C. In particular, FIG. 17A, which shows the elongate member 1710A in a first (curved) configuration, comprises markings 1740A, which may be evenly spaced along the elongate member 1710A. Each marking 1740A may a comprise the same pattern, such as a single symbol (e.g., line, dot, circle, etc.) or a different pattern. In some variations, the pattern may be formed at an angle (e.g., as a chevron) to preserve mechanical properties of the elongate member 1710A. Moreover, in some variations, a distance (or length of the elongate member 1710A) between each marking 1740A may correspond to a distance of travel of around the eye (e.g., Schlemm's canal). For example, the first (i.e., distalmost) marking may be positioned to indicate a number of clock hours traveled by a portion of the elongate member 1710A that is distal to the first marking. In some variations, the first marking may represent about 3 clock hours of travel, about 6 clock hours of travel, or about 9 clock hours of travel of the elongate member 1710A. The first (i.e., distalmost) marking may be positioned to indicate a number of quadrants traveled by a portion of the elongate member 1710A that is distal to the first marking. In some variations, the first marking may represent one quadrant (i.e., 90 deg or 3 clock hours), two quadrants (i.e., 180 deg or 6 clock hours), or three quadrants (i.e., 270 deg or 9 clock hours). As shown in FIG. 17A, the first marking may indicate two quadrants of travels around Schlemm's canal. Additionally, subsequent markings distal to the first marking may correspond to additional clock hours or quadrants of travel by elongate member 1710A. A distance (or length of the elongate member 1710A) between each marking 1740A may correspond to about 1 clock hour of travel (i.e., 30 deg), about 2 clock hours of travel (i.e., 60 deg), or about 3 clock hours of travel (i.e., 90 deg or 1 quadrant) of the elongate member 1710A around the eye.

FIG. 17B shows the elongate member 1710B in a first (curved) configuration, where the elongate member 1710B comprises markings 1740B. The markings 1740B may be evenly spaced along the elongate member 1710B. Each marking 1740B may a comprise a unique pattern. For example, as shown in FIG. 17B, each pattern may have a unique number of symbols (e.g., line, dot, circle, etc.). In some variations, a number of symbols patterned for each marking 1740B′, 1740B″, 1740B′″ may equal a number of quadrants traveled. In some variations, the pattern may be formed at an angle (e.g., as a chevron) to preserve mechanical properties of the elongate member 1710B. Additionally, a distance (or length of the elongate member 1710B) between each marking 1740B′, 1740B″, 1740B″−′ may correspond to one quadrant (i.e., 90 deg or 3 clock hours), and the first (i.e., distalmost) marking may be positioned to indicate a number of quadrants traveled by a portion of the elongate member 1710B that is distal to the first marking. For example, the first marking 1740B′ may represent one quadrant (i.e., 90 deg or 3 clock hours), two quadrants (i.e., 180 deg or 6 clock hours), or three quadrants (i.e., 270 deg or 9 clock hours). However, in some variations (and in contrast to the markings 1740A of FIG. 17A), the first marking 1710B′ may indicate one quadrant of travel around Schlemm's canal.

Similarly, FIG. 17C, which shows the elongate member 1710C in a second (elongated) configuration, comprises markings 1740C′, 1740C″, 1740C″, which may be evenly spaced along the elongate member 1710C. Here, each marking 1740C′, 1740C″, 1740C″ may comprise a unique pattern. For example, as shown in FIG. 17C, each pattern may have a unique number of symbols (e.g., line, dot, circle, etc.). In some variations, a number of symbols patterned for each marking 1740C′, 1740C″, 1740C′″ may equal a number of quadrants traveled. Additionally, or alternatively, each marking 1740C′, 1740C″, 1740C′″ may comprise a unique color. Like markings 1740A of FIG. 17A, the patterns may be formed at an angle to preserve mechanical properties of the elongate member 1710C. Additionally, a distance (or length of the elongate member 1710C) between each marking 1740C′, 1740C″, 1740C″″′ may correspond to one quadrant (i.e., 90 deg or 3 clock hours), and the first (i.e., distalmost) marking may be positioned to indicate a number of quadrants traveled by a portion of the elongate member 1710C that is distal to the first marking. For example, the first marking 1740C′ may represent one quadrant (i.e., 90 deg or 3 clock hours), two quadrants (i.e., 180 deg or 6 clock hours), or three quadrants (i.e., 270 deg or 9 clock hours). However, in some variations (and in contrast to the markings 1740A of FIG. 17A), the first marking 1710C′ may indicate one quadrant of travel around Schlemm's canal.

In some variations, the marked portion (e.g., etched portion) of the elongate member comprising the one or more markings may have a marking density (e.g., number of markings along a unit of length). The marked portion may have a fixed marking density (the gap between adjacent markings remains constant) and/or may have a variable marking density (gaps between adjacent markings vary) (e.g., a fixed marking density in one or more first sections of the marked portion and a variable marking density in one or more other sections, a fixed marking density for the entirety of the marked portion, a variable marking density for the entirety of the marked portion). In some variations, where the marked portion has a variable marking density, the number of markings along the unit of length may increase or may decrease. For example, the distance between the markings axially along the length of the marked portion (or a section thereof) may increase or decrease. In some variations, the distance between the markings may decrease along the length of the marked portion (or a section thereof) to indicate to the user how much the elongate member has traveled (e.g., out of the cannula, around the canal). For example, in variations in which the marking density increases (e.g., gaps between adjacent markings decrease), a user may understand how much of the elongate member has been extended from the cannula and/or traversed the canal based on the markings and in particular the marking density and/or changes thereto. In some variations, the marking density may decrease (a gap or distance between adjacent markings may increase along the length of the marked portion or a section thereof) to indicate to the user that further travel of the elongate member has occurred. In some variations, the one or more marking may include gradients of color(s) along the length of the marked portion (or a section thereof) to indicate further travel of the elongate member.

In some variations, the elongate member (310) may be sized to be advanced atraumatically (e.g., without trabecular meshwork disruption) through Schlemm's canal. In other variations, the elongate member 310 may be sized to have an outer diameter sufficient to disrupt Schlemm's canal and surrounding trabeculocanalicular tissues. The outer diameter may range from about 25 microns to about 1000 microns, from about 25 microns to about 500 microns, from about 50 microns to about 500 microns, from about 150 microns to about 500 microns, from about 200 microns to about 500 microns, from about 300 microns to about 500 microns, from about 200 microns to about 250 microns, from about 150 microns to about 200 microns, or from about 180 microns to about 300 microns. In some instances, it may be beneficial for the elongate member to have an outer diameter of about 240 microns, although portions (e.g., a distal tip) of the elongate member may differ in size and shape.

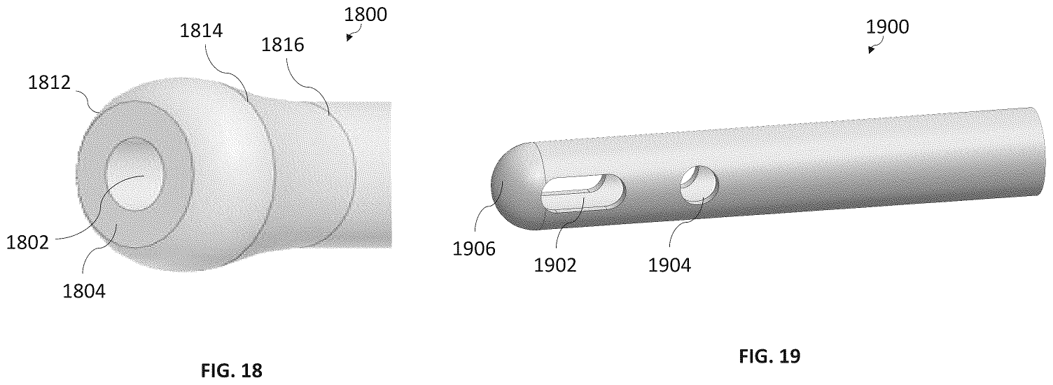

In some variations, the distal end of the elongate member 310 may be configured as a curved tip, a compound curved tip, an atraumatic tip, an enlarged atraumatic tip, a tapered tip, or the like, to help the elongate member (308) advance through Schlemm's canal. In some of these variations, the distal end may comprise a blunt parasol-shaped atraumatic tip. For example, FIG. 18 depicts a perspective view of an elongate member with enlarged atraumatic tip 1800 and a lumen 1802 configured for fluid delivery. The tip 1800 may comprise a football-like or diamond-like shape, a tapered distal end comprising a first outer diameter 1812, an enlarged central portion comprising a second outer diameter 1814, and a tapered proximal portion comprising a third outer diameter 1816. The first outer diameter 1812 may be less than the second outer diameter 1814. For example, the first outer diameter may be about 150 microns to about 260 microns while the second outer diameter may be about 240 microns to about 300 microns. Similarly, the third outer diameter 1816 may be less than the second outer diameter 1814. For example, the third outer diameter may be about 200 microns to about 260 microns while the second outer diameter may be about 240 microns to about 300 microns. The tapered distal end of the tip may facilitate atraumatic advancement of the elongate member within Schlemm's canal. The enlarged central portion (relative to the third outer diameter) may provide sufficient resistance for tearing of the meshwork using the elongate member. For example, the enlarged portion may partially anchor the tip such that the distal tip remains in place in the canal while the cannula is moved through/out of the anterior chamber. In some variations, the elongate member tip 1800 may comprise a flat surface 1804 at the distal end. Alternatively, the elongate tip 1800 may comprise a blunt or rounded surface at the distal end. For example, the first outer diameter 1812 may approach an inner diameter of the lumen 1802 such that the distal end of the tip 1800 comprises a more rounded profile.

In some variations, the elongate member (e.g., the distal tip of the elongate member) may comprise a surface feature configured to receive at least a portion of an ocular device (e.g., implant) for delivery to and/or placement in the eye (e.g., Schlemm's canal). The surface feature may comprise, for example, one or more notches, hooks, slots, recesses, openings and the like that may receive at least a portion of the ocular device so that the ocular device may be advanced to, for example, Schlemm's canal, and positioned circumferentially therein to maintain the patency of at least a portion of the canal and/or otherwise facilitate fluid flow within/through the canal (e.g., transmural fluid flow across the canal). In some variations, the elongate member may comprise a circumferential groove configured to engage with the ocular device.

In some variations, the elongate member tip may be configured as a capped tip and fluid may exit the elongate member laterally. The elongate member may include a plurality of lateral opening (e.g., one to four openings, two to eight openings, four to sixteen openings, or more than sixteen openings) configured to deliver fluid to the eye. The plurality of openings may be spaced along the length or a portion of the length (e.g., a distal portion, central portion, or distal portion) of the elongate member. FIG. 19 depicts a perspective view of an elongate member 1900 with a capped tip 1906. The capped tip may facilitate atraumatic advancement and retraction of the elongate member through Schlemm's canal. The elongate member 1900 may comprises a plurality of lateral openings 1902, 1904 configured to deliver fluid and an internal lumen (not shown) fluidly coupled to each of the plurality of openings. The cross-sectional shape of each of the plurality of openings may include a circle, oval, slot, triangle, square, star, pentagon, hexagon, octagon, any other polygon or the like. As shown in FIG. 19, a first set of lateral openings may comprise a slot shape and a second set of lateral openings 1904 may comprise a circular shape. In some variations, the plurality of lateral openings may comprise, and array or pattern of openings along the length or a portion of the length of the elongate member configured to evenly deliver fluid to the canal. For example, one or more openings may be placed in a helical pattern on the elongate member. In some variations, the lateral openings may be combined with an elongate member tip comprising an opening to an internal lumen at the distal end.

In other variations, a distal portion of the elongate member (308) may optionally include a disruptive component, e.g., a notch, hook, barb, a rough surface, or combination thereof, to disrupt the juxtatrabecular portion of Schlemm's canal or juxtatrabecular meshwork. One or more projections emanating from the elongate member (308) may further disrupt the juxtatrabecular portion of Schlemm's canal or juxtatrabecular meshwork and thus increase permeability of aqueous humor through the trabecular meshwork into Schlemm's canal. In some instances, the elongate member (308) may also deliver energy to the trabeculocanalicular tissues (e.g., ultrasonic energy, radiofrequency energy (e.g., for electrocautery, electroablation), electromagnetic radiation, light energy (e.g., via a fiber optic)). In some variations, the distal end of the elongate member may be configured as any of the configurations or combinations of the configurations previously described in U.S. Patent Publication No: US 2024/0225894 A1 published on Jul. 11, 2024, the contents of which are incorporated herein in their entirety. In some variations, a distal portion of the elongate member may be configured to be disassociated from the remainder of the elongate member and retained in Schlemm's canal as an ocular device (e.g., implant) configured to maintain patency of at least a portion of Schlemm's canal. The distal portion of the elongate member may be configured to be disassociated in any suitable manner, including, for example, by cutting, releasing or otherwise separating the distal portion from the remainder of the elongate member using the distal tip of the cannula or a separate device (e.g., separate cutting tool).

### Drive Assembly

As described above, the devices described herein may generally comprise a drive assembly that may be used to advance and retract the elongate member and deliver a fluid composition to the eye. Turning back to FIG. 3, depicted there is an exemplary delivery device 302 comprising a drive assembly. In some variations, the delivery device may comprise a handle 304 having a housing 306. A cannula 308 may be coupled to and may extend from the distal end of the housing 306 of the handle 304. The fluid delivery device 302 may further include an elongate member 310 slidably positioned within the cannula 308. The fluid delivery device 302 may further comprise a drive assembly 330 at least partially contained within the housing 306 of the handle 304. The drive assembly 330 may comprise one or more actuators configured to be contacted by a user and one or more additional actuators, such as linear gears, configured to translate motion from the one or more actuators configured to be contacted by a user into motion of internal components of the delivery device to ultimately move the elongate member and/or deliver a fluid composition from the fluid reservoir. In some variations, the one or more linear gears may be configured to translate rotational movement of the one or more actuators into linear movement, which may be used to move one or more internal components of the delivery device.

For example, as shown in FIG. 3, the drive assembly 330 may comprise a first linear gear 340 and a second linear gear 350. In some variations, the first linear gear 340 and the second linear gear 350 may be configured to move in opposing directions to facilitate movement of the elongate member and delivery of a fluid composition during the movement of the elongate member. In some variations, the first linear gear 340 and the second linear gear 350 may be configured to move the same distance in opposing directions that may result in advancement of the elongate member and delivery of a fluid composition from the fluid reservoir or retraction of the elongate member and delivery of a fluid composition from the fluid reservoir. Conversely, in some variations, the first and second linear gears 340, 350 may be configured to move in a same direction to facilitate movement of the elongate member and delivery of a fluid composition during the movement of the elongate member. In some variations, the first and second linear gears 340, 350 may be configured to move simultaneously. For example, in some variations, the first linear gear 340 may be configured to move in a first direction and the second linear gear 350 may be configured to move simultaneously in a second direction (which may or may not be opposite the first direction) upon actuation of the one or more actuators configured to be contacted by the user. Such actuation of the one or more linear gears may move the elongate member and/or deliver a fluid composition from the fluid reservoir. In particular, movement of the first and/or second linear gears may advance and/or retract the elongate member. In some variations, the movement of the first and/or second linear gears may be configured to advance and/or retract the elongate member a distance from the cannula corresponding to between about a 1 degree arc and about a 540 degrees arc of Schlemm's canal, such as between about a 10 degrees arc and about a 420 degrees arc, between about a 30 degrees arc and about a 360 degrees arc, or between about a 60 degrees arc and about a 300 degrees arc (including all values and subranges therebetween). Additionally, or alternatively, movement of the first and/or second linear gears may deliver a fluid composition from the fluid reservoir to the eye. For example, movement of the first linear gear may move a displacement rod into the fluid reservoir to deliver fluid and movement of the second linear gear may move a plunger tube into the fluid reservoir to deliver fluid. In some variations, both the first and the second linear gears may move during one or more of: advancement of the elongate member, retraction of the elongate member, re-advancement of the elongate member, re-retraction of the elongate member, and delivery of a fluid composition from the fluid reservoir to the eye. Put differently, in some variations, actuation of the actuator configured to be contacted by the user may result in movement of both the first and the second linear gears, which may result in one or more of: advancement of the elongate member or retraction of the elongate member, and delivery of a fluid composition from the fluid reservoir to the eye. In some variations, actuation of the actuator configured to be contacted by the user may result in movement of the both the first and second linear gears (e.g., simultaneously and in opposing directions), which may result in advancement of the elongate member and delivery of a fluid composition from the fluid reservoir or retraction of the elongate member and delivery of a fluid composition from the fluid reservoir. In some variations, the volume of fluid delivered during advancement and fluid delivery may be different than (e.g., less than, more than) the volume of fluid delivered during retraction and fluid delivery.

In some variations, the drive assembly 330 (e.g., via the one or more actuators configured to be actuated by a user) may be configured to be actuated by the hand of a user to both generate movement (e.g., advancement and retraction) of the elongate member 310 and deliver a fluid composition to the eye. The drive assembly 330 may comprise one or more actuators (e.g., one, two, three, four, or more) configured to be contacted by a user, such as, for example, a rotatable element (e.g., wheel), slide, button, or the like, actuation of which (e.g., rotation, translation, depression) may advance and retract the elongate member and/or may deliver the fluid composition by way of the first linear gear and the second linear gear. For example, as shown in FIG. 3, the drive assembly 330 may include a first actuator configured to be contacted by a user 332A and a second actuator configured to be contacted by a user 332B. In some variations, such as that shown in FIG. 3, the first and second actuators 332A, 332B may both be rotatable wheels. The first actuator 332A and the second actuator 332B may each contact one or more gears 334, 336 to translate rotational movement of the actuators 332A-332B into linear movement of the linear gears 340, 350, as will be described in more detail herein.

In some variations, the one or more actuators configured to be contacted by a user 332A-332B may extend out of the housing 306 to facilitate user access. For example, the one or more actuators 332A-332B may extend out of the housing 306 of the handle 304, such as, on opposing sides of the handle 304 as depicted in the variation shown in FIG. 3. The handle 304 may be configured (e.g., the size, shape including curvatures, placement of portions/components (e.g., actuators of the handle, etc.) to ergonomically fit within the hand of a user and provide easy and comfortable access to the actuators 332A-332B, and optionally rotation of the handle 304 itself along a longitudinal axis of the handle 304, thus allowing the user to easily control the movement of the elongate member 310 and fluid delivery.

### Cannula Actuator

In some variation, the handle may include a cannula actuator configured to be contacted by a user to rotate the cannula about a longitudinal axis of the handle. The cannula may be rotatably coupled to the distal end of the handle and may be rotated by actuation the canula actuator by a hand of a user. The rotation of the cannula may accommodate a user preference of an angle of the cannula relative to the handle. Furthermore, the cannula actuator may allow a user to change between delivering a fluid composition to an eye in a clockwise direction and delivering a fluid composition to the eye in a counterclockwise direction by rotating the cannula about 180 degrees. The cannula actuator may be configured as one or more of a knob, a rotatable hub, a button, a dial, a wheel, a slide, and the like configured to be engaged by the hand of the user.

FIG. 20A depicts a perspective view of a delivery device including a cannula actuator 2010 coupled to the handle 2002. As seen in FIG. 20A, the cannula 2008 may be coupled to a cannula actuator 2010. In some variations, the cannula actuator 2010 may comprise a secured configuration and a rotatable configuration. For example, the cannula actuator 2010 may be transitioned from the secured configuration to the rotatable configuration by distal movement of the cannula actuator 2010 and may be transitioned from the rotatable configuration to the secured configuration by proximal movement of the cannula actuator 2010. In some variations, the handle 2002 may comprise a plurality of teeth 2004 (e.g., two, three, four, six, eight, more than eight teeth) configured to secure the orientation of the cannular actuator 2010 in the secured configuration. The plurality of teeth 2002 may engage one or more teeth of the cannula actuator 2012 to define a plurality of predetermined cannula orientations. In some variations, the cannula actuator 2010 may be biased toward the secured configuration. For example, the cannula actuator 2010 may comprise a spring (not shown) configured to bias the actuator toward the secured configuration (e.g., engagement with the teeth of the handle). In other variations, the cannula actuator may comprise one or more detents to secure the rotation of the cannula. In some variations, rotation of the cannula 2008 rotates the elongate member. Rotation of the cannula 2008 and elongate member together my preserve an orientation of the elongate member relative to the cannula 2008. As show by FIG. 20A, a device with a rotatable cannula 2008 may facilitate use by the left and right hand with a single actuator 2032. A handle 2002 with a single actuator 2032 may comprise a lower profile configured to improve visualization of the cannula 2008 by the user.

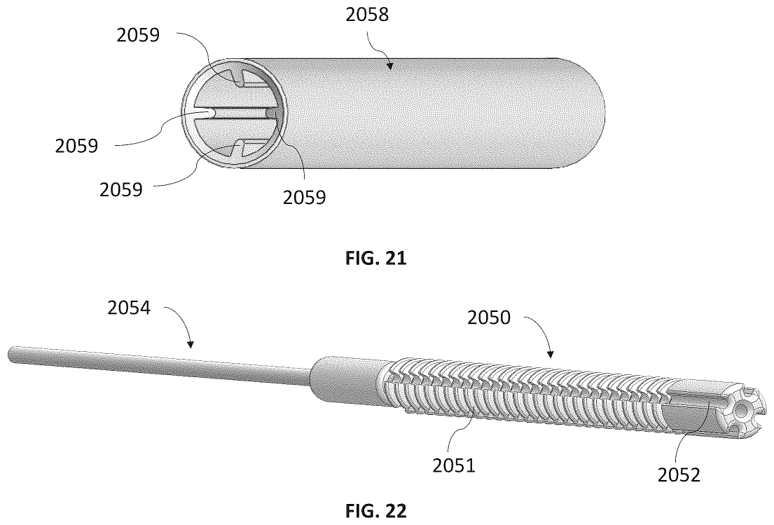

In some variations, rotation of the cannula rotates at least a portion of the drive assembly. More specifically, actuation of the cannula actuator may cause one or more of the cannula, the elongate member (e.g., conduit, such as a slidable conduit), the second linear gear (e.g., plunger tube rack), and the plunger tube to rotate about a longitudinal axis of the handle. In some variation, the cannular actuator may engage one or more of the plunger tube and the second linear gear to rotate the elongate member. FIG. 20B depicts a perspective view of the drive assembly and cannula actuator 2010 of the delivery device of FIG. 20A. FIG. 20C depicts a cross-sectional view of the drive assembly and cannula actuator 2010 of the delivery device of FIG. 20A. In some variations, as shown by FIGS. 20B-20C, the drive assembly may comprise a sheath 2058 configured to couple the cannula actuator 2010 to the second linear gear 2050. The cannula actuator 2010 may be coupled to the sheath 2058 and the sheath 2058 may be slidably coupled to the second linear gear 2050 such that rotation of the cannula actuator 2010 rotates the second linear gear 2050. As shown in FIG. 21, in some variations, the sheath 2058 may comprise one or more internal splines 2059. The one or more internal splines 2059 may be configured to engage and rotate the second linear gear 2050. In some variations, as shown in FIG. 22, the second linear gear 2050 may comprise one or more grooves 2052 configured to engage one or more internal splines 2059 of the sheath. The linear gear 2050 may be coupled to the plunger tube 2054. In some variations, the second linear gear 2050 may comprise a plurality of circumferential teeth 2051 configured to maintain engagement with a pinion gear 2036 of the drive assembly. Additionally or alternatively, the second linear gear may comprise a helical gear configured to engage the pinion gear.

The fluid delivery device 302 of the delivery system 300 may further comprise a fluid assembly 360 comprising a fluid reservoir 362. The fluid reservoir 362 may be at least partially contained within the housing 306 of the handle 304 and in this manner, may be configured to store a fluid composition for delivery to the eye at least partially within the handle of the delivery device. In some variations, the fluid reservoir 362 may be completely contained within the housing 306 of the handle 304, while in other variations, the fluid reservoir 362 may extend out of the housing 306 of the handle (e.g., proximally beyond a proximal end of the housing 306, from the top of the housing 306, from the bottom of the housing 306, laterally out of the housing 306 at any angle between the top and bottom of the housing 306). In some variations, the fluid reservoir 362 may be stationary within the handle 304 and other components of the fluid assembly may be configured to move relative to the fluid reservoir 362 to facilitate delivery of the fluid composition from the fluid reservoir 362 to the eye. In other variations, the fluid reservoir 362 may be moveable within the handle 304 and one or more other components of the fluid assembly may be configured to remain stationary within the handle during fluid delivery. In some variations, the fluid reservoir 362 may include a fluid reservoir connector 364 configured to detachably couple to an external fluid delivery device. The external fluid delivery device may be configured to deliver a first volume of fluid to the fluid reservoir 362, then detach from the fluid reservoir connector 364 upon completion of delivering the volume of fluid to the fluid reservoir 362. The fluid reservoir 362 may then hold and store the volume of fluid until delivery the volume of fluid to the eye. In other variations, the fluid reservoir may be provided pre-loaded with a fluid composition such that a fluid reservoir connector 364 may not be needed or included. In some variations, the fluid reservoir may be incrementally and/or controllable moveable to achieve displacement of the fluid from the fluid reservoir to the eye. For example, movement of the fluid reservoir may be controlled by a user, where a user actuates movement of the fluid reservoir such as, for example, via an actuator configured to be contacted by a user positioned on and/or within the handle. In some variations, the fluid reservoir may be moved in an incremental fashion (e.g., through the aforementioned actuator), with movement of the fluid reservoir displacing a predefined volume of fluid at a user-selected location per certain amount of movement of the actuator.

The fluid assembly 360 may further comprise components configured to interact with the fluid reservoir and thereby assist with delivering the fluid composition from the fluid reservoir to the eye. For example, the fluid assembly 360 may comprise a plunger tube 352 and a displacement rod 342, each configured to move relative to (e.g., within) the fluid reservoir 362 to deliver the fluid composition from the fluid reservoir to the eye. Generally, each of the plunger tube and the displacement rod may independently move within the fluid reservoir to deliver a volume of fluid from the fluid reservoir to the eye. In some variations, the plunger tube and the displacement rod may be actuated by the drive assembly to move relative to the fluid reservoir to deliver the fluid composition from the fluid reservoir to the eye. In some variations, as will be described in more detail herein, the displacement rod may move in a first direction a single time, while the plunger tube may move in the first direction, a second direction opposite the first direction, and may cycle between the two directions numerous times. For example, in some variations, the displacement rod may only move into the fluid reservoir a single time but not out of the fluid reservoir, while the plunger tube may move into and out of the fluid reservoir any number of times.

In some variations, one or more geometric differences between the displacement rod 342 and the plunger tube 352 may be used to generate a volume of fluid delivered from the fluid reservoir that will be described in more detail herein.

The drive assembly may convert an external input (e.g., motion of a user's thumb or finger) into motion of one or more components of the fluid delivery system. More specifically, actuation of the drive assembly may cause the elongate member (e.g., conduit, such as a slidable conduit) to extend distally out of the cannula and to retract proximally into the cannula. The drive assembly may also cause a fluid composition to be delivered from the fluid reservoir through the elongate member 410 and/or cannula 408 and ultimately to the eye. As described above, the drive assembly may generally comprise one or more first actuators (e.g., rotatable components, translatable components, depressible components) configured to be contacted by the user and one or more additional actuators (e.g., gears such as circular gears, linear gears) configured to translate motion from the one or more first actuators into motion of the one or more additional actuators (e.g., rotational motion of a circular gear, linear motion of a linear gear) to move the elongate member and/or deliver fluid from the reservoir.

Generally, the fluid reservoir may be stationary within the handle and may be positioned proximally within the handle (e.g., at a proximal end) and the drive assembly may be positioned distally of the fluid reservoir. Actuation of the drive assembly may move components of the drive assembly away from and towards the fluid reservoir. For example, actuation of the drive assembly via the one or more actuators configured to be contacted by a user in a first direction may move a first linear gear towards the fluid reservoir and a second linear gear away from the fluid reservoir, while actuation of the drive assembly via the one or more actuators in a second direction opposite the first direction may move the first linear gear away from the fluid reservoir and the second linear gear towards the fluid reservoir.

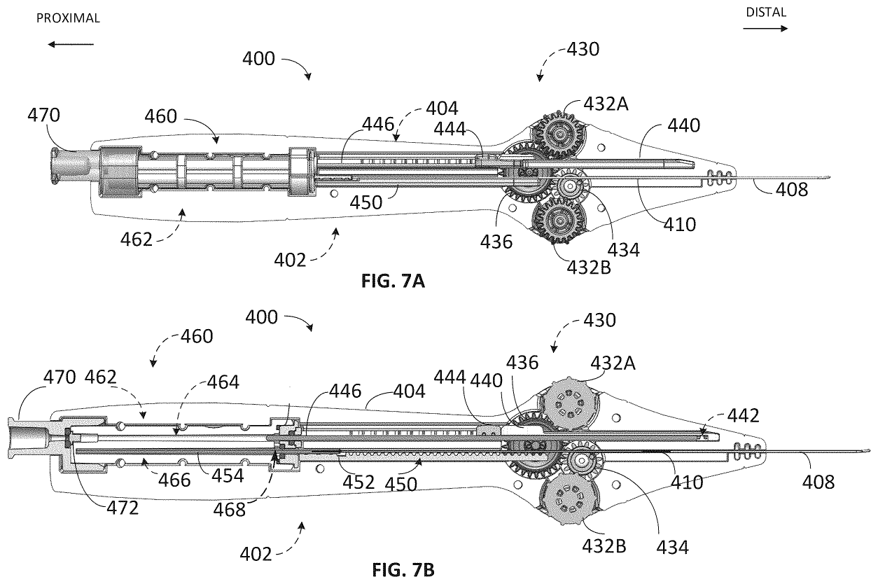

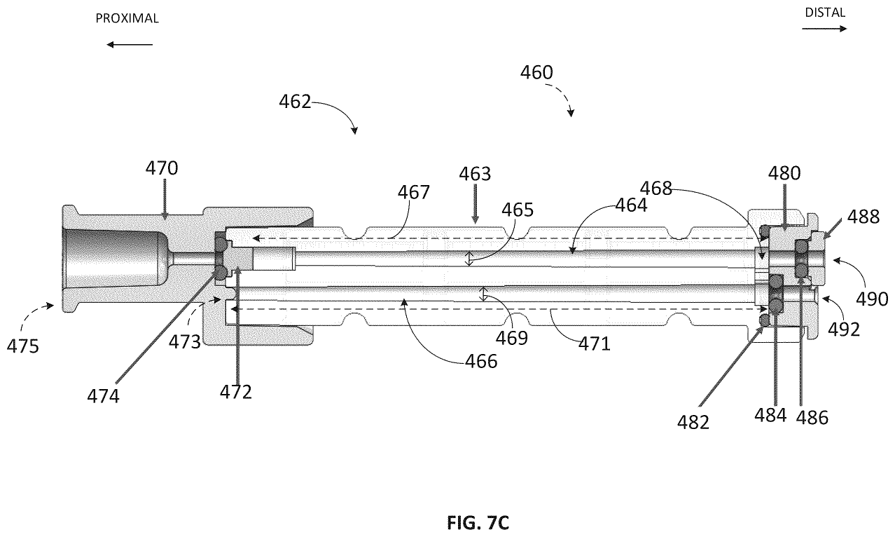

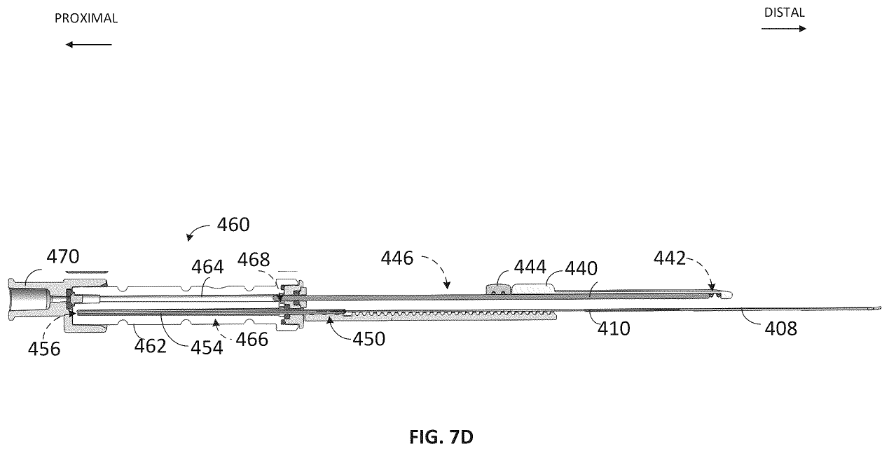

As seen in FIG. 7A, the delivery device 400 may include a drive assembly 430 configured to move the elongate member 410 and/or deliver a fluid composition into Schlemm's canal. The drive assembly 430 may be at least partially contained within the housing 404 and may include any suitable component or combination of components capable of providing the handle 402 with universal functionality, such that a user may use the device in either hand.

The first linear gear may be in contact with a ratchet coupled to the displacement rod. The second linear gear may be coupled to the elongate member and the plunger tube so that movement of the first linear gear in a first direction moves the ratchet and the displacement rod. Movement of the first linear gear in a second direction opposite the first direction does not move the ratchet and the displacement rod. Movement of the second linear gear in a first direction may move the plunger tube and the elongate member is a first direction. Movement of the second linear gear in a second direction opposite the first direction may move both the plunger tube and the elongate member in the second direction. With the first linear gear and second linear gear moving simultaneously in opposing directions, movement of the first linear gear in a first direction may move the ratchet and displacement rod in the first direction. The second linear gear moves in a second direction opposite direction with the plunger tube and elongate member moving in the second direction. Movement of the first linear gear in the second direction opposite the first direction may not lead to movement of the ratchet and the displacement rod, while the second linear gear, plunger tube, and elongate member may move in the first direction opposite the second direction.

Each of these effects (i.e., advancement of the elongate member 410, retraction of the slidable elongate member 410, and delivery of a fluid composition), or any combination of these effects, may be actuated using the same actuator or actuators. Utilizing the same actuator or actuators may allow for easier and more precise single-handed use of the delivery device. For example, movement of the actuator in a first direction (e.g., rotation of a rotatable element in a first direction, linear movement of an actuator, or the like) may cause extension (e.g., advancement in a distal direction) of the elongate member 410, and movement of the actuator in a second direction opposite the first direction (e.g., rotation of a rotatable element in a second direction, linear movement of an actuator, or the like) may cause retraction (e.g., movement of in the proximal direction) of the elongate member 410. Movement of the actuator in the first direction may actuate a first linear gear 440 in a first direction and may simultaneously actuate a second linear gear 450 in a second direction opposite the first direction of the first linear gear 440. Movement of the actuator in the second direction opposite the first direction may actuate the first linear gear 440 in a second direction and may simultaneously actuate the second linear gear 450 in a first direction opposite the second direction of the first linear gear 440. In some variations, moving the actuator in a first direction may move the first linear gear 440 and the second linear gear 450 equal distances in opposing directions. The movement of the first linear gear 440 and the second linear gear 450 equal distances in opposing directions may be configured to allow the device 400 to deliver a fluid composition during advancement of the elongate member 410 and retraction of the elongate member 410 as will be described in more detail herein. It can be appreciated that movement of the actuator in a first direction may allow the first linear gear 440 to be fully distally positioned within the handle 402 and the second linear gear 450 to be fully proximally positioned within the handle 402, such as, adjacent to the fluid reservoir 462. Movement of the actuator in a second direction opposite the first direction may allow the first linear gear 440 to be fully proximally positioned within the handle 402, adjacent to the fluid reservoir 462, and the second linear gear 450 to be fully distally positioned within the handle 402. Movement of the actuators may move the first linear gear 440 and the second linear gear 450 between these two configurations (fully distally positioned within the handle and fully proximally positioned within the handle (e.g., adjacent to the fluid reservoir)) and thus, may advance the elongate member 410 and retract the elongate member 410.

In some variations, the first time (e.g., a first pass) the first linear gear 440 is moved from fully distally positioned within the handle 402 to fully proximally positioned within the handle 402 adjacent to the fluid reservoir 462, the first linear gear 440 may advance the displacement rod 446 within a first lumen as will be discussed in more detail herein. The first linear gear may advance the displacement rod 446 by contacting a ratchet 444 coupled to the displacement rod 446. Continuing to move the first linear gear 440 from fully distally positioned within the handle to fully proximally positioned within the handle 402 after the first full pass between the proximal-most and distal-most positions may not move the displacement rod within the fluid reservoir 462. The position of the displacement rod 446 with in the fluid reservoir may be maintained by the ratchet 444.

When the second linear gear 450 is moved from fully proximally positioned within the handle 402 to fully distally positioned within the handle 402 adjacent to the fluid reservoir 462, the second linear gear 450 may advance the plunger tube 454 out of the first lumen to deliver fluid during advancement of the elongate member 410. When the second linear gear 450 is moved from fully distally positioned within the handle 402 to fully proximally positioned within the handle 402, the second linear gear 450 may retract the plunger tube 454 into the second lumen to deliver fluid during retraction of the elongate member 410.

In some variations, the drive mechanism 430 may be configured to allow the delivery system to be used multiple times—that is, the drive mechanism 430 may allow for, for example, advancement of the elongate member 410, retraction of the elongate member 410, re-advancement of the elongate member 410, and re-retraction of the elongate member. In some variations, the elongate member may only be advanced a predetermined amount before retraction is required before further advancement can occur. In some variations, some portion of movement (e.g., advancement, retraction, re-advancement, re-retraction) of the elongate member may be coupled with fluid delivery and some portions of movement of the elongate member may be decoupled from fluid delivery, while in other variations all movement of the elongate member 410 may be coupled with fluid delivery or all movement of the elongate member may be decoupled from fluid delivery.

For example, in some variations, advancement of the elongate member a first predetermined distance may be coupled with fluid delivery while re-advancement of the elongate member, after having been advanced the first predetermined distance, may be decoupled from fluid delivery. Similarly, retraction of the elongate member a first predetermined distance may be coupled with fluid delivery, while re-retraction of the elongate member, after having been retracted the first predetermined distance, may be de-coupled from fluid delivery. Put differently, after a predetermined amount of advancement and/or retraction of the elongate member, the elongate member may be re-advanced and/or re-retracted without any additional fluid delivery.

However, in some variations, advancement of the elongate member a first predetermined distance may be coupled with fluid delivery, retraction of the elongate member a first predetermined distance may be coupled with fluid delivery, re-advancement of the elongate member after the predetermined distance may be decoupled with fluid delivery, but re-retraction of the elongate member after the predetermined distance may still be coupled with fluid delivery. Put differently, after a predetermined amount of advancement and/or retraction of the elongate member, the elongate member may be re-retracted while still providing additional fluid delivery. In other variations, as will be discussed in more detail herein advancement of the elongate member a first predetermined distance may be coupled with fluid delivery, retraction of the elongate member a first predetermined distance may be coupled with fluid delivery, re-advancement of the elongate member after the predetermined distance may be coupled with fluid delivery, and re-retraction of the elongate member after the predetermined distance may be coupled with fluid delivery. Put differently, at least a portion of all movements of the elongate member (e.g., advancement, retraction, re-advancement, re-retraction) may be coupled with fluid delivery such that fluid may be delivered during each of advancement, retraction, re-advancement, and re-retraction.

In some variations, as seen in FIG. 7A, the drive assembly 430 may comprise components that translate rotational motion into linear motion. For example, the drive assembly 430 may include a first linear gear 440, a second linear gear 450, a first pinion gear 434, and a second pinion gear 436. The first pinion gear 434 may be in contact with or otherwise engage a second actuator configured to be contacted by a user (e.g., the second rotatable component 432B) and the second pinion gear 436 may be in contact with or otherwise engage a first actuator configured to be contacted by a user (e.g., the first rotatable component 432B). The first pinion gear 434 may be in contact with or otherwise engage the second pinion gear 436. In some variations, the first actuator (e.g., rotatable component 432A) and the second actuator (e.g., rotatable component 432B) may each comprise a plurality of teeth. The plurality of teeth of the first actuator may directly engage corresponding teeth of the second pinion gear 436 and the plurality of teeth of the second actuator may directly engage corresponding teeth of the first pinion gear 434. Moreover, the teeth of the first pinion gear 434 may directly engage the corresponding teeth of the second pinion gear 436 and the teeth of the second pinion gear 436 may directly engage corresponding teeth of each of the first linear gear 440 and the second linear gear 450. In this manner, movement (e.g., rotation) of each of the actuators (e.g., rotatable components) 432A-432B rotates the first and second pinion gears 434, 436 and moves each of the first linear gear 440 and the second linear gear 450. In some variations, the first linear gear 440 and the second linear gear 450 may move along offset, parallel paths, such that actuation of the first linear gear 440 and the second linear gear 450 by one or more of the actuators (e.g., the rotatable components 432A-432B) may generate movement of the first linear gear 440 and the second linear gear 450 simultaneously and in opposite directions. In some variations, the first actuator (e.g., rotatable component 432A) and the second actuator (e.g., rotatable component 432B) may be substantially similar. For example, the first actuator (e.g., rotatable component 432A) and the second actuator (e.g., rotatable component 432B) may have similar features, such as a similar size and/or similar number of teeth that engage either the first pinion gear 434 or the second pinion gear 436 such that actuation of each of the first actuator (e.g., rotatable component 432A) and the second actuator (e.g., rotatable component 432B) leads to movement of each of the first linear gear 440 and the second linear gear 450 the same distance from a midpoint defined by the midpoint the first pinion gear 434, but in opposing directions. In some variations, the first actuator (e.g., rotatable component 432A) and the second actuator (e.g., rotatable component 432B) may have different features, such as, different sizes and/or number of teeth that engage either the first pinion gear 434 or the second pinion gear 436 such that actuation of each of the first actuator (e.g., rotatable component 432A) or the second actuator (e.g., rotatable component 432B) leads to movement of the first linear gear 440 or the second linear gear 450 different distances from midpoint as defined by the midpoint of the first pinion gear 434, in opposing directions. For example, the first actuator (e.g., rotatable component 432A) may be have fewer teeth that directly engage the second pinion gear 436 than the number of teeth of the second actuator (e.g., rotatable component 432B) that directly engage the first pinion gear 434, leading to the first linear gear 440 moving a greater distance from the midpoint as defined by a midpoint of the first pinion gear 434 than the second linear gear 450, in opposing directions. In some variations, the first pinion gear 434 and second pinon gear 436 may have different sizes, where the second pinion gear 436 may have a larger diameter than the first pinion gear 434, or the first pinion gear 434 may have a larger diameter than the second pinion gear 436, or may have the same size, wherein the first pinion gear 434 and the second pinion gear 436 have the same diameter. The differences in diameters of the first pinion gear and the second pinion gear may lead to the first linear gear or the second linear gear moving a greater distance from the midpoint as defined by the first pinion gear than the second linear gear, in opposing directions. It can be appreciated that the drive assembly 430 may be engineered in any number of ways so that each of the first linear gear 440 and the second linear gear 450 travel the same distance in opposing directions, or different distances in opposing directions.

In some variations, one or more components of the drive assembly and/or fluid assembly may be motorized. The device (e.g., drive assembly, fluid assembly) may comprise one or more motors (e.g., servo motor) configured to move one or more components of the drive assembly or fluid assembly, including, for example, a linear gear, a pinion gear, a displacement rod, a plunger tube, a fluid reservoir, an elongate member, and the like. The one or more motors may be configured to allow components of the drive assembly and/or fluid assembly to move independently of one another. In some variations, a motor may be operatively coupled to an actuator and configured to receive signals from the actuator. For example, the actuator may comprise an encoder configured to measure an input from a user. The encoder may be configured to signal the motor to move one or more components of the drive assembly and/or fluid assembly based on the user input.

In some variations, the motor may automatically move one or more components of the drive assembly and/or fluid assembly in response to a user input. One or more motors may be configured to move the elongate member and one or motors may be configured for deliver of fluid to the elongate member. In some variations, a single motor may be configured to both move the elongate member and deliver a fluid composition. In some variations, a first motor may be configured to move the displacement rod into the fluid reservoir and may be operably coupled to the displacement rod. For example, the first motor may be couple to (e.g., positioned at least partially within) a handle and may be operatively coupled to the displacement rod. Movement of an actuator (e.g., rotation of the actuator in a first direction) may signal the motor to move the displacement rod into the fluid reservoir. In some variations, a second motor may be configured to move the elongate member independently, such as, for example, independently of the displacement rod. For example, the second motor may be coupled to (e.g., positioned at least partially within) the handle and may be operatively coupled to the elongate member. Movement of an actuator may signal the motor to move the elongate member. For example, movement of an actuator in a first direction may signal the motor to advance the elongate member and movement of the actuator in a second direction (e.g., second opposite direction) may signal the motor to retract the elongate member.

### Linear Gear Stop

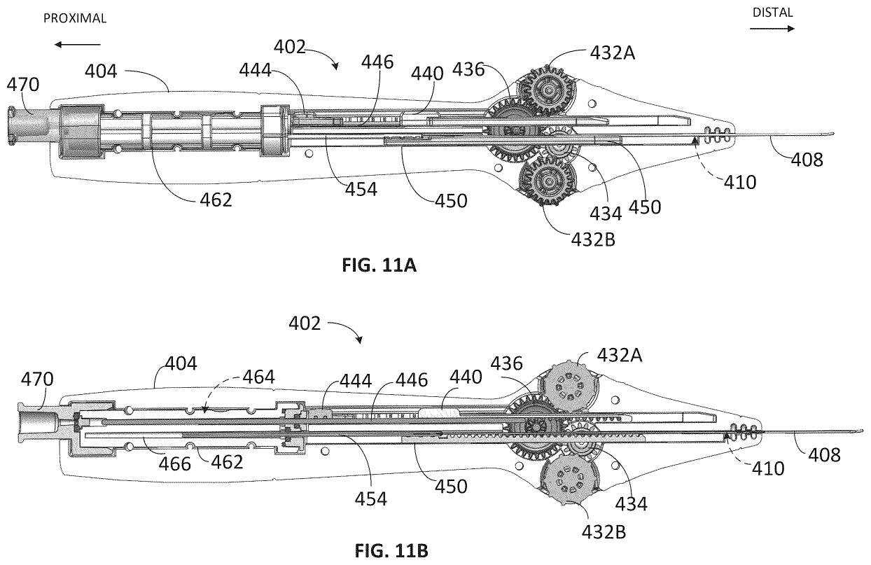

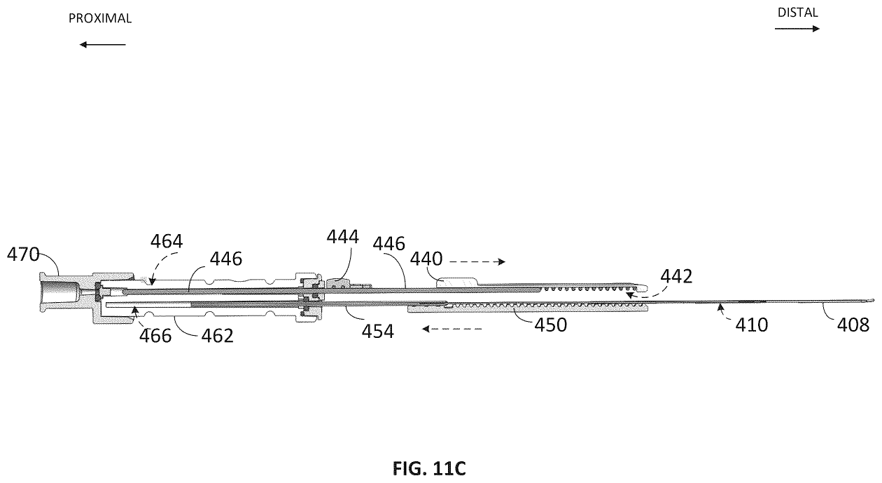

In some treatments, re-advancement of the elongate member coupled with fluid delivery may be desired. However, retraction of the elongate member may cause the first linear gear 440 to operatively decouple from the ratchet 444, as shown in FIG. 11B. Fluid delivery during re-advancement then may not occur or may not occur until the first linear gear moves and again operatively couples to the ratchet. Thus, in some variations, it may be desirable that a linear gear be configured to engage the displacement rod directly to move the displacement rod into the fluid reservoir to deliver fluid during re-advancement of the elongate member. Advancement of the elongate member a first predetermined distance may move a first linear gear in a first direction and move a displacement rod in the first direction toward the fluid reservoir. The first linear gear may releasably couple to the ratchet coupled to the displacement rod to move the displacement rod toward the fluid reservoir. The displacement rod may at least partially fit within a channel of the first linear gear during advancement. The first linear gear may receive a first length of the displacement rod in the channel during advancement of the elongate member. Retraction of the elongate member a first predetermined distance may move the first linear gear in a second opposite direction. During retraction, the first linear gear may not move the displacement rod and the ratchet coupled to the displacement rod may maintain the position of the displacement rod within the fluid reservoir. Thus, after retraction of the elongate member a first predetermined distance, the linear gear may be decoupled from the ratchet coupled to the displacement rod. However, a portion of the displacement rod may remain received in the channel of the first linear gear. The first linear gear may comprise a stop configured to engage this portion of the displacement rod during re-advancement of the elongate member. Thus, re-advancement of the elongate member after the first predetermined distance may again move the first linear gear in the first direction. The stop of the first linear gear may engage the displacement rod before the first linear gear would have otherwise again releasably coupled to the ratchet. The linear gear may move the displacement rod and optionally the ratchet coupled to the displacement rod in the first direction toward the fluid reservoir. The first linear gear may receive a second length of the displacement rod in the channel during re-advancement of the elongate member.

In some variations, a second length of the displacement rod received by the first linear gear may be less than a first length of the displacement rod. The first linear gear may receive a first length of the displacement rod during advancement of the elongate member and may receive a short second length of the displacement rod during re-advancement to account for a length of the displacement rod already moved into the fluid reservoir. Both of the second and the first length of the displacement rod may be received in the channel of first linear gear. A difference between the first length and the second length of the displacement rod may correspond to about 1 clock hour to about 8 clock hours of elongate member travel around the eye, such as about 2 clock hours to about 6 clock hours or about 3 clock hours to about 5 clock hours of elongate member travel around the eye. Similarly, the second length of the displacement rod may be about 3 mm to about 24 mm less than the first length of the displacement rod, such as 6 mm to about 18 mm or about 9 mm to about 15 mm less than the first length of the displacement rod. In some variations, a ratio of the first length of displacement rod to the second length is between about 10 to 7 and about 2 to 1. In some variation, the ratio of the first length of displacement rod to the second length is about 5 to 3. The displacement rod may be configured to deliver fluid during about 1 to about 10 clock hours of travel of the elongate member around the eye during re-advancement, such as about 2 clock hours of re-advancement of the elongate member, about 4 clock hours of re-advancement of the elongate member, about 6 clock hours of re-advancement of the elongate member, and about 10 clock hours of re-advancement of the elongate member.

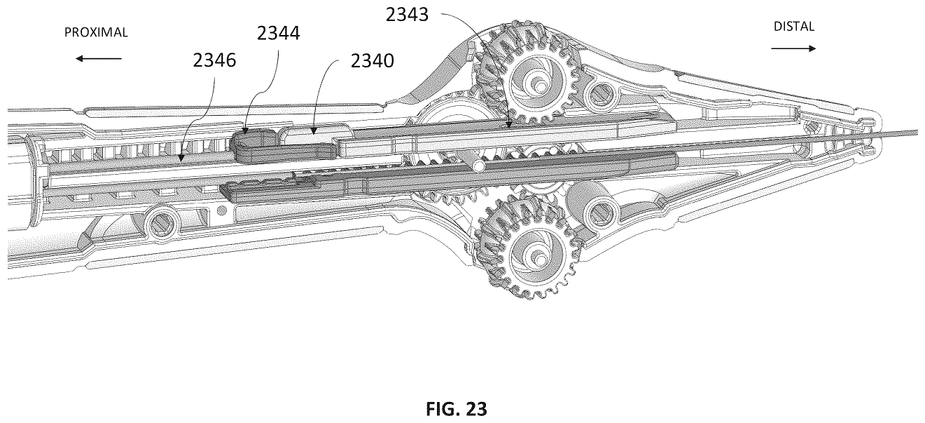

In some variations, the first linear may comprise a channel including a stop configured to engage a portion of the displacement rod. For example, FIG. 23 depicts a perspective view of a drive assembly comprising a first linear gear 2340 comprising a channel including a stop 2343, a displacement rod 2346, and a ratchet coupled to the displacement rod 2344. As in FIG. 23, the channel may be positioned on a bottom face of the first linear gear 2340. The channel may be configured to receive a portion of the displacement rod. In some variations, the channel may comprise a stop configured to contact the displacement rod during a re-advancement of the elongate member. In some variations, the stop 2343 may be positioned a distance proximal from a distal end of the channel corresponding to about 1 clock hour to about 8 clock hours of elongate member travel around the eye, such as about 2 clock hours to about 6 clock hours or about 3 clock hours to about 5 clock hours of elongate member travel around the eye. The stop 2343 may be about 3 mm to about 24 mm proximal from a distal end of the channel, such as 6 mm to about 18 mm or about 9 mm to about 15 mm proximal from a distal end of the channel (including all ranges and subranges therebetween). The linear gear 2740 may releasably couple to the ratchet coupled to the displacement rod 2344 and/or the displacement rod 2346 to move the displacement rod 2346 toward the fluid reservoir. Retraction of the elongate member a first predetermined distance may decoupled the first linear gear 2340 from the ratchet coupled to the displacement rod 2344 and position a distal end of the displacement rod (not show) proximal to the stop 2343 of the first linear gear 2340. Re-advancement of the elongate member may again move the first linear gear 2340 such that the first linear gear 2340 may receive the displacement rod 2346 in the stop 2343 and again move the displacement rod 2346. In some variations, the stop may limit movement of the first linear gear, and thus limit the movement of the second linear gear, the elongate member, and/or a plunger tube. For example, during re-advancement, the elongate member may be limited to advancement of between about 1 to about 10 clock hours of travel around the eye, such as about 2 clock hours around the eye, about 4 clock hours around the eye, about 6 clock hours around the eye, or about 10 clock hours around the eye. Movement of the plunger tube may be limited an amount corresponding to the limited re-advancement length of the elongate member.

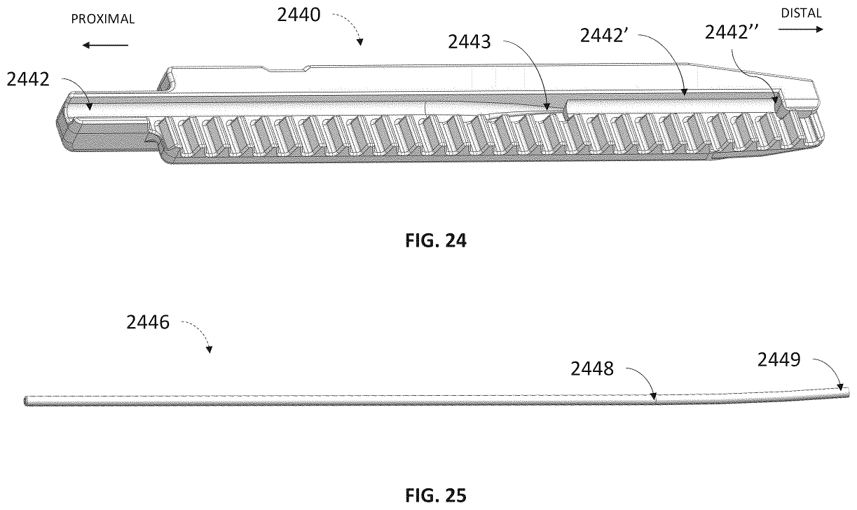

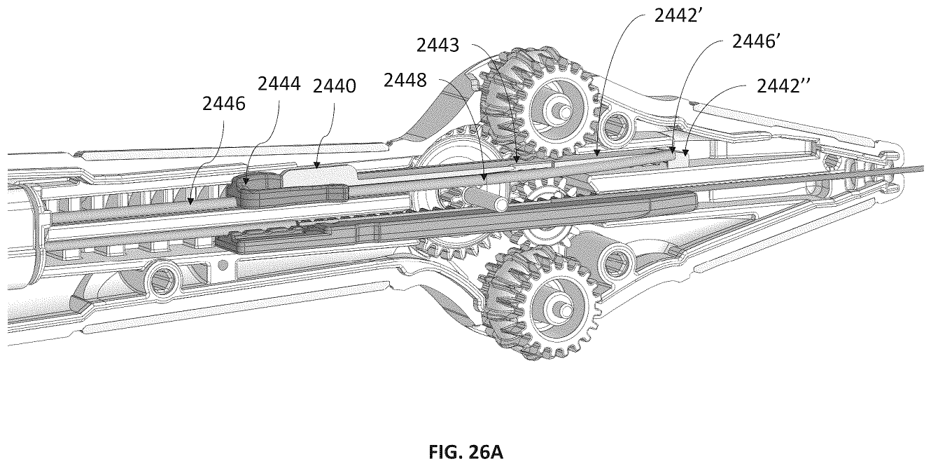

In some variations, the stop may comprise an opening in the channel of the linear gear. FIG. 24 depicts a first linear gear 2440 comprises a stop 2443 configured as an opening in a channel 2442. In some variations, as shown in FIG. 25, the displacement rod 2446 may comprise a bend 2448 and a distal end 2449. The first linear gear 2440 may receive a first length of the displacement rod 2446 in the channel 2442 during advancement of the elongate member. The first length of the displacement rod may be received in a distal portion of the channel 2442′ up to a distal end of the channel 2742″ during advancement. During retraction of the elongate member, the first linear gear 2440 may move such that a distal end 2449 of the displacement rod 2446 may be positioned proximal to the stop 2443. The bend 2448 of the displacement rod may be configured to bias the distal end of the displacement rod 2449 toward engagement with the stop 2443. Thus, during re-advancement of the elongate member, the linear gear 2440 may receive a portion of the displacement rod 2446 in the stop 2443 and move the displacement rod 2446 toward the fluid reservoir.

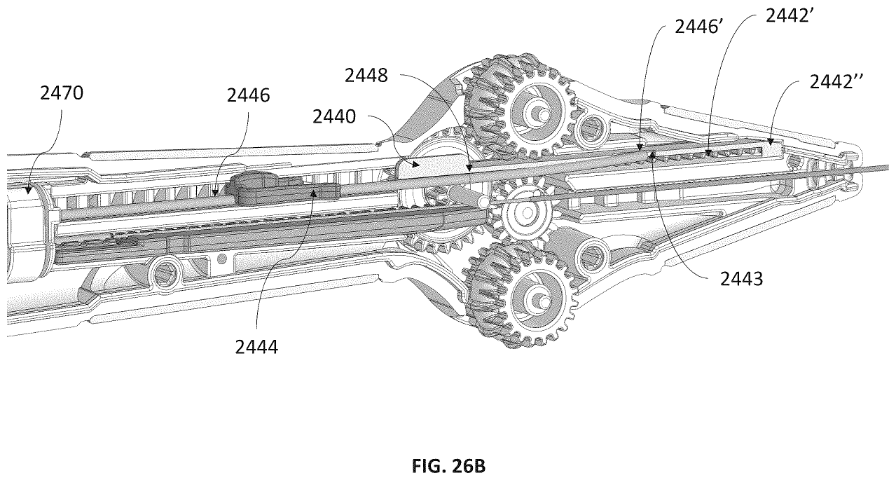

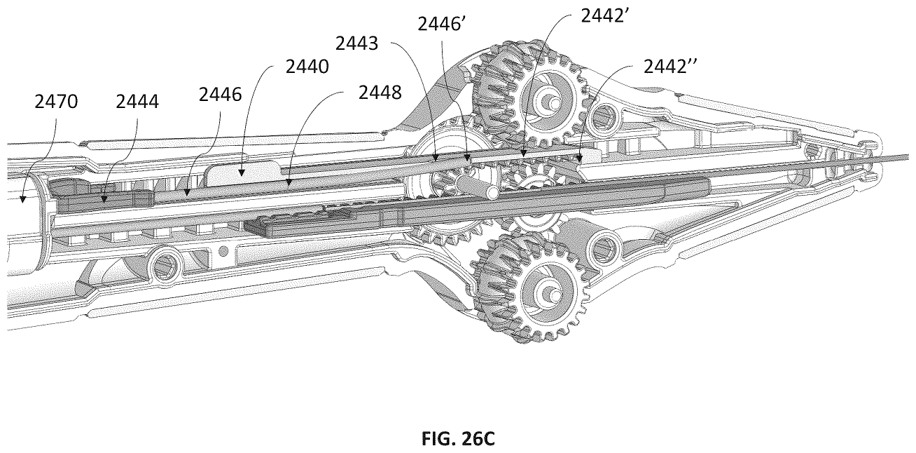

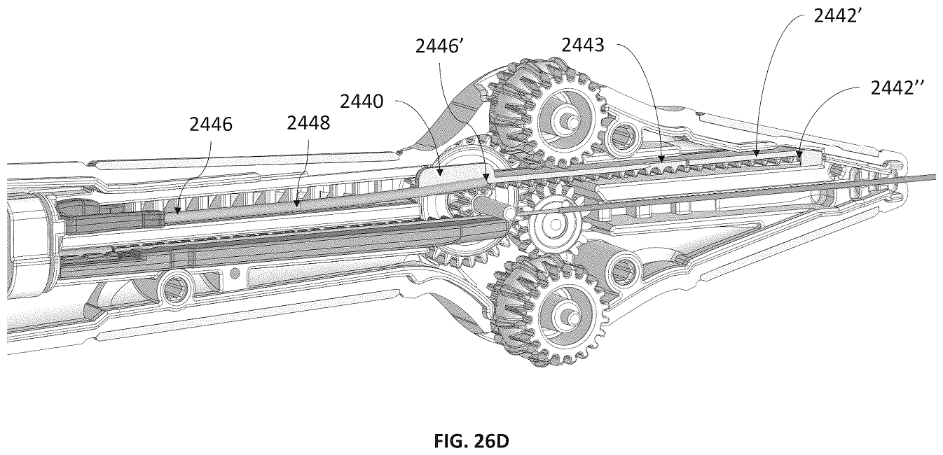

FIGS. 26A-26D depict perspective views of a device including a cross-section of a first linear gear 2440 and a displacement rod 2446 at different configurations during a fluid delivery cycle. FIG. 26A depicts a view of an advancement configuration of the device. As show in FIG. 26A, in an advancement configuration, the first linear gear 2740 may releasably couple to the ratchet coupled to the displacement rod 2444 and/or the displacement rod 2446 to move the displacement rod 2246 toward the fluid reservoir and deliver fluid. The first linear gear 2440 may receive a portion of the displacement rod 2446 in a distal portion of a channel 2442′ up to a distal end of the channel 2442″. FIG. 26B depicts a perspective view of a retraction configuration of the device. As shown in FIG. 26B, in a retraction configuration, the first linear gear 2440 may not move the displacement rod 2446 and the ratchet coupled to the displacement rod 2444 may maintain the position of the displacement rod 2446 within the fluid reservoir 2470. The linear gear 2440 may be decoupled from the ratchet 2444. In the retracted configuration, the distal end of the displacement rod 2446′ may be proximal to the stop 2443. FIG. 26C depicts a perspective view of a re-advancement configuration of the device. As shown in FIG. 26C, in a re-advancement configuration, the first linear gear 2440 may receive the distal end of the displacement rod 2446′ in the stop 2443 (e.g., opening) and move the displacement rod 2446 and the ratchet coupled to the displacement rod 2444 toward the fluid reservoir 2470. Finally, FIG. 26D depicts a perspective view of a re-retraction configuration of the device. In a re-retracted configuration, the linear gear 2440 may decouple the stop 2443 from the distal end of the displacement rod 2446′.

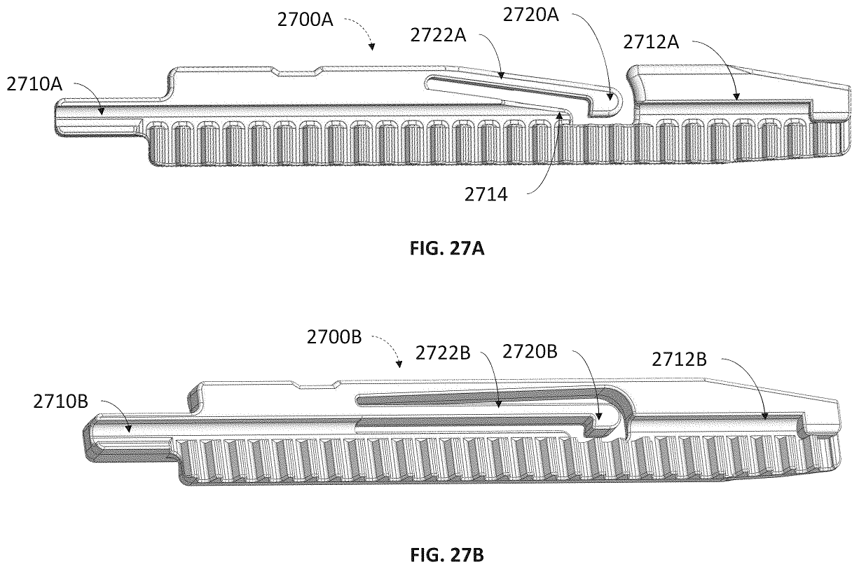

In other variations, the stop may comprise a pawl in a channel of the first linear gear. FIGS. 27A-27B depict linear gears 2700A, 2700B comprising a pawl 2720A, 2720B. FIG. 27A depicts a first linear gear 2700A comprising a channel 2710A comprising a distal portion 2712A, a pawl 2720A, and a pawl arm 2722A. During advancement of the elongate member, the first linear gear 2700A may receive a portion of a displacement rod in the distal portion 2712A of the channel 2710A. The pawl arm 2722A may be configured to deflect the pawl 2720A laterally away from the channel 2710A as the displacement rod is received in the distal end of the channel 2712A. The pawl arm 2722A may bias the pawl 2720A toward an opening 2714 in the channel 2710A. The opening 2714 may be configured to allow the pawl 2720A to partially enter the channel to receive the displacement rod. During retraction, the displacement rod 2746 may move proximally past the pawl 2720A and the pawl arm 2722A may be configured to move the pawl 2720A into the channel. The pawl 2720A may be configured to receive a distal end of the displacement rod during re-advancement of the elongate member. Thus, the pawl 2720A may releasably couple the linear gear 2700A to the displacement rod to move the displacement rod during re-advancement.

Similarly, FIG. 27B depicts another linear gear 2700B comprising a channel 2710B comprising a distal portion 2712B, a pawl 2720B, and a pawl arm 2722B. In this variation, the pawl arm 2722B may be configured as a portion of the channel 2710B. For example, the pawl arm 2722B may about parallel to the channel 2710B. During advancement of the elongate member, the first linear gear 2700B may receive a portion of the displacement rod in the distal portion 2712B of the channel 2710B. The pawl arm 2722B may be configured to deflect the pawl 2720B laterally away from the channel 2710B such that the displacement rod may be received in the distal end of the channel 2712B. The pawl arm 2722B may bias the pawl 2720B toward the channel 2710B. During retraction, when the displacement rod has moved proximally past the pawl 2720B, the pawl arm 2722B may move the pawl 2720B into the channel. For example, the pawl arm 2722B may be angled relative to the channel 2710B during advancement of the elongate member and approximately parallel to the channel 2710B during re-advancement of the elongate member. The pawl 2722B may be configured to receive a distal end of the displacement rod during re-advancement of the elongate member. Configured as a portion of the channel 2710B, the pawl arm 2722B may guide the displace rod to the pawl 2720B. Thus, the pawl 2720B may releasably couple the linear gear 2700B to the displacement rod to move the displacement rod during re-advancement.

### Displacement Rod Ratchet

In some variations, the ratchet coupled to the displacement rod may be configured to engage a first linear gear and move the displacement rod into the fluid reservoir to deliver fluid during re-advancement of the elongate member. The ratchet coupled to the displacement rod may be releasably coupled to a first linear gear in a first configuration during advancement of the elongate member such that movement of the first linear gear moves the displacement rod toward the fluid reservoir. During retraction of the elongate member, the first linear gear may not move the displacement rod and the ratchet coupled to the displacement rod may maintain the position of the displacement rod within the fluid reservoir. Thus, in some variations, after retraction of the elongate member a predetermined distance, the linear gear may be decoupled from the ratchet coupled to the displacement rod. However, in other variations, the ratchet coupled to the displacement rod may comprise an arm configured to engage the first linear in a second configuration during re-advancement of the elongate member. In the second configuration, the linear gear may be releasably coupled to the arm of the ratchet. The second configuration may allow the linear gear to engage the ratchet (via the arm) earlier in the re-advancement of the elongate member than the linear gear would have otherwise been able to engage the ratchet during re-advancement if the linear gear were to engage the ratchet in the first configuration. In some variations, the second configuration may allow the delivery of fluid at the beginning of re-advancement. In the second configuration, the first linear gear may engage the arm of the ratchet coupled to the displacement rod and again move the displacement rod toward the fluid reservoir. The ratchet coupled to the displacement rod may be further configured to disengage from the first linear gear during a portion of re-advancement and re-retraction of the elongated member.

In some variations, the arm of the ratchet may be configured to allow full advancement of the elongate member during re-advancement. It may be desirable in some procedures to partially advance the elongate member during advancement and fully advance (e.g., advance the elongate member the maximum length allowed by the drive assembly) the elongate member during re-advancement. The arm of the ratchet may be configured to allow complete movement of first linear gear during re-advancement so that the drive assembly may fully advance the elongate member during re-advancement. The arm of the ratchet coupled to the displacement rod may be configured to disengage the ratchet from the first linear gear when the displacement rod may not be inserted further into the fluid reservoir (e.g., the ratchet contacts the fluid reservoir and cannot be moved further toward the reservoir). Disengagement of the arm from the first linear gear may allow the first linear gear to continue to move towards the fluid reservoir during re-advancement of the elongate member.

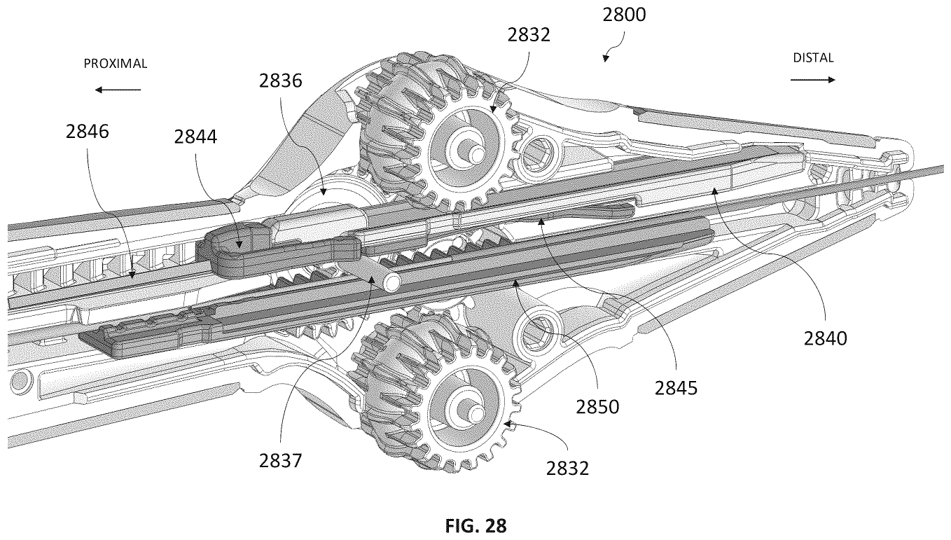

In some variations, the ratchet coupled to the displacement rod may comprise an arm configured to engage the first linear gear during a first portion of re-advancement of the elongate member and disengage the first linear gear during a second portion of re-advancement. For example, FIG. 28 depicts a perspective view of a device 2800 comprising a first linear gear 2840, a displacement rod 2846, and a ratchet coupled to the displacement rod 2844 including an arm 2845. FIG. 28 shows device 2800 during advancement of the elongate member (not shown). During advancement of the elongate member, movement of one or more of actuators 2832 in a first direction may move the first linear gear 2840, the ratchet coupled to the displacement rod 2844, and the displacement rod 2846 in a proximal direct and may move the second linear gear 2850 and the elongate member in a distal direction. The first linear 2840 may be releasably coupled to the ratchet coupled to the displacement rod 2844 and/or the arm 2845 in a first configuration, during advancement as shown in FIG. 28. Movement of the one or more actuators 2832 in second opposite direction may retract the elongate member and move the first linear gear 2840 in a distal direction. During retraction, the ratchet 2844 and the arm 2845 may disengage from the first linear gear 2840. Movement of the one or more actuators 2832 again in the first direction may re-advance the elongate member and move the first linear gear in a proximal direction. During a first portion of re-advancement, the arm 2845 of the ratchet 2844 may engage the first linear gear 2840 in a second configuration. In the second configuration, the first linear gear 2840 may be releasably coupled to the arm 2845 such that displacement rod 2846 and the ratchet coupled to the displacement rod 2844 move in the proximal direction. If during re-advancement the displacement rod 2846 has moved a predetermined maximum distance in the proximal direction (e.g., second portion of re-advancement), the arm 2845 may be configured to disengage the ratchet coupled to the displacement rod 2844 from the first linear gear 2840.

In some variations, the arm 2845 may be configured to contact at least a portion of the drive assembly or handle (not shown) to disengage the ratchet 2844 from the first linear gear 2840. For example, the arm 2845 may contact a shaft 2837 of the pinion gear 2836 to disengage the ratchet 2844 from the first linear gear 2840. After the arm 2845 disengages the first linear gear 2840, the first linear gear 2840 may be free to continue moving in the proximal direction, during the second portion of readvancement. With the first linear gear 2840 free to continue moving in the proximal direction, the drive assembly may continue to advance the elongate member during the re-advancement.

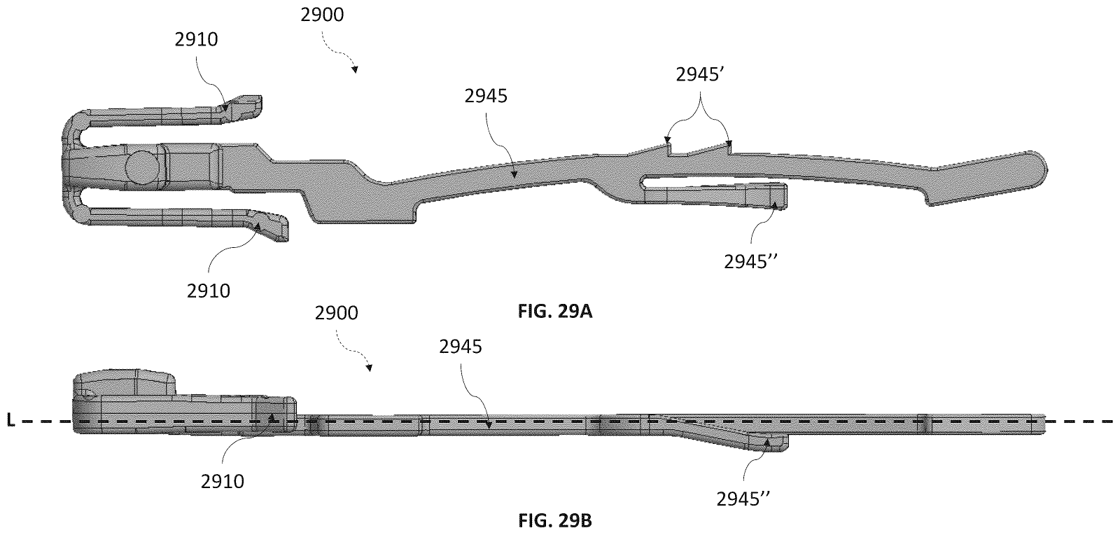

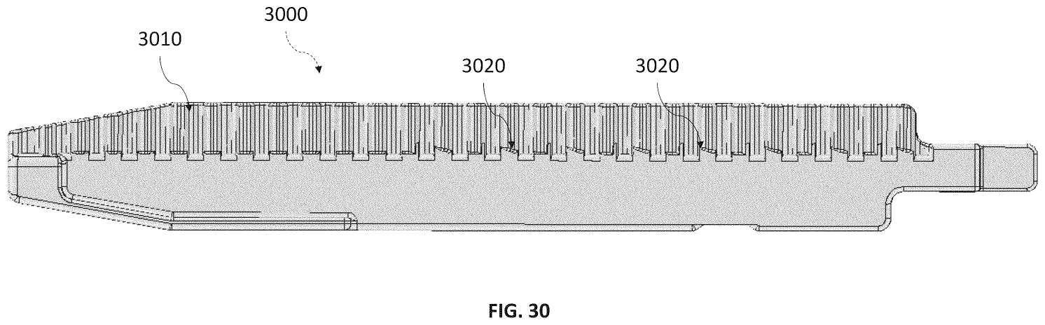

FIGS. 29A-29B depict a top and a side view of a ratchet 2900 configured to be coupled to a displacement rod comprising an arm 2945 configured to engage a first linear gear. The ratchet may comprise one or more ratchet teeth 2910 configured to maintain a position of the displacement rod in a fluid reservoir. The arm 2945 of the ratchet 2900 may comprise one or more engagement features 2945′ configured to engage the first linear gear. The one or more engagement features 2945′ may comprise one or more notches, teeth, protrusions, indentations, bumps, and the like. The arm 2945 may further comprise a protrusion 2945″ configured to disengage the arm 2945 from the first linear gear. For example, the protrusion 2945″ may contact a portion of the drive assembly causing the arm 2945 to move such that engagement features 2945′ no longer engage the first linear gear. In some variations, the protrusion 2945″ may be at angled relative to a longitudinal axis (L) of the arm 2945 such that movement of the displace rod and ratchet a predetermined distance toward the fluid reservoir cause the protrusion 2945″ to contact an axel of the drive assembly and disengage the arm. FIG. 30 depicts a linear gear 3000 configured for use with the ratchet 2900 of FIGS. 29A-29B. The linear gear 3000 may comprise a plurality of teeth 3010 configured to engage a pinion gear of the drive assembly and move the first linear gear 3000. The linear gear 3000 may further comprise one or more notches 3020 configured to engage the engagement features 2945′ of the arm 2945. In some variations, as shown in FIG. 30, the one or more notches 3020 may be configured as part of the plurality of teeth 3010.

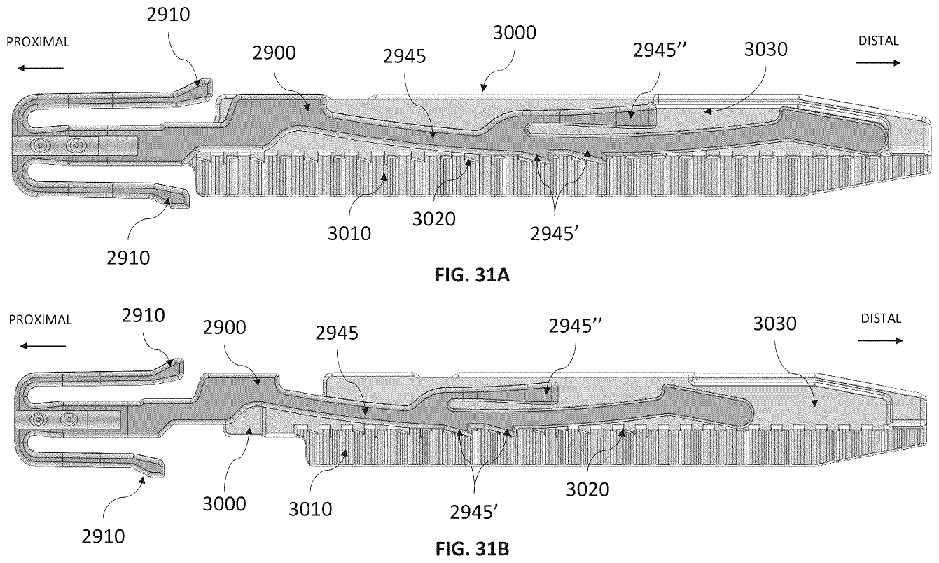

FIG. 31A depicts the ratchet 2900 and the linear gear 3000 in a first engagement configuration (e.g., during advancement of the elongate member), and FIG. 31B depicts the ratchet 2900 and linear gear 3000 in a second engagement configuration (e.g., during re-advancement of the elongate member). In the first engagement configuration, as shown in FIG. 31A, the ratchet 2900 may be releasably coupled to the first linear gear 3000 allowing the first linear gear to move the ratchet 2900 and the displacement rod (not show) in a first direction. In the first configuration, the linear gear 3000 may be releasably coupled to the ratchet 2900 by one or more of contact between a proximal portion of the ratchet and the linear gear 3000, engagement of the engagement features 2945′ with one or more notches 3020 of the linear gear 3000, and contact between a distal end of the ratchet arm 2945 and a distal portion of channel 3030 of the linear gear 3000. During retraction of the elongate member, the ratchet teeth 2910 may maintain the position of the ratchet relative to the linear gear 2900. The linear gear 3000 may move in a second opposite direction relative to the ratchet 2900. The ratchet 2900 may remain in channel 3030 during retraction. Repeat movement of the linear gear in the first direction (e.g., re-advancement of the elongate member) may engage the ratchet in the second engagement configuration depicted in FIG. 31B. In the second engagement configuration, the ratchet 2900 may be positioned proximally relative to its position in first engagement configuration. In the second engagement configuration, the linear gear 3000 may be releasably coupled to the ratchet 2900 by the engagement features 2945′ engaging one or more notches 3020 of the linear gear 3000.

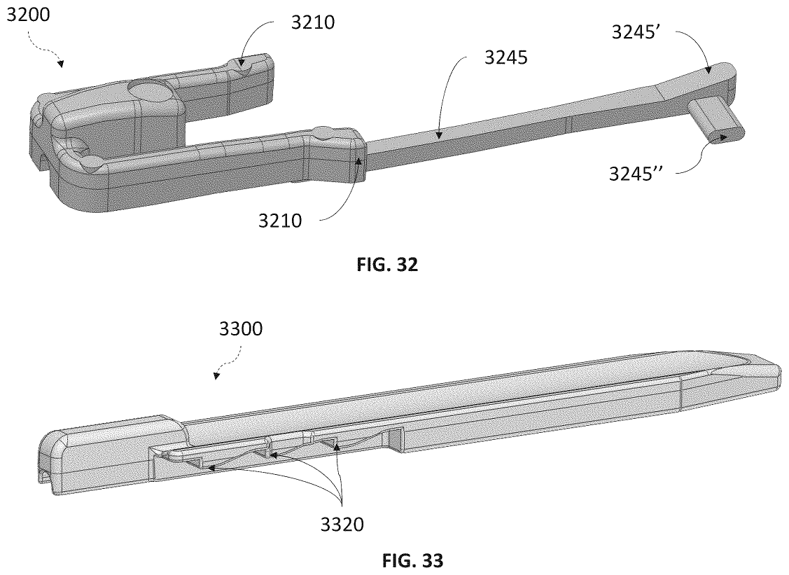

FIG. 32 depicts a perspective view of another variation of a ratchet 3200 configured to be coupled to a displacement rod and comprising an arm 3245 configured to engage a first linear gear. The ratchet 3200 may comprise one or more ratchet teeth 3210 configured to maintain a position of the displacement rod relative to a fluid reservoir. The arm 3245 of the ratchet 3200 may comprise one or more engagement features 3245′ configured to engage the first linear gear. The one or more engagement features 2945′ may comprise one or more notches, teeth, protrusions, indentations, bumps, wedge, and the like. The arm 3245 may further comprise a protrusion 3245″ configured to disengage the arm 3245 from the first linear gear. For example, the protrusion 3245″ may engage a slot in the handle configured to move (e.g., bend, deflect) the arm 3245 such that the engagement the feature 3245′ is no longer engaged with the first linear gear. FIG. 33 depicts a linear gear 3300 configured for use with the ratchet 3200 of FIG. 32. The linear gear 3300 may comprise one or more engagement features 3320 configured to engage the engagement features 3245′ of the arm 3245.

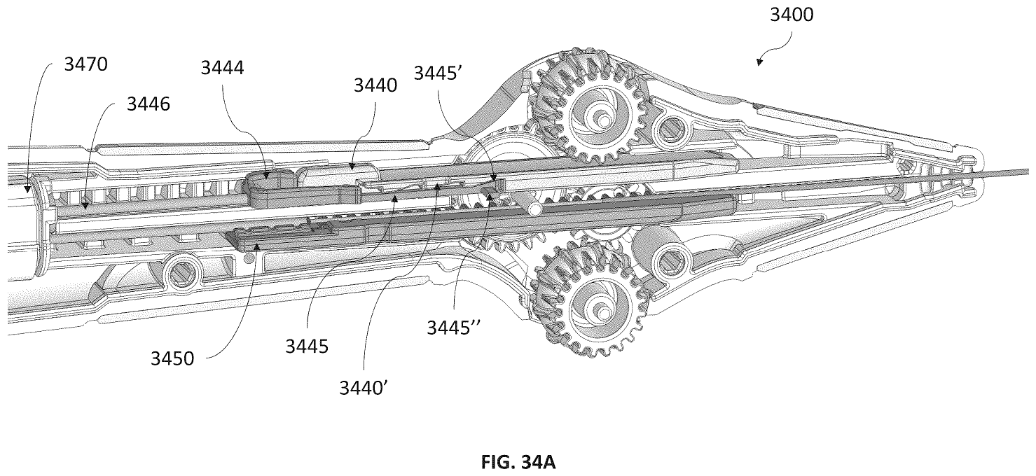

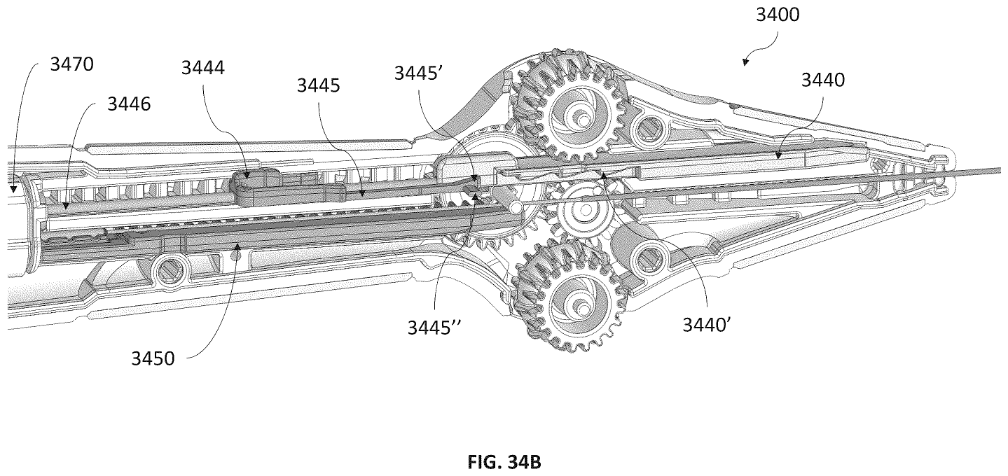

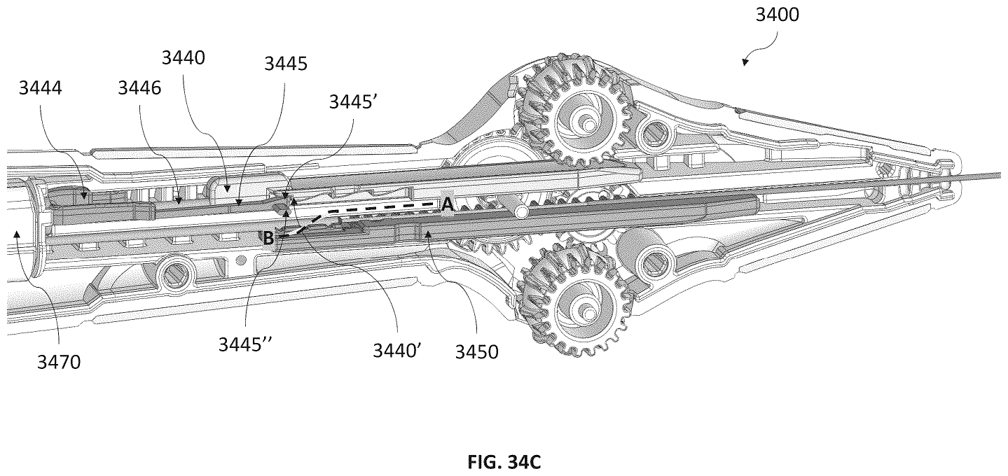

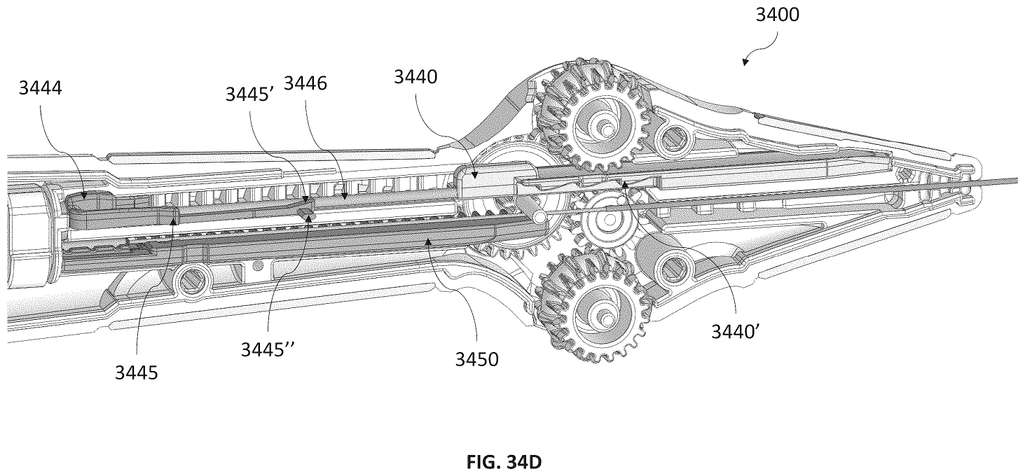

FIGS. 34A-34D depict perspective views of a delivery device 3400 including a first linear gear 2440 and a displacement rod 3446 with an arm 3445 at different configurations during a fluid delivery cycle. FIG. 34A depicts a perspective view of an advancement configuration of the device 3400 with one side of the handle removed. As show in FIG. 34A, in an advancement configuration, the first linear gear 3440 may directly couple to either the ratchet coupled to the displacement rod 3444 or the arm 3445 to move the displacement rod 3446 toward the fluid reservoir 3470 and deliver fluid. FIG. 34B depicts a perspective view of a retraction configuration of the device 3400 with one side of the handle removed. As shown in FIG. 34B, in a retraction configuration, the first linear gear 3440 may not move the displacement rod 3446 and the ratchet coupled to the displacement rod 3444 may maintain the position of the displacement rod 3446 within the fluid reservoir 3470. In the retracted configuration, the linear gear 3440 may be decoupled from the ratchet 3444 and the arm 3445. In the retracted configuration, the engagement feature 3445′ of the arm 3445 may be proximal to one or more engagement features 3440′ of the first linear gear 3440. FIG. 34C depicts a perspective view of a re-advancement configuration of the device 3400 with one side of the handle removed. In a re-advancement configuration, the engagement feature 3445′ of the arm 3445 may engage the one or more engagement features 3440′ of the first linear gear 3440. As shown in FIG. 34C, the arm 3445 may prevent the first linear gear 3440 from further travel toward the fluid reservoir 3470. In some variations, the arm protrusion 3445″ may engage a slot in the handle (not shown) and moved along path (A-B) such that the first linear gear 3440 is not prevent from traveling towards the reservoir. Lastly, FIG. 34D depicts a perspective view of a re-retraction configuration of the device 3400 with one side of the handle removed. In a re-retracted configuration, the linear gear 3440 may decouple from the arm 3445.

### Clutch

In some variations, the drive assembly may comprise a clutch configured to facilitate delivery of fluid to the elongate member during advancement and, optionally, re-advancement of the elongate member. The clutch may allow for delivery of fluid at the start of re-advancement. For example, the clutch may reduce mechanical delays in delivery of fluid during re-advancement of the elongate member. The clutch may selectively transfer the rotational motion to the linear gears. In particular, the clutch may be configured to move the first linear gear during the advancement or re-advancement of the elongate member. The clutch may be configured to not move (e.g., disengage) the first linear gear during retraction or re-retraction of the elongate member. In this manner, the clutch may keep the first linear gear coupled to the displacement rod ratchet. In some variations, the clutch may be engaged by movement of an actuator in a first direction. When engaged, the clutch may move the first and second linear gears in opposite directions. The clutch may be disengaged by movement of the actuator in a second opposite direction. When disengaged, the clutch may move one of the first and second linear gears. When engaged, the clutch may move both the first and second linear gears. In some variations, the clutch may decouple movement of a first linear gear from movement of a second linear gear. In doing so, the second linear gear may advance or retract the elongate member without the first linear gear moving the displacement rod. The clutch may be configured to prevent the displacement rod from moving out of the fluid reservoir during retraction of the elongate member. In some variations, the clutch may be configured to maintain a volume of a lumen of the fluid reservoir while the elongate member is retracted.

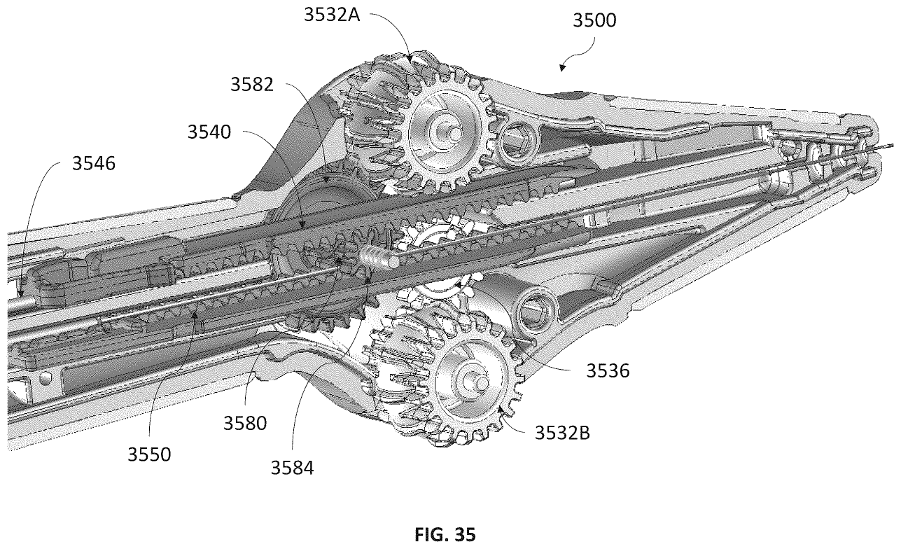

FIG. 35 depicts a perspective view of device 3500 including a drive assembly comprising a first linear gear 3540, a second linear gear 3550, and an exemplary clutch 3580 including a first pinion gear 3582 and second pinion gear 3584. The first pinion gear 3582 may be in contact with or otherwise engage one or more actuators 3532A-3532B, configured to be contacted by a user. The first pinion gear 3582 may be in contact with or otherwise selectively engage the second pinion gear 3584. In some variations, the one or more actuators 3532A-3532B may each comprise a plurality of teeth. The plurality of teeth of a first actuator 3532A may directly engage corresponding teeth of the first pinion gear 3582. The plurality of teeth of the second actuator 3532B may directly engage a plurality of teeth of an intermediate gear 3536 and the intermediate gear 3536 may directly engage the first pinon gear 3582. The second pinion gear 3584 may not directly engage either actuator 3532A-3532B. The first pinion gear 3582 may engage the second linear gear 3550 and the second pinion gear 3584 may engage the first linear gear 3540. In some variations, movement of each of the actuators 3532A-3532B in a first direction may engage the clutch 3580. Put another way, rotation of either of the actuators in a first direction may engage the first pinion gear 3582 with the second pinion gear 3584. In this manner, movement (e.g., rotation) of each of the actuators (e.g., rotatable components) 3532A-3532B in a first direction rotates the first and second pinion gears 3582, 3584 and moves each of the first linear gear 3540 and the second linear gear 3550. The first and second linear gears 3540, 3550 may move in opposite directions. In some variations, movement in the first direction may be associated with advancement or re-advancement of an elongate member. Movement of the first linear gear by the movement of actuators in the first direction may move a displacement rod into a fluid reservoir and deliver fluid to the elongate member during the advancement or re-advancement.

In some variations, movement of each of the actuators 3532A-3532B in a second direction opposite the first direction may disengage the clutch 3580. Put another way, rotation of each of the actuators 3532A-3532B in a second opposite direction may disengage the first pinion gear from the second pinion gear, disengaging the clutch 3580. In this manner, movement of each of the actuators 3532A-3532B in a second direction rotates the first pinion gear 3582 and moves the second linear gear 3550. Movement of each of the actuators 3532A-3532B in a second direction may not rotate the second pinion gear 3584 and may not move the first linear gear 3540. In some variations, movement of the actuators 3532A-3532B in the second direction may be associated with retraction or re-retraction of the elongate member.

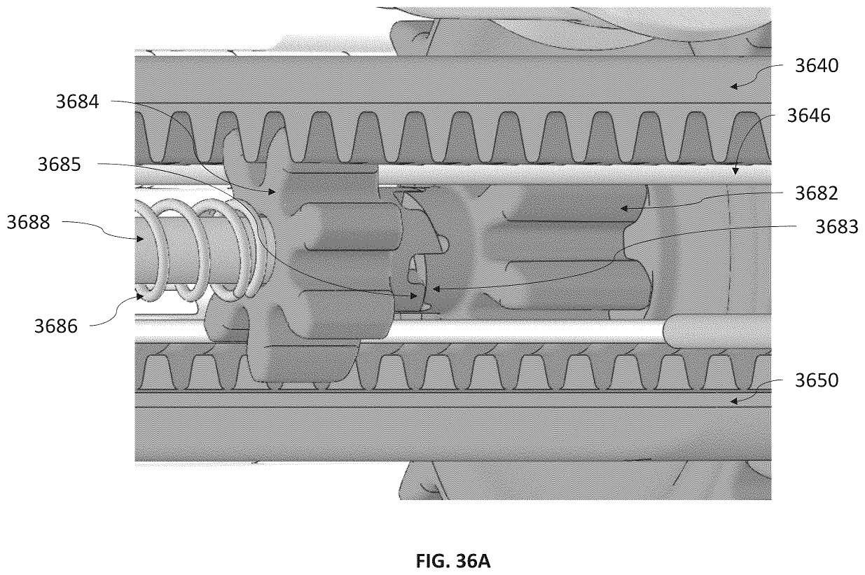

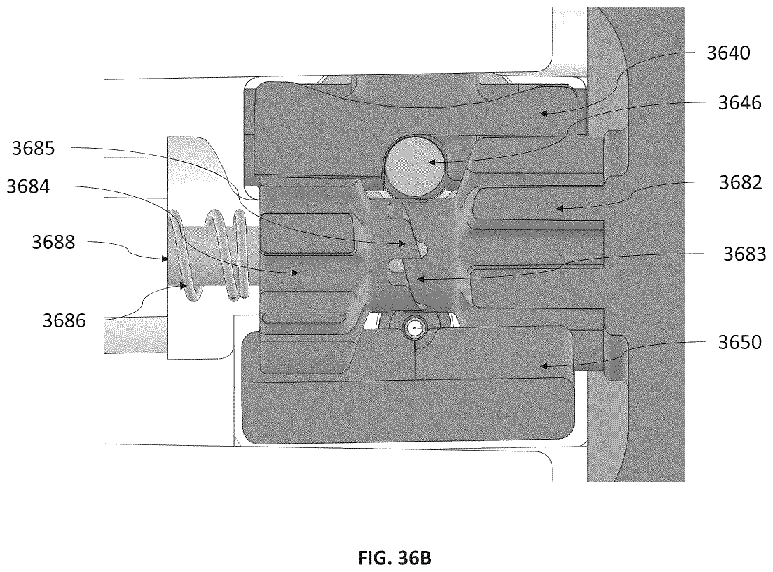

In some variations, the clutch may comprise a first pinion gear comprising a first set of teeth configured to engage a second set of teeth of a second pinion gear. For example, FIG. 36A-36B depict a drive assembly including a first linear gear 3640, second linear gear 3650, and clutch 3680. FIG. 36A shows a perspective view the drive assembly and FIG. 36B shows a side view. The clutch 3680 may comprise a first pinion gear 3682, a second pinion gear 3684, a spring 3686, and shaft 3688. The first pinion gear 3682 and the second pinion gear 3684 may each be rotatably coupled to the shaft 3688. In some variations, the first pinion gear 3682 and the second pinion gear 3684 may be biased toward engagement. For example, the spring 3686 may provide an axial force biasing the first and second pinion gears 3682, 3684 toward one another. A plurality of teeth of the first pinion gear 3682 may engage a plurality of teeth of the second linear gear 3650. In some variations, movement of an actuator in a first direction or second opposite direction may engage the first pinion gear 3682 and move the second linear gear 3650 in a distal or a proximal direction. A plurality of teeth of the second pinion gear 3684 may engage a plurality of teeth of the first linear gear 3640. In some variations, the first pinion gear 3682 may comprise one or more teeth 3683 (e.g., first set of teeth, face teeth) configured to engage the second pinion gear 3684, and the second pinion gear 3684 may comprise one or more teeth 3685 (e.g., second set of teeth, face teeth) configured to engage the first pinion gear 3682. In some variations, rotation of the first pinion in a first direction (e.g., rotation of an actuator engaged with the first pinion gear in a first direction) engages the one or more teeth of the first pinion gear 3683 with the one or more teeth of the second pinion gear 3685. Thus, the clutch (i.e., first and second pinion gear) may engage and move the first linear gear 3640 and the second linear gear 3650 simultaneously. For example, rotation of the first pinion gear 3682 in the first direction may cause the second linear gear 3650 to move in a distal direction while the first linear gear 3640 may move in a proximal direction. The first linear gear 3640 may be coupled to the displacement rod 3646 such that movement of the first linear gear 3640 in a proximal direction moves the displacement rod 3646 into the fluid reservoir and delivers fluid to the elongate member. In some variations, rotation of the first pinion gear in a second opposite direction disengages the one or more teeth of the first pinion gear 3683 from the one or more teeth of the second pinion gear 3685. Thus, the clutch (e.g., first and second pinion gear) may disengage from the first linear gear 3640 and move only the second linear gear 3650. For example, rotation of the first pinion gear 3682 in the second direction may cause the second linear gear 3650 to move in a proximal direction while the first linear gear may be stationary relative to the clutch. In some variations, disengaging the clutch may decouple the displacement rod 3646 from movement of the first pinion gear 3682 or actuators coupled thereto. Thereby, the clutch may maintain the position of the displacement rod in the fluid reservoir while advancing the elongate member.

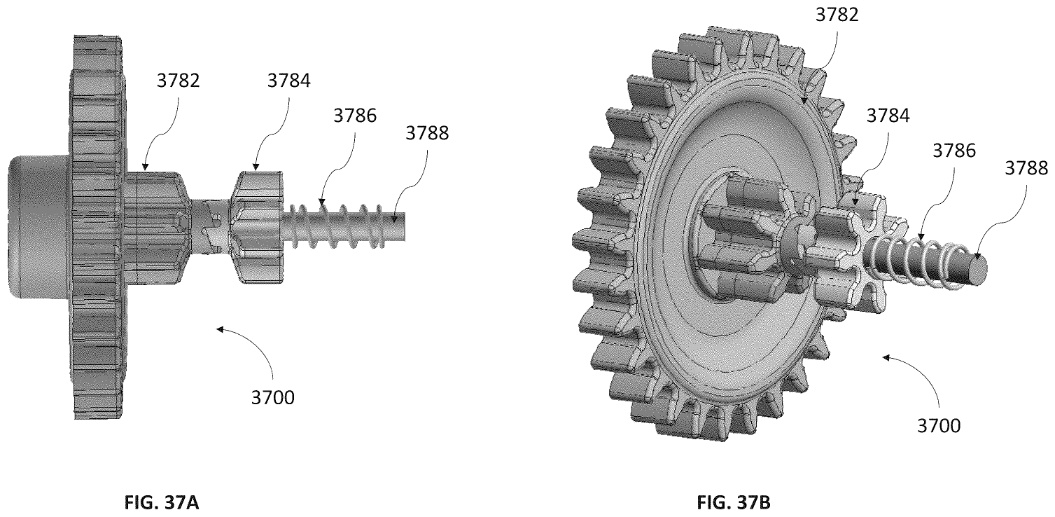

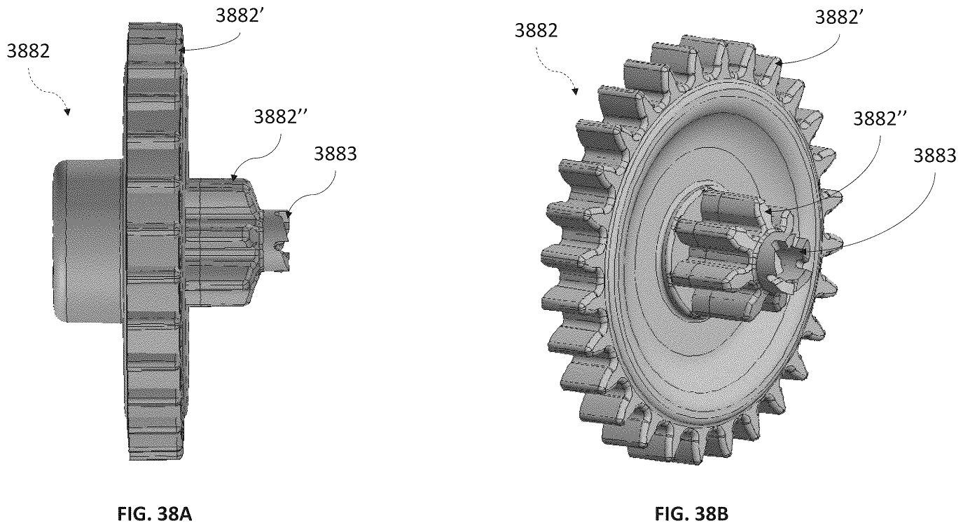

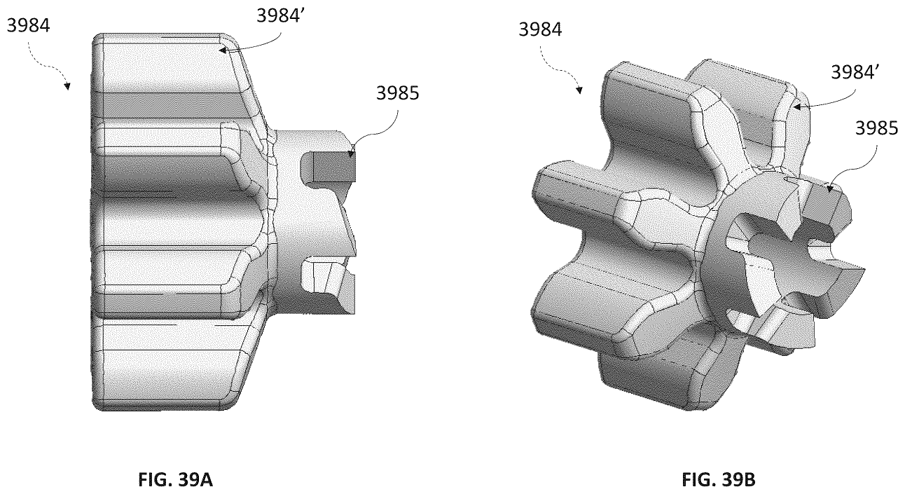

FIGS. 37A-37B depict a clutch 3700 comprising a first pinion gear 3782, a second pinion gear 3784, a spring 3786, and shaft 3788. FIG. 37A depicts a side view of the clutch 3700. FIG. 37B depicts a perspective view of the clutch 3700. The first pinion gear 3782 and the second pinion gear 3784 may each be rotatably coupled to the shaft 3788. In some variations, the first pinion gear 3782 and the second pinion gear 3784 may be biased toward engagement by the spring 3788. As shown in FIG. 37A, the first pinion gear 3782 may comprise one or more teeth (e.g., first set of teeth, face teeth) configured to engage the second pinion gear 3684, and the second pinion gear 3684 may comprise one or more teeth (e.g., second set of teeth, face teeth) configured to engage the first pinion gear 3682. In some variations, rotation of the first pinion in a first direction (e.g., rotation of an actuator engaged with the first pinion gear in a first direction) engages the one or more teeth of the first pinion gear with the one or more teeth of the second pinion gear. FIGS. 38A-38B depict a first pinion gear 3882. FIG. 38A depicts a side view of the first pinion gear 3882. FIG. 38B depicts a perspective view of the first pinion gear 3882. The first pinion gear may comprise a first plurality of teeth 3882′ configured to engage one or more of an actuator or intermediate gear of a drive assembly and a second plurality of teeth 3882″ configured to engage a second linear gear of the drive assembly. In some variations, the first pinion gear may comprise one or more teeth 3883 configured to selectively engage teeth of a second pinion gear. The one or more teeth 3883 may comprise face teeth with sloped ends configured to engage similar teeth of a second pinion gear when rotated in a first direction and to allow the first pinion gear 3882 to disengage from the second pinion gear when rotated in a second opposite direction. FIGS. 39A-39B depict a second pinion gear 3984. FIG. 39A depicts a side view of the second pinion gear 3984. FIG. 39B depicts a perspective view of the first pinion gear 3984. The second pinion gear may comprise a plurality of teeth 3984′ configured to engage a first linear gear of the drive assembly. In some variations, the second pinion gear may comprise one or more teeth 3985 configured to selectively engage the teeth of a first pinion gear. The one or more teeth 3885 may comprise face teeth with sloped ends configured to be engaged by similar teeth of the first pinion gear when the first pinion gear is rotated in a first direction. The one or more teeth 3985 may allow the first pinion gear to disengage from the second pinion gear 3984 when the first pinion gear is rotated in a second opposite direction.

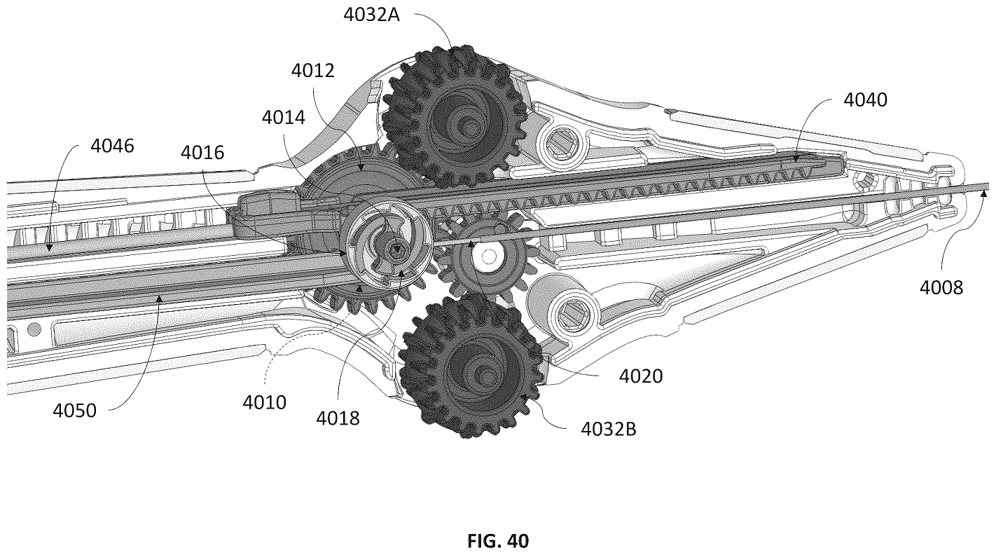

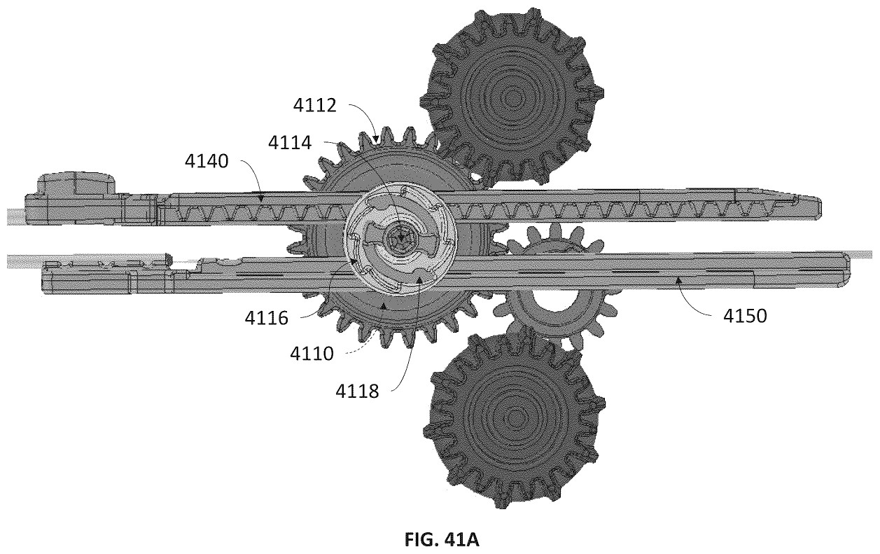

In another variation of a clutch, the clutch may comprise a pawl wheel and a ratchet hub configured to selectively engage the first linear gear. A clutch comprising a pawl wheel and ratchet hub may reduce wear of components of the clutch. Such a clutch may also be less expensive to manufacture. A clutch comprising a pawl wheel and ratchet hub may place less stress on the clutch and may have decreased sensitivity to frictional interference in the clutch mechanism. For example, a clutch comprising a pawl wheel and ratchet hub may not require more expensive metallic components and may be made of or comprise less expensive plastic materials. FIG. 40 depicts a drive assembly including a clutch 4010 comprising a first pinion gear 4012, a shaft 4014, a ratchet hub 4016 including a second pinion gear, and a pawl wheel 4018. The first pinion gear 4012 and pawl wheel 4018 may be coupled to the shaft 4014 such that rotation of the first pinion gear 4012 rotates the shaft 4014 and pawl wheel 4018. The ratchet hub 4016 may be rotatably coupled to the shaft 4014. In some variations, the shaft 4014 may be a component of the first pinion gear 4012. A plurality of teeth of the first pinion gear 4012 may engage a plurality of teeth of the second linear gear 4050. In some variations, movement an actuator 4032A-4032B in a first direction may engage the first pinion gear 4012 with the second linear gear 4050 and move second linear gear 4050 in a distal direction. Movement of the second linear gear 4050 in a distal direction may advance an elongate member 4020. Movement an actuator 4032A-4032B in a second opposite direction may also engage the first pinion gear 4012 with the second linear gear 4050 and move second linear gear 4050 in a proximal direction. Movement of the second linear gear 4050 in a proximal direction may retract the elongate member 4020. The ratchet hub 4016 may include a second pinion gear (not shown). The second pinion gear may comprise a plurality of teeth configured to engage a plurality of teeth of the first linear gear 4040. In some variations, rotation of the shaft 4014 in a first direction (e.g., rotation of an actuator engaged with the first pinion gear in a first direction) may engage the pawl wheel 4018 with the ratchet hub 4016. Thus, the clutch 4010 (i.e., first pinion gear, shaft, hub, pawl wheel) may engage and move the first linear gear 4040 and the second linear gear 4050 simultaneously. For example, rotation of the first pinion gear 4012 or the shaft 4014 in the first direction may cause the second linear gear 4050 to move in a distal direction while the first linear gear 3640 may move in a proximal direction. The first linear gear 4040 may be coupled to the displacement rod 4046 such that movement of the first linear gear 4040 in a proximal direction moves the displacement rod 4046 into the fluid reservoir and delivers fluid to the elongate member 4020. The second linear gear 4050 may advance the elongate member when moved in the distal direction. In some variations, rotation of the shaft 4014 in a second opposite direction disengages the pawl wheel 4018 from the ratchet hub 4016. Thus, the clutch 4010 may disengage from the first linear gear 4040 and move only the second linear gear 4050. For example, rotation of the first pinion gear 4012 in the second direction may cause the second linear gear 4050 to move in a proximal direction (retracting or re-retracting the elongate member 4020) while the first linear gear 4040 may be stationary relative to the clutch 4010. In some variations, disengaging the clutch 4010 may decouple the displacement rod 4046 from movement of the first pinion gear 4012 or actuators 4032A-4032B coupled thereto. Thereby, the clutch 4010 may maintain the position of the displacement rod 4046 in the fluid reservoir while advancing the elongate member.

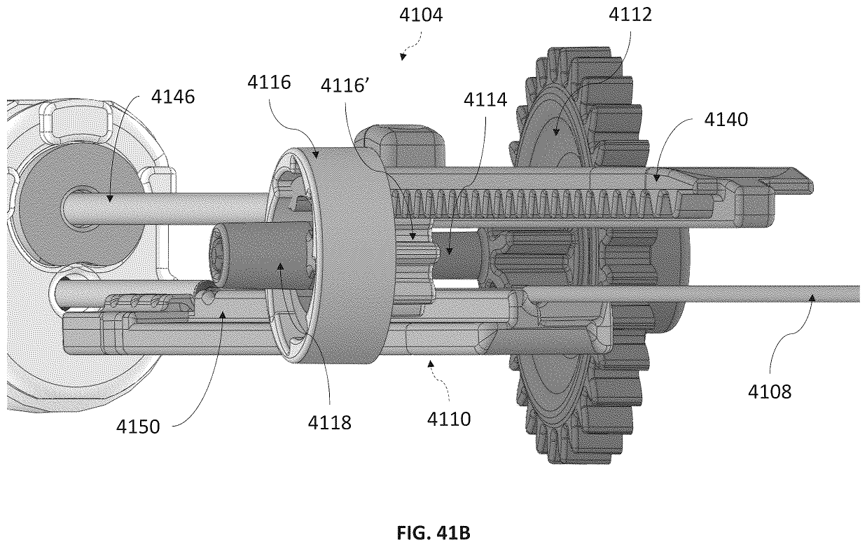

For example, FIGS. 41A-41B depict a drive assembly including a clutch 4110 comprising a first pinion gear 4112, a ratchet hub 4116, a pawl wheel 4118, and shaft 4114. FIG. 41A depicts a side view of the clutch 4110. FIG. 41B depicts a perspective view of a clutch 4110. As shown in FIG. 41B, the first pinion gear 4112 may be coupled to the shaft 4114 and the ratchet hub 4116 may be rotatably coupled to shaft 4114. In some variations, the pawl wheel 4118 may couple to a lateral end of the shaft 4114 extending from the ratchet hub 4116. The pawl wheel 4118 may be positioned at least partially within the ratchet hub 4116. As shown in FIG. 41B, the first pinion gear 4012 may comprise a plurality of teeth configured to engage the second linear gear 4150 and the ratchet hub may comprise a plurality of teeth 4116′ configured to engage the first linear gear 4140. In some variations, the ratchet hub 4116 may comprise one or more ratchet teeth configured to engage the pawl wheel 4114. For example, as shown in FIG. 41A, rotation of the first pinion 4112 in a first direction (e.g., rotation of an actuator engaged with the first pinion gear in a first direction) engages the one or more ratchet teeth of the first pinion gear with the pawl wheel. Rotation of the first pinion 4112 in a second opposite direction may disengage the one or more ratchet teeth of the first pinion gear from the pawl wheel. In some variations, one or more pawl arms of the pawl wheel may be configured to deflect radially inward to disengage the pawl wheel 4118 from the ratchet hub 4116.

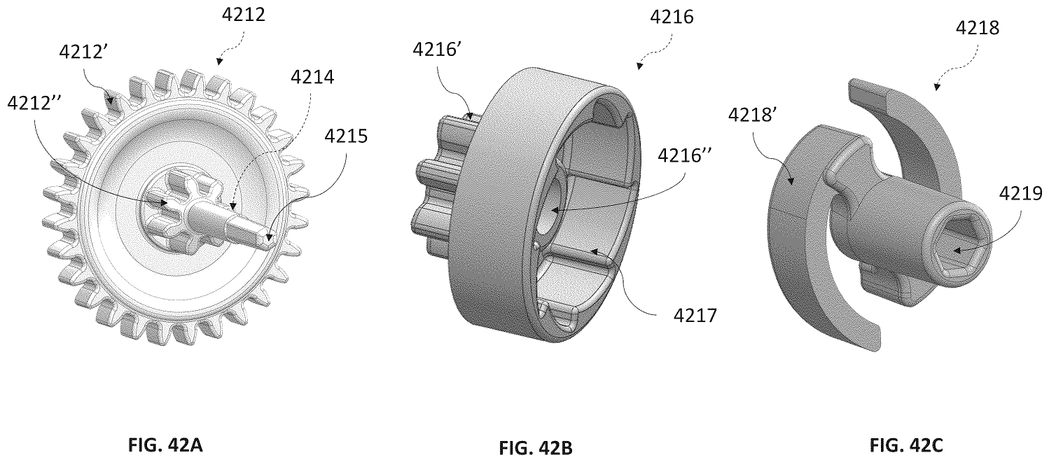

FIG. 42A depicts a perspective view of the first pinion gear 4212 and shaft 4214. The first pinion gear 4212 may be configured for use in a clutch comprising a ratchet hub or pawl wheel. The first pinion gear 4212 may comprise a first plurality of teeth 4212′ configured to engage one or more of an actuator or an intermediate gear of a drive assembly and a second plurality of teeth 4212″ configured to engage a second linear gear of the drive assembly. The shaft 4214 may be configured to couple to a pawl wheel or rotatably couple to a ratchet hub. In some variations, a lateral portion of the shaft 4215 may comprise one or more flat surfaces or asymmetrical features configured to rotate the pawl wheel with the shaft. For example, the lateral portion may comprise a cross-section configured to rotationally fix the pawl wheel to the shaft (e.g., triangle, square, star, pentagon, hexagon, octagon, any other polygon or similar cross-sectional shape). FIG. 42B depicts a perspective view of a ratchet hub 4216. The ratchet hub 4216 may comprise a plurality of teeth 4216′ configured to engage a first linear gear of the drive assembly. In some variations, the ratchet hub 4216 may not rotate with the shaft unless the clutch is engaged (e.g., the pawl wheel engages the ratchet hub). The ratchet hub 4216 may comprise a lumen 4216″ configured to rotatably couple the ratchet wheel 4216 to the shaft. In some variations, the ratchet wheel 4216 may comprise one or more ratchet teeth 4217 configured to be engaged by the pawl wheel.

FIG. 42C depicts a perspective view of a pawl wheel 4218. The pawl wheel 4218 may comprise a lumen 4219 configured to receive a clutch shaft therein. The lumen 4219 may comprise a cross-sectional opening similar to a cross-sectional shape of a lateral portion of the shaft (e.g., triangle, square, star, pentagon, hexagon, octagon, any other polygon or the like cross-sectional shape) configured to couple the pawl wheel to the shaft such that the pawl wheel rotates with the shaft. In some variations, the pawl wheel 4218 may comprise one or more pawl arms 4218′ configured to selectively engage the ratchet hub. For example, the rotation of the pawl wheel 4218 (e.g., rotation of the first pinion gear and the shaft in a first direction) may engage a tip of the one or more pawl arms with one or more ratchet teeth of the ratchet hub. Rotation of the pawl wheel 4218 in an opposite direction (e.g., rotation of the first pinion gear and the shaft in a second opposite direction) may disengage the pawl wheel 4218 from the ratchet hub and thus disengage the clutch. For example, the one or more pawl arms may be configured deflect (e.g., bend, deform, hinge, compress) radially inward when the pawl wheel 4218 is rotated in an opposite direction. In some variations, contact between a surface (e.g., outer surface) of the pawl arm and one or more ratchet teeth of the ratchet hub may cause the pawl arm to deflect.

### Extendable Fluid Coupler

In some variations, it may be desirable to reduce to the number of components of the drive assembly or to reduce the number of moving components while preserving the fluid delivery capabilities of the device. Improving the drive assembly in this manner may facilitate motorization of the movement of one or more components as described in more detail herein. Additionally or alternatively, in some instances, a drive assembly with fewer moving components may provide increased control over fluid delivery. For example, utilizing an extendable fluid coupler instead of a plunger tube or decoupling the plunger tube from movement of the second linear gear may reducing the number of moving components of the device, thereby decreasing the complexity of the device while maintaining the ability to accurately control fluid delivery. In some variations, utilizing the expandable fluid coupler may also increase control over fluid deliver by allowing the displacement rod alone to control the volume of the fluid reservoir.

The drive assembly of a device including an extendable fluid coupler may configured to move a displacement rod to deliver fluid instead of a plunger tube. In some variations, input motion in both a first direction and second opposite direction may move the displacement rod into the fluid reservoir. Movement of an actuator in either a first or a second opposite direction may cause movement of the displacement rod into the fluid reservoir and delivery of fluid to the elongate member. Input motion in both the first direction and the second opposite direction may decrease a volume of a lumen of the fluid reservoir, thereby eliminating the need for a second element (e.g., plunger tube) to decrease the volume of the reservoir in response to movement in one of the two directions. In some variations, movement of an actuator in both a first direction and second opposite may correspond to a linear motion of a portion of the drive assembly in a single direction. In some variations, the displacement rod may be configured to move into the fluid reservoir to deliver fluid to the elongate member during both advancement and retraction of the elongate member. The drive assembly may comprise components that translate rotational input motion in both a first direction and second opposite into translation of the displacement rod into the fluid reservoir. In some variations, input movement (e.g., movement of an actuator) in a first direction engages a first ratchet wheel with a linear gear and disengages a second ratchet wheel from the linear gear. Input movement (e.g., movement of an actuator) in a second direction engages the second ratchet wheel with the linear gear and disengages the first ratchet wheel from the linear gear. In some variations, a drive assembly including an extendable fluid coupler may be configured to move the fluid reservoir towards the displacement rod to deliver fluid to the elongate member. The displacement rod may remain fixed relative to the handle and the fluid reservoir may move toward the displacement rod in response to movement of an actuator in a first direction and/or a second direction opposite the first.

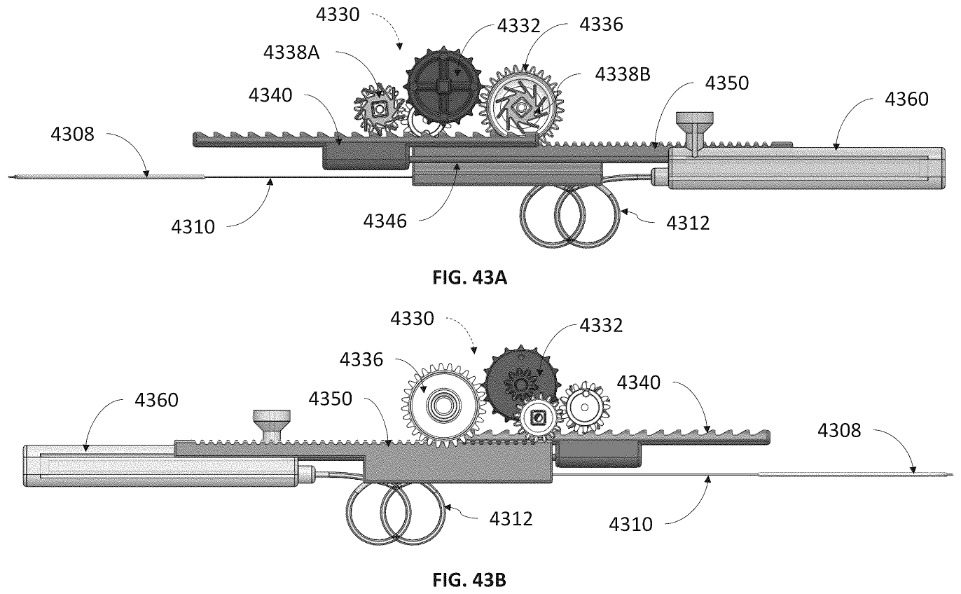

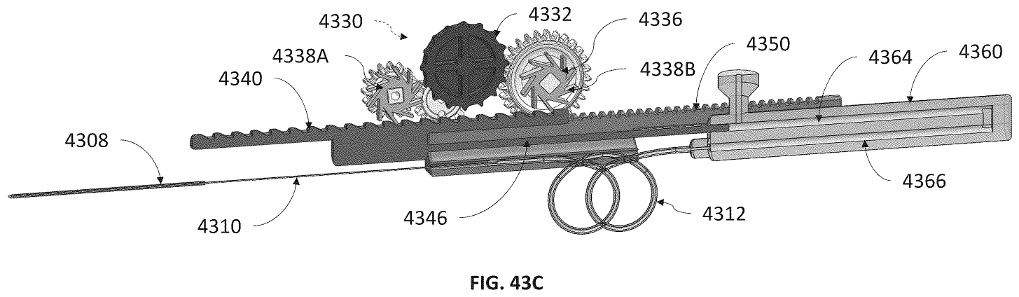

For example, as shown in FIGS. 43A-43C, a drive assembly 4330 may include a first linear gear 4340, a first ratchet wheel 4338A, and a second ratchet wheel 4338B. FIGS. 43A-43C show two side views and a cross-section perspective views, respectively. The first linear gear 4340 may be operatively coupled to a displacement rod 4346 configured to move into the fluid reservoir 4360. In particular, the displacement rod 4346 may move into a first lumen 4364 of the fluid reservoir 4360. Movement of the first linear gear 4340 may move the displacement rod 4346. In some variations, the first linear gear 4340 may be configured to move in a first direction (e.g., towards the fluid reservoir) and to not move in a second opposite direction (e.g., away from the fluid reservoir). Thus, in some variations, the first linear gear may be configured to move the displacement rod into the fluid reservoir and not out of the fluid reservoir. The first ratchet wheel 4338A may be configured to move the first linear gear 4340 in the first direction in response to input movement in a first direction (e.g., rotational motion in a first direction). The second ratchet wheel 4338B may be configured to move the first linear gear 4340 in the first (same) direction in response to input movement in a second opposite direction (e.g., rotational motion in a second opposite direction). In some variations, rotational movement of an actuator 4332 in a first direction engages the first ratchet wheel 4338A with the first linear gear 4340 and disengages the second ratchet wheel 4338B from the first linear gear 4340. In some variations, rotational movement of the actuator 4332 in the second opposite direction may engage the second ratchet wheel 4338B with the first linear gear 4340 and disengage the first ratchet wheel 4338A from the first linear gear 4340. In some variations, one ratchet wheel may engage the first linear gear at a time. The first and second ratchet wheel 4338A, 4338B may be in contact with or otherwise engage (e.g., through one or more intermediate gears) the actuator 4332 configured to be contacted by a user. Rotation of the actuator 4332 may be configured to rotate the first and second ratchet wheels 4338A, 4338B in opposite directions. In some variations, the first ratchet wheel 4338A may be configured to move the first linear gear 4340 towards the fluid reservoir 4360 at a first rate and the second ratchet 4338B may be configured to advance the first linear gear 4340 towards the fluid reservoir 4360 at a second different rate. For example, one or more of the pinion gear 4336 or intermediate gears may be configured to rotate the first and second ratchet wheels 4338A, 4338B at different rates such that each moves the first linear gear 4340 at a different rate. In some variations, the second rate (e.g., rate of movement during retraction) may be greater than the first rate (e.g., rate of movement during advancement).

In some variations, movement of the actuator 4332 in a first direction may advance the elongate member and movement of the actuator 4332 in a second opposite direction may retract the elongate member. The elongate member 4310 may be slidably positioned in a cannula 4308 and coupled to a second linear gear 4350. The elongate member 4310 may be configured to slidably advance from, and retract into, the cannula 4308. Movement of the second linear gear 4350 may advance and retract the elongate member 4310. In some variations, an extendable fluid coupler 4312 may fluidly couple the elongate member 4310 to the fluid reservoir 4360. The extendable fluid coupler may be fluidly coupled to a distal or proximal portion of the fluid reservoir 4360. In some variations, the extendable fluid coupler 4312 may be configured to allow advancement and retraction of the elongate member 4310 without changing a volume of a second lumen 4366 of the fluid reservoir 4360. The extendable fluid coupler 4312 may comprise a contracted configuration and an extended configuration. In some variations, advancement of the elongate member 4310 may move the expandable fluid coupler 4312 from the contracted configuration to the extended configuration. Retraction of the elongate member may move the fluid coupled from the extended configuration to the contracted configuration. In some variations, a proximal portion of the extendable fluid coupler 4312 may be coupled to the second linear gear 4350 such that movement of the second linear gear may transition the extendable fluid coupler between the contracted and extended configurations. The extendable fluid coupler may comprise, for example, looped or coiled tubing. In some variations, the extended fluid coupler may replace the plunger tube or decouple the plunger tube from movement of the second linear gear. The extended fluid coupler may be configured to allow fluid delivery to the elongate member similar to the plunger tube but without changing the volume of a second lumen of the fluid reservoir. In such variations, the displacement rod may be configured to deliver fluid to the elongate member during retraction by changing a volume of a first lumen of the fluid reservoir during advancement and retraction of the elongate member.

In some variations, the displacement rod 4346 may be configured to move toward the fluid reservoir to deliver fluid to the elongate member during both advancement and retraction of the elongate member. The actuator 4332 may be configured to move the displacement rod and advance or retract or re-advance or re-retract the elongate member simultaneously. The actuator 4332 may engage a pinion gear 4336 configured to move the second linear gear 4350 to advance or retract the elongate member 4310. Furthermore, the actuator may engage one or more of the ratchet wheels 4338A, 4338B to move the displacement rod 4346 toward the fluid reservoir 4360. The pinion gear 4336 may be coupled to the second ratchet wheel 4338B configured to move the first linear gear 4340, and the first linear gear 4340 may be operatively coupled to the displacement rod 4346. Additionally, the actuator 4332 may directly or via one or more intermediate gears engage the first ratchet wheel 4338A also configured to move the first linear gear 4340. In some variations, the movement of the actuator 4332 in a first direction may advance the elongate member 4310 and move the displacement rod 4346 into the fluid reservoir. For example, rotation of the actuator 4332 in the first direction may engage the pinion gear 4336 to move the second linear gear 4350 and advance the elongated member 4320 while also engaging the first ratchet wheel 4338A to move the displacement rod 4346 into the fluid reservoir. In some variations, the movement of the actuator 4332 in a second opposite direction may retract the elongate member 4310 and move the displacement rod 4346 into the fluid reservoir 4360. For example, rotation of the actuator 4332 in the second opposite direction may engage the pinion gear 4336 to move the second linear gear 4350 and retract the elongated member 4310 while also engaging the second ratchet wheel 4338B to move the displacement rod 4346 into the fluid reservoir 4360.

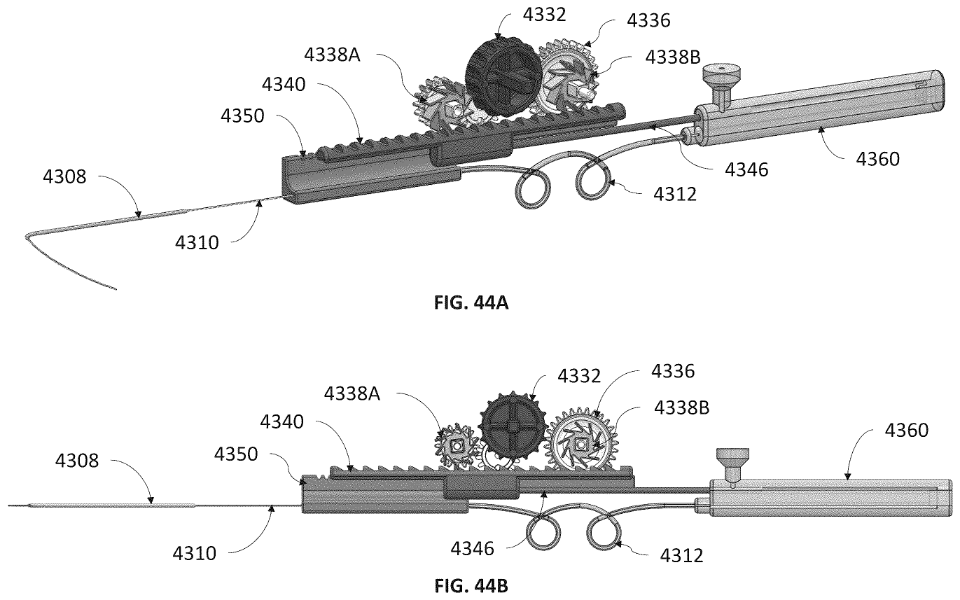

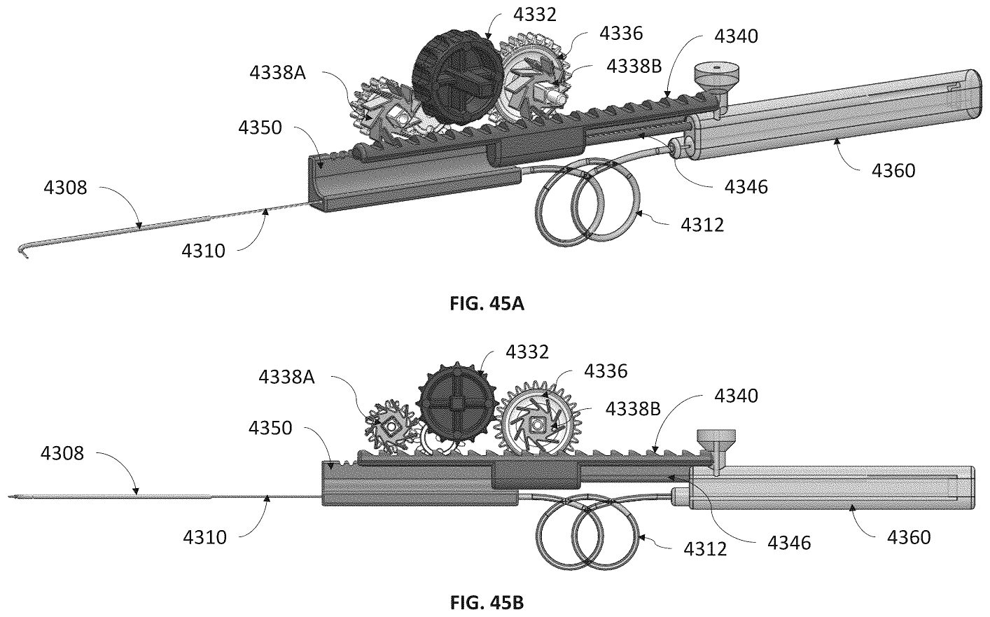

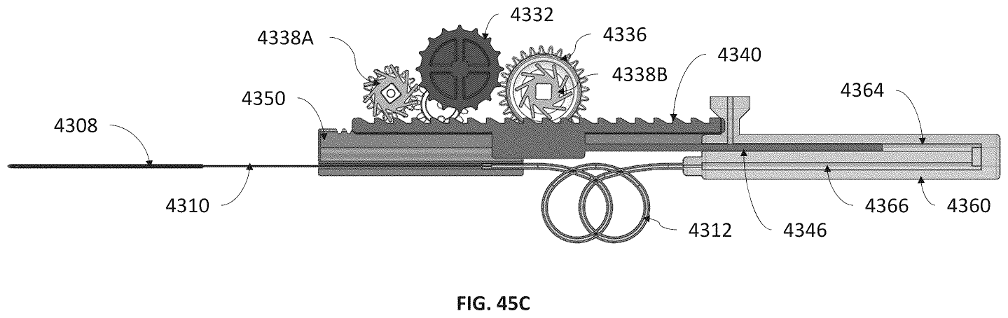

FIGS. 43A-45C depict various side, cross-sectional, and perspective views of a device configured to move the displacement rod into the fluid reservoir in different configurations during a fluid delivery cycle. FIGS. 43A-43C depict two side views and a cross-sectional perspective view of a pre-advancement (i.e., primed) configuration of the device before advancement. In the primed configuration, the displacement rod 4346 and the first linear gear 4340 may be in a distal position. The displacement rod 4346 may be partially within the fluid reservoir 4360 but may not yet be configured to advance into the fluid reservoir 4360 and dispense fluid. The elongate member 4310 may be entirely positioned within the cannula 4308 (e.g., completely retracted) in the primed configuration. The extendable fluid coupler 4312 may be in a contracted configuration. FIGS. 44A-44B depict perspective and side views respectively of an advancement configuration of the device. In the advancement configuration, the actuator 4332 may be rotated in a first direction. The actuator 4332 may be configured to engage the first ratchet wheel 4338A to move the first linear gear 4340 and may be configured to engage the second ratchet wheel 4338B to disengage the second ratchet wheel 4338B from the first linear gear 4340. The elongate member 4310 may be advanced by the second linear gear 4350. As shown in FIGS. 44A-44B, the displacement rod 4346 and the first linear gear 4340 may move in a proximal direction toward the fluid reservoir. More of the displacement rod 4346 may be positioned within (advanced into) the fluid reservoir 4360 in the advancement configuration compared to the primed configuration. The displacement rod 4346 may deliver fluid to the elongate member 4310 in the advancement configuration. The extendable fluid coupler 4312 may be in an extended configuration. FIGS. 45A-45C depict a perspective view, a side view, and a cross-sectional view respectively of a retracted configuration of the device. In the retracted configuration, the actuator 4332 may be rotated in a second direction opposite the first direction and may be configured to engage the second ratchet wheel 4338B to move the first linear gear 4340 and disengage the first ratchet wheel 4338A from the first linear gear 4340. The elongate member 4310 may be retracted by the second linear gear 4350. As shown in FIGS. 45A-45C, the displacement rod 4346 and the first linear gear 4340 may continue to move in a proximal direction toward the fluid reservoir. The displacement rod 4346 may be partially or fully advanced into the fluid reservoir 4346 in the retracted configuration. The extendable fluid 4312 coupler may return to the contracted configuration.

### Linear Actuator

Further variations of the drive assembly may not employ translation of rotational motion (e.g., around a central axis of the actuator, such as an wheel) to linear motion. For example, a slide (e.g., a finger slide) on the handle may be fixed or detachably coupled to a gear within the housing of the handle (e.g., a linear gear as previously described). The drive assembly may be configured so that advancement or retraction of the slide causes advancement or retraction of an elongate member and/or delivery of a fluid composition into Schlemm's canal. The slide may move in an arc, which may, in some instances, increase user comfort during use and make the device more ergonomic. A finger of the user may follow the arc movement of the slide, which may, in some instances, additionally or alternatively provide improved control over the device (e.g., movement of the elongate member, fluid delivery). In some variations, utilizing a slide may, additionally or alternatively, reduce a risk of a hand of the user unintentionally obstructing visualization of the device.

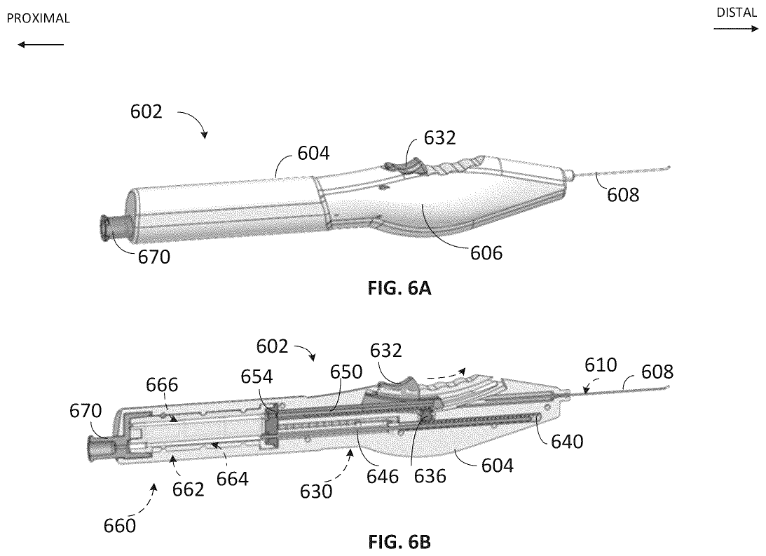

FIGS. 6A-6B depict an exemplary variation of a delivery device comprising an actuator that utilizes linear or otherwise non-rotational motion around an axis of an actuator (e.g., movement along an arc) to move other components of the drive assembly. As seen there, the delivery device may comprise a handle 602 having a housing 604 with a grip portion 606, a cannula 608 comprising an elongate member 610 therein, a drive assembly 630 that may comprise an actuator (e.g., slide 632), a first linear gear 650, a second linear gear 640, and a first pinion gear 636. The device may comprise a fluid assembly 660 that comprises a fluid reservoir 662 comprising a first lumen 664 in fluid communication with a fluid reservoir connector 670, a second lumen 666 in fluid communication with a first lumen 664 by a passageway, as described above. The fluid assembly 660 may include a displacement rod 646 configured to be moved by the first linear gear 650 and a plunger tube 654 coupled to the second linear gear 650 where the second linear gear is also coupled to the elongate member 610, so that movement of the second linear gear correspondingly moves the elongate member 610 and the plunger tube 654.

The actuator may move along a linear or non-rotational path along the handle to move other components of the drive assembly to move the elongate member and/or deliver fluid from the delivery device to the eye. For example, the slide 632 as seen in FIG. 6A-6B may move along a linear path along or within the handle. In some variations, the slide 632 may move along an arc path within the handle. In other variations, the slide may move along a linear path along or within the handle. Moving the slide 632 distally along the curved path may result in proximal movement of the first linear gear 640 and distal movement of the second linear gear 650, resulting in advancement of the elongate member 610 with fluid delivery due to proximal movement of the displacement rod 646 into the first lumen 664 and distal movement of the plunger tube 654 out of the second lumen 666. Moving the slide 632 proximally along the curved path, may result in distal movement of the first linear gear 640 and proximal movement of the second linear gear 650, resulting in retraction of the elongate member 610 with fluid delivery with proximal movement of the plunger tube 654 into the second lumen 666 with the displacement rod 646 stationary within the first lumen 664. The slide 632 may translate along a track within the housing 604 of the handle 602, which in some variations, may form an arc or may otherwise have a curved shape. In some variations, the curved shape of the track within the housing of the handle may allow a user to naturally move the slide in a distal direction or in a proximal direction. For example, the curved shape of the track may optimize the ergonomics of a finger of a user sweeping through a pushing motion. The ergonomics of the finger of the user may allow for extended and comfortable actuation by a user's finger, while maintaining applied pressure for a controlled grip. In variations in which a curved track is employed, the track may have a first angle of curvature. For example, the first angle of curvature may include an angle of curvature of about 5 degrees to about 45 degrees including about 10 degrees, about 15 degrees, about 20 degrees, about 25 degrees, about 30 degrees, about 35 degrees, about 40 degrees, or about 45 degrees through a 2.0″ radius arc. In some variations, the handle may comprise a gear multiplier for slider arc length that may be configured to yield an increased distance of travel for the elongate member compared to handles without a gear multiplier. In some variations, the curved track may have a concave shape, while in other variations the curved track may have a convex shape. In some variations, the track may have one or more variations therein to provide a tactile feel or audible sound for the user to indicate the length of travel of the slide along the track.

### Lock Mechanism

In some variations, the handle may comprise a lock configured to prevent premature actuation of the device. For example, the lock may prevent movement of one or more components of the device (e.g., components of the drive assembly (e.g., actuators, gears), components of the fluid assembly (e.g., fluid reservoir, plunger, displacement rod)) during transport of the device. The lock may be configured to preserve a configuration (e.g., retracted configuration) of the device prior to use. In some variations, the lock may comprise an engaged configuration and a disengaged configuration. In the engaged configuration, the lock may prevent the actuation of the device and thus prevent the delivery of fluid to the elongate member. For example, the lock may prevent movement of a displacement rod into the reservoir. In some variations, the lock may be configured to prevent one or more components of the drive assembly from moving while the lock is in the engaged configuration. In the disengaged configuration, the lock may allow the device to function normally. For example, the device may advance and retract the elongate member and deliver fluid to the elongate member while the lock is in the disengaged configuration. In some variations, the lock may only be configured to transition between the engaged and disengaged configuration once. Put differently, the lock may be configured to transition from the engaged configuration to the disengaged configuration, but not from the disengaged configuration back to the engaged configuration. Additionally or alternatively, in some variations, the lock may be transitioned from the disengaged configuration to the engaged configuration. For example, the lock may be transitioned to the engaged configuration after use of the device to prevent further delivery of a fluid composition.

In some variations, the lock may be configured to be actuated by a user. Actuation by the user may transition the lock between the engaged and the disengaged configuration. For example, the lock may be slidably coupled to the handle. In the engaged configuration, at least a portion of the lock may extent outwardly from the handle. This portion of the lock may be configured to be actuated by a user to transition the lock to the disengaged configuration. For example, the lock may be configured to slide at least partially into the handle when actuated. In some variations, the lock may be flush with the handle in the disengaged configuration. In some variations, the lock may comprise one or more dents, detents, and/or protrusions corresponding to one or more corresponding dents detents, and/or protrusions in the handle configured to maintain the position (e.g., engage configuration, disengaged configuration) of the lock. Additionally or alternatively, friction between the lock and the handle and/or drive assembly may maintain the position of the lock in the engaged and/or disengaged configuration. In some variations, movement of one or more actuators may transition the lock from the engaged configuration to the disengaged configuration or vice vera. In some variations, movement of one or more actuators configured to advance or retract the elongate member may disengage the lock (e.g., a detent of the lock) and allow the lock to be transitioned between configurations.

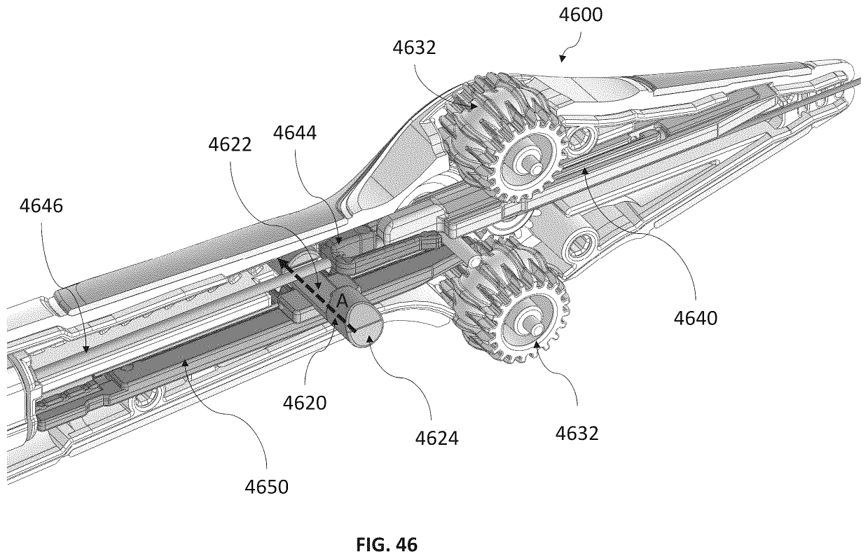

For example, FIG. 46 depicts a perspective view of a device 4600 including a lock 4620. FIG. 46A depicts the lock 4620 in the engaged configuration. In the engaged configuration, the lock 4620 may be configured to prevent the movement of the displacement rod 4646 into the fluid reservoir and thus prevent the delivery of fluid to the elongate member. The lock may be further configured to prevent the advancement or retraction of the elongate member. To prevent the movement of the displacement rod 4646, the lock may be configured to prevent movement of one or more components of a drive assembly including a ratchet coupled to a displacement rod 4644, a first linear gear 4640, a second linear gear 4650, and one or more actuators 4632. For example, as shown in FIG. 46, the lock 4620 may contact the ratchet coupled to the displacement rod 4644 preventing the ratchet from moving and advancing the displacement rod 4646 into the reservoir. The ratchet may be releasably coupled to the first linear gear 4640 such that the lock 4620 preventing ratchet 4644 from moving also prevents the first linear gear 4640 from moving. Likewise, the lock 4620 preventing the first linear gear 4640 from moving may prevent one or more pinion gears, one or more actuators 4632, and the second linear gear 4650 from moving. Thus, in the engaged configuration the lock may be configured to prevent the drive assembly from moving. Furthermore, the lock 4620 preventing the second linear gear 4650 from moving may prevent advancement and retraction of the elongate member.

In some variations, a lateral portion 4624 of the lock may be actuated by a user to transition the lock from the engaged configuration, shown in FIG. 46, to a disengage configuration (not shown). In the disengaged configuration, a slot of the lock 4622 may be configured to allow movement of the ratchet coupled to the displacement rod 4644. The lock allowing movement of the ratchet 4644 may allow movement of one or more other components of the drive assembly. For example, actuation of the lateral portion of the lock 4624 may move the lock 4620 along path (A) to the disengaged configuration. In the disengaged configuration, the slot 4622 may be aligned with the ratchet coupled to the displacement rod 4644 and configured to allow the ratchet 4644 to pass through the slot 4622 and move the displacement rod 4646.

In some variations, a portion of the handle and one or more components of the drive assembly may be reusable. A reusable portion of the device may include one or more of a linear gear, a pinion gear, a ratchet, a clutch, a motor, a ratchet wheel, and an actuator. One or more of these components may be coupled to a reusable handle. The reuseable portion of the device (e.g., handle, drive assembly) may receive one or more disposable elements. For example, a disposable element may comprise an elongate member and/or a cannula. In some variations, the disposable element may additionally or alternatively include one or components of the fluid assembly (e.g., a plunger tube, fluid reservoir). In some variations, the one or more disposable elements (which may or may not be coupled to one another) may be configured to releasably couple to the handle (e.g., handle housing) prior to a procedure and decouple from the handle (e.g., handle housing) following the procedure.

When the delivery device is configured to deliver a fluid composition, actuating the actuator configured to be contacted by a user (e.g., rotating the rotatable elements 432A-432B or moving a slide 632) may also cause delivery of a fluid composition. The delivery of the fluid composition may be simultaneous with movement (e.g., advancement and/or retraction) of the slidable elongate member 410. In some of these instances, the fluid composition may be delivered to the portion of Schlemm's canal in which the slidable elongate member 410 is advanced. That is, the fluid composition may be delivered to the same arc length of Schlemm's canal as the extension of the elongate member 410. When delivery of the fluid composition is simultaneous with retraction of the elongate member 410, fluid composition may take the place of the slidable elongate member 410 as it is retracted and may dilate Schlemm's canal and/or the collector channels and/or the juxtacanalicular meshwork at that location in Schlemm's canal.

In other variations, the quantity or volume of the fluid composition delivered may not be tied to the amount of movement of the elongate member. Put differently, the quantity or volume of the fluid composition delivered may be independent of the amount of movement of the elongate member. In some variations, the device may be configured to deliver a bolus of fluid based on a user input separate from actuation of the drive assembly and movement of the elongate member. For example, input from a user may dispense a predetermined quantity of fluid (e.g., about 5 μL, about 10 μL, about 20 μL, about 30 μL, about 40 μL, or about 50 μL of fluid). In some variations, the fluid reservoir may be compressed to deliver the bolus of fluid. The user may engage an actuator (e.g., button) that may contact the fluid reservoir to compress the reservoir and deliver fluid. In some variations, may control the amount of fluid delivered independent from advancement of the elongate member. For example, an actuator (e.g., button, slide, wheel) not coupled to the drive assembly may allow a user to control the volume in a lumen of the fluid reservoir. The actuator may directly couple to one or more of the displacement rod, the plunger tube, or another displacement rod to move the respectively component independently of the rest of the drive assembly and/or the elongate member.

### Fluid Assembly

Some variations of the delivery devices may include a fluid assembly such that the devices are configured to deliver a fluid composition to the eye as described in more detail herein. Referring again to FIG. 7C, the delivery device 400 may comprise a fluid assembly 460 comprising one or more of: a fluid reservoir 462 in fluid communication with the elongate member 410, a displacement rod 446, and a plunger tube 450. The fluid assembly 460 may be at least partially contained within the housing 404 of the handle 402. For example, in some variations, the fluid reservoir 462 may be fully contained within the housing 404 of the handle 402, while in other variations, a portion of the fluid reservoir 462 may be contained within the housing 404 of the handle 402 and a portion may extend (e.g., proximally, upwardly, downwardly, laterally) beyond or outside of the housing 404 of the handle 402. In some variations, the fluid reservoir 462 may include a fluid reservoir connector 470 configured to receive fluid therein from an external fluid device.

As seen in FIG. 7C, the fluid assembly 460 may include a proximal end cap 475 that covers the proximal end of the fluid reservoir 462. In variations comprising a fluid reservoir connector 470, the proximal end cap 475 may include the fluid reservoir connector 470 therein and the fluid reservoir connector 470 may be in fluid communication with one or more lumens of the fluid reservoir 462. In some variations, the fluid reservoir connector may include a Leur fitting, configured to detachably couple to an external fluid device. In some variations, the proximal end cap 475 may include one or more abutments 473 configured to seal one end (the proximal end) of the one or more lumens. For example, the one or more abutments 473 may be received within, surround, or otherwise form a seal with the interior surface of the one or more lumens and/or the surface surrounding a proximal opening of the one or more lumens. The proximal end cap 475 may include a sealing member 474 and a valve 472 configured to seal fluid within the one or more lumens and direct fluid into the one or more lumens, respectively. In some variations, the valve 472 may include a one-way valve including a duckbill valve, a check valve, or another type of one-way valve. In some variations, the sealing member 474 may include an O-ring.

The fluid assembly 460 may further comprise a distal end cap 480. The distal end cap 480 may cover the distal end of the fluid reservoir 462. The distal end cap 480 may comprise a plurality of lumens (e.g., 2, 3, 4, or more). For example, the distal end cap 480 may comprise a first lumen 490 therethrough and a second lumen 492 therethrough. The first lumen 490 may be configured to receive a displacement rod therethrough and the second lumen 492 may be configured to receive a plunger tube therethrough. The distal end cap 480 may comprise a plurality of lumen sealing members, the number of lumen sealing members corresponding to the number of lumens in the fluid reservoir. For example, the distal end cap 480 may include a first lumen sealing member 486 for the first lumen 490 and a second lumen sealing member 484 for the second lumen 492. The first and second lumen sealing members 484, 486 may be configured to prevent fluid from moving out of the fluid reservoir 462 through the first lumen 490 and the second lumen 492 respectively, aligned with the first lumen 464 and the second lumen 466. The lumen sealing members 484, 486 may further be configured to allow for movement of the displacement rod and the plunger tube into and out of the fluid reservoir 462, as will be described in more detail herein. The distal end cap 480 may also include a main sealing member 482 positioned proximal the distal end cap 480. Each of the proximal end cap 475 and the distal end cap 480 may be coupled to the fluid reservoir 462 is any number of ways. For example, the proximal end cap 475 and the distal end cap 480 may be coupled to the fluid reservoir 462 by one or more of a press fit, an interference fit, a snap fit, adhesive, or the like. In some variations, the lumen sealing members 484, 486, and the main sealing member 482 may be combined into a single sealing member (integral seal) that may seal each of the first lumen, the second lumen, and the distal end cap.

### Fluid Reservoir

As noted above, the fluid assembly may comprise a fluid reservoir configured to contain, store, provide, or otherwise house fluid for delivery to the eye. The fluid reservoir may comprise a fluid reservoir body and one or more lumens configured to contain, store, provide, or otherwise house fluid therein. In order to provide flexibility in the amount of fluid delivered to the eye during a procedure, and when the fluid is delivered, especially relative to movement of the elongate member, the fluid reservoir 462 may comprise a plurality of lumens (e.g., 2, 3, 4, or more), which may or may not be fluidically coupled to one another. In some variations, the fluid reservoir having multiple lumens may allow the device to deliver specific volume of the fluid composition during both advancement of the elongate member and retraction of the elongate member. In some variations, it may be beneficial to fluidically couple the one or more lumens to one another via one or more passageways to easily allow transfer of fluid between the lumens during loading, priming, and/or a procedure. For example, in some variations, the fluid reservoir may comprise two passageways between a first lumen and a second lumen, about three passageways between a first lumen and a second lumen, about four lumens between the first lumen and the second lumen or the like. Two or more passageways may provide multiple ways for a fluid composition to flow from one lumen to another to ensure that a fluid composition may always flow from one lumen to another lumen without getting blocked. In some variations, the one or more passageways may fluidically couple the first lumen to the second lumen at a proximal end, a distal end, or a place therebetween. In some variations, the passageway may fluidically couple the first lumen from a proximal end to a distal end of a second lumen, or from a distal end of a first lumen to a proximal end of a second lumen. In some variations, the fluid reservoir may be unitary (a single piece) or may otherwise have the one or more lumens formally integrally with the fluid reservoir body.

Turning back to FIG. 7C, shown there is an exemplary fluid reservoir 462 for the delivery devices described herein. The fluid reservoir 462 comprises a first lumen 464 and a second lumen 466 that are fluidically coupled to one another by a passageway 468. Fluid may flow from the first lumen 464 to the second lumen 466 via the passageway 468, and from the second lumen 466 out of the fluid reservoir 462. It can be appreciated that in some variations, fluid may flow from the second lumen 466 to the first lumen 464 and from the first lumen 464 out of the fluid reservoir 462.

The plurality of lumens may be arranged in any configuration within the fluid reservoir body that allows for delivery of the fluid from the lumens to the eye. For example, the first and second lumens 464, 466 may be arranged parallel and may be adjacent to one another. In some variations, the first and second lumens 464, 466 may be arranged laterally adjacent one another, while in other variations the first and second lumens 464, 466 may be arranged vertically adjacent one another (e.g., the first lumen 464 may be positioned above the second lumen 466, or vice versa). The first and second lumens may each have a longitudinal axis that is parallel to a longitudinal axis of the fluid reservoir body, or may otherwise extend from a proximal to a distal end of the fluid reservoir body (lengthwise through the fluid reservoir body). The passageway 468 fluidically coupling the first and second lumens may be positioned perpendicularly to the first and second lumens and/or may comprise a longitudinal axis that traverses the longitudinal axes of the first and second lumens.

Generally, each of the plurality of lumens of the fluid reservoir may have any length suitable for delivering a volume of fluid to the eye. In some variations, each lumen of the plurality of lumens may have the same length, while in other variations one or more of the plurality of lumens may have a different length than a length of another of the one or more of the plurality of lumens. For example, in some variations as seen in FIG. 7C, the first lumen 464 may have a first length 467 and the second lumen 466 may have a second length 471. The first length 467 of the first lumen 464 may be the same as the second length 471 of the second lumen 466 or the first length 467 of the first lumen 464 may be different from the second length 471 of the second lumen 466. In some variations, the first length 467 of the first lumen 464 may be greater than the second length 471 of the second lumen 466, while in other variations, the second length 471 of the second lumen 466 may be greater than the first length 467 of the first lumen 464.

Additionally, each of the plurality of lumens of the fluid reservoir may have any diameter or height suitable for delivering a volume of fluid to the eye. In some variations, each lumen of the plurality of lumens may have the same diameter or height, while in other variations, one or more of the plurality of lumens may have a different diameter or height than a diameter or height of another lumen of the plurality of lumens. Moreover, the diameter or height of one or more of the plurality of lumens may be constant along the length of the lumen or the diameter or height may vary along all or a portion of the length of the lumen. For example, in some variations, the first lumen 464 may have a first diameter 465 and the second lumen 466 may have a second diameter 469 different from the first diameter 465. The first diameter 465 of the first lumen 464 may be greater than the second diameter 469 of the second lumen 466, while in other variations, the second diameter 469 of the second lumen 466 may be greater than the first diameter 465 of the first lumen 464. In some variations, the first diameter 465 of the first lumen 464 may be constant along the first length 467 of the first lumen 464, while in other variations, the first diameter 465 of the first lumen 464 may be variable along at least a portion of the first length 467 of the first lumen 466. For example, in some variations, the first diameter 465 of the first lumen 464 may decrease from a proximal end of the first lumen 464 to a distal end of the first lumen 464, while in other variations, the first diameter 465 of the first lumen 464 may decrease from a distal end of the first lumen 464 to a proximal end of the first lumen 464. In some variations, the second diameter 469 of the second lumen 466 may be consistent along the second length 471 of the second lumen 466, while in other variations, the second diameter 469 of the second lumen 466 may be variable along at least a portion of the second length 471 of the second lumen 466. For example, in some variations, the second diameter 469 of the second lumen 466 may decrease from a proximal end of the second lumen 466 to a distal end of the second lumen, while in other variations, the second diameter 469 of the second lumen 466 may decrease from a distal end of the second lumen 466 to a proximal end of the second lumen 466.

Generally, the each of the lumens of the plurality of lumens may have a cross-sectional shape suitable for delivering a fluid to the eye. In some variations, each lumen of the plurality of lumens may have the same cross-sectional shape, while in other variations, one or more of the plurality of lumens may have a different cross-sectional shape than a cross-sectional shape of another lumen of the plurality of lumens. Moreover, the cross-sectional shape of one or more of the plurality of lumens may be constant along the length of the lumen or the cross-sectional shape may vary along all or a portion of the length of the lumen. For example, in some variations, such as that depicted in FIG. 7C, the first lumen 464 may have a first cross-sectional shape and the second lumen 466 may have a second cross-sectional shape. In some variations, the first cross-sectional shape of the first lumen 464 may be the same as, or similar to, the second cross-sectional shape of the second lumen 466, while in other variations, the first cross-sectional shape of the first lumen 464 may be different from the second cross-sectional shape of the second lumen 466. The cross-sectional shape of each of the plurality of lumens may include a circle, triangle, square, star, pentagon, hexagon, octagon, any other polygon or the like. As seen in FIG. 7C, the both the first and second lumens 464, 466 have a constant, circular cross-sectional shape.

It can be appreciated that one or more geometric differences (e.g., differences in length, diameter or height, and/or cross-sectional shape) between the plurality of lumens may allow the lumens to have a specific volume, including different volumes or the same volume. For example, in some variations, the first lumen 464 may have a first volume and the second lumen 466 may have a second volume. the first volume of the first lumen 464 may be the same as the second volume of the second lumen 466, or the first volume of the first lumen 464 may be different from the second volume of the second lumen 466. For example, in some variations, the first volume of the first lumen 464 may be greater than the second volume of the second lumen 466, while in other variations, the first volume of the first lumen 464 may be less than the second volume of the second lumen 466. It can be appreciated that the difference in the volumes of the first lumen 464 and the second lumen 466 may allow different volumes of fluid to be delivered from the first lumen 464 and the second lumen 466 to the eye, such as, for example, during different portions of a procedure. In some variations, the first lumen may have a first volume from about 4 μL to about 50 μL and the second lumen may have a second volume from about 10 μL to about 60 μL. For example, the first lumen may have a first volume of about 4 μL, about 5 μL, about 6 μL, about 7 μL, about 8 μL, about 9 μL, about 10 μL, about 11 μL, about 12 μL, about 13 μL, about 14 μL, about 15 μL, about 16 μL, about 17 μL, about 18 μL, about 19 μL, about 20 μL, about 21 μL, about 22 μL, about 23 μL, about 24 μL, about 25 μL, about 26 μL, about 27 μL, about 28 μL, about 29 μL, about 30 μL, about 31 μL, about 32 μL, about 33 μL, about 34 μL, about 35 μL, about 36 μL, about 37 μL, about 38 μL, about 39 μL, about 40 μL, about 41 μL, about 42 μL, about 43 μL, about 44 μL, about 45 μL, about 46 μL, about 47 μL, about 48 μL, about 49 μL, or about 50 μL. For example, the second lumen may have a second volume of about 10 μL, about 11 μL, about 12 μL, about 13 μL, about 14 μL, about 15 μL, about 16 μL, about 17 μL, about 18 μL, about 19 μL, about 20 μL, about 21 μL, about 22 μL, about 23 μL, about 24 μL, about 25 μL, about 26 μL, about 27 μL, about 28 μL, about 29 μL, about 30 μL, about 31 μL, about 32 μL, about 33 μL, about 34 μL, about 35 μL, about 36 μL, about 37 μL, about 38 μL, about 39 μL, about 40 μL, about 41 μL, about 42 μL, about 43 μL, about 44 μL, about 45 μL, about 46 μL, about 47 μL, about 48 μL, about 49 μL, about 50 μL, about 51 μL, about 52 μL, about 53 μL, about 54 μL, about 55 μL, about 56 μL, about 57 μL, about 58 μL, about 59 μL, or about 60 μL. In some variations, the first lumen and the second lumen may have a volume about 30 μL.

In variations comprising a plurality of lumens, the total volume of the fluid reservoir may be the volume of the plurality of lumens. For example, in variations in which the fluid reservoir comprises a first lumen and a second lumen, the total volume of the fluid reservoir may be the sum of the volumes of the first and second lumens. In some variations, the total volume of the fluid reservoir may be about 30 μL to about 2000 μL including from about 30 μL to about 60 μL, or from about 30 μL to about 100 μL, or from about 30 μL to about 200 μL, or from about 30 μL to about 300 μL, or from about 30 μL to about 400 μL, or from about 30 μL to about 500 μL, or from about 30 μL to about 600 μL, or from about 30 μL to about 700 μL, or from about 30 μL to about 800 μL, or from about 30 μL to about 900 μL, or from about 30 μL to about 1000 μL, from about 30 μL to about 1100 μL, from about 30 μL to about 1200 μL, from about 30 μL to about 1300 μL, from about 30 μL to about 1400 μL, from about 30 μL to about 1500 μL, from about 30 μL to about 1600 μL, from about 30 μL to about 1700 μL, from about 30 μL to about 1800 μL, from about 30 μL to about 1900 μL, from about 30 μL to about 2000 μL. In some variations, the total volume of the fluid reservoir may be about 30 μL, about 31 μL, about 32 μL, about 33 μL, about 34 μL, about 35 μL, about 36 μL, about 37 μL, about 38 μL, about 39 μL, about 40 μL, about 41 μL, about 42 μL, about 43 μL, about 44 μL, about 45 μL, about 46 μL, about 47 μL, about 48 μL, about 49 μL, about 50 μL, about 51 μL, about 52 μL, about 53 μL, about 54 μL, about 55 μL, about 56 μL, about 57 μL, about 58 μL, about 59 μL, or about 60 μL. It can be appreciated that any of the plurality of lumens, such as, for example, the first lumen and the second lumen, may be specifically constructed through adjustment of one of more of the length, the diameter or height, and the cross-sectional shape such that the lumen may hold a specific, predetermined volume of fluid.

### Plunger Tube

The fluid assembly may include a plunger tube in fluid communication with the elongate member. The plunger tube may be coupled to the second linear gear such that movement of the second linear gear may move the plunger tube. The plunger tube may be positioned within a lumen of the fluid reservoir. Generally, a portion of the plunger tube may move within (e.g., advance into and retract out of) the fluid reservoir to deliver fluid from the fluid reservoir to the elongate member. FIG. 7D depicts a cross-sectional view of the fluid assembly 460 with some components of the drive assembly. The fluid assembly 460 may comprise the fluid reservoir 462 comprising a first lumen 464 and a second lumen 466 with a passageway 468 fluidically connecting the first lumen 464 and the second lumen 466. The fluid assembly 460 may further include a fluid reservoir connector 470 in fluid communication with the first lumen 464. The drive assembly may comprise a first linear gear 440 configured to move a ratchet 444 coupled to the displacement rod 446 and a second linear gear 450 coupled to a plunger tube 454. The plunger tube 454 may comprise a plunger tube lumen 456 in fluid communication with the elongate member 410 coupled to the second linear gear 450. Movement of the second linear gear 450 may correspondingly move the plunger tube 454 distally out of the second lumen 466 or proximally into the second lumen 466. Movement of the first linear gear 440 proximally may move the ratchet 444 and may correspondingly move the displacement rod 446. However, distal movement of the first linear gear 440 may not move the displacement rod 446 as will be described in more detail herein.

As described in detail above, the fluid assembly 460 may comprise a plunger tube 454. The plunger tube may be slidable within at least one of the two or more lumens of the fluid reservoir. For example, as seen in FIG. 7D, the plunger tube 454 may be moveable within the second lumen 466 of the fluid reservoir. In some variations, the plunger tube 454 may include a plunger tube lumen 456 therethrough. The plunger tube lumen 456 may be in fluid communication with each of the second lumen 466 and the elongate member 410, so that fluid may flow from the second lumen 466 through the plunger tube lumen 456 and through the elongate member 410. The plunger tube 454 may have a length and a diameter and, in some variations, the diameter of the plunger tube 454 may be consistent along a length of the plunger tube. The diameter of the plunger tube may be less than the diameter or height of the second lumen 466, so that fluid may flow around the plunger tube 454 while a portion of the plunger tube 454 resides within the second lumen 466. The plunger tube 454 may have a length that is greater than the length of the second lumen 466, so that a portion of the plunger tube 454 resides outside the second lumen 466. In some variations, the diameter of the plunger tube 454 may be varied along a length of the plunger tube 454. For example, in some variations, the diameter of the plunger tube 454 may decrease from a distal end of the plunger tube 454 to a proximal end of the plunger tube 454. In some variations, the diameter of the plunger tube 454 may decrease from a proximal end of the plunger tube to a distal end of the plunger tube 454. In some variations, the diameter of the plunger tube may be user adjustable, where a user may be able to adjust the diameter of the plunger tube for the needs of a specific patient and/or procedure.

The plunger tube may be constructed of any suitable material. For example, the plunger tube may be constructed of metal, plastic, rubber, polymers, or composites thereof. In some variations, the plunger tube 454 may be coupled to the second linear gear 450 and movement of the second linear gear 450 may move the plunger tube 454 within the second lumen 466. For example, distal movement of the second linear gear 450 may cause distal movement of the plunger tube 454, wherein a greater portion of the plunger tube 454 may reside outside of the second lumen 466 than a portion of the plunger tube 454 residing within the second lumen 466. Proximal movement of the second linear gear 450 may cause proximal movement of plunger tube 454 into the second lumen 466. Movement of the second linear gear 450 may be configured to deliver fluid from the second lumen 466 through the plunger tube lumen 456 to the elongate member 410.

### Displacement Rod

The fluid assembly may include a displacement rod configured to move within the fluid reservoir to deliver fluid from the fluid reservoir to the eye. The displacement rod may be coupled to a ratchet configured to be moved in a first direction by the first linear gear. The ratchet may be configured not to be moved in a second direction opposite the first direction by the first linear gear. Generally, the displacement rod and the plunger tube may be moved simultaneously to deliver fluid from the fluid reservoir during movement of the elongate member in a first direction, and the plunger tube but not the displacement rod may be moved to deliver fluid from the fluid reservoir during movement of the elongate member in a second direction opposite the first direction. For example, the displacement rod may move in a first direction opposite a second direction of the plunger tube during a first full advancement of the elongate member. After completion of the first full advancement of the elongate member, the displacement rod may be stationary while the plunger tube may move in both the first direction and the second direction.

In some variations, the displacement rod may be configured to move in the same direction as the plunger tube. For example, in some variations, the displacement rod may be proximal the fluid reservoir, where a proximal end of the fluid reservoir may be configured to receive the displacement rod. The displacement rod and plunger tube may move in the same direction (e.g., parallel) during advancement of the elongate member.

As seen in FIG. 7D, the fluid assembly 460 includes a displacement rod 446. The displacement rod may be slidable within at least one of the plurality of lumens of the fluid reservoir. For example, as seen in FIG. 7D, the displacement rod 446 may be at least partially received within the first lumen 464. The displacement rod 446 may be solid and constructed of any suitable material, such as, for example metal, plastic, rubber, polymers, or composites thereof.

The displacement rod 446 may have a length that is greater than a length of the first lumen 464 such that a portion of the displacement rod 446 may reside outside the first lumen 464 to be coupled to a component of the drive assembly for actuation and movement. For example, the displacement rod 446 may be coupled to the ratchet 444 at some point along a length of the displacement rod 446, so that movement of the ratchet 444 moves the displacement rod 446. For example, the ratchet 444 may be coupled to the displacement rod 446 at a location along the displacement rod 446 so that a length of the displacement rod 446 may be positioned within the first lumen 464 when the ratchet 444 is moved by the first linear gear 440.

In some variations, the diameter of the displacement rod may be constant along the length of the displacement rod 446, while in other variations, the diameter of the displacement rod may vary along at least a portion of the length of the displacement rod. For example, the diameter of the displacement rod may increase from a proximal end to a distal end or may decrease from a proximal end to a distal end. In some variations, the displacement rod may have a first diameter. The first diameter of the displacement rod may be less than the first diameter of the first lumen to ensure that fluid may flow around the displacement rod while a portion of the displacement rod is occupying the first lumen, to allow fluid to flow from the first lumen to the second lumen and from the second lumen to the eye. In some variations, the displacement rod 446 may have a cross sectional shape. In some variations, the displacement rod 446 may a consistent cross-sectional shape along the first length, while in other variations, the displacement rod may have a varied cross-sectional shape along at least a portion of the first length. In some variations, the diameter of the displacement rod may be user adjustable, where a user may be able to adjust the diameter of the displacement rod for the needs of a specific patient and/or procedure.

As described above, in some variations, proximal movement of the first linear gear 440 may move the ratchet 444 distally and may correspondingly move the displacement rod 446 proximally, while distal movement of the first linear gear 440 may not move the ratchet 444 and thus may not correspondingly move the displacement rod 446 distally. In some variations, the first linear gear 440 may have a channel 442 therein that may be configured to receive at least a portion of the displacement rod 446, and at least a portion of the displacement rod 446 may be slidable within the channel 442. Proximal movement of the first linear gear 440 may push the ratchet 444 proximally and may move the displacement rod 446 into the first lumen 464. Since the first linear gear 440 is not coupled to the displacement rod 446 but instead receives the displacement rod 446 within the channel 442, any distal movement of the first linear gear 440 that follows proximal movement of the first linear gear 440, the ratchet 444 and the displacement rod 446 allows the first linear gear 440 to move distally without moving the ratchet 444 and/or the displacement rod 446.

Proximal movement of the first linear gear 440 may move the ratchet 444 and the displacement rod 446 toward the fluid reservoir 462. Put another way, proximal movement of the first linear gear may correspondingly move the ratchet 444. Proximal movement of the first linear gear may decrease the distance between the ratchet 444 and the fluid reservoir 462 with a greater portion of the displacement rod 446 occupying the first lumen 464. With the ratchet 444 and the first linear gear 440 being in physical contact, only movement of the first linear gear 440 in a proximal direction may move the ratchet 444 and the displacement rod 446 proximally into the first lumen 464. Actuation of the first linear gear 440 in a first direction may cause proximal movement of the displacement rod 446 so that the first lumen 464 is completely occupied by the displacement rod 446. The channel 442 of the first linear gear 440 may allow the displacement rod 446 to stay seated within the first lumen 464, while actuation of the first linear gear 440 in a second direction opposite the first direction allows for the first linear gear 440 to move distally without the displacement rod 446 moving out of the first lumen 464. In some variations, any proximal movement of the displacement rod 446 within the first lumen 464 may be final, in that once the displacement rod 446 moves proximally, the displacement rod 446 cannot be moved distally. Put different, in some variations, the device may be configured to allow for proximal movement of the displacement rod 446 but may prevent distal movement of the displacement rod 446, since the distal movement of the first linear gear 440 may not contribute to distal movement of the ratchet 444 and thus the displacement rod 446. As the displacement rod 446 is moved proximally within the first lumen 464, fluid within the first lumen 464 may be displaced from the first lumen 464, through the passageway 468 to the second lumen 466, to be further displaced from the second lumen 466 through the plunger tube lumen 456 to the elongate member 410 and then the eye.

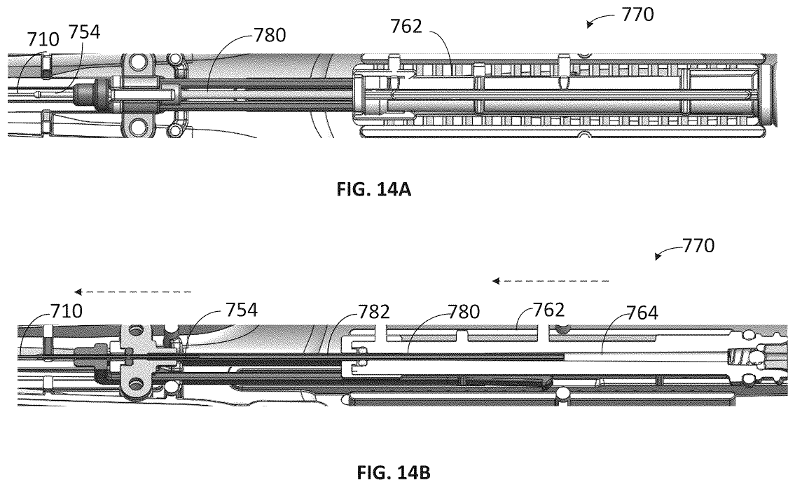

In some variations, as described above, the fluid reservoir may be moveable within the handle. In some variations, as seen in FIGS. 14A-14B, the fluid assembly 770 may include an inner tube positioned at least partially within an outer tube. For example, the fluid assembly 770 may comprise concentric tubes, wherein an outer tube 780 is concentric with the plunger tube 754. The plunger tube 754 may be as described herein. The fluid reservoir 762 may be as described herein. In some variations, the fluid reservoir 762 may comprise a single lumen 764 configured to receive the plunger tube 754 and/or the outer tube 780 therein and the outer tube 780 may be sealed relative to the plunger tube 754. The outer tube 780 may have a lumen 782 therethrough that includes the plunger tube 754 therein and the plunger tube 754 may be moveable within the lumen 782. The outer tube 780 may be stationary within the handle, while the plunger tube 754 may be moveable within the handle, within the outer tube 780, and within the lumen of the fluid reservoir 764. In some variations, the outer tube 780 may be coupled to the housing or one or more portions of the handle, which may constrain movement of the outer tube 780 relative to the handle (e.g., to the outer tube 780 may be stationary within the handle). In some variations, the plunger tube 754 and/or the fluid reservoir 762 may be coupled to one or more of the components of the drive assembly, so that movement of the drive assembly may move one or more of the plunger tube 754 and the fluid reservoir 762 within the handle.

In some variations, the plunger tube may be coupled to one of: the first linear gear and the second linear gear. In some variations, the fluid reservoir may be directly or indirectly coupled to the one of: the first linear gear and the second linear gear, where movement of the first linear gear or second linear gear correspondingly move the fluid reservoir within the handle. In some variations, the fluid reservoir may be configured to move within the handle in a first direction but not a second direction opposite the first direction. It can be appreciated that the fluid reservoir may be configured in any number of ways to achieve this movement.

In some embodiments, movement of the first linear in a first direction may advance the fluid reservoir 762 and/or the plunger tube 754 in the first direction, towards the outer tube 780, which may remain stationary within the handle. Movement of the fluid reservoir 762 and the plunger tube 754 towards the stationary outer tube 780 may displace a volume of fluid from the lumen 764 of the fluid reservoir 762. The fluid may be delivered from the fluid reservoir 762 through a lumen of the plunger tube 754 to the elongate member 710 and to the eye. Movement of the fluid reservoir 762 and the plunger tube 754 in a second direction opposite the first direction (e.g., away from the outer tube 780) may also displace fluid from the fluid reservoir 762 as the plunger tube 754 is advanced into the lumen 764.

### External Fluid Device and Fluid Compositions

The delivery devices described herein may be configured to releasably coupled with an external fluid device to transfer fluid into the fluid reservoir of the delivery devices or otherwise provide fluid before and/or during a procedure. The external fluid device may deliver fluid to or through the fluid reservoir, and may detachably couple with the fluid reservoir, for example, via the fluid reservoir connector. The external fluid device may be coupled with the delivery device before or during a procedure to one or more of initially provide fluid to fill the fluid reservoir and provide a continuous source, or an additional source, of fluid during a procedure. In variations in which the external delivery device is decoupled from the delivery device after transfer of fluid to the fluid reservoir but before advancing the cannula to a target treatment location in the eye (e.g., Schlemm's canal), it can be appreciated that once fluid is delivered from the fluid reservoir to the eye, the external fluid device may be recoupled to the fluid reservoir connector to deliver additional volumes of fluid to the eye (via the fluid reservoir).

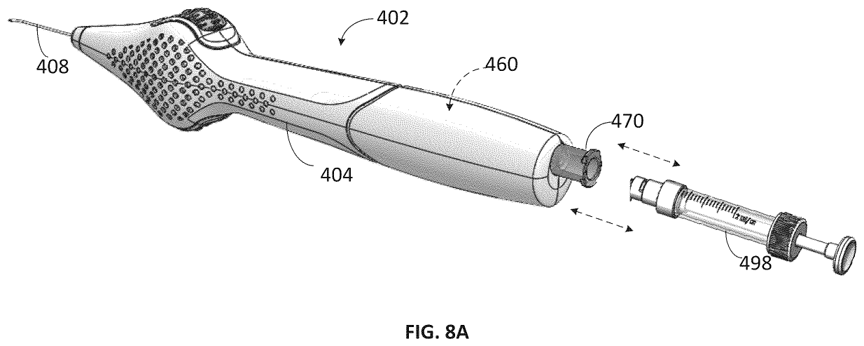

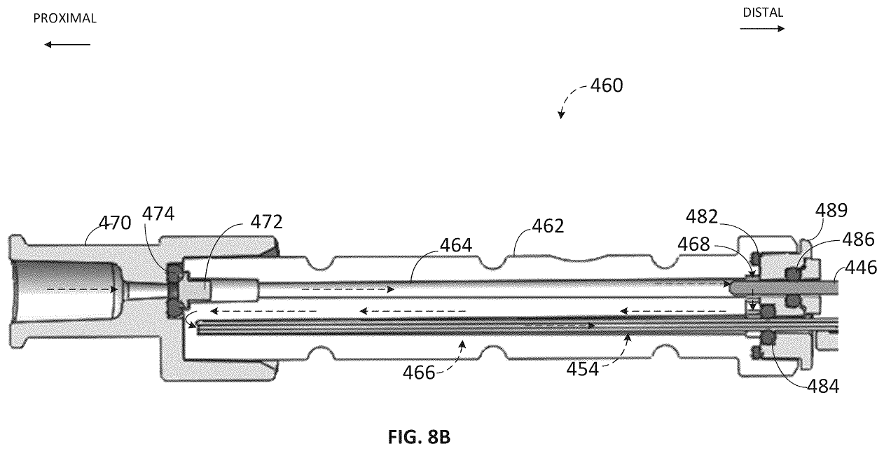

FIG. 8A depicts a perspective view of a delivery device 402 receiving an external fluid device 498. In some variations, the external fluid device 498 may be detachably coupled to the fluid reservoir connector 470 to deliver a fluid composition to the fluid reservoir 462. In some variations, the external fluid device may deliver the fluid composition through the fluid reservoir connector to the first lumen of the fluid reservoir and/or the second lumen of the reservoir. In some variations, the external fluid device may completely fill the second lumen of the fluid reservoir and then fill (e.g., fully, partially) the first lumen of the fluid reservoir. The external fluid device 498 may include a syringe, a vial, or another container used to store fluid. The external fluid device may be provided as part of the delivery system in a kit, or the external fluid device may be separately provided by the user. In some variations, the external fluid device may be pre-loaded with a fluid composition and may be packaged, e.g., in a kit, with a fluid composition disposed therein. In other variations, the external fluid device may not be pre-loaded, and may instead be packaged empty and loaded with a fluid composition by the user prior to a procedure. In these variations, the kits described herein may also include a separate fluid container (e.g., vial, syringe) containing fluid for use with the external fluid devices and/or fluid delivery devices described herein. The external fluid device may include volume markings. In some variations, the external fluid device may be partially or entirely transparent or otherwise see through, to allow the user to easily distinguish the volume of the fluid composition within the external fluid device. In some variations, once the first volume of fluid has been delivered to the eye, the external fluid device 498 may reattach to the fluid reservoir connector 470 to deliver a second volume of fluid to the fluid reservoir 462. In some variations, the second volume of fluid may be less than, equal to, or greater than the first volume of fluid.

The fluid composition may comprise fluid compositions including but not limited to saline and viscoelastic fluids. The viscoelastic fluids may comprise hyaluronic acid, chondroitin sulfate, cellulose, derivatives or mixtures thereof, or solutions thereof. In some variations, the viscoelastic fluid may comprise sodium hyaluronate. Additionally, or alternatively, the viscoelastic fluid may further include a drug. For example, the viscoelastic fluid may include a drug suitable for treating glaucoma, reducing or lowering intraocular pressure, reducing inflammation, fibrosis neovascularization or scarring, and/or preventing infection. For example, in some variations, the viscoelastic fluid may include the therapeutic agents described herein, such as but not limited to, Rho kinase (ROCK) inhibitors and agents for gene therapy, DNA, RNA, or stem cell-based approaches. The fluid compositions may include custom drug formulations.

Fluid Flow through the Fluid Reservoir

FIG. 8B depicts a cross sectional view of the fluid assembly 460 including a portion of the plunger tube 454 residing within the second lumen 466 with arrows depicting the flow of fluid through the fluid reservoir 460 during loading of fluid into the reservoir and priming of the delivery device. As shown there, after fluid is transferred through the fluid reservoir connector 470 into the fluid reservoir 460 for example, from an external fluid device, fluid enters the first lumen 464 of the fluid reservoir 462 through the valve 472. Fluid flows through the first lumen 464, around a portion of the displacement rod 446 within the first lumen 464 and through the passageway 468 to the second lumen 466. As described above, the diameter of the displacement rod 446 may be less than the diameter of the first lumen 464 so that fluid may flow around the displacement rod 446 in the first lumen 464 to the passageway 468. Once the fluid flows through the passageway 468 into the second lumen 466, the fluid may flow around the plunger tube 454 through the plunger tube lumen to the elongate member (not depicted) and the eye. Proximal movement of the displacement rod 446 may displace fluid from the first lumen 464 to the second lumen 466, while proximal movement of the plunger tube 454 may displace fluid from the second lumen 466 to the lumen of the elongate member. In some variations, the second lumen 466 may be filled completely first with the fluid composition before the first lumen 464 is filled (e.g., partially, fully) with the fluid composition.

### Fluid Volume Delivery

With fluid in the fluid reservoir 462 being displaced from the first lumen 464 to the second lumen 466 by proximal movement of the displacement rod 446 and fluid being displaced from the second lumen 466 to the eye by either proximal movement or distal movement of the plunger tube 454, the volumes of fluid delivered during advancement of the elongate member 410 and during retraction of the elongate member 410 may be different. The differences in the volumes of fluid delivered from the fluid reservoir 462 to the eye may be determined by and due to one or more geometric differences between the one or more lumens, one or more geometric differences between the displacement rod and the plunger tube, or a combination thereof.

### Fluid Reservoir Lumen Geometry Differences

With reference back to FIG. 7C, in some variations, geometric differences between the lumens of the plurality of lumens of the fluid reservoir, such as the first and second lumens 464, 466 depicted in FIG. 7C, may impact the volume of fluid delivered during each of advancement and retraction of the elongate member. As described above, generally, since the first linear gear and the second linear gear may move in opposing directions, advancement of the elongate member may corresponding retract the displacement rod into the first lumen. Since the first linear gear and second linear gear may move in opposing directions, advancement of the elongate member may also advance the plunger tube out of the second lumen, while retraction of the elongate member retracts the plunger tube into the second lumen while the displacement rod is stationary within the first lumen.

These geometric differences between the first lumen 464 and the second lumen 466 may include but are not limited to: a difference in diameters or heights 465, 469 of the first and second lumens 464, 466, a difference in volumes of the first and second lumens 464, 466, a difference in the lengths 467, 471 of the first and second lumens 464, 466, or a combination thereof. For example, the volume of fluid displaced from the first lumen 464 to the second lumen 466 when the displacement rod 446 moves proximally in the first lumen 464 may be due to the differences in lengths 467, 471 of the first lumen 464 and the second lumen 466. The volume of fluid displaced from the first lumen 464 to the second lumen 466 when the displacement rod 446 moves proximally in the first lumen 464 may be due to the differences in the diameters or heights 465, 469 of the first lumen 464 and the second lumen 466. The volume of fluid displaced from the first lumen 464 to the second lumen 466 when the displacement rod 446 moves proximally in the first lumen 464 may be due to the difference in the volumes of the first lumen 464 and the second lumen 466. For example, if the volume of the first lumen 464 is greater than the volume of the second lumen 466, the volume of fluid displaced from the first lumen 464 to the second lumen 466 during proximal movement of the displacement rod 446 within the first lumen 464 may be less than the volume of fluid displaced from the first lumen 464 to the second lumen 464 if the volume of the first lumen 464 is smaller than the volume of the second lumen 466. For example, if the volume of the first lumen 464 is smaller than the volume of the second lumen 466, the volume of fluid displaced from the first lumen 464 to the second lumen 466 during proximal movement of the displacement rod 466 within the first lumen may be greater than the volume of fluid displaced from the first lumen 464 to the second lumen 464 if the volume of the first lumen 464 is greater than the volume of the second lumen 466.

### Plunger Tube and Displacement Rod Geometric Differences

Additionally, or alternatively, geometric differences between the plunger tube and the displacement rod may impact the volume of fluid delivered during advancement of the elongate member. These geometric differences between the plunger tube and the displacement rod may include but are not limited: a difference in diameters of the displacement rod and the plunger tube, a difference in lengths of the displacement rod and the plunger tube, a difference in cross-sectional shapes of the displacement rod and the plunger tube, or a combination thereof. For example, looking at FIG. 7D, the volume of fluid displaced from the first lumen 464 to the second lumen 466 when the displacement rod 446 moves proximally in the first lumen 464 may be due to the differences in diameters of the displacement rod 446 and the plunger tube 454, where the diameter of the displacement rod 446 is greater than the diameter of the plunger 454. The displacement rod 446 may be advanced into the first lumen 464 while the plunger tube 454 may be retracted out of the second lumen 466, and the displacement rod 446 may move at the same rate as the plunger tube 454. The volume of fluid delivered from the fluid reservoir during the movement of the displacement rod 446 into the first lumen 464 and the movement of the plunger tube 454 out of the second lumen 466 may be defined by the difference in the diameter of the displacement rod 446 compared to the diameter of the plunger tube 454 and the distance of travel of the displacement rod 446 and the plunger tube 454. In some variations, the diameter of the displacement rod 446 may be greater than the diameter of the plunger tube 454. If the displacement rod 446 has a larger diameter than the plunger tube 454, a greater volume of fluid may be displaced from the first lumen 464 to the second lumen 466 during proximal movement of the displacement rod 446 within the first lumen 464. The volume of fluid displaced from the first lumen 464 to the second lumen 466 when the displacement rod 446 moves proximally in the first lumen 464 may be due to the differences in lengths of the displacement rod 446 and the plunger tube 454 that reside with each lumen. If the displacement rod 446 has a shorter length that resides within the first lumen 464 than the length of the plunger tube 454 that resides within the second lumen 466, a smaller volume of fluid may be displaced from the first lumen 464 to the second lumen 446 when the displacement rod 446 moves proximally in the first lumen 464. In some variations, during retraction of the elongate member, the displacement rod 446 is static (e.g., does not move within the first lumen 464), while the plunger tube 454 may be retracted into the second lumen 466 to displace fluid from the second lumen 466 into the eye.

It can be appreciated that any of these geometric differences between the first lumen and the second lumen, the displacement rod and the plunger tube, or the combination thereof may be used to generate a specific volume of fluid being delivered from the fluid reservoir to the eye during advancement or retraction of the elongate member. Additionally, it can be appreciated that the volume of fluid delivered may be delivered at different rates during advancement of the elongate member or retraction of the elongate member due to the one or more geometric differences between the first lumen and the second lumen, the displacement rod and the plunger tube, or the combination thereof. For example, in some variations, if the displacement rod and/or the plunger tube have a variable diameter (e.g., the diameter decreases or increases along the length), the rate of the volume of fluid delivered to the eye during advancement and retraction of the elongate member may differ, depending on the location of the distal end (e.g., distal tip) of the elongate member within the eye.

Delivery of Fluid with Different Rates

It can be appreciated that the volume of fluid delivered to the eye may be delivered at different rates during advancement of the elongate member and retraction of the elongate member due to one or more geometric differences between the first lumen and the second lumen, the displacement rod and the plunger tube, or the combination thereof. For example, in some variations, the volume of fluid may be delivered at a first rate during advancement of the elongate member, and may be delivered at a second rate different during retraction of the elongate member. In some variations, the first rate may be greater than the second rate, while in other variations, the second rate may be greater than the first rate. In some variations, the first rate and the second rate may be equal. For example, for a full 360 degree of travel within Schlemm's canal, the volume of fluid may be delivered at a first rate of about 0.5 μL per clock hour of the eye (e.g., about 3 mm/0.12 inch) during advancement. The volume of fluid may be delivered at a second rate of about 2 μL per clock hour (e.g., 12 mm/0.12 inch) during retraction.

Loading A Fluid Composition into The Delivery Device and “Priming the Device”

Before the fluid composition is delivered to the eye, the fluid composition may be loaded into a delivery device from an external fluid device. In some variations, the fluid composition may be loaded into the fluid reservoir prior to insertion of the fluid reservoir into the device handle. In other variations, the fluid composition may be loaded into the fluid reservoir after the reservoir is positioned in the device handle, prior to advancing the cannula to a target treatment area (e.g., Schlemm's canal) within the eye. In yet other variations, fluid composition may be loaded into the reservoir prior to insertion of the fluid reservoir into the device handle, and after delivery of some or all of the total volume of the fluid reservoir, additional fluid may be delivered to or through the fluid reservoir from an external device. In devices utilizing an external fluid device, the external fluid device may be releasably or permanently coupled to the fluid delivery device. For example, the external fluid device may be releasably or permanently coupled to the device handle such that the external fluid device is in fluid communication with the fluid reservoir, and thus with the elongate member. For example, the external fluid device may be coupled (e.g., releasably) to a fluid reservoir connector and the fluid composition may be introduced into the fluid reservoir from the external fluid device via the fluid reservoir connector. In variations in which an external fluid device is utilized, the delivery device may be configured to maintain sterility of the fluid composition while allowing for quick transfer of the fluid composition from the external fluid device to the fluid assembly, as the fluid composition is delivered directly to the fluid reservoir. The fluid delivery device may further be configured to transfer fluid into the fluid reservoir without introducing any additional air into the fluid assembly and while removing any existing air within the fluid assembly. Once primed, the displacement rod may be primarily outside of the fluid reservoir (e.g., a majority of the displacement rod may be located outside of the first lumen), and the plunger tube may be fully retracted within the fluid reservoir (e.g., a majority of the plunger tube may be located within the second lumen), with the fluid pathway from the plunger tube to the distal end of the elongate member filled with the fluid composition.

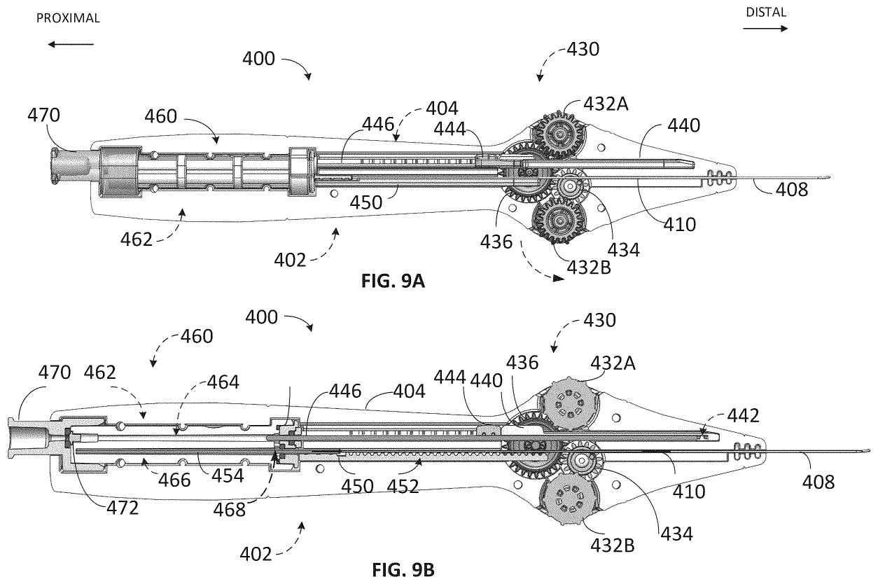

FIG. 9A depicts a perspective view of the device with the housing of the handle removed and FIG. 9B depict cross sectional views of a pre-advance or primed configuration of a variation of the delivery devices described herein when the fluid composition from an external fluid device is delivered to the fluid reservoir and the device is ready to deliver the fluid composition to the eye. As seen in FIG. 9A, each of the first linear gear 440, the ratchet 444 and the displacement rod 446 is fully distally positioned, and the second linear gear 450 is fully proximally positioned, with the plunger tube 454 residing within the second lumen 466. In some variations, the handle 402 may include a locking mechanism to lock the actuator configured to be actuated by a user during delivery of fluid from an external fluid device to the fluid reservoir. For example, the handle 402 may comprise a lock configured to engage with one or more of the rotatable components 432A-432B to prevent rotation thereof, and thus actuation of the delivery assembly and delivery of fluid from the fluid reservoir. In some variations, the drive assembly 430 may comprise a lock configured to engage the first linear gear 440 and/or second linear gear 450 to prevent linear movement in the proximal direction before the fluid composition is ready to be delivered to the eye. As seen in FIG. 9B, a majority of the displacement rod 446 may not be located within the first lumen 464 and a majority of the plunger tube 454 may be located within the second lumen 466. Similarly, a majority of the first lumen 464 may be empty or not otherwise retaining the displacement rod 446, while a majority of the second lumen may be retaining the plunger tube 454. In this configuration, the fluid composition may flow from the external fluid device through the first lumen 464, through the passageway 468, through the second lumen 466 (around the plunger tube 454), through the plunger tube lumen, through the lumen of the elongate member 410, and out of an opening at the distal tip of the elongate member to prepare or “prime” the device for a fluid delivery procedure and purge any existing air within the fluid assembly 460. Once the fluid composition has been transferred from the external fluid device to the fluid reservoir 462, the external fluid device may or may not be decoupled from the fluid reservoir connector 470. In variations in which the external fluid device has been decoupled from the fluid delivery device, once a portion of, or the entirety of the fluid composition within the fluid reservoir has been delivered to the eye, the external fluid device may be re-coupled with the fluid reservoir connector and additional fluid may be delivered to the fluid reservoir, and/or through the fluid reservoir and to the eye.

### Actuation of the Device in a First Direction

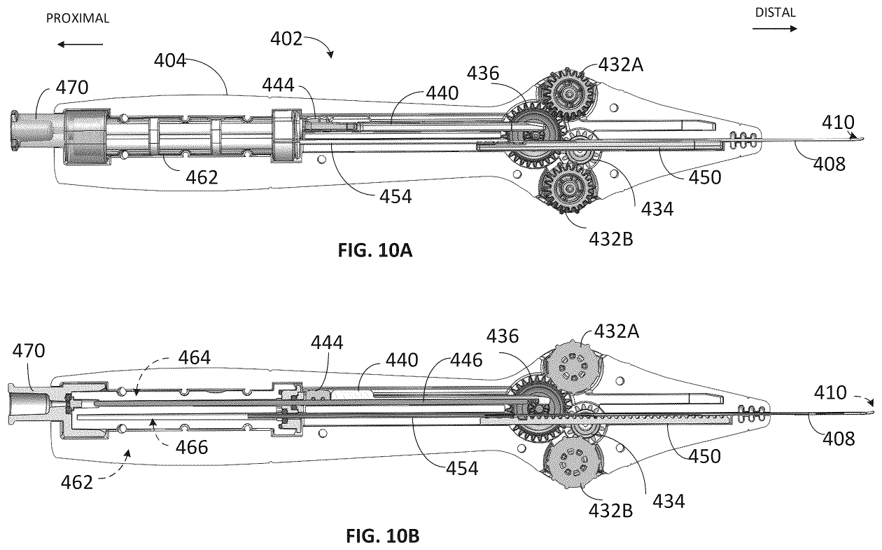

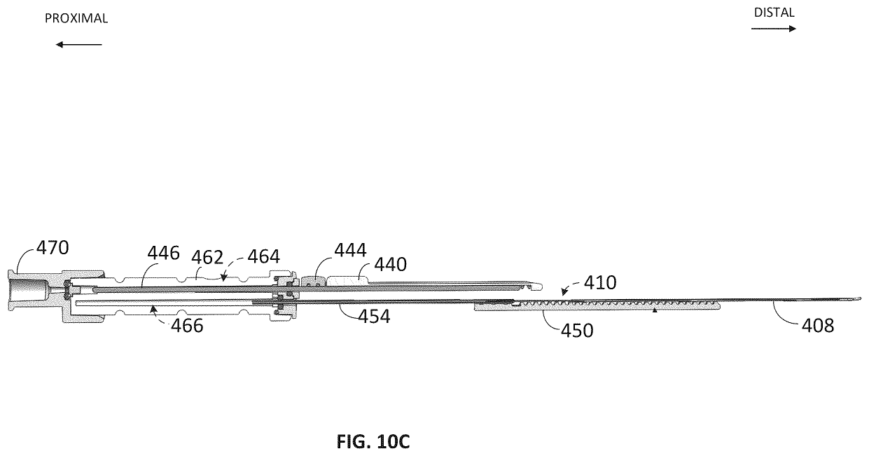

FIG. 10A depicts a perspective view of the device with the housing of the handle removed and FIG. 10B depicts a cross sectional view of a post-advance/pre-retract configuration of the device. The post-advance/pre-retract configuration of the device may be the configuration of the device after actuation of the actuator in a first direction, movement of the first linear gear in a first direction and movement of the second linear gear in a second direction opposite of the first direction. The post-advance/pre-retract configuration may also include the ratchet is fully proximally positioned within the handle adjacent to the fluid reservoir, corresponding advancement of the elongate member, and delivery of fluid during advancement of the elongate member. As seen in FIG. 9A, actuation of the actuators 432A-432B in a first direction may move the first linear gear 440 in the proximal direction. Proximal movement of the first linear gear 440 may move the ratchet 444 in the proximal direction. Correspondingly, actuation of the actuators 432A-432B in the first direction may move the second linear gear 450 in a distal correction. For example, actuation of the actuators 432A-432B in the first direction may move the second linear gear 450 a distance distally that corresponds to the proximal movement of the first linear gear 440. Moreover, actuation of the actuators 432A-432B in the first direction may advance the elongate member 410 distally within and from the cannula 408 into an arc of Schlemm's canal as the elongate member 410 is coupled to the second linear gear 450. In the fluid reservoir, as seen in FIG. 9B the displacement rod 446 may have proximally advanced into the first lumen 464 by way of the first linear gear 440 moving the ratchet 444 in the proximal direction. The proximal movement of the displacement rod 446 (and, in some variations, the greater diameter of the displacement rod 446 compared to the diameter of the plunger tube 454, and in other variations, the greater diameter of the first lumen compared to the diameter of the second lumen) may displace a volume of fluid from the first lumen 464 through the passageway 468 into the second lumen 466. The volume of fluid may then be delivered to the eye by way of the plunger tube lumen and the lumen of the elongate member 410, which are in fluid communication with one another, while the elongate member 410 may be advancing within Schlemm's canal. A majority of the plunger tube 454 may have moved distally out of the second lumen 466 as the second linear gear 450 moves distally, as seen in FIG. 10C.

### Actuation of the Device in a Second Direction Opposite the First Direction

FIG. 11A depicts a perspective view of the device with the housing of the handle removed and FIG. 11B depicts a cross sectional view of the mid-retract configuration of the device. The mid-retract configuration of the device may be the configuration during actuation of the actuator in a second direction opposite of the first direction (detailed above), movement of the first linear gear in a second direction opposite the first and movement of the second linear gear in a first direction opposite the second direction, corresponding retraction of the elongate member, and delivery of fluid during retraction of the elongate member. As seen in FIG. 11A, actuation of the actuators 432A-432B in a second direction opposite the first direction may move the first linear gear 440 in the distal direction. The ratchet 444 may stay stationary, adjacent to the fluid reservoir 462. The second linear gear 450 may move in the proximal direction with the elongate member 410 retracting (e.g., within Schlemm's canal) towards or within the cannula 408. In the fluid reservoir 462, as seen in FIG. 11B, the displacement rod 446 may not have moved within the first lumen 464. Since the displacement rod 446 is not coupled to the first linear gear 440, the channel 442 (as seen in FIG. 11C) within the first linear gear 440 may allow for distal movement of the first linear gear 440 without movement of the displacement rod 446. Proximal movement of the second linear gear 450 may move the plunger tube 454 proximally into the second lumen 464, displacing a volume of fluid from the second lumen 464 through the plunger tube lumen, and into the lumen of the elongate member 410. In this manner, the volume of fluid from the second lumen may be delivered to the eye while the elongate member 410 is retracting.

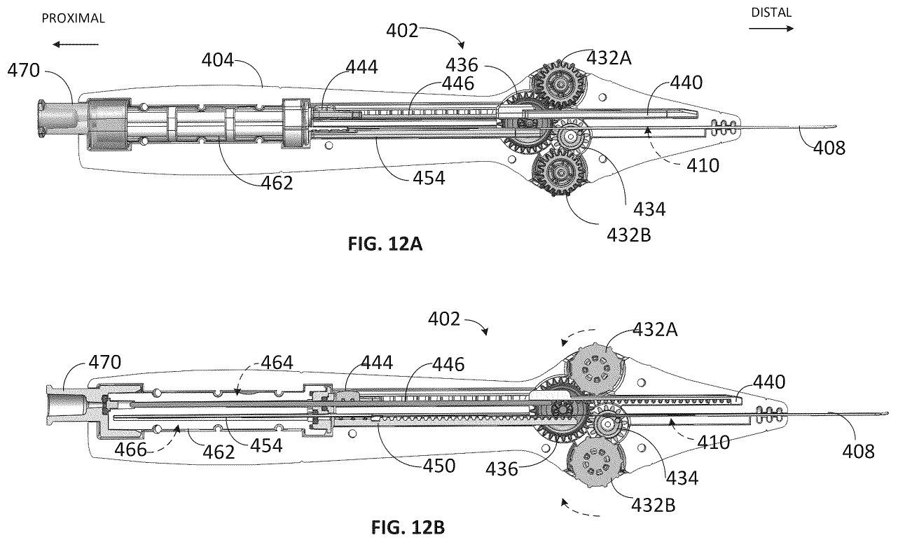

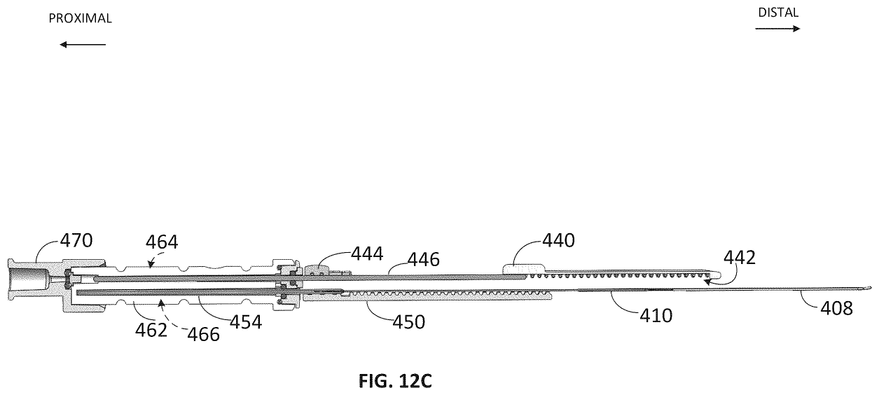

FIG. 12A depicts a perspective view of the device with the housing of the handle removed and FIG. 12B depicts a cross sectional view of the post-retract configuration of the device. The post-retract configuration of the device may be the configuration after actuation of the actuator in the second direction opposite of the first direction and after fluid delivery during retraction of the elongate member 410. The first linear gear 440 may be fully distally positioned within the handle 402 while the ratchet 444 may be proximally located adjacent the fluid reservoir 462. The second linear gear 450 may be fully proximally positioned within the handle 402. In the fluid reservoir, as seen in FIG. 12B, the displacement rod 446 may not have moved within the first lumen 464. The channel 442 within the first linear gear 442 may allow for the first linear gear 440 to be fully distally extended within the handle 402 without any distal movement of the displacement rod 446. The second linear gear 450 may be fully proximally extended within the handle 402, adjacent to the fluid reservoir 462. A portion of the plunger tube 454 may fully or nearly fully occupy the second lumen 466, as seen within FIG. 12C. In some variations, a portion of the plunger tube 454 may nearly fully occupy about 50% to about 95% of the second lumen 466 including about 55%, about 60%, about 65%, about 70%, about 75%, about 80%, about 85%, about 90%, or about 95%.

If the entirety of the volume of fluid within the fluid reservoir has not been delivered to the eye after a full cycle of travel (e.g., both full distal travel and full proximal travel within the handle) of the first linear gear and the second linear gear, the first linear gear and the second linear gear may be actuated in the first and second directions respectively, to advance the elongate member without delivery of fluid (e.g., the displacement rod within the first lumen may prevent additional delivery of fluid during advancement) while the elongate member may be retracted with delivery of fluid (e.g., the plunger tube may be advanced from and retracted into the second lumen as many times as needed to deliver the entirety of the volume of fluid within the second lumen). For example, in some variations, once a majority of the displacement rod is occupying the first lumen, no additional fluid may be delivered from the fluid lumen during advancement of the elongate member.

In some variations, a full cycle of travel (e.g., both full distal travel and full proximal travel within the handle) of the first linear gear and the second linear may occur in a single action (e.g., full distal travel happens in a single continuous motion, and/or full proximal travel happens in a single continuous motion) or may occur in discontinuous motions (e.g., partial distal travel happens and/or partial proximal travel happens with subsequent partial distal travel and subsequent partial proximal travel) until a single full cycle of travel occurs.

In some variations, the delivery devices may be configured to allow for a fixed cumulative amount of extension and/or retraction of the slidable elongate member. The fixed cumulative amount of extension/retraction may correspond, for example, to the full circumference of Schlemm's canal, two full circumferences of Schlemm's canal, or any desired distance. Exemplary fixed cumulative amounts may be, but are not limited to, about 33 mm to about 40 mm, about 39 mm to about 41 mm, about 38 mm to about 40 mm, about 35 mm to about 45 mm, about 78 mm to about 82 mm, about 76 mm to about 80 mm, or about 70 mm to about 90 mm. The delivery systems may additionally or alternatively be configured to allow for a fixed cumulative delivery of fluid. For example, in some variations, as described in more detail herein, the displacement rod may be configured to move proximally within the handle housing to deliver fluid from the fluid reservoir during advancement of the elongate member but not distally within the handle housing, and the first linear gear and second linear gear may be configured to move toward and away from the fluid reservoir. As such, with each extension/advancement of the slidable elongate member, the first linear gear may move proximally but the displacement rod and ratchet may remain fixed.

In some variations, the delivery devices may comprise a stop (e.g., a protrusion on an interior wall of the housing) that may prevent the first linear gear and/or second linear gear from moving distally beyond a certain point. For example, in some instances, once the first linear gear and/or second linear gear has reached its distal-most position, neither the first linear gear nor the second linear gear may be moved distally or proximally, and the actuators may no longer move.

The distance between the initial position of the first linear gear and/or the second linear gear and its final distal-most position may dictate the fixed cumulative amount of extension/retraction of the elongate member and the fixed cumulative delivery of fluid. It should be appreciated, however, that other variations of the delivery systems may not have a limited cumulative amount of extension and/or retraction of the elongate member. That is, some delivery systems may be able to be repeatedly extended and retracted without a fixed limit.

It should be appreciated that, in some variations, the delivery devices described herein may allow the elongate member to be advanced and retracted multiple times, so long as the total, cumulative amount of extension is below the limit. Indeed, in some variations, the maximum amount that the elongate member may be advanced without retraction may be less than that total, cumulative amount of extension. For example, the elongate member may be advanced a first time approximately halfway around Schlemm's canal (i.e., 180 degrees, or approximately 19 mm to about 20 mm) in a first direction, which may be the maximum amount that the elongate member may be advanced without retraction. The elongate member may then be fully retracted (during which fluid may be delivered). After this first extension, the displacement rod may have moved half of its maximum distance into the first lumen, and distance of the ratchet to fluid reservoir may have decreased by approximately half of its total possible decrease. The elongate member may be advanced a second time approximately halfway around Schlemm's canal in a second direction. The elongate member may then be retracted (during which fluid may be delivered). At the conclusion of the second extension, the displacement rod and ratchet may be located at their distal-most positions, and the distance from the ratchet to the fluid reservoir may be at its minimum. At this point, in some variations, the elongate member may no longer be advanced, no further fluid may be deliverable, and the actuator may no longer move.

In some variations, the fluid assembly may prevent loading of additional fluid composition into the device after treatment. In some variations, the device and systems described herein, or components thereof, may be intended for use in a single treatment. To prevent reuse of the device, the fluid assembly may be configured to prevent the fluid reservoir from being reloaded with additional fluid composition. For example, after a treatment cycle (e.g., full advancement and retraction of the elongate member) the lumens of the fluid reservoir may partially occupied. In such a configuration, attempting to re-prime the device by transferring additional fluid composition may not load the proper amount of the fluid composition. In some variations, the proximal end cap of the fluid reservoir may be configured to allow the fluid reservoir to be loaded with fluid composition a single time and to prevent subsequent fluid loading. For example, the valve may be configured to sealed after one or more of the plunger tube and the displacement rod has moved into a lumen of the fluid reservoir.

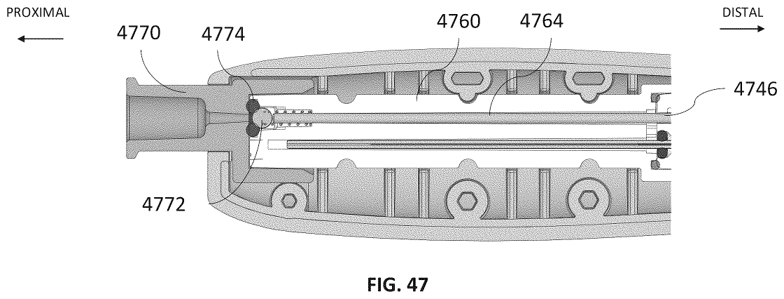

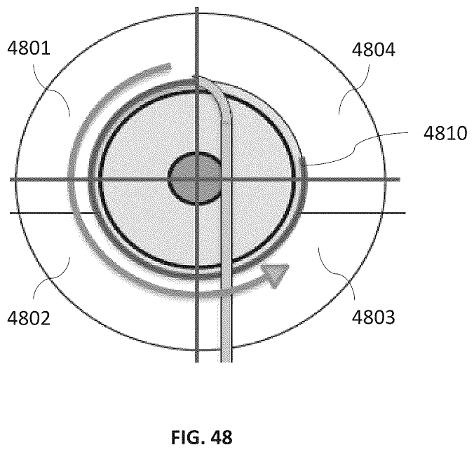

In some variations, the displacement rod may be configured to prevent additional fluid composition from entering the device after a first treatment. FIG. 47, depicts a device 4700 after treatment. Before treatment, the device 4700 may have been loaded with a fluid composition. An external fluid device may have been coupled to the fluid reservoir connector 4770 and the fluid composition may have been transferred through a valve 4772. As shown after treatment, the device 4700 may have completed a treatment cycle such that a majority of the displacement rod 4746 has moved into the fluid reservoir 4760. As shown in FIG. 47, when moved into the first lumen 4764 of the fluid reservoir 4760 the displacement rod 4746 may contact the valve 4772 and press the valve into a sealing member 4774, thereby prevent additional fluid composition from being loaded into the device. The displacement rod 4746 may remain in contact with the valve 4772 to prevent re-priming of the fluid reservoir 4760. Contact between the displacement rod 4746 and valve 4772 may be maintained by a ratchet coupled to the displacement rod as described herein. In other variations, the device may be configured to be reloaded with the fluid composition such as for example by again transferring fluid from an external fluid device through the fluid connector to the fluid reservoir.

### Kits

Also disclosed herein are kits that may comprise one or more devices of the disclosure. In some variations, kits may comprise a fluid delivery device as described herein and one or more additional devices that may be configured to deliver a fluid composition to the eye, deliver an ocular implant to the eye, and/or tear the trabecular meshwork. For example, in some variations, the one or more devices of the disclosure may be packaged together with one or more devices described in the co-pending U.S. application filed on Feb. 27, 2025 titled “Intraocular Systems, Devices, Kits, and Methods” to Daniel O'Keeffe et al., having an attorney docket number of SGHT-014/01US 328518-2191, which was previously incorporated by reference in its entirety, and/or kits may comprise one or more delivery devices described herein and any of the devices and/or systems described in U.S. application filed on Feb. 27, 2025 titled “Intraocular Systems, Devices, Kits, and Methods” to Daniel O'Keeffe et al., having an attorney docket number of SGHT-014/01US 328518-2191.

### Methods of Use

Methods of treating one or more conditions of the eye and/or methods for delivering a fluid composition into the eye (e.g., into Schlemm's canal) and/or methods of disrupting the trabeculocanicular tissues (e.g., dilating Schlemm's canal and/or tearing the trabecular meshwork) using the devices described herein are also provided. In some instances, treating conditions of the eye may result in increased aqueous humor drainage, reduced resistance to aqueous outflow, and/or reduced intraocular pressure. Some methods described herein may dilate Schlemm's canal, dilate the collector channels, and/or break any septae that may obstruct circumferential flow through Schlemm's canal. Dilation of Schlemm's canal may disrupt obstructed inner walls of the canal, stretch the trabecular meshwork, and/or increase the trabecular meshwork's porosity. This may improve the natural aqueous outflow pathway. The dilation may be performed by delivery of a fluid composition (e.g., a viscoelastic fluid as described herein). Some methods described herein may comprise performing a trabeculotomy to cut or tear trabecular meshwork. Some methods described herein may comprise implanting an ocular device within Schlemm's canal. In some instances, the systems described herein may be used in performing ab-interno trabeculotomy, ab-interno transluminal trabeculotomy, clear corneal trabeculotomy, clear corneal transluminal trabeculotomy, ab-interno canaloplasty, and/or clear corneal canaloplasty.

Methods for reducing intraocular pressure are provided below. The methods may be for one or more of delivering an ocular device (e.g., an implant) within the eye and disrupting ocular (e.g., trabeculocanalicular) tissues using a disruptive volume of fluid composition (e.g., viscoelastic fluid) and/or a disruptive tool. Methods for delivering implants, such as the implants described herein, may generally include using a delivery device to position an implant at least partially within Schlemm's canal. Implant-free tissue-disrupting methods may generally be achieved by using a delivery device to provide a force sufficient to disrupt trabeculocanalicular tissues. The methods may generally be single-handed, single-operator controlled methods that are minimally invasive, e.g., they are tailored for an ab-interno procedure, which as previously mentioned, can be advantageous over the more invasive ab-externo approach. However, use of the ocular systems in an ab-externo method may be contemplated in some instances and thus, are not excluded here. The methods for delivering an ocular device or fluid, and/or for providing a disruptive force, may be used to treat glaucoma, pre-glaucoma, and/or ocular hypertension. When treating glaucoma, the methods may also be used in conjunction with a cataract surgery (before or after) using the same incision. The methods may be used alone or in combination to reduce intraocular and thus provide treatment for an ocular condition or disorder. Further, one or more additional treatment modalities may be used with the methods herein, including medication, incisional surgery, laser surgery, cryosurgery, other forms of surgery, and combinations thereof.

In some variations, the methods may generally include steps of creating an incision in the ocular wall (e.g., the sclera or cornea) that provides access to the anterior chamber of the eye, advancing a cannula of the delivery system through the incision and at least partially across the anterior chamber to the trabecular meshwork, and accessing Schlemm's canal with the cannula. The methods may also include the step of priming or flushing the system with fluid (e.g., to remove air from the system) and/or the step of irrigating the operative field to clear away blood or otherwise improve visualization of the field. The surgeon may first view the anterior chamber and trabecular meshwork (with underlying Schlemm's canal) using an operating microscope and a gonioscope or gonioprism. Using a 0.5 mm or greater corneal, limbal, or sclera incision, the surgeon may then gain access to the anterior chamber. A saline solution or viscoelastic composition may then be introduced into the anterior chamber to prevent its collapse. Here the saline solution or viscoelastic composition may be delivered through the cannula of the delivery system or by another mode, e.g., by infusion through an irrigating sleeve on the cannula. The surgeon, under direct microscopic visualization, may then advance the cannula of a delivery device through the incision towards the anterior chamber angle. When nearing the angle (and thus the trabecular meshwork), the surgeon may apply a gonioscope or gonioprism to the cornea to visualize the angle. The application of a viscous fluid (e.g., a viscoelastic composition as previously described) to the cornea and/or gonioscope or gonioprism may help to achieve good optical contact and negate total internal reflection thereby allowing visualization of the anterior chamber angle. As the surgeon visualizes the trabecular meshwork, the cannula may then be advanced so that the distal tip of the cannula is adjacent to the meshwork, in contact with the meshwork, cannulates (e.g., traverses and/or pierces) the meshwork to be communication with the lumen of Schlemm's canal.

### Performing Ocular Procedure

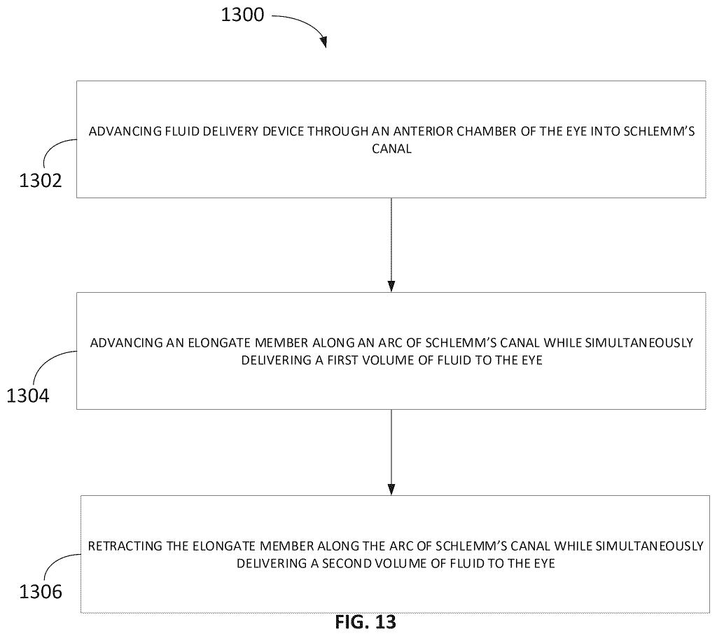

FIG. 13 illustrates a flow chart of an exemplary method 1300 of treating one or more conditions of the eye generally comprising advancing a fluid deliver device through an anterior chamber of the eye and into Schlemm's canal 1302, advancing an elongate member along an arc of Schlemm's canal while delivering a first volume of fluid to Schlemm's canal 1304, and retracting the elongate member along the arc of Schlemm's canal while delivering a second volume of fluid to the eye 1306.

In some variations, advancing a fluid deliver device through an anterior chamber and into Schlemm's canal 1302 may generally comprise creating an incision in the ocular wall (e.g., the sclera or cornea) that provides access to the anterior chamber of the eye, advancing a cannula of the fluid delivery device through the incision and at least partially across the anterior chamber to the trabecular meshwork, and accessing Schlemm's canal with the cannula. Prior to advancing the fluid delivery device through the anterior chamber, the methods described herein may further comprise priming or flushing the fluid assembly with the fluid composition (e.g., to remove air from the system) and/or irrigating the operative field to clear away blood or otherwise improve visualization of the field. The user may first view the anterior chamber and trabecular meshwork (with underlying Schlemm's canal) using an operating microscope and a gonioscope or gonioprism. Using a 0.5 mm or greater corneal, limbal, or sclera incision, the user may then gain access to the anterior chamber. A saline solution or viscoelastic composition may then be introduced into the anterior chamber to prevent its collapse. Here the saline solution or viscoelastic composition may be delivered through the cannula of the delivery system or by another mode, e.g., by infusion through an irrigating sleeve on the cannula. The user, under direct microscopic visualization, may then advance the cannula of the delivery system through the incision towards the anterior chamber angle. When nearing the angle (and thus the trabecular meshwork), the user may apply a gonioscope or gonioprism to the cornea to visualize the angle. The application of a viscous fluid (e.g., a viscoelastic composition as previously described) to the cornea and/or gonioscope or gonioprism may help to achieve good optical contact and negate total internal reflection thereby allowing visualization of the anterior chamber angle. As the user visualizes the trabecular meshwork, the cannula may then be advanced so that the distal tip of the cannula traverses (e.g., cannulates and/or pierces) the meshwork and is in communication with the lumen of Schlemm's canal. The distal tip of the cannula may be advanced to pierce the meshwork at an angle relative to a tangent of Schlemm's canal of about 5 degrees to about 45 degrees, such as about 10 degrees to about 30 degrees. Furthermore, the cannula may be tilted upward at angle of about 5 degrees to about 45 degrees, such as about 15 degrees to about 30 degrees to bias the elongate member into Schlemm's canal. Once the elongate member has entered the canal the angle of upward tilt may be reduced to between about 0 degrees to about 5 degrees.

In some variations, advancing the fluid deliver device through an anterior chamber and into Schlemm's canal 302 may comprise advancing a distal end of a cannula of the fluid delivery device through the anterior chamber of the eye and into Schlemm's canal. The delivery device may have any of the components and/or features described herein. In some variations, the fluid delivery device may be advanced through the anterior chamber of the eye and into Schlemm's canal 1302 when the fluid delivery device is in a pre-advance configuration. In the first configuration, first linear gear may be fully distally positioned within the handle and the first linear gear may be in contact with a ratchet, the second linear gear may be fully proximally positioned within the handle and may be adjacent to the fluid reservoir. The fluid reservoir may have a volume of fluid therein including within the first lumen and the second lumen, and a majority of the plunger tube may occupy the second lumen.

After the fluid delivery device (e.g., a distal tip of the cannula of the fluid delivery device) is advanced into Schlemm's canal, methods may further comprise advancing an elongate member along an arc of Schlemm's canal. The elongate member may be advanced any suitable amount and direction about the canal. For example, the elongate member may be advanced between about 1 degree and about 360 degrees about the canal, between about 10 degrees and about 360 degrees about the canal, between about 150 and about 210 degrees about the canal, about 360 degrees about the canal, about 270 degrees about the canal, about 180 degrees about the canal, about 120 degrees about the canal, about 90 degrees about the canal, about 60 degrees about the canal, about 30 degrees about the canal, or about 5 degrees about the canal. In some variations, the elongate member may be advanced between about 1 clock hour and about 12 clock hours about the canal, between about 2 clock hours and about 11 clock about the canal, between about 6 clock hours and about 10 clock hours about the canal, about 12 clock hours about the canal, about 10 clock hours about the canal, about 8 clock hours about the canal, about 6 clock hours about the canal, about 4 clock hours about the canal, about 2 clock hours about the canal, or about 1 clock hour about the canal. FIG. 48 depicts advancement of the elongate member 4810 along the arc of Schlemm's canal in a counterclockwise direction. As shown in FIG. 48, the elongate member 4810 may be advanced into at least a first quadrant of the canal 4801, at least a second quadrant of the canal 4802, at least a third quadrant of the canal 4803, or at least a fourth quadrant of the canal 4804. It should be appreciated that the elongate member may similarly be advanced in a clockwise direction.

In some variations, the elongate member may be advanced in two steps, e.g., first in a clockwise direction (e.g., about 180 degrees, about 90 degrees, about 6 clock hours, about 4 clock hours, etc.) and second in a counterclockwise direction (e.g., about 180 degrees, about 90 degrees, about 6 clock hours, about 4 clock hours, etc.) about the canal (e.g., to thereby achieve a 360- or 180-degree or 12 clock hour or 8 clock hour ab-interno viscocanalostomy or canaloplasty). In some variations, the elongate member may be advanced in one step (e.g., about 90 degrees in a clockwise direction, about 180 degrees in a clockwise direction, about 270 degrees in a clockwise direction, about 360 degrees in a clockwise direction, about 90 degrees in a counterclockwise direction, about 180 degrees in a counterclockwise direction, about 270 degrees in a counterclockwise direction, about 360 degrees in a counterclockwise direction) about the canal to thereby achieve a corresponding degree ab-interno viscocanalostomy or canaloplasty.

In some variations, advancing the elongate member may comprise visualizing one or more markings present on the elongate member. The distance advanced by the elongate member may be indicated by one or more markings on the elongate member as described herein. The one or more markings may become visible as the markings exit the cannula. For example, a marking may indicate a distal tip of elongate member has been advanced to a second hemisphere of the eye (e.g., advanced into a third quadrant, advanced about 180 degrees) or has been advanced a number of clock hours of travel (e.g., about 1 clock hour, about 2 clock hours, about 4 clock hours, about 6 clock hours, about 8 clock hours, or about 10 clock hours of travel) of the distal tip of the elongate member along the canal.

Advancing the elongate member around Schlemm's canal may comprise using the drive assembly to advance the elongate member around Schlemm's canal. In some variations, using the drive assembly to advance the elongate member around Schlemm's canal may comprise using the one or more actuators as described here to advance the elongate member around Schlemm's canal. Advancing the elongate member along an arc of the canal may comprise advancing the elongate member from the cannula and may include actuating the actuators in a first direction, causing the first linear gear to move in a first linear direction (e.g., proximally) within the handle and the second linear gear to move in a second, opposite linear direction (e.g., distally) within the handle. Actuating the actuators in the first direction may cause the first linear gear to move towards the fluid reservoir and the second linear gear to move away from the fluid reservoir. The second linear gear may be coupled to the plunger tube and the elongate member such that movement of the second linear gear may correspondingly move both the plunger tube and the elongate member. Movement of the second linear gear in the second linear direction (e.g., distally) may advance the elongate member from the cannula. The first linear gear may be in contact with the ratchet, which may be coupled to the displacement rod. Movement of the first linear gear in a first direction may corresponding move the ratchet and the displacement rod in the in the first direction, and the displacement rod may proximally advance into the first lumen of the fluid reservoir. Movement of the second linear gear in a second direction may move the plunger tube out of the second lumen. In some variations, an extendable fluid coupler may replace the plunder tube or decouple the plunger tube from movement of the second linear gear such that the volume of the second lumen is unchanged. Advancement of the displacement rod into the first lumen may displace a volume of fluid from the first lumen to the second lumen by the passageway fluidically coupling the first lumen to the second lumen. Simultaneously, the plunger tube may be retracted from the second lumen in a second direction. In some variations, advancing the elongate member from the cannula and along an arc of Schlemm's canal while simultaneously delivering a first volume of fluid to Schlemm's canal may include the simultaneous movement of the displacement rod advancing into the first lumen and the plunger tube retracting from the second lumen delivering the first volume of fluid to Schlemm's canal. In some variations, the first volume of fluid delivered to Schlemm's canal may be determined by one or more geometric differences between the first lumen and the second lumen, the displacement rod and the plunger tube, or the combination thereof. In some variations, the one or more geometric differences between the first lumen and the second lumen are as described above, and the one or more geometric differences between the displacement rod and the plunger tube are as described above.

Methods may further comprise delivering a fluid composition to Schlemm's canal. In some variations, methods may comprise simultaneously delivering a first volume of fluid to Schlemm's canal while advancing the elongate member along the arc of Schlemm's canal. The fluid composition may be delivered via the lumen of the elongate member and may exit the lumen through an opening in the distal end of the elongate member (e.g., the through the distal tip), or through one or more openings or fenestrations provided along the elongate member shaft, or a combination of both. The openings or fenestrations may be spaced along the axial length of the elongate member in any suitable manner, e.g., symmetrically or asymmetrically along its length.

The viscoelastic fluid may be delivered while advancing the elongate member of a single-handed, single-operator controlled device from Schlemm's canal in the clockwise direction, counterclockwise direction, or both, or during withdrawal of the elongate member from Schlemm's canal. As previously stated, the viscoelastic fluid may be delivered to disrupt Schlemm's canal and surrounding trabeculocanalicular tissues. For example, the delivered viscoelastic fluid may cause disruption by dilating Schlemm's canal, increasing the porosity of the trabecular meshwork, stretching the trabecular meshwork, forming microtears or perforations in juxtacanalicular tissue, removing septae from Schlemm's canal, dilating collector channels, or a combination thereof. The elongate member may be loaded with the viscoelastic fluid at the start of an ocular procedure so that the fluid can be delivered by a single device. This is in contrast to other systems that use forceps or other advancement tool to advance a fluid delivery catheter into Schlemm's canal and/or devices containing viscoelastic fluid that are separate or independent from a delivery catheter or catheter advancement tool, and which require connection to the delivery catheter or catheter advancement tool during a procedure by an assistant while the delivery catheter or catheter advancement tool is held by the surgeon.

The elongate member and/or fluid delivery may dilate Schlemm's canal, and fluid delivery may additionally dilate the collector channels. The entire length of Schlemm's canal or a portion thereof may be dilated by the fluid. For example, at least 75%, at least 50%, at least 25%, at least 10%, or at least 5% of the canal may be dilated. As another example, between about 1 degree and about 360 degrees of the canal may be dilated by the fluid, such as between about 10 degrees and about 350 degrees, between about 50 degrees and about 310 degrees, between about 90 degrees and about 290 degrees, between about 110 degrees and about 250 degrees, between about 150 degrees and about 210 degrees, between about 150 degrees and about 190 degrees (including all values and sub-ranges therein). In some variations, about 45 degrees, about 90 degrees, about 135 degrees, about 180 degrees, about 225 degrees, about 270 degrees, about 315 degrees, or about 360 degrees of the canal may be dilated by the fluid.

The fluid compositions may be delivered to treat one or more medical conditions of the eye, including but not limited to, glaucoma, pre-glaucoma, anterior or posterior segment neovascularization diseases, anterior or posterior segment inflammatory diseases, ocular hypertension, uveitis, age-related macular degeneration, diabetic retinopathy, genetic eye disorders, complications of cataract surgery, vascular occlusions, vascular disease, or inflammatory disease.

The method 1300 may further comprise retracting the elongate member along the arc of Schlemm's canal. In some variations, retracting the elongate member along the arc of Schlemm's canal may comprise actuating the one or more actuators in a second direction opposite the first direction. Actuation of the one or more actuators in the second direction opposite the first direction may cause the first linear gear to move in the second linear direction (e.g., distally) within the handle and the second linear gear to move in the first linear direction (e.g., proximally) within the handle. Actuation of the one or more actuators in the second direction opposite the first direction may cause the first linear gear to move away from the fluid reservoir and the second linear gear to move toward the fluid reservoir. Alternatively in some variations, actuation of the one or more actuators in the second opposite direction may disengage the actuators from the first linear gear (e.g., by disengage a clutch of the drive assembly) and may not move the first linear gear. In some variations, the displacement rod is seated within the first lumen and does not move distally during distal movement of the first linear gear. Since the second linear gear is coupled to the elongate member and the plunger tube, proximal movement of the second linear gear within the handle may retract the elongate member along the arc of Schlemm's canal and may retract the plunger tube proximally into the second lumen. In some variations, such as those including an extendable fluid coupler, actuation of the one or more actuators in the second opposite direction may move the first linear gear in the first linear direction (e.g., proximally) within the handle. Since the first linear gear may be releasably coupled to the displacement rod and the displacement rod ratchet, proximal movement of the first linear gear within the handle may deliver fluid during retraction of the elongate member along the arc of Schlemm's canal.

In some variations, methods may further comprise delivering fluid to Schlemm's canal while retracting the elongate member along an arc of Schlemm's canal. Retracting the elongate member along the arc of Schlemm's canal while simultaneously delivering the second volume of fluid to the eye may comprise retracting the plunger tube in the first linear direction and into the second lumen of the fluid reservoir thereby delivering the second volume of fluid to the eye. In some variations, retracting the elongate member while simultaneously delivering the second volume of fluid to the eye may comprise retracting the displacement rod in the first linear direction and into the first lumen of the fluid reservoir thereby delivering the second volume of fluid to the eye. In some variations, the second volume of fluid delivered to Schlemm's canal may be determined by one or more geometric differences between the first lumen and the second lumen, the displacement rod and the plunger tube, or the combination thereof, as described in more detail herein

In some variations, methods may further comprise re-advancing the elongate member along an arc of Schlemm's canal, as described above. In some of these variations, the elongate member may be readvanced along an arc of Schlemm's canal without delivering a volume of fluid to Schlemm's canal. In some of these variations, the elongate member may be readvanced along an arc of Schlemm's canal while simultaneously delivering additional volumes of fluid. Actuating the actuator in the first direction again may cause the first linear gear to move towards the fluid reservoir and the second linear gear to move away from the fluid reservoir. Movement of the second linear gear in the second linear direction (e.g., distally) may advance the elongate member from the cannula. The first linear gear may be in contact the ratchet (e.g., an arm of the ratchet) coupled to the displacement rod, or the displacement rod itself. The linear gear may receive less of the displacement rod during re-advancement compared to advancement. Movement of the first linear gear in a first direction may move the ratchet and the displacement rod in the in the first direction, and the displacement rod may proximally advance into the first lumen of the fluid reservoir to deliver fluid. An arm of the ratchet may disengage the ratchet from the first linear gear to allow continued movement the first linear gear in the first direction and continued movement of the second linear gear the second linear direction. Moreover, in some of these methods, the methods may further comprise re-retracting the elongate member along the arc of Schlemm's canal. In some of these variations, the elongate member may be re-retracted while simultaneously delivering additional volumes of fluid to the eye. In some variations, method may further comprise re-advancing the elongate member along an arc of Schlemm's canal while delivering a volume of fluid to Schlemm's canal. In some variations, methods may further comprise re-retracting the elongate member along an arc of Schlemm's canal without delivering an additional volume of fluid to Schlemm's canal.

More generally, in methods described herein, exemplary volumes of viscoelastic fluid that may be delivered may in some instances be between about 1 μl and about 200 μl, or in some instances be between about 1 μl and about 100 μl. In some instances, sufficient volumes to provide a disruptive force may range from about 1 μl to about 50 μl, from about 1 μl to about 30 μl, from about 2 μl to about 16 μl, from about 5 μl to about 25 μl, or from about 8 μl to about 21 μl. In one variation, a volume of about 4 μl is sufficient to disrupt Schlemm's canal and/or the surrounding tissues. In other variations, the volume of viscoelastic fluid sufficient to disrupt trabeculocanalicular tissues may be about 2 μl, about 3 μl, about 4 μl, about 5 μl, about 6 μl, about 7 μl, about 8 μl, about 9 μl, about 10 μl, about 11 μl, about 12 μl, about 13 μl, about 14 μl, about 15 μl, about 16 μl, about 17 μl, about 18 μl, about 19 μl, about 20 μl, about 21 μl, about 22 μl, about 23 μl, about 24 μl, about 25 μl, about 26 μl, about 27 μl, about 28 μl, about 29 μl, about 30 μl, about 31 μl. about 32 μl, about 33 μl, about 34 μl, about 35 μl, about 36 μl, about 37 μl, about 38 μl, about 39 μl, about 40 μl, about 41 μl, about 42 μl, about 43 μl, about 44 μl, about 45 μl, about 46 μl, about 47 μl, about 48 μl, about 49 μl or about 50 μl. In other variations, the volume of viscoelastic fluid sufficient to disrupt trabeculocanalicular tissues may be between about 50 μl and about 100 μl, including, for example, about 55 μl, about 60 μl, about 65 μl, about 70 μl, about 75 μl, about 80 μl, about 85 μl, about 90 μl, about 95 μlor about 100 μl.

In some variations, the volumes of viscoelastic fluid that is delivered during retraction of the elongate member may be different than the volumes of fluid that may be delivered during advancement of the elongate member. In some variations, the total volume of fluid that may be delivered during advancement may be less than the total volume of fluid that may be delivered during retraction, or vice versa. The volume of fluid that may be delivered during a single advancement may be the same as the volume of fluid that may be delivered during a single retraction of the same arc length of Schlemm's canal, or the volume of fluid that may be delivered during a single advancement may be different than (e.g., less than, greater than) the volume of fluid that may be delivered during a single retraction of the same arc length of Schlemm's canal.

Exemplary cumulative volumes of a fluid composition (e.g., viscoelastic fluid) that may be delivered to Schlemm's canal during advancement of the elongate member may include volumes between about 1 μl and 20 μl, including about 2 μl, about 3 μl, about 4 μl, about 5 μl, about 6 μl, about 7 μl, about 8 μl, about 9 μl, about 10 μl, about 11 μl, about 12 μl, about 13 μl, about 14 μl, about 15 μl, about 16 μl, about 17 μl, about 18 μl, about 19 μl, or about 20 μl. In some variations, exemplary volumes of fluid that may be delivered during advancement of the elongate member may include volumes between about 3 μl to about 9 μl including about 3 μl, about 4 μl, about 5 μl, about 6 μl, about 7 μl, about 8 μl, or about 9 μl.

Exemplary cumulative volumes of a fluid composition (e.g., viscoelastic fluid) that may be delivered during retraction of the elongate member may include volumes between about 1 μl and about 100 μl including about 2 μl, about 3 μl, about 4 μl, about 5 μl, about 6 μl, about 7 μl, about 8 μl, about 9 μl, about 10 μl, about 11 μl, about 12 μl, about 13 μl, about 14 μl, about 15 μl, about 16 μl, about 17 μl, about 18 μl, about 19 μl, about 20 μl, about 21 μl, about 22 μl, about 23 μl, about 24 μl, about 25 μl, about 26 μl, about 27 μl, about 28 μl, about 29 μl, about 30 μl, about 31 μl, about 32 μl, about 33 μl, about 34 μl, about 35 μl, about 36 μl, about 37 μl, about 38 μl, about 39 μl, about 40 μl, about 41 μl, about 42 μl, about 43 μl, about 44 μl, about 45 μl, about 46 μl, about 47 μl, about 48 μl, about 49 μl, about 50 μl, about 51 μl, about 52 μl, about 53 μl, about 54 μl, about 55 μl, about 56 μl, about 57 μl, about 58 μl, about 59 μl, about 60 μl, about 61 μl, about 62 μl, about 63 μl, about 64 μl, about 65 μl, about 66 μl, about 67 μl, about 68 μl, about 69 μl, about 70 μl, about 71 μl, about 72 μl, about 73 μl, about 74 μl, about 75 μl, about 76 μl, about 77 μl, about 78 μl, about 79 μl, about 80 μl, about 81 μl, about 82 μl, about 83 μl, about 84 μl, about 85 μl, about 86 μl, about 87 μl, about 88 μl, about 89 μl, about 90 μl, about 91 μl, about 92 μl, about 93 μl, about 94 μl, about 95 μl, about 96 μl, about 97 μl, about 98 μl, about 99 μl, or about 100 μl. Exemplary volumes of fluid that may be delivered during retraction of the elongate member may include volumes between about 18 μl and about 25 μl including about 18 μl, about 19 μl, about 20 μl, about 21 μl, about 22 μl, about 23 μl, about 24 μl, or about 25 μl.

Total exemplary volumes of viscoelastic fluid that may be delivered to the eye during advancement of the elongate member and retraction of the elongate member (e.g., one full advancement and one full retraction of the elongate member) may include volumes between about 21 μl and about 34 μl, or about 24 μl and about 30 μl, or about 24 μl to about 31 μl including about 21 μl, about 22 μl, about 23 μl, about 24 μl, about 25 μl, about 26 μl, about 27 μl, about 28 μl, about 29 μl, about 30 μl, about 31 μl, about 32 μl, about 33 μl, or about 34 μl. Total cumulative volumes of viscoelastic fluid that may be delivered to the eye during advancement of the elongate member and retraction of the elongate member may include volumes between 1 μl to about 120 μl including about 2 μl, about 3 μl, about 4 μl, about 5 μl, about 6 μl, about 7 μl, about 8 μl, about 9 μl, about 10 μl, about 11 μl, about 12 μl, about 13 μl, about 14 μl, about 15 μl, about 16 μl, about 17 μl, about 18 μl, about 19 μl, about 20 μl, about 21 μl, about 22 μl, about 23 μl, about 24 μl, about 25 μl, about 26 μl, about 27 μl, about 28 μl, about 29 μl, about 30 μl, about 31 μl, about 32 μl, about 33 μl, about 34 μl, about 35 μl, about 36 μl, about 37 μl, about 38 μl, about 39 μl, about 40 μl, about 41 μl, about 42 μl, about 43 μl, about 44 μl, about 45 μl, about 46 μl, about 47 μl, about 48 μl, about 49 μl, about 50 μl, about 51 μl, about 52 μl, about 53 μl, about 54 μl, about 55 μl, about 56 μl, about 57 μl, about 58 μl, about 59 μl, about 60 μl, about 61 μl, about 62 μl, about 63 μl, about 64 μl, about 65 μl, about 66 μl, about 67 μl, about 68 μl, about 69 μl, about 70 μl, about 71 μl, about 72 μl, about 73 μl, about 74 μl, about 75 μl, about 76 μl, about 77 μl, about 78 μl, about 79 μl, about 80 μl, about 81 μl, about 82 μl, about 83 μl, about 84 μl, about 85 μl, about 86 μl, about 87 μl, about 88 μl, about 89 μl, about 90 μl, about 91 μl, about 92 μl, about 93 μl, about 94 μl, about 95 μl, about 96 μl, about 97 μl, about 98 μl, about 99 μl, about 100 μl, about 101 μl, about 102 μl, about 103 μl, about 104 μl, about 105 μl, about 106 μl, about 107 μl, about 108 μl, about 109 μl, about 110 μl, about 111 μl, about 112 μl, about 113 μl, about 114 μl, about 115 μl, about 116 μl, about 117 μl, about 118 μl, about 119 μl, about 120 μl, about 121 μl, about 122 μl, about 123 μl, about 124 μl, about 125 μl, about 126 μl, about 127 μl, about 128 μl, about 129 μl or about 130 μl.

In some variations, the cumulative volume of fluid composition delivered during retraction of the elongate member compared to the cumulative volume of fluid composition delivered during advancement of the elongate member may have a ratio of about 2:1, about 2.5:1, about 3:1, about 3.5:1, about 4:1, about 4.5:1, about 5:1, about 5.5:1, about 6:1, about 6.5:1, about 7:1, about 7.5:1, about 8:1, about 8.5:1, about 9:1, about 9.5:1: about 10:1, about 10.5:1, about 11:1, about 11.5:1, about 12:1, about 12.5:1, about 13:1, about 13.5:1, about 14:1, about 14.5:1, about 15:1, about 15.5:1, about 16:1, about 16.5:1, about 17:1, about 18:1, about 18.5:1, about 19:1, about 19.5:1, or about 20:1.

In some variations, the rate at which the volumes of viscoelastic fluid are delivered during advancement of the elongate member and retraction of the elongate member may be different. For example, in some variations, the volume of fluid delivered during advancement of the elongate member (e.g., a first volume) may be delivered at a first rate and the volume of fluid delivered during retraction of the elongate member (e.g., a second volume) may be delivered at a second rate different from the first rate. In some variations, the first rate may be greater than the second rate, while in other variations, the second rate may be greater than the first rate. In some variations, the rate of fluid delivered may be proportional to the travel of the elongate member within the eye. For example, as described above, the first rate of fluid delivery during advancement of the elongate member may be about 0.5 μl per clock hour of the eye during advancement, while the second rate of fluid delivery during retraction of the elongate member may be about 2.0 μl per clock hour of the eye during retraction.

In some variations, wherein fluid has not been delivered to the entirety of Schlemm's canal, the method may generally include optional steps of rotating the cannula 180 degrees and reinserting the distal tip into Schlemm's canal. In some variations, rotating the cannula 180 degrees includes rotating the handle 180 degrees to rotate the cannula 180 degrees. In some variations, rotating the cannula 180 degrees may comprise rotating the cannula itself 180 degrees independently of (e.g., without) rotating the handle. In some variations, rotating the cannula 180 may comprise actuating a cannula actuator, which may comprise, for example, a rotatable knob.

In some variations, the fluid composition may be delivered to a portion of Schlemm's canal (e.g., less than 360 degrees) or in other variations, the entirety of Schlemm's canal (e.g., about 360 degrees) without rotating the cannula and reinserting the distal tip into Schlemm's canal or rotating the handle 180 degrees to rotate the cannula 180 degrees. For example, in some variations, the fluid composition may be delivered to about 90 degrees of Schlemm's canal, about 120 degrees of Schlemm's canal, about 150 degrees of Schlemm's canal, about 180 degrees of Schlemm's canal, about 210 degrees of Schlemm's canal, about 240 degrees of Schlemm's canal, about 270 degrees of Schlemm's canal, about 300 degrees of Schlemm's canal, or about 330 degrees of Schlemm's canal. Put differently, in some variations, the entirety of the fluid composition delivered may be delivered via advancement and/or retraction of the elongate member in a single clockwise and/or counterclockwise direction from the to access the canal, and rotation of the cannula and/or the handle may not be necessary to deliver the desired volume of fluid to the canal.

The method may generally further include advancing the elongate member around Schlemm's canal and delivering fluid to Schlemm's canal. Advancing the elongate member around Schlemm's canal may include advancing the elongate member around Schlemm's canal including for example between about 0 degrees to about 360 degrees around Schlemm's canal. Advancing the elongate member around Schlemm's canal includes using the drive assembly to advance the elongate member around Schlemm's canal. As described above, using the drive assembly to advance the elongate member around Schlemm's canal includes using the one or more mechanical actuators to advance the elongate member around Schlemm's canal including the one or more wheels, the slide, or the button. Retracting the elongate member as described above, delivering fluid to Schlemm's canal may include delivering fluid to Schlemm's canal during retraction of the elongate member.

Some of the delivery systems described herein may be configured such that the cumulative amount of advancement and/or retraction of the slidable elongate member is limited. For example, as described above, after the elongate member may be advanced and retracted a particular cumulative distance (e.g., about 39 mm to about 40 mm each of advancement and retraction, corresponding to the approximate circumference of Schlemm's canal; or about 78 mm to about 80 mm each of advancement and retraction, corresponding to approximately twice the circumference of Schlemm's canal; or any other suitable distance), the elongate member may no longer be able to be advanced. This advancement and retraction may occur over multiple advancement-retraction cycles. For example, the elongate member may be advanced about 20 mm, then retracted by about 20 mm, then advanced by about 20 mm, then retracted by about 20 mm. When the cumulative distance is limited to about 40 mm, after these two cycles of advancement and retraction, the elongate member may no longer be able to be advanced. In other variations, the delivery systems may not limit the cumulative amount of advancement and/or retraction of the elongate member.

The fluid compositions that may be delivered by the systems described herein include but are not limited to saline and viscoelastic fluids. The viscoelastic fluids may comprise hyaluronic acid, chondroitin sulfate, cellulose, derivatives or mixtures thereof, or solutions thereof. In one variation, the viscoelastic fluid comprises sodium hyaluronate. In another variation, the viscoelastic composition may further include a drug. For example, the viscoelastic composition may include a drug suitable for treating glaucoma, reducing or lowering intraocular pressure, reducing inflammation, fibrosis neovascularization or scarring, and/or preventing infection. For example, in some variations, the viscoelastic composition may include the therapeutic agents described herein, such as but not limited to Rho kinase (ROCK) inhibitors and agents for gene therapy, DNA, RNA, or stem cell-based approaches.

The viscoelastic fluids may also include agents that aid with visualization of the viscoelastic fluids. For example, dyes such as but not limited to fluorescein, trypan blue, or indocyanine green may be included. In some variations, a fluorescent compound or bioluminescent compound is included in the viscoelastic composition to help with its visualization. In other variations, the system may deliver the drug alone, without the viscoelastic composition. In this case, the drug may be loaded onto or into a sustained release biodegradable polymer that elutes drug over a period of weeks, months, or years. It is also contemplated that air or a gas could be delivered with the systems, as described herein.

Some of the methods, described in more herein, may comprise dilating Schlemm's canal and/or aqueous collector channels (e.g., with viscoelastic fluid) using the delivery devices described herein. The methods may also comprise tearing or cutting the trabecular meshwork of Schlemm's canal. These methods may be carried out separately (using separate devices), or they may be combined into a single procedure. For example, in some instances a portion (e.g., half) of Schlemm's canal may be dilated (using a fluid composition, for example), and the trabecular meshwork of the same or a different portion of Schlemm's canal may be torn or cut, within the same eye. As another example, all of Schlemm's canal may be dilated, and then all or a portion of the trabecular meshwork may subsequently be torn or cut. This may be desirable, for example, in order to both dilate the collector channels and tear or cut the trabecular meshwork.

In some of these variations, dilation and tearing or cutting may be performed using a single delivery system, such as one described herein configured to deliver a fluid composition. For example, the elongate member of a delivery system configured to deliver a fluid composition may first be used to deliver a fluid composition to a portion of Schlemm's canal (e.g., about a 180-degree arc of the canal, about a 90-degree arc of the canal) as described herein, and subsequently to tear or cut the trabecular meshwork in the same portion of the canal as described herein. As another example, the conduit of a delivery system configured to deliver a fluid composition may first be used to deliver a fluid composition to a portion of Schlemm's canal (e.g., about a 180-degree arc of the canal, about a 90-degree arc of the canal, etc.) and subsequently to tear or cut the trabecular meshwork in another portion of the canal (e.g., the other about-180 degree arc, another 90 degree arc, etc.). As yet another example, the elongate member of a delivery system configured to deliver a fluid composition may first be used to deliver fluid composition to all of Schlemm's canal (e.g., by delivering about 180 degrees of fluid composition in a first direction and then delivering about 180 degrees of fluid composition in a second direction), and then subsequently to tear or cut the full 360 degrees of trabecular meshwork (e.g., by tearing or cutting about 180 degrees of trabecular meshwork in a first direction and then tearing or cutting about 180 degrees of trabecular meshwork in a second direction). In another example, the elongate member of a delivery system configured to deliver a fluid composition may first be used to deliver fluid composition to all of Schlemm's canal in one step (i.e., by delivering about 360 degrees of fluid composition to Schlemm's canal in a single direction), and then subsequently to tear or cut the full 360 degrees of trabecular meshwork in a single step (i.e., by tearing or cutting about 360 degrees of trabecular meshwork in a single direction).

In other variations, dilation and tearing or cutting may be performed using different delivery systems (e.g., the dilation may be performed using a delivery system configured to deliver a fluid composition, and the tearing or cutting may be performed using a delivery system not configured to deliver a fluid). In another example, in some instances, dilation may be performed in one eye of a patient, while the trabecular meshwork may be torn or cut in the other eye of the patient.

Procedures comprising dilating Schlemm's canal and/or tearing or cutting the trabecular meshwork may also be combined with procedures delivering an ocular device, either in the same eye or in a different eye of the same patient. For example, all or a portion of Schlemm's canal may be dilated, followed by insertion of an ocular device. As another example, a portion of the trabecular meshwork may be torn or cut, while an ocular implant may be delivered to another portion of Schlemm's canal. In another example, a portion of Schlemm's canal may be dilated, while an ocular implant may be delivered to another portion of Schlemm's canal. In another example, an ocular implant may be delivered to a portion of Schlemm's canal, and then Schlemm's canal may be subsequently dilated to improve the function of the ocular implant.

Any suitable ocular device that maintains the patency of Schlemm's canal and/or improves outflow of aqueous humor may be implanted. For example, ocular devices that maintain the patency of Schlemm's canal without substantially interfering with fluid flow across, along, or out of the canal may be implanted. Such devices may comprise a support having at least one fenestration, as disclosed in U.S. Pat. Nos. 7,909,789, 8,529,622, 9,095,412, and co-pending U.S. application filed on Feb. 27, 2025 titled “Intraocular Systems, Devices, Kits, and Methods” to Daniel O'Keeffe et al., having an attorney docket number of SGHT-014/01US 328518-2191, each of which has been previously incorporated by reference in its entirety. Ocular devices that disrupt the juxtacanalicular trabecular meshwork or adjacent inner wall of Schlemm's canal may also be implanted. In addition to ocular devices made from metal or metal alloys, sutures (e.g., modified sutures), modified polymers, polymeric filaments, solid viscoelastic structures, and the like may be delivered. The sutures/modified sutures may be configured to facilitate fluid flow across, along, and/or out of the canal. In some variations, the ocular device may include one or more biodegradable (e.g., bioabsorbable) polymers. Such biodegradable polymers may include, for example, collagen, a collagen derivative, a poly(lactide); a poly(glycolide); a poly(lactide-co-glycolide); a poly(lactic acid); a poly(glycolic acid); a poly(lactide)/poly(ethylene glycol) copolymer; a poly(glycolide)/poly(ethylene glycol) copolymer; a poly(lactide-co-glycolide)/poly(ethylene glycol) copolymer; a poly(lactic acid)/poly(ethylene glycol) copolymer; a poly(glycolic acid)/poly(ethylene glycol) copolymer; a poly(lactic acid-co-glycolic acid)/poly(ethylene glycol) copolymer; a poly(caprolactone); a poly(caprolactone)/poly(ethylene glycol) copolymer; a polyorthoester; a poly(phosphazene); a poly(hydroXYbutyrate) or a copolymer including a poly(hydroXYbutyrate); a poly(lactide-co-caprolactone); a polycarbonate; a poly(esteramide); a polyanhydride; a poly(dioxanone); a poly(alkylene alkylate); a copolymer of polyethylene glycol and a polyorthoester; a biodegradable polyurethane; a poly(amino acid); a polyetherester; a polyacetal; a polycyanoacrylate; a poly(oXYethylene)/poly(oXYpropylene) copolymer; or a blend or copolymer thereof. In some variations, the ocular device may be entirely made of and/or comprise modified (e.g., processed/decellularized) scleral tissue.

Other variations of the ab-interno method include the use of an endoscope. Similar to the method described directly above, access to the anterior chamber is first made by incising the cornea, limbus, or sclera. Again, this may be done in combination with cataract surgery in one sitting, either before or after cataract surgery, or independently. The anterior chamber may be infused with saline solution or a viscoelastic composition may be placed in the anterior chamber to prevent its collapse. The saline or viscoelastic may be delivered as a separate step or it may be infused with the elongate member of the delivery system, an irrigating sleeve on the elongate member or cannula, or with a separate infusion cannula. The surgeon, under direct microscopic visualization, then advances the endoscope through the incision and towards the angle and trabecular meshwork. As the surgeon visualizes the trabecular meshwork via the endoscope or any associated display, the bevel of the cannula is advanced to traverse (e.g., canulate and/or pierce) the meshwork. The elongate member is then advanced under endoscopic visualization. The elongate member may be advanced any suitable amount and direction about the canal. For example, the elongate member may be advanced between about 10 degrees to about 360 degrees about the canal, or it may be advanced in two steps, e.g., 180 degrees in a clockwise direction and 180 degrees in a counterclockwise direction about the canal (to thereby achieve a full 360 degree ab-interno viscocanalostomy). Once the elongate member has been positioned within the canal, a fluid composition, e.g., a viscoelastic fluid, may be continuously or intermittently delivered through the lumen of the elongate member. The fluid composition may exit the lumen of the elongate member through its distal end (e.g., the through the distal tip), or through openings or fenestrations provided along its shaft, or a combination of both. The openings or fenestrations may be spaced along the axial length of the elongate member in any suitable manner, e.g., symmetrically or asymmetrically along its length. Other substances such as drugs, air, or gas may be delivered in the same manner if desired. The elongate member may be repositioned by retraction or repeated advancement and retraction. In some variations of the method, the same or different incision may be used, but the delivery system cannula is employed to access and dilate Schlemm's canal from a different direction (e.g., counterclockwise instead of clockwise). Once a sufficient amount of fluid has been delivered, the surgeon may retract the slidable elongate member into the cannula. In some variations the surgeon may then remove the delivery system from the eye; in other variations the surgeon may keep the delivery system within the eye and perform a trabeculotomy, as described in more detail herein.

Depending on factors such as the type or severity of the condition being treated, the disruptive force may be generated to partially or completely destroy and/or remove the trabecular meshwork and may be adjusted by varying the volume of viscoelastic fluid delivered.

Additionally, the fluid compositions may be delivered to restore the tubular anatomy of Schlemm's canal, to clear obstructions within the canal, to disrupt juxtacanalicular trabecular meshwork or the inner wall of Schlemm's canal within the canal, or to expand the canal. Here the delivery systems may include wires, tubes, balloons, instruments that deliver energy to the tissues, and/or other features to help with these methods. It is contemplated that glaucoma may be treated using such systems with additional features. The surface of these systems may also be roughened or have projections to further disrupt the inner wall of Schlemm's canal and juxtacanalicular trabecular meshwork to enhance aqueous humor outflow or permeability. Additionally, it should be appreciated that the delivery systems described herein may be used to deliver the fluid compositions to the anterior chamber or anterior segment.

A canaloplasty procedure may be performed in accordance with the methods described herein. For example, preoperative or intraoperative miotics may be administered to improve anterior chamber angle visibility. A corneal or scleral opening at least 0.5 mm wide may be created. The cannula may be advanced into the anterior chamber through the opening. The anterior chamber may be maintained. For example, viscoelastic or continuous balanced salt solution irrigation may be used to maintain the chamber. The cannula may be advanced into the anterior chamber and towards the iridocorneal angle. The cannula, elongate member, and/or anatomy of the eye may be visualized. For example, gonioscope or gonioprism may be applied to the cornea to visualize the angle. A viscous fluid may be used to couple the gonioscope or gonioprism to the cornea. One or more anatomic landmarks may be visualized including the anterior chamber angle, ciliary body, scleral spur, trabecular meshwork, Schwalbe's line, and the like. The cannula may approach the angle with the cannula tip and pierce the meshwork at an angle of about 5 degrees to about 30 degrees from tangential with the canal. In some variations, the cannula may be angled upwards (e.g., rotated about a longitudinal axis of the cannula) about 5 degrees to about 45 degrees to bias the elongate member into the canal. Once the elongate member has entered the canal, this upward angle may be adjusted to about 0 degrees to about 15 degrees. The elongate member may be guided into the canal. An actuator of the device may be rotated in a first direction advance the elongate member into the canal. In some variations, the elongate member may advance the maximum distance allowed by the device within Schlemm's Canal. The elongate member may be advanced up to a fourth quadrant of Schlemm's Canal. As the elongate member is advanced a volume of a fluid composition may be delivered from the distal tip of the elongate member. In some variations, one or more markings may be visualized to confirm the location of the elongate member within the canal. For example, after the elongate member has reached a third quadrant of the canal (e.g., reach a second hemisphere), white markings may become visible as the elongate member exits the cannula. These markings may indicate that the elongate member has entered the second hemisphere of the eye. Additional markings may be visualized and may indicate incremental distance of travel of the elongate member of about 1 clock hour. The elongate member may be retracted from the canal. An actuator of the device may be rotated in a second opposite direction to retract the elongate member. As the elongate member is retracted, a second volume of fluid composition may be delivered from the tip of the elongate member to viscodilate the eye along the length of Schlemm's canal. In some variations, about 23 microliters of viscoelastic fluid may be delivered in total.

Optionally, a canaloplasty procedure may be completed by advancement and retraction in either and/or both directions (e.g., two passes). More specifically, canaloplasty may be performed in either or both of a clockwise and counterclockwise direction. To repeat the procedure in a second direction, the elongate member may be retracted within the cannula. The cannula may be removed the anterior chamber and rotated 180-degrees outside of the eye. The device may be reintroduced and the previous steps repeated.

### Trabeculotomy

The methods described herein may comprise performing a trabeculotomy. The methods (as well as systems and devices) described herein, including the method for providing a disruptive force to trabeculocanalicular tissues, may be highly suitable for ab-interno trabeculotomy and goniotomy given that they avoid the use of electrocautery, and are capable of advancing elongate members over larger degrees of arc of Schlemm's canal. In some instances, disruptive tools may comprise disruptive components on their distal portions. Exemplary disruptive components include, without limitation, notches, hooks, barbs, balloons, or combinations thereof. In other instances, the disruptive tools may not comprise disruptive components on their distal portions, and indeed may have atraumatic blunt distal portions. Exemplary atraumatic distal portions include, without limitation, parasol or dome shaped distal portions.

A trabeculectomy may be performed using the delivery devices described herein, such as with the elongate member of the fluid delivery devices described herein. The outer diameter of the elongate member or tool may be variously sized for disruption of tissues, analogous to how fluid volumes may be varied to vary the level of disruption. For example, an elongate member or tool having an outer diameter ranging from about 50 to about 100 microns may be advanced through the canal to slightly dilate the canal and break or remove septae obstructing circumferential canalicular flow. An elongate member or tool having an outer diameter ranging from about 100 to 200 microns may be employed to perform the foregoing and may also to begin to stretch the trabecular meshwork and juxtacanalicular tissues. An elongate member or tool having an outer diameter ranging from about 200 to about 300 microns may be able to perform the above but may also create microtears in the trabecular meshwork and juxtacanalicular tissues and may maximally dilate the collector channels. An elongate member or tool having an outer diameter ranging from about 300 to about 500 microns may maximally disrupt the tissues and may create tears or perforations all along the trabecular meshwork and juxtacanalicular tissues. The elongate member or tool may be advanced out from the tip of the cannula and into the canal about a 30 degree arc of the canal (e.g., advanced about 3 to 4 mm out of the cannula), advanced about a 60 degree arc of the canal (e.g., advanced about 6 to 8 mm out of the cannula), advanced about a 90 degree arc of the canal (e.g., advanced about 10 mm out of the cannula), advanced about a 120 arc of the canal (e.g., advanced about 15 mm out of the cannula), advanced about a 180 degree arc of the canal (e.g., advanced about 20 mm out of the cannula), or advanced about a full 360 degrees of the canal (e.g., advanced about 36 to 40 mm out of the cannula), for maximal intraocular pressure reduction. In some variations, the elongate member may have a non-uniform outer diameter. For example, the elongate member may have a tapered outer diameter, such that the outer diameter increases from the distal to proximal end.

To perform the trabeculotomy, the elongate member (or other tool) may be advanced out from the tip of the cannula and into the canal about a 30 degree arc of the canal (e.g., advanced about 3 to 4 mm out of the cannula), advanced about a 60 degree arc of the canal (e.g., advanced about 6 to 8 mm out of the cannula), advanced about a 90 degree arc of the canal (e.g., advanced about 10 mm out of the cannula), advanced about a 120 arc of the canal (e.g., advanced about 15 mm out of the cannula), advanced about a 180 degree arc of the canal (e.g., advanced about 20 mm out of the cannula), or advanced about a full 360 degrees of the canal (e.g., advanced about 36 to 40 mm out of the cannula), for maximal intraocular pressure reduction. In some variations, the elongate member may be advanced between about a 5-degree arc of Schlemm's canal and about a 360-degree arc. In some variations, the methods may include advancement of the elongate member (or tool) about a 360-degree arc of Schlemm's canal, about a 270-degree arc of Schlemm's canal, about a 120-degree arc of Schlemm's canal, about a 180-degree arc of Schlemm's canal, or about a 90-degree arc of Schlemm's canal. In yet further variations, advancement of the elongate member (or a tool) may be about a 0-to-5-degree arc of Schlemm's canal, about a 30-degree arc of Schlemm's canal, or about a 60-degree arc of Schlemm's canal. In some variations, advancement of the elongate member may about a 1 clock hour to about 12 clock hours arc of the canal, about a 2 clock hours to about an 11 clock hours arc of the canal, about a 2 clock hours to about a 6 clock hours arc of the canal, or about a 12 clock hours arc of the canal, about a 10 clock hours arc of the canal, about an 8 clock hours arc of the canal, about a 6 clock hours arc of the canal, about a 4 clock hours arc of the canal, about a 2 clock hours arc of the canal, or about a 1 clock hour arc of the canal. In some variations of the trabeculotomy, the elongate member may be advanced into at least a first quadrant of the canal, at least a second quadrant of the canal, at least a third quadrant of the canal, or at least a fourth quadrant of the canal. It should be appreciated that the elongate member may be advanced in a clockwise or counterclockwise direction. It may be beneficial to advance the elongate member in both clockwise and counterclockwise directions about a 180-degree arc (e.g., about a 6 clock hours arc of the canal, about 2 quadrants) of Schlemm's canal from a single access point (e.g., the opening created in step 1702 of method 1700) in the canal. In other variations, the elongate member may be advanced in a single (clockwise or counterclockwise) direction about 360 degrees of Schlemm's canal from a single access point in the canal.

In some variations, the methods disclosed herein may include advancement of the elongate member between about a 5-degree arc of Schlemm's canal and about a 360-degree arc. In some variations, the methods may include advancement of the elongate member (or tool) about a 360-degree arc of Schlemm's canal, about a 270-degree arc of Schlemm's canal, about a 120-degree arc of Schlemm's canal, about a 180-degree arc of Schlemm's canal, or about a 90-degree arc of Schlemm's canal. In yet further variations, advancement of the elongate member (or a tool) may be about a 0-to-5-degree arc of Schlemm's canal, about a 30-degree arc of Schlemm's canal, or about a 60-degree arc of Schlemm's canal. Advancement may occur from a single access point in Schlemm's canal or from multiple access points in the canal. It may be beneficial to advance the elongate member in both clockwise and counterclockwise directions about a 180-degree arc of Schlemm's canal from a single access point in the canal. In other variations, the elongate member may be advanced in a single (clockwise or counterclockwise) direction about 360 degrees of Schlemm's canal from a single access point in the canal.

Depending on factors such as the type or severity of the condition being treated, the disruptive force may be generated to partially or completely destroy and/or remove the trabecular meshwork and may be adjusted by varying the tool configuration. In some methods, the trabecular meshwork may be disrupted during advancement of the slidable elongate member. Customizing a body segment of the elongate member proximal to the tip with one or more notches, barbs, or balloons that catch the meshwork as the distal tip is being guided and advanced along Schlemm's canal may also be used, thereby disrupting, partially tearing, fully tearing, and/or removing trabecular meshwork upon advancement. Additionally, an implant with edges specifically designed to cut the meshwork may be used.

In yet other methods, the trabecular meshwork may be disrupted during retraction of the slidable elongate member. The methods for disrupting tissues may involve customizing the system (e.g., the elongate member, any catheters or wires, probe tips, etc.) to catch or grasp the meshwork upon retraction after advancement through the canal. This may be done using a wire with a bent tip, hook, notch, or barb on its end that is advanced through the lumen of the catheter that then snags the meshwork upon retraction, tearing it along its length or removing it altogether, or solely with a metal or polymer wire or suture (no catheter) whose tip (and/or body) is hooked, notched, or barbed in such a way that it can be advanced into Schlemm's canal without tearing the meshwork but snags the meshwork upon retraction, tearing the meshwork and/or removing it completely. The elongate member may be provided with a disruptive tool, e.g., a sharp-edged element, that can cut or tear the trabecular meshwork while being retracted into the cannula, which is held stationary. Exemplary sharp-edged elements may be a hook, wire, or any other suitable shape memory component that can extend from the cannula to tear, cut, or remove trabecular meshwork.

Another method for disrupting tissues may include using oversized elongate members (e.g., having an outside diameter of 300-500 microns) to tear the meshwork upon delivery, or inflating or expanding the elongate member once it has been fully advanced into Schlemm's canal to stretch, disrupt, rupture, or fully tear the meshwork. For example, a catheter/elongate member, probe, or wire (with or without a lumen) whose tip is 200-250 microns in outer diameter, but having a shaft that begins to flare outwards after 3 clock hours of Schlemm's canal (i.e., at about the 5 or 10 mm mark on the catheter/elongate member) up to about 300, up to about 400, or up to about 500 microns, may be used, so that as the tip advances comfortably within Schlemm's canal, the enlarged shaft trails behind and ruptures the trabecular meshwork as it is advanced.

In another method, cutting, destruction, removal, or the like of the trabecular meshwork may be accomplished by removing the cannula from the eye while leaving the elongate member in the canal, thereby tearing through the meshwork. For example, a cannula may be inserted into the anterior chamber and Schlemm's canal, and a tool (e.g., a slidable elongate member) may be advanced within the canal. The cannula may be removed from the anterior chamber without retracting the elongate member. This action by itself may tear the trabecular meshwork. As the cannula is removed from the anterior chamber, the elongate member may begin tearing the trabecular meshwork from the point at which the cannula was inserted into Schlemm's canal and may continue tearing around the trabecular meshwork toward the distal end of the elongate member.

The methods described here may be used to access the trabecular outflow system using a single clear corneal incision and may allow for transluminal trabeculotomy of up to 360 degrees. The method may use a flexible elongate member that may be advanced and retracted using a single-handed disposable manual instrument. In one variation of the method, the cannula may be held securely against the angle while the flexible elongate member is advanced into Schlemm's canal. An exposed portion of one or more of the wheels may be rotated proximally to advance the flexible elongate member up to about 180 degrees around Schlemm's canal (about 20 mm of circumferential canal travel). For example, the elongate member may be advance about 90, 135, or 180 degrees. At this point, the flexible elongate member may in some instances be fully extended, and the wheel may no longer be able to be rotated. During this procedure, direct microscopic or gonioscopic visualization of the cannula tip may be maintained, and the anterior chamber may be maintained with viscoelastic or continuous balanced salt solution infusion. Once the flexible elongate member is advanced, the cannula may be removed from the eye through the incision without retracting the flexible elongate member. This may cause the body of the flexible elongate member to tear or cut through the trabecular meshwork. In some instances, it may be desirable to bias the distal tip of the cannula toward the trabecular meshwork being cut; this may in some instances help to prevent the flexible elongate member from slipping out of the canal during cannula removal.

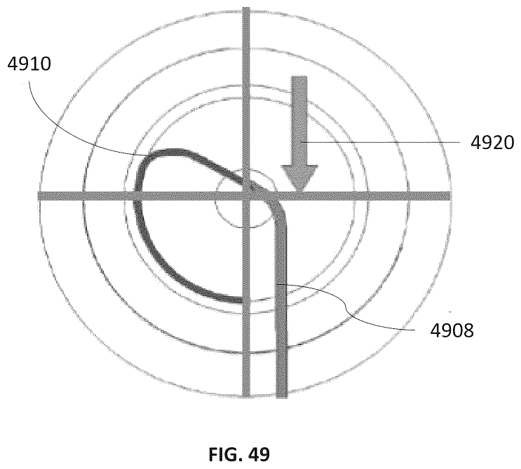

A trabeculotomy procedure may be performed according to the methods described herein. For example, Schlemm's canal may be viscodialated as described above. The cannula may be introduced through an opening in the cornea or sclera into the anterior chamber. The cannula, elongate member, and/or anatomy may be visualized by one or more of microscopic and gonioscopic visualization during the procedure. The anterior chamber may be maintained during the procedure. For example, viscoelastic or continuous balanced salt solution (BSS) irrigation be used to maintain the anterior chamber. The cannula tip may be advance through the anterior chamber and towards the iridocorneal angle. In some variations, a viscous fluid may be used to couple a gonioscope or gonioprism to the cornea for visualization. The cannula may approach the angle with the cannula tip. The cannula tip may pierce the meshwork at an angle of about 5 degrees to about 45 degrees from tangential with the canal. In some variations, the cannula may be angled upwards (e.g., rotated about a longitudinal axis of the cannula) to about 5 degrees to about 45 degrees to bias the elongate member into the canal. Once the elongate member has entered the canal, this upwards angle may be adjusted to an angle of about 0 degrees to about 10 degrees. The elongate member may be advanced into Schlemm's canal. For example, the elongate member may be advanced into the third quadrant of Schlemm's Canal, as shown in FIG. 48. A marking of the elongate member (e.g., second marking) may be visualized to confirm the advancement. The elongate member may be advanced by movement in a first direction of an actuator of the device. With the elongate member remaining in the canal, the cannula may be removed from the corneal or sclera incision and out of the eye causing the elongate member to cut or tear the trabecular meshwork and to perform the trabeculotomy. For example, FIG. 49 shows removal of the cannula 4908 along path 4920 and cutting or tearing of the meshwork by the elongate member 4910. The elongate member may be retracted while cutting the trabecular meshwork to reduce a length of the elongate member between the meshwork and the cannula tip. The elongate member may be retracted by movement in a second opposite direction of the actuator of the device by the user. The elongate member may be retracted until the elongate member tip is in close proximity to the corneal wound. The cannula and device may be removed from the eye.

Optionally, additional trabeculotomy may be performed. Outside the eye, the cannula may be rotated 180 degrees such that the cannula tip faces the opposite direction. The cannula tip may be advanced into the anterior chamber and through the pre-existing corneal wound. As was done before, introduce the elongate member into Schlemm's Canal. The previous steps may then be repeated as described to perform the second trabeculotomy.

### Implanting Ocular Devices

In some variations, the methods described herein may comprise implanting an ocular device (e.g., implant) completely or partially within Schlemm's canal. Ocular devices may be implanted as a standalone procedure or in conjunction with delivering a fluid composition into the canal and/or tearing the trabecular meshwork. For example, ab-interno canaloplasty using any of the methods described herein may be performed and followed by the implanting of any of the ocular devices described herein in the same or an overlapping region of the eye as treated by the canaloplasty. As another example, canaloplasty and trabeculotomy may be performed on a first portion of the eye (e.g., first hemisphere, quadrant) and canaloplasty followed by insertion of an ocular device may be performed on a second, different portion of the eye (e.g., second hemisphere, quadrant). Devices implanted in Schlemm's canal may generally be configured to maintain the patency of the canal without substantially interfering with transmural fluid flow across the canal. This may restore, enable, or enhance normal physiologic efflux of aqueous humor through the trabeculocanalicular tissues. In some variations, methods may include delivering a suture circumferentially to the canal and applying tension to the suture (e.g., to the ends of the suture) to help maintain patency of the canal. In some variations, a plurality of sutures or suture-based devices may be delivered to the canal to facilitate transmural flow. For example, an ocular device may comprise a singular suture or a plurality of sutures intertwined with one another to form a singular multi-suture device, such as a device comprising a plurality of sutures (e.g., 2, 3, 4, 5, or more) twisted and/or braided to form a solid or hollow structure. A plurality of sutures or multi-suture devices may be positioned within (e.g., at various locations) to facilitate transmural flow. Additionally or alternatively, as will be described in more detail below, methods may include delivering a suture circumferentially to the canal where the properties of the suture (e.g., geometry) are configured to apply tension the surrounding tissues of the canal to assist in maintaining patency of at least a portion of the canal while the suture is retained within the canal (e.g., as an implant).

In some variations, as noted above, the ocular device (e.g., implant) may be configured to maintain patency of the canal and/or tension the canal. The ocular device may comprise one or more of fenestrations, beads, knots, and grooves, configured to maintain patency of at least a portion of the canal while still allowing fluid flow within/through the canal. The ocular device may occupy between about a 30 degrees arc of the canal and about a 360 degrees arc of the canal such as, between about a 60 degrees arc and about a 180 degrees arc or about a 60 degrees arc and about at a 120 degrees arc. The ocular device may comprise a length corresponding to about a 30 degrees arc, about a 60 degrees arc, about a 90 degrees arc, about a 120 degrees arc, about a 180 degrees arc, or, about a 360 degrees arc of the canal. In some variations, a diameter or width of the ocular device may be configured to apply tension to the meshwork. For example, the ocular device may comprise a diameter of about 50 microns, about 100 microns, about 200 microns, about 300 microns, about 400 microns, or about 500 microns. The ocular device may be configured to stretch the trabecular meshwork upon its delivery to the canal. In some variations, a curve of the ocular device may tension the sides of the canal to facilitate fluid flow. For example, the ocular device may comprise a radius of curvature greater than the radius of curvature of the canal, or alternatively, the ocular device may comprise a radius of curvature less than the radius of curvature of the canal. In some variations, the ocular device may be made entirely from and/or may comprise suture and/or a biological material, such as, for example, collagen or a collagen derivative. In some variations, the ocular device may be entirely made of and/or comprise modified (e.g., processed/decellularized) scleral tissue.

Any of the delivery devices described herein may be used to deliver the ocular device (e.g., implant) to the canal. For example, in some variations, the cannula may deliver the ocular device (e.g., implant) to the canal via the lumen of the cannula. For example, the ocular device may be positioned within a distal portion of the lumen of the cannula and may be advanced out of the distal portion using, for example, the elongate member. More specifically, the ocular device may be delivered by advancing the elongate member within the cannula such that the ocular device is moved distally out of the cannula tip and into the canal. In some variations, a portion of the delivery device (e.g., the cannula and/or the elongate member) may comprise a surface feature configured to receive, retain or otherwise engage with the ocular device during placement of the ocular device in the eye. The surface feature may comprise one or more notches, slots, recesses, and the like in the cannula and/or the elongate member. The cannula and/or the elongate member may comprise a circumferential groove configured to engage with the ocular device. In some variations, the ocular device may comprise an engagement feature configured to releasably couple (e.g., be releasably received in) to the surface feature of the delivery device. For example, the engagement feature of the ocular device may comprise one or more of: a knot, a bead, a protrusion, and a hook, that may engage with (e.g., be received within) the surface feature of the delivery device. A portion of the ocular device may comprise a larger diameter forming a ring-like protrusion. The portion may be configured to retain or engage the ocular device with the cannula and/or the elongate member. In some variations, the elongate member may be or may comprise a guidewire, and the ocular device may be delivered using (e.g., positioned over) the guidewire. Additionally or alternatively to utilizing the surface features of the delivery device and the engagement features of the ocular to facilitate delivery of the ocular device to the eye (e.g., disengagement of the ocular device from the delivery device), a device, fluid composition (e.g., viscoelastic) may be utilized to deliver the ocular device.

In some variations, the ocular device may comprise a distal tip or a distal portion of the elongate member, which may be detached from the remainder of the elongate member for implantation in the eye (e.g., canal). In these variations, the ocular device may be delivered to the canal by at least partially extending the elongate member from the cannula into the canal and then disassociating a portion of the elongate member from the device and leaving the portion in the canal. As described herein, in some variations, the cannula may comprise a sharp distal tip, which may be used to cut the portion of the elongate member forming the ocular device. Additionally or alternatively, a separate tool (e.g., cutting tool) may be introduced to detach (e.g., cut) the portion of the elongate member to remain in the eye. While described above in the context of the delivery devices described herein, it should be appreciated that a separate ocular device delivery device comprising the same or similar features to those described herein with respect to the fluid delivery device may be used to deliver an ocular device. For example, in some variations, such an ocular device delivery device may comprise any of the handles, cannulas, and elongate members described herein, and all or portions of any of the drive assemblies described herein. These ocular device delivery devices may or may not comprise the fluid assemblies described herein.

The procedures described herein may be used in any suitable combination. In some variations, one or more combinations of the aforementioned procedures may be used to reduce intraocular pressure in one or both eyes of a patient. Generally, a goniotomy may be performed prior to or simultaneously with one or more of a fluid delivery (e.g., canaloplasty), trabeculotomy, or implantation procedure. For example, a tool may be used to create an opening in the trabecular meshwork, and one or more additional procedures may subsequently be performed to reduce intraocular pressure, or an opening may be created via a distal tip of a delivery device as one or more of the remaining procedures are being performed. In some variations, two or more procedures may be performed via the same opening within the trabecular meshwork (e.g., via the same incision made during goniotomy). In some variations, two or more procedures may be performed via two or more different openings within the trabecular meshwork. As will be described below, a combination of procedures may be achieved by applying a first procedure in a first direction of the canal (e.g., in a clockwise or counterclockwise direction), and then applying at least a second procedure in a second direction of the canal (e.g., in a counterclockwise or clockwise direction). For example, the first procedure may be a fluid delivery procedure (e.g., canaloplasty) or a trabeculotomy procedure that is performed in a clockwise direction, and the second procedure may be an implantation procedure that is applied in a counterclockwise direction. In some variations, one or more of the procedures (e.g., a fluid delivery procedure and a trabeculotomy procedure) may be applied in a same direction within the canal. Moreover, in some variations, one or more procedures may be performed a plurality of times (e.g., two times, three times, four times, five times, or more than five times).

In some variations, a first portion of Schlemm's canal may be viscodilated, and a second (same, different, or overlapping) portion of the canal may receive an ocular device therein. For example, the fluid delivery may occur within about 10 degrees and about 360 degrees of Schlemm's canal (e.g., within about 45 degrees, about 90 degrees, about 135 degrees, about 180 degrees, about 225 degrees, about 270 degrees, about 315 degrees, or about 360 degrees of the canal), and the implantation may also occur within about 10 degrees and about 360 degrees of Schlemm's canal (e.g., within about 45 degrees, about 90 degrees, about 135 degrees, about 180 degrees, about 225 degrees, about 270 degrees, about 315 degrees, or about 360 degrees of the canal). In some variations, the fluid delivery may be performed in a first direction (e.g., clockwise or counterclockwise through the canal), and the implantation may be performed in the same first direction. In alternative variations, the fluid delivery may be performed in a first direction (e.g., clockwise or counterclockwise through the canal), and the implantation may be performed in a second, different direction (e.g., counterclockwise or clockwise). The implantation may occur prior to or following the fluid delivery. As another example, a first portion of Schlemm's canal may be viscodilated, and a second (same, different, or overlapping) portion of the trabecular meshwork of the canal may be cut, torn, or removed. For example, the fluid delivery may occur within about 10 degrees and about 360 degrees of Schlemm's canal (e.g., within about 45 degrees, about 90 degrees, about 135 degrees, about 180 degrees, about 225 degrees, about 270 degrees, about 315 degrees, or about 360 degrees of the canal), and the trabeculotomy may also occur within about 10 degrees and about 360 degrees of Schlemm's canal (e.g., within about 45 degrees, about 90 degrees, about 135 degrees, about 180 degrees, about 225 degrees, about 270 degrees, about 315 degrees, or about 360 degrees of the canal). In some variations, a first portion of the trabecular meshwork may be cut, torn, or removed, and a second portion the canal still retaining trabecular meshwork (e.g., the trabecular meshwork was torn therefrom) may be viscodilated. In some variations, the fluid delivery may be performed in a first direction (e.g., clockwise or counterclockwise), and the trabeculotomy may be performed in the same first direction. In alternative variations, the fluid delivery may be performed in the first direction (e.g., clockwise or counterclockwise), and the trabeculotomy may be performed in a second, different direction (e.g., counterclockwise or clockwise). The trabeculotomy may be performed prior to, following, or simultaneously with the fluid delivery. As another example, a first portion of the trabecular meshwork may be cut, torn, or removed, and a second, different portion of the canal may receive an ocular device (e.g., implant) therein. For example, the trabeculotomy may occur within about 10 degrees and about 360 degrees of Schlemm's canal (e.g., within about 45 degrees, about 90 degrees, about 135 degrees, about 180 degrees, about 225 degrees, about 270 degrees, about 315 degrees, or about 360 degrees of the canal), and the implantation may also occur within about 10 degrees and about 360 degrees of Schlemm's canal (e.g., within about 45 degrees, about 90 degrees, about 135 degrees, about 180 degrees, about 225 degrees, about 270 degrees, about 315 degrees, or about 360 degrees of the canal). In some variations, a first portion of the trabecular meshwork may be cut, torn, or removed, and a second portion the canal still retaining trabecular meshwork may receive the ocular device (e.g., implant). In some variations, the trabeculotomy may be performed in a first direction (e.g., clockwise or counterclockwise), and the implantation may be performed in the same first direction but in a different location. In alternative variations, the trabeculotomy may be performed in the first direction (e.g., clockwise or counterclockwise), and the implantation may be performed in a second, different direction (e.g., counterclockwise or clockwise). The implantation may be performed prior to or following trabeculotomy. As yet another example, a first portion of the canal may be viscodilated, a second (same, different, or overlapping) portion of the trabecular meshwork of the canal may be cut, torn, or removed, and a third (same, different, or overlapping with respect to the first portion, and/or different with respect to the second portion) portion of the canal may receive an implant therein. For example, the fluid delivery may occur within about 10 degrees and about 360 degrees of Schlemm's canal, the trabeculotomy may also occur within about 10 degrees and about 360 degrees of Schlemm's canal, and the implantation may occur within about 10 degrees and about 360 degrees of the canal. The fluid delivery and the implantation may generally be performed within portions of the canal still retaining trabecular meshwork. The fluid delivery may be performed in a first direction (e.g., clockwise or counterclockwise), the trabeculotomy may be performed in the first direction or in a second, different direction, and the implantation may be performed in the first direction or the second direction. The implantation may be performed prior to or following trabeculotomy and/or the fluid delivery, and the trabeculotomy may be performed prior to, following, or simultaneously with the fluid delivery.

The methods are generally single-handed, single-operator controlled methods that are minimally invasive, e.g., they are tailored for an ab-interno procedure, which as previously mentioned, can be advantageous over the more invasive ab-externo approach. However, use of the systems in an ab-externo method may be contemplated in some instances and thus, are not excluded here. The methods may be used to treat or prevent glaucoma, pre-glaucoma, or ocular hypertension. When treating glaucoma, the methods may also be used in conjunction with a cataract surgery (before or after) using the same incision during the same session or at another time.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

While variations of the present invention have been shown and described herein, those skilled in the art will understand that such variations are provided by way of example only. Numerous variations, changes, and substitutions will now occur to those skilled in the art without departing from the invention. It should be understood that various alternatives to the variations of the invention described herein may be employed in practicing the invention. It is intended that the following claims define the scope of the invention and that methods and structures within the scope of these claims and their equivalents be covered thereby.

1. A device for delivering fluid to the eye, comprising:
a handle comprising a fluid assembly at least partially contained therein, the fluid assembly comprising a fluid reservoir, a plunger tube, and a displacement rod;
a cannula coupled to a distal end of the handle; and
an elongate member slidably positioned within the cannula and configured to be advanced into Schlemm's canal,
wherein the plunger tube and the displacement rod are configured to move relative to the fluid reservoir to deliver fluid from the fluid reservoir during advancement of the elongate member.

2. The device of claim 1, wherein the handle further comprises a drive assembly and actuation of the drive assembly moves the plunger tube and the displacement rod in opposite directions during advancement of the elongate member.

3. (canceled)

4. The device of claim 2, wherein the drive assembly comprises a first linear gear and a second linear gear, and wherein the first linear gear is configured to move in a direction opposite the second linear gear.

5. The device of claim 4, wherein displacement rod is releasably coupled to the first linear gear and the plunger tube is coupled to the second linear gear.

6.-17. (canceled)

18. The device of claim 2, wherein movement of the displacement rod towards the fluid reservoir delivers fluid during advancement of the elongate member.

19. The device of claim 12, wherein movement of the plunger tube towards the fluid reservoir delivers fluid during retraction of the elongate member.

20.-21. (canceled)

22. The device of claim 1, wherein the fluid reservoir comprises a first lumen configured to receive the displacement rod and a second lumen configured to receive the plunger tube, wherein the first lumen is in fluid communication with the second lumen.

23. The device of claim 1, wherein the device is configured to deliver a first volume of fluid during advancement of the elongate member and a second, different volume of fluid during retraction of the elongate member.

24.-49. (canceled)

50. The device of claim 1, further comprising a drive assembly comprising an actuator configured to be contacted by a user, wherein the actuator comprises one or more of: a slide, a wheel, and a button.

51. (canceled)

52. A device for delivering fluid to the eye, comprising:
a handle comprising a drive assembly and a fluid assembly each at least partially contained in the handle, the fluid assembly comprising a fluid reservoir, a plunger tube, and a displacement rod;
a cannula coupled to a distal end of the handle; and
an elongate member slidably positioned within the cannula,
wherein actuation of the drive assembly moves the plunger tube and the displacement rod in opposite directions during advancement of the elongate member.

53. A device for delivering fluid to the eye, comprising:
a handle comprising a drive assembly and a fluid reservoir each at least partially contained therein, the drive assembly comprising an actuator configured to be contacted by a user, a first linear gear and a second linear gear;
a cannula coupled to a distal end of the handle; and
an elongate member slidably positioned within the cannula,
wherein actuation of the actuator moves the first and second linear gears in opposite directions during advancement of the elongate member.

54.-160. (canceled)

161. The device of claim 1, wherein the device is configured to deliver fluid from the fluid reservoir at a first rate during advancement of the elongate member and a second, different rate during retraction of the elongate member.

162. The device of claim 2, wherein the drive assembly comprises a clutch.

163. The device of claim 52, wherein the drive assembly comprises a first linear gear, a second linear gear, and a clutch operatively coupled to the first and second linear gears.

164. The device of claim 163, wherein the clutch is configured to selectively engage the first linear gear.

165. The device of claim 163, wherein the clutch comprises a first pinion gear, a second pinion gear, and a spring configured to bias the first pinion gear and the second pinion gear of the clutch toward engagement.

166. The device of claim 52, wherein actuation of the drive assembly in a first direction moves the displacement rod towards the fluid assembly and the plunger tube away from the fluid assembly.

167. The device of claim 166, wherein actuation of the drive assembly in a second, opposite direction moves the plunger tube towards the fluid assembly.

168. The device of claim 52, wherein the device is configured to deliver fluid from the fluid reservoir during advancement of the elongate member and upon retraction of the elongate member.

169. The device of claim 52, wherein the device is configured to deliver a first total volume of fluid during advancement of the elongate member and a second, different total volume of fluid during retraction of the elongate member.

170. The device of claim 52, wherein the fluid reservoir is stationary within the handle.
